(19) United States
(12) Patent Application Publication
(10) Pub. No.: US 2002/0048763 A1
(43) Pub. Date: Apr. 25, 2002

(54) Human genome-derived single exon nucleic acid probes useful for gene expression analysis

(76) Inventors: Sharron Gaynor Penn, San Mateo, CA (US); David Russell Rank, Fremont, CA (US); Wensheng Chen, Mountain View, CA (US); David Kagen Hanzel, Palo Alto, CA (US)

Correspondence Address: FISH & NEAVE, 1251 AVENUE OF THE AMERICAS, 50TH FLOOR, NEW YORK, NY, 10020-1105, US

(21) Appl. No.: 09/864,761

(22) Filed: May 23, 2001

Related U.S. Application Data
(63) Continuation-in-part of application No. 09/774,203, filed on Jan. 29, 2001.
(63) Continuation-in-part of application No. 09/632,366, filed on Aug. 3, 2000.
(63) Continuation-in-part of application No. 09/608,408, filed on Jun. 30, 2000.
(63) Continuation-in-part of application No. PCT/US01/00666, filed on Jan. 30, 2001.
(63) Continuation-in-part of application No. PCT/US01/00667, filed on Jan. 30, 2001.
(63) Continuation-in-part of application No. PCT/US01/00664, filed on Jan. 30, 2001.
(63) Continuation-in-part of application No. PCT/US01/00669, filed on Jan. 30, 2001.
(63) Continuation-in-part of application No. PCT/US01/00665, filed on Jan. 30, 2001.
(63) Continuation-in-part of application No. PCT/US01/00668, filed on Jan. 30, 2001.
(63) Continuation-in-part of application No. PCT/US01/00663, filed on Jan. 30, 2001.
(63) Continuation-in-part of application No. PCT/US01/00662, filed on Jan. 30, 2001.
(63) Continuation-in-part of application No. PCT/US01/00661, filed on Jan. 30, 2001.
(63) Continuation-in-part of application No. PCT/US01/00670, filed on Jan. 30, 2001.
(60) Provisional application No. 60/180,312, filed on Feb. 4, 2000.
(60) Provisional application No. 60/207,456, filed on May 26, 2000.
(60) Provisional application No. 60/234,687, filed on Sep. 21, 2000.
(60) Provisional application No. 60/236,359, filed on Sep. 27, 2000.

(30) Foreign Application Priority Data
Oct. 4, 2000 (GB) 00242636

(51) Int. Cl. C12Q 1/68; C07H 21/04

(52) U.S. Cl. 435/6; 536/24.3

(57) ABSTRACT

Methods and apparatus for predicting, confirming and displaying functional regions from genomic sequence data are used to identify 16,834 unique human genome-derived single exon probes useful for gene expression analysis, particularly gene expression analysis by microarray. Also presented are genome-derived single exon microarrays that include such probes, peptides encoded by the exons, and antibodies thereto.

### CROSS REFERENCE TO RELATED APPLICATIONS

This application claims priority under 35 U.S.C. § 365(c) to international patent application nos. PCT/US01/00666, PCT/US01/00667, PCT/US01/00664, PCT/US01/00669, PCT/US01/00665, PCT/US01/00668, PCT/US01/00663, PCT/US01/00662, PCT/US01/00661, PCT/US01/00670, all filed Jan. 30, 2001; claims priority under 35 U.S.C. § 119(e) to provisional applications for U.S. Pat. Ser. No. 60/180,312, filed Feb. 4, 2000, No. 60/207,456, filed May 26, 2000, No. 60/234,687, filed Sep. 21, 2000, and No. 60/236,359, filed Sep. 27, 2000; claims priority under 35 U.S.C. § 119(a) to GB 24263.6, filed Oct. 4, 2000; and is a continuation-in-part of U.S. patent application Ser. No. 09/608,408, filed Jun. 30, 2000, Ser. No. 09/632,366, filed Aug. 3, 2000, and Ser. No. 09/774,203, filed Jan. 29, 2001, the disclosures of which are incorporated herein by reference in their entireties.

### INCORPORATION-BY-REFERENCE OF MATERIALS FILED ON COMPACT DISC

The present application includes a Sequence Listing and ten (10) tables filed herewith on a single (CD-R) compact disc, filed herewith in duplicate, having volume label AEOMICAR52.

The Sequence Listing is presented in a single file named Sequence.txt, last modified May 22, 2001 09:30a, and having 48,662,849 bytes.

Table 4 is presented in a single file named table4.txt, last modified May 21, 2001 04:52p, and having 6,690,357 bytes.

Table 5 is presented in a single file named table5.txt, last modified May 21, 2001 04:52p, and having 5,155,245 bytes.

Table 6 is presented in a single file named table 6.txt, last modified May 21, 2001 04:52p, and having 6,815,259 bytes.

Table 7 is presented in a single file named table7.txt, last modified May 21, 2001 04:52p, and having 6,579,954 bytes.

Table 8 is presented in a single file named presented in a single file named table8.txt, last modified May 21, 2001 04:52p, and having 6,877,305 bytes.

Table 9 is presented in a single file named table9.txt, last modified May 21, 2001 04:52p, and having 6,545,772 bytes.

Table 10 is presented in a single file named table10.txt, last modified May 21, 2001 04:52p, and having 6,822,063 bytes.

Table 11 is presented in a single file named table 11.txt, last modified May 21, 2001 04:52p, and having 4,773,006 bytes.

Table 12 is presented in a single file named table12.txt, last modified May 21, 2001 04:52p, and having 2,675,997 bytes.

Table 13 is presented in a single file named table 13.txt, last modified May 21, 2001 04:52p, and having 2,609,253 bytes.

The disclosure of each of the aforesaid files is incorporated herein by reference in its entirety.

### FIELD OF THE INVENTION

The present invention is in the fields of molecular biology and bioinformatics. In particular, the invention relates to 16,834 genome-derived single exon nucleic acid probes expressed in one or more of ten tested human tissues, which are useful for gene expression analysis as by microarray hybridization. The invention further relates to single exon nucleic acid microarrays that include such probes.

### BACKGROUND OF THE INVENTION

For almost two decades following the invention of general techniques for nucleic acid sequencing, Sanger et al., Proc. Natl. Acad. Sci. USA 70(4):1209-13 (1973); Gilbert et al., Proc. Natl. Acad. Sci. USA 70(12):3581-4 (1973), these techniques were used principally as tools to further the understanding of proteins—known or suspected—about which a basic foundation of biologic knowledge had already been built. In many cases, the cloning effort that preceded sequence identification had been both informed and directed by that antecedent biological understanding.

For example, the cloning of the T cell receptor for antigen was predicated upon its known or suspected cell type-specific expression, by its suspected membrane association, and by the predicted assembly of its gene via T cell-specific somatic recombination. Hedrick et al., Nature 308(5955):149-53 (1984). Subsequent sequencing efforts at once confirmed and extended understanding of this family of proteins. Hedrick et al., Nature 308(5955):153-8 (1984).

More recently, however, the development of high throughput sequencing methods and devices, in concert with large public and private undertakings to sequence the human and other genomes, has altered this investigational paradigm: today, sequence information often precedes understanding of the basic biology of the encoded protein product.

One of the approaches to large-scale sequencing is predicated upon the proposition that expressed sequences—that is, those accessible through isolation of mRNA—are of greatest initial interest. This “expressed sequence tag” (“EST”) approach has already yielded vast amounts of sequence data. Adams et al., Science 252:1651 (1991); Williamson, Drug Discov. Today 4:115 (1999); Strausberg et al., Nature Genet. 15:415 (1997); Adams et al., Nature 377(suppl.):3 (1995); Marra et al., Nature Genet. 21:191 (1999). For nucleic acids sequenced by this approach, often the only biologic information that is known a priori with any certainty is the likelihood of biologic expression itself. By virtue of the species and tissue from which the mRNA had originally been obtained, most such sequences are also annotated with the identity of the species and at least one tissue in which expression appears likely.

More recently, the pace of genomic sequencing has accelerated dramatically. When genomic DNA serves as the initial substrate for sequencing efforts, expression cannot be presumed; often the only a priori biologic information about the sequence includes the species and chromosome (and perhaps chromosomal map location) of origin.

With the ever-accelerating pace of sequence accumulation by directed, EST, and genomic sequencing approaches—and in particular, with the accumulation of sequence information from multiple genera, from multiple species within genera, and from multiple individuals within a species—there is an increasing need for methods that rapidly and effectively permit the functions of nucleic sequences to be elucidated. And as such functional information accumulates, there is a further need for methods of storing such functional information in meaningful and useful relationship to the sequence itself; that is, there is an increasing need for means and apparatus for annotating raw sequence data with known or predicted functional information.

Although the increase in the pace of genomic sequencing is due in large part to technological changes in sequencing strategies and instrumentation, Service, Science 280:995 (1998); Pennisi, Science 283: 1822-1823 (1999), there is an important functional motivation as well.

While it was understood that the EST approach would rarely be able to yield sequence information about the noncoding portions of the genome, it now also appears the EST approach is capable of capturing only a fraction of a genome's actual expression complexity.

For example, when the C. elegans genome was fully sequenced, gene prediction algorithms identified over 19,000 potential genes, of which only 7,000 had been found by EST sequencing. C. elegans Sequencing Consortium, Science 282:2012 (1998). Analogously, the recently completed sequence of chromosome 2 of Arabidopsis predicts over 4000 genes, Lin et al., Nature, 402:761 (1999), of which only about 6% had previously been identified via EST sequencing efforts. Although the human genome has the greatest depth of EST coverage, it is still woefully short of surrendering all of its genes. One recent estimate suggests that the human genome contains more than 146,000 genes, which would at this point leave greater than half of the genes undiscovered. It is now predicted that many genes, perhaps 20 to 50%, will only be found by genomic sequencing.

There is, therefore, a need for methods that permit the functional regions of genomic sequence—and most importantly, but not exclusively, regions that function to encode genes—to be identified.

Much of the coding sequence of the human genome is not homologous to known genes, making detection of open reading frames (“ORFs”) and predictions of gene function difficult. Computational methods exist for predicting coding regions in eukaryotic genomes. Gene prediction programs such as GRAIL and GRAIL II, Uberbacher et al., Proc. Natl. Acad. Sci. USA 88(24):11261-5 (1991); Xu et al., Genet. Eng. 16:241-53 (1994); Uberbacher et al., Methods Enzymol. 266:259-81 (1996); GENEFINDER, Solovyev et al., Nucl. Acids. Res. 22:5156-63 (1994); Solovyev et al., Ismb 5:294-302 (1997); and GENSCAN, Burge et al., J. Mol. Biol. 268:78-94 (1997), predict many putative genes without known homology or function. Such programs are known, however, to give high false positive rates. Burset et al., Genomics 34:353-367 (1996). Using a consensus obtained by a plurality of such programs is known to increase the reliability of calling exons from genomic sequence. Ansari-Lari et al., Genome Res. 8(l):29-40 (1998)

Identification of functional genes from genomic data remains, however, an imperfect art. For example, in reporting the full sequence of human chromosome 21, the Chromosome 21 Mapping and Sequencing Consortium reports that prior bioinformatic estimates of human gene number may need to be revised substantially downwards. Nature 405:311-199 (2000); Reeves, Nature 405:283-284 (2000).

Thus, there is a need for methods and apparatus that permit the functions of the regions identified bioinformatically—and specifically, that permit the expression of regions predicted to encode protein—readily to be confirmed experimentally.

Recently, the development of nucleic acid microarrays has made possible the automated and highly parallel measurement of gene expression. Reviewed in Schena (ed.), DNA Microarrays: A Practical Approach (Practical Approach Series), Oxford University Press (1999) (ISBN: 0199637768); Nature Genet. 21(1)(suppl):1-60 (1999); Schena (ed.), Microarray Biochip: Tools and Technology, Eaton Publishing Company/BioTechniques Books Division (2000) (ISBN: 1881299376), the disclosures of which are incorporated herein by reference in their entireties.

It is common for microarrays to be derived from cDNA/EST libraries, either from those previously described in the literature, such as those from the I.M.A.G.E. consortium, Lennon et al., “The I.M.A.G.E. Consortium: an Integrated Molecular Analysis of Genomes and Their Expression, Genomics 33(1):151-2 (1996), or from the construction of “problem specific” libraries targeted at a particular biological question, R. S. Thomas et al., Cancer Res. (in press). Such microarrays by definition can measure expression only of those genes found in EST libraries, and thus have not been useful as probes for genes discovered solely by genomic sequencing.

The utility of using whole genome nucleic acid microarrays to answer certain biologic questions has been demonstrated for the yeast Saccharomyces cerevisiae. De Risi et al., Science 278:680 (1997). The vast majority of yeast nuclear genes, approximately 95% however, are single exon genes, i.e., lack introns, Lopez et al., RNA 5:1135-1137 (1999); Goffeau et al., Science 274:563-67 (1996), permitting coding regions more readily to be identified. Whole genome nucleic acid microarrays have not generally been used to probe gene expression from more complex eukaryotic genomes, and in particular from those averaging more than one intron per gene.

### SUMMARY OF THE INVENTION

The present invention solves these and other problems in the art by providing, in a first aspect, human genome-derived single exon nucleic acid probes. The probes are useful, inter alia, for gene expression analysis, and particularly for gene expression analysis by microarray.

In preferred embodiments, the invention provides genome-derived single-exon probes known to be expressed in one or more human tissues or cell types, particularly human brain, heart, liver, fetal liver, placenta, lung, bone marrow, BT 474 and other human mammary epithelial cells, HeLa and other human cervical epithelial cells, HBL 100 and other human mammary epithelial cells. In particular embodiments, the invention provides human single-exon probes that comprise a nucleotide sequence as set forth in any one of SEQ ID NOs: 16,835-33,299, the complement thereof, or a fragment of the referenced SEQ ID NO: or complement thereof, wherein the probe hybridizes at high stringency (i.e., under high stringency conditions) to a nucleic acid expressed in human cells, and wherein the probe includes portions of no more than one human exon. In certain embodiments, the single exon nucleic acid probe comprises any one of SEQ ID NOs. 1-16,834 or the complement thereof.

In a second aspect, the invention provides an amplifiable nucleic acid composition, comprising a single exon nucleic acid probe of the present invention and at least one nucleic acid primer, wherein the at least one primer is sufficient to prime enzymatic amplification of the probe.

In a third aspect, the invention provides a spatially-addressable set of single exon nucleic acid probes, comprising: a plurality of single exon nucleic acid probes of the present invention, wherein each of the plurality of probes is separately and addressably isolatable and/or amplifiable from the plurality.

In a fourth aspect, the invention provides a single exon nucleic acid probe attached to a substrate. The substrate can, e.g., be a filter membrane, such as nitrocellulose or nylon, or can be a solid, such as glass, amorphous silicon, crystalline silicon, or plastic.

In a fifth aspect, the invention provides a single exon nucleic acid microarray, comprising: a plurality of nucleic acid probes addressably disposed upon a substrate, wherein at least 50% of the nucleic acid probes include a fragment of no more than one exon of a eukaryotic genome, the fragment being selectively hybridizable at high stringency (under high stringency conditions) to an expressed gene, wherein the plurality of nucleic acid probes averages at least 50 bp, 75 bp, or 100 bp in length, and wherein the eukaryotic genome averages at least one intron per gene.

In certain embodiments of the nucleic acid microarrays of the present invention, at least 50% of the exon-including nucleic acid probes further comprise, contiguous to a first end of the fragment, a first intronic and/or intergenic sequence that is identically contiguous to the fragment in the human genome. In certain embodiments, at least 50% of the exon-including nucleic acid probes further comprise, contiguous to a second end of the fragment, a second intronic and/or intergenic sequence that is identically contiguous to the fragment in the human genome.

In preferred embodiments, the microarray includes genome-derived single-exon probes known to be expressed in one or more human tissues or cell types, including human brain, heart, liver, fetal liver, placenta, lung, bone marrow, BT 474 and other human mammary epithelial cells, HeLa and other human cervical epithelial cells, HBL 100 and other human mammary epithelial cells.

In particular embodiments, the microarrays of the present invention include a plurality of human single-exon probes that comprise a nucleotide sequence as set forth in any one of SEQ ID NOs: 16,835-33,299, the complement thereof, or a fragment of the referenced SEQ ID NO: or complement thereof, wherein the probe hybridizes at high stringency (i.e., under high stringency conditions) to a nucleic acid expressed in human cells, and wherein the probe includes portions of no more than one human exon. In certain embodiments, the single exon nucleic acid probe comprises any one of SEQ ID NOs. 1-16,834 or the complement thereof.

In a sixth aspect, the invention provides a method of measuring eukaryotic gene expression, comprising: contacting the single exon microarray of the invention with a first collection of detectably labeled nucleic acids, the first collection of nucleic acids derived from mRNA of at least one eukaryotic tissue or cell type; and then measuring the label detectably bound to each probe of the microarray. In certain embodiments the method further comprises comparing the measurement to a second measurement, the second measurement identically obtained using a second, control, collection of nucleic acids.

In certain embodiments of this aspect of the invention, the first and second collections of detectably labeled nucleic acids are distinguishably labeled, often by fluorophores, and are contacted simultaneously to the microarray.

In a seventh aspect, the invention provides a method of selling and/or licensing genome-derived single-exon microarrays to a customer desiring to measure gene expression, comprising: making available for computerized query a database having a record corresponding to each genome-derived single exon microarray available for sale and/or license; responding to a customer query of the database by returning to the customer at least one record, or an identifier of that record, that best meets the customer query criteria; and offering for sale or license to the querying customer the genome-derived single exon microarray identified in that at least one record. In certain embodiments, the single exon microarrays includes the probes of the present invention.

In an eighth aspect, the invention provides a method of designing and/or manufacturing a genome-derived single exon microarray that has genome-derived single exon probes that have at least one common attribute desired by a customer, the method comprising: receiving from a customer at least one criterion for common probe attribute; using the at least one criterion to identify, within a database having records corresponding to available genome-derived single exon probes, those that meet the criterion; and then disposing such identified probes on a substrate capable of functioning in microarray hybridization experiments. In typical embodiments, the common attribute is common expression in a tissue and/or cell type. In presently preferred embodiments, the tissue and/or cell type is selected from the group consisting of: human brain, heart, liver, fetal liver, placenta, lung, bone marrow, HeLa cells, BT 474 cells, and HBL 100 cells.

In a ninth aspect, the invention provides a method for making available by subscription expression data obtained from use of the genome-derived single exon probes and genome-derived single exon microarrays of the present invention to customers that desire such information, comprising making available to a subscription client for computerized query a database, the database having records containing expression data generated using single exon probes disposed upon microarrays; and then responding to a customer query of the database by returning to the customer at least one record, or an identifier of the at least one record, that best meets the customer query criteria.

In a tenth aspect, the invention provides a method of attracting investment to a company that makes and sells microarrays or expression data obtained from microarrays, comprising: advertising the availability for distribution, sale, or license of genome-derived single exon probes, microarrays, and/or data therefrom; and then selling stock in the company.

In an eleventh aspect, the invention provides a protein, polypeptide, or peptide comprising: at least 8 amino acids of the sequence set forth in any one of SEQ ID NOs: 33,300-49,117. In typical embodiments, the protein, polypeptide, or peptide comprises at least 15 amino acids of the sequence set forth in any one of SEQ ID NOs: 33,300-49,117. In certain embodiments, the protein, polypeptide or peptide is detectably labeled.

In a twelfth aspect, the invention provides an isolated nucleic acid that encodes the protein, polypeptide, or peptide of the present invention.

In a thirteenth aspect, the invention provides an antibody, wherein the antibody is specific for the protein, polypeptide, or peptide of the present invention.

Various further aspects and embodiments of the present invention are set forth in the following numbered paragraphs:

1. A single exon nucleic acid probe for measuring human gene expression, comprising:

a nucleotide sequence as set forth in any one of SEQ ID NOs: 16,835-33,299, the complement thereof, or a fragment of said SEQ ID NO: or said complement,

wherein said probe hybridizes at high stringency to a nucleic acid expressed in human cells and includes portions of no more than one human exon.

2. A single exon nucleic acid probe for measuring gene expression in human brain, comprising:

a nucleotide sequence as set forth in any one of the exon SEQ ID NOs: set forth in Table 4, the complement thereof, or a fragment of said SEQ ID NO: or said complement,

wherein said probe hybridizes at high stringency to a nucleic acid expressed in human brain and includes portions of no more than one human exon.

3. A single exon nucleic acid probe for measuring gene expression in human heart, comprising:

a nucleotide sequence as set forth in any one of the exon SEQ ID NOs: set forth in Table 5, the complement thereof, or a fragment of said SEQ ID NO: or said complement,

wherein said probe hybridizes at high stringency to a nucleic acid expressed in human heart and includes portions of no more than one human exon.

4. A single exon nucleic acid probe for measuring gene expression in human liver, comprising:

a nucleotide sequence as set forth in any one of the exon SEQ ID NOs: set forth in Table 6, the complement thereof, or a fragment of said SEQ ID NO: or said complement,

wherein said probe hybridizes at high stringency to a gene expressed in human liver and includes portions of no more than one human exon.

5. A single exon nucleic acid probe for measuring gene expression in human fetal liver, comprising:

a nucleotide sequence as set forth in any one of the exon SEQ ID NOs: set forth in Table 7, the complement thereof, or a fragment of said SEQ ID NO: or said complement,

wherein said probe hybridizes at high stringency to a gene expressed in human fetal liver and includes portions of no more than one human exon.

6. A single exon nucleic acid probe for measuring gene expression in human placenta, comprising:

a nucleotide sequence as set forth in any one of the exon SEQ ID NOs: set forth in Table 8, the complement thereof, or a fragment of said SEQ ID NO: or said complement,

wherein said probe hybridizes at high stringency to a gene expressed in human placenta and includes portions of no more than one human exon.

7. A single exon nucleic acid probe for measuring gene expression in human lung, comprising:

a nucleotide sequence as set forth in any one of the exon SEQ ID NOs: set forth in Table 9, the complement thereof, or a fragment of said SEQ ID NO: or said complement,

wherein said probe hybridizes at high stringency to a gene expressed in human lung and includes portions of no more than one human exon.

8. A single exon nucleic acid probe for measuring gene expression in human bone marrow, comprising:

a nucleotide sequence as set forth in any one of the exon SEQ ID NOs: set forth in Table 10, the complement thereof, or a fragment of said SEQ ID NO: or said complement,

wherein said probe hybridizes at high stringency to a gene expressed in human bone marrow and includes portions of no more than one human exon.

9. A single exon nucleic acid probe for measuring gene expression in HeLa or other human cervical epithelial cells, comprising:

a nucleotide sequence as set forth in any one of the exon SEQ ID NOs: set forth in Table 11, the complement thereof, or a fragment of said SEQ ID NO: or said complement,

wherein said nucleic acid hybridizes at high stringency to a gene expressed in HeLa cells and includes portions of no more than one human exon.

10. A single exon nucleic acid probe for measuring gene expression in BT 474 or other human mammary epithelial cells, comprising:

a nucleotide sequence as set forth in any one of the exon SEQ ID NOs: set forth in Table 12, the complement thereof, or a fragment of said SEQ ID NO: or said complement,

wherein said probe hybridizes at high stringency to a gene expressed in BT 474 cells and includes portions of no more than one human exon.

11. A single exon nucleic acid probe for measuring gene expression in HBL 100 or other human mammary epithelial cells, comprising:

a nucleotide sequence as set forth in any one of the exon SEQ ID NOs: set forth in Table 13, the complement thereof, or a fragment of said SEQ ID NO: or said complement,

wherein said probe hybridizes at high stringency to a gene expressed in HBL 100 cells and includes portions of no more than one human exon.

12. The single exon nucleic acid probe of any one of paragraphs 1-11, wherein said fragment includes at least 20 contiguous nucleotides of said SEQ ID NO: or the complement thereof.

13. The single exon nucleic acid probe of any one of paragraphs 1-11, wherein said fragment includes at least 25 contiguous nucleotides of said SEQ ID NO: or the complement thereof.

14. The single exon nucleic acid probe of any one of paragraphs 1-11, wherein said fragment includes at least 50 contiguous nucleotides of said SEQ ID NO: or the complement thereof.

15. The single exon nucleic acid probe of any one of paragraphs 1-11, wherein said fragment includes the entirety of said SEQ ID NO: or the complement thereof.

16. The single exon nucleic acid probe of paragraph 1, comprising any one of SEQ ID NOs. 1-16,834 or the complement thereof.

17. The single exon nucleic acid probe of paragraph 2, comprising any one of the probe SEQ ID NOs: set forth in Table 4, or the complement thereof.

18. The single exon nucleic acid probe of paragraph 3, comprising any one of the probe SEQ ID NOs: set forth in Table 5, or the complement thereof.

19. The single exon nucleic acid probe of paragraph 4, comprising any one of the probe SEQ ID NOs: set forth in Table 6, or the complement thereof.

20. The single exon nucleic acid probe of paragraph 5, comprising any one of the probe SEQ ID NOs: set forth in Table 7, or the complement thereof.

21. The single exon nucleic acid probe of paragraph 6, comprising any one of the probe SEQ ID NOs: set forth in Table 8, or the complement thereof.

22. The single exon nucleic acid probe of paragraph 7, comprising any one of the probe SEQ ID NOs: set forth in Table 9, or the complement thereof.

23. The single exon nucleic acid probe of paragraph 8, comprising any one of the probe SEQ ID NOs: set forth in Table 10, or the complement thereof.

24. The single exon nucleic acid probe of paragraph 9, comprising any one of the probe SEQ ID NOs: set forth in Table 11, or the complement thereof.

25. The single exon nucleic acid probe of paragraph 10, comprising any one of the probe SEQ ID NOs: set forth in Table 12, or the complement thereof.

26. The single exon nucleic acid probe of paragraph 11, comprising any one of the probe SEQ ID NOs: set forth in Table 13, or the complement thereof.

27. The single exon nucleic acid probe of any one of paragraphs 1-26, wherein said probe further comprises, contiguous to a first end of said fragment, a first intronic and/or intergenic sequence that is identically contiguous to said fragment in the human genome.

28. The single exon nucleic acid probe of paragraph 27, wherein said probe comprises, contiguous to a first end of said fragment, a first intronic and/or intergenic sequence that is identically contiguous to said fragment in the human genome, and wherein said probe further comprises, contiguous to a second end of said fragment, a second intronic and/or intergenic sequence that is identically contiguous to said fragment in the human genome.

29. The single exon nucleic acid probe of any one of paragraphs 1-28, wherein said probe further includes at least a first priming sequence not found in contiguity with the rest of said probe sequence in the human genome.

30. The single exon nucleic acid probe of paragraph 29, wherein said first priming sequence is at the 5′ or 3′ end of said probe.

31. The single exon nucleic acid probe of paragraph 29, wherein said probe includes a first priming sequence and a second priming sequence, neither of which priming sequences is found in contiguity with the rest of said probe sequence in the human genome.

32. The single exon nucleic acid probe of paragraph 31, wherein said first and second priming sequences are respectively at the 5′ and 3′ ends of said probe.

33. The single exon nucleic acid probe of either of paragraphs 31 or 32, wherein said first priming sequence is nonidentical to said second priming sequence.

34. The single exon nucleic acid probe of any of paragraphs 29-33, wherein said priming sequences are at least 15 nt in length.

35. The single exon nucleic acid probe of any one of paragraphs 1-34, wherein said probe is no more than 25 kb in length.

36. The single exon nucleic acid probe of any one of paragraphs 1-34, wherein said probe is no more than 20 kb in length.

37. The single exon nucleic acid probe of any one of paragraphs 1-34, wherein said probe is no more than 15 kb in length.

38. The single exon nucleic acid probe of any one of paragraphs 1-34, wherein said probe is no more than 10 kb in length.

39. The single exon nucleic acid probe of any one of paragraphs 1-34, wherein said probe is no more than 5 kb in length.

40. The single exon nucleic acid probe of any one of paragraphs 1-34, wherein said probe is no more than 3 kb in length.

41. The single exon nucleic acid probe of any one of paragraphs 1-40, wherein said probe is DNA.

42. The single exon nucleic acid probe of paragraph 41, wherein said DNA is single-stranded.

43. The single exon nucleic acid probe of any one of paragraphs 1-40, wherein said probe is RNA.

44. The single exon nucleic acid probe of any one of paragraphs 1-40, wherein said probe is PNA.

45. The single exon nucleic acid probe of any one of paragraphs 1-44, wherein said probe is detectably labeled.

46. The single exon nucleic acid probe of paragraph 45, wherein said probe is labeled with a radionuclide.

47. The single exon nucleic acid probe of paragraph 45, wherein said probe is labeled with a fluorophore.

48. The single exon nucleic acid probe of paragraph 45, wherein said probe is labeled with a first member of a specific binding pair.

49. The single exon nucleic acid probe of any one of paragraphs 1-48, wherein said probe lacks prokaryotic and bacteriophage vector sequence.

50. The single exon nucleic acid probe of any one of paragraphs 1-49, wherein said probe lacks homopolymeric stretches of A or T.

51. An amplifiable nucleic acid composition, comprising:

the single exon nucleic acid probe of any one of paragraphs 1-50; and

at least one nucleic acid primer,

wherein said at least one primer is sufficient to prime enzymatic amplification of said probe.

52. The amplifiable nucleic acid composition of paragraph 51, wherein said composition comprises a first nucleic acid primer and a second nucleic acid primer, and wherein said first and second nucleic acid primers are sufficient to prime enzymatic amplification of said probe.

53. The amplifiable nucleic acid composition of paragraph 52, wherein said enzymatic amplification is a polymerase chain reaction.

54. A single exon nucleic acid probe kit, comprising:

the single exon nucleic acid probe of any one of paragraphs 1-50; and

at least one nucleic acid primer,

wherein said at least one primer is sufficient to prime enzymatic amplification of said probe.

55. The single exon nucleic acid probe kit of paragraph 54, wherein said kit comprises a first nucleic acid primer and a second nucleic acid primer, and wherein said first and second nucleic acid primers are sufficient to prime enzymatic amplification of said probe.

56. The single exon nucleic acid probe kit of paragraph 55, wherein said enzymatic amplification is a polymerase chain reaction.

57. A spatially-addressable set of single exon nucleic acid probes, comprising:

a plurality of single exon nucleic acid probes according to any one of paragraphs 1-50,

wherein each of said plurality of probes is separately and addressably isolatable from said plurality.

58. A spatially-addressable set of single exon nucleic acid probes, comprising:

a plurality of single exon nucleic acid probes according to any one of paragraphs 1-50,

wherein each of said plurality of probes is separately and addressably amplifiable.

59. A spatially-addressable set of single exon nucleic acid probes for measuring gene expression in human brain, comprising:

a plurality of single exon nucleic acid probes according to any one of paragraphs 2, 12-15 as dependent from paragraph 2, 17, and 27-50 as dependent from paragraph 2,

wherein each of said plurality of probes is separately and addressably amplifiable.

60. A spatially-addressable set of single exon nucleic acid probes for measuring gene expression in human brain, comprising:

a plurality of single exon nucleic acid probes according to any one of paragraphs 2, 12-15 as dependent from paragraph 2, 17, and 27-50 as dependent from paragraph 2,

wherein each of said plurality of probes is separately and addressably isolatable from said plurality.

61. A spatially-addressable set of single exon nucleic acid probes for measuring gene expression in human heart, comprising:

a plurality of single exon nucleic acid probes according to any one of paragraphs 3, 12-15 as dependent from paragraph 3, 18, and 27-50 as dependent from paragraph 3,

wherein each of said plurality of probes is separately and addressably amplifiable.

62. A spatially-addressable set of single exon nucleic acid probes for measuring gene expression in human heart, comprising:

a plurality of single exon nucleic acid probes according to any one of paragraphs 3, 12-15 as dependent from paragraph 3, 18, and 27-50 as dependent from paragraph 3,

wherein each of said plurality of probes is separately and addressably isolatable from said plurality.

63. A spatially-addressable set of single exon nucleic acid probes for measuring gene expression in human liver, comprising:

a plurality of single exon nucleic acid probes according to any one of paragraphs 4, 12-15 as dependent from paragraph 4, 19, and 27-50 as dependent from paragraph 4,

wherein each of said plurality of probes is separately and addressably amplifiable.

64. A spatially-addressable set of single exon nucleic acid probes for measuring gene expression in human liver, comprising:

a plurality of single exon nucleic acid probes according to any one of paragraphs 4, 12-15 as dependent from paragraph 4, 19, and 27-50 as dependent from paragraph 4,

wherein each of said plurality of probes is separately and addressably isolatable from said plurality.

65. A spatially-addressable set of single exon nucleic acid probes for measuring gene expression in human fetal liver, comprising:

a plurality of single exon nucleic acid probes according to any one of paragraphs 5, 12-15 as dependent from paragraph 5, 20, and 27-50 as dependent from paragraph 5,

wherein each of said plurality of probes is separately and addressably amplifiable.

66. A spatially-addressable set of single exon nucleic acid probes for measuring gene expression in human fetal liver, comprising:

a plurality of single exon nucleic acid probes according to any one of paragraphs 5, 12-15 as dependent from paragraph 5, 20, and 27-50 as dependent from paragraph 5,

wherein each of said plurality of probes is separately and addressably isolatable from said plurality.

67. A spatially-addressable set of single exon nucleic acid probes for measuring gene expression in human placenta, comprising:

a plurality of single exon nucleic acid probes according to any one of paragraphs 6, 12-15 as dependent from paragraph 6, 21, and 27-50 as dependent from paragraph 6,

wherein each of said plurality of probes is separately and addressably amplifiable.

68. A spatially-addressable set of single exon nucleic acid probes for measuring gene expression in human placenta, comprising:

a plurality of single exon nucleic acid probes according to any one of paragraphs 6, 12-15 as dependent from paragraph 6, 21, and 27-50 as dependent from paragraph 6,

wherein each of said plurality of probes is separately and addressably isolatable from said plurality.

69. A spatially-addressable set of single exon nucleic acid probes for measuring gene expression in human lung, comprising:

a plurality of single exon nucleic acid probes according to any one of paragraphs 7, 12-15 as dependent from paragraph 7, 22, and 27-50 as dependent from paragraph 7,

wherein each of said plurality of probes is separately and addressably amplifiable.

70. A spatially-addressable set of single exon nucleic acid probes for measuring gene expression in human lung, comprising:

a plurality of single exon nucleic acid probes according to any one of paragraphs 7, 12-15 as dependent from paragraph 7, 22, and 27-50 as dependent from paragraph 7,

wherein each of said plurality of probes is separately and addressably isolatable from said plurality.

71. A spatially-addressable set of single exon nucleic acid probes for measuring gene expression in human bone marrow, comprising:

a plurality of single exon nucleic acid probes according to any one of paragraphs 8, 12-15 as dependent from paragraph 8, 23, and 27-50 as dependent from paragraph 8,

wherein each of said plurality of probes is separately and addressably amplifiable.

72. A spatially-addressable set of single exon nucleic acid probes for measuring gene expression in human bone marrow, comprising:

a plurality of single exon nucleic acid probes according to any one of paragraphs 8, 12-15 as dependent from paragraph 8, 23, and 27-50 as dependent from paragraph 8,

wherein each of said plurality of probes is separately and addressably isolatable from said plurality.

73. A spatially-addressable set of single exon nucleic acid probes for measuring gene expression in HeLa or other human cervical epithelial cells, comprising:

a plurality of single exon nucleic acid probes according to any one of paragraphs 9, 12-15 as dependent from paragraph 9, 24, and 27-50 as dependent from paragraph 9,

wherein each of said plurality of probes is separately and addressably amplifiable.

74. A spatially-addressable set of single exon nucleic acid probes for measuring gene expression in HeLa or other human cervical epithelial cells, comprising:

a plurality of single exon nucleic acid probes according to any one of paragraphs 9, 12-15 as dependent from paragraph 9, 24, and 27-50 as dependent from paragraph 9,

wherein each of said plurality of probes is separately and addressably isolatable from said plurality.

75. A spatially-addressable set of single exon nucleic acid probes for measuring gene expression in BT 474 or other human mammary epithelial cells, comprising:

a plurality of single exon nucleic acid probes according to any one of paragraphs 10, 12-15 as dependent from paragraph 10, 24, and 27 -50 as dependent from paragraph 10,

wherein each of said plurality of probes is separately and addressably amplifiable.

76. A spatially-addressable set of single exon nucleic acid probes for measuring gene expression in BT 474 or other human mammary epithelial cells, comprising:

a plurality of single exon nucleic acid probes according to any one of paragraphs 10, 12-15 as dependent from paragraph 10, 24, and 27 -50 as dependent from paragraph 10,

wherein each of said plurality of probes is separately and addressably isolatable from said plurality.

77. A spatially-addressable set of single exon nucleic acid probes for measuring gene expression in HBL 100 or other human mammary epithelial cells,

a plurality of single exon nucleic acid probes according to any one of paragraphs 11, 12-15 as dependent from paragraph 11, 25, and 27 -50 as dependent from paragraph 11,

wherein each of said plurality of probes is separately and addressably amplifiable.

78. A spatially-addressable set of single exon nucleic acid probes for measuring gene expression in HBL 100 or other human mammary epithelial cells, comprising:

a plurality of single exon nucleic acid probes according to any one of paragraphs 11, 12-15 as dependent from paragraph 11, 25, and 27 -50 as dependent from paragraph 11,

wherein each of said plurality of probes is separately and addressably isolatable from said plurality.

79. The spatially-addressable set of single exon nucleic acid probes of any one of paragraphs 57-78, wherein each of said plurality of probes is amplifiable using at least one common primer.

80. The spatially-addressable set of single exon nucleic acid probes of any one of paragraphs 57-78, wherein each of said plurality of probes is amplifiable using a first and a second common primer.

81. A kit for measuring human gene expression, comprising:

the spatially-addressable set of single exon nucleic acid probes of paragraph 79 or paragraph 80; and

at least one primer sufficient commonly to amplify each of said plurality of probes.

82. The kit of paragraph 81, comprising a first primer and a second primer, wherein said first and second primers are together sufficient commonly to amplify each of said plurality of probes.

83. The single exon nucleic acid probe of any one of paragraphs 1-50, wherein said probe is attached to a substrate.

84. The substrate-bound single exon nucleic acid probe of paragraph 83, wherein said substrate is a filter membrane.

85. The substrate-bound single exon nucleic acid probe of paragraph 84, wherein said filter membrane is nitrocellulose.

86. The substrate-bound single exon nucleic acid probe of paragraph 84, wherein said filter membrane is nylon.

87. The substrate-bound single exon nucleic acid probe of paragraph 84, wherein said filter membrane is positively-charged nylon.

88. The substrate-bound single exon nucleic acid probe of paragraph 83, wherein said substrate is selected from the group consisting of glass, amorphous silicon, crystalline silicon, and plastic.

89. The substrate-bound single exon nucleic acid probe of paragraph 88, wherein said substrate is glass.

90. The substrate-bound single exon nucleic acid probe of paragraph 88, wherein said substrate is amorphous or crystalline silicon.

91. The substrate-bound single exon nucleic acid probe of paragraph 88, wherein said substrate is a plastic.

92. The substrate-bound single exon nucleic acid probe of paragraph 91, wherein said plastic is selected from the group consisting of polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, and mixtures thereof.

93. A single exon nucleic acid microarray, comprising:

a plurality of nucleic acid probes addressably disposed upon a substrate,

wherein at least 50% of said nucleic acid probes include a fragment of no more than one exon of a eukaryotic genome, said fragment selectively hybridizable at high stringency to an expressed gene, wherein said plurality of nucleic acid probes averages at least 100 bp in length, and wherein said eukaryotic genome averages at least one intron per gene.

94. The single exon nucleic acid microarray of paragraph 93, wherein at least 60% of said nucleic acid probes include a fragment of no more than one exon of said eukaryotic genome, said fragment selectively hybridizable at high stringency to an expressed gene.

95. The single exon nucleic acid microarray of paragraph 93, wherein at least 70% of said nucleic acid probes include a fragment of no more than one exon of said eukaryotic genome, said fragment selectively hybridizable at high stringency to an expressed gene.

96. The single exon nucleic acid microarray of paragraph 93, wherein at least 75% of said nucleic acid probes include a fragment of no more than one exon of said eukaryotic genome, said fragment selectively hybridizable at high stringency to an expressed gene.

97. The single exon nucleic acid microarray of paragraph 93, wherein at least 80% of said nucleic acid probes include a fragment of no more than one exon of said eukaryotic genome, said fragment selectively hybridizable at high stringency to an expressed gene.

98. The single exon nucleic acid microarray of paragraph 93, wherein at least 85% of said nucleic acid probes include a fragment of no more than one exon of said eukaryotic genome, said fragment selectively hybridizable at high stringency to an expressed gene.

99. The single exon nucleic acid microarray of paragraph 93, wherein at least 95% of said nucleic acid probes include a fragment of no more than one exon of said eukaryotic genome, said fragment selectively hybridizable at high stringency to an expressed gene.

100. The single exon nucleic acid microarray of paragraph 93, wherein at least 99% of said nucleic acid probes include a fragment of no more than one exon of said eukaryotic genome, said fragment selectively hybridizable at high stringency to an expressed gene.

101. The single exon nucleic acid microarray of any one of paragraphs 93-100, wherein said fragment includes at least 20 contiguous nucleotides of said exon.

102. The single exon nucleic acid microarray of any one of paragraphs 93-100, wherein said fragment includes at least 25 contiguous nucleotides of said exon.

103. The single exon nucleic acid microarray of any one of paragraphs 93-100, wherein said fragment includes at least 50 contiguous nucleotides of said exon.

104. The single exon nucleic acid microarray of any one of paragraphs 93-100, wherein said fragment includes the entirety of said exon.

105. The single exon nucleic acid microarray of any one of paragraphs 93-104, wherein at least 50% of said exon-including nucleic acid probes further comprise, contiguous to a first end of said fragment, a first intronic and/or intergenic sequence that is identically contiguous to said fragment in the human genome.

106. The single exon nucleic acid microarray of any one of paragraphs 93-104, wherein at least 60% of said exon-including nucleic acid probes further comprise, contiguous to a first end of said fragment, a first intronic and/or intergenic sequence that is identically contiguous to said fragment in the human genome.

107. The single exon nucleic acid microarray of any one of paragraphs 93-104, wherein at least 70% of said exon-including nucleic acid probes further comprise, contiguous to a first end of said fragment, a first intronic and/or intergenic sequence that is identically contiguous to said fragment in the human genome.

108. The single exon nucleic acid microarray of any one of paragraphs 93-104, wherein at least 75% of said exon-including nucleic acid probes further comprise, contiguous to a first end of said fragment, a first intronic and/or intergenic sequence that is identically contiguous to said fragment in the human genome.

109. The single exon nucleic acid microarray of any one of paragraphs 93-104, wherein at least 80% of said exon-including nucleic acid probes further comprise, contiguous to a first end of said fragment, a first intronic and/or intergenic sequence that is identically contiguous to said fragment in the human genome.

110. The single exon nucleic acid microarray of any one of paragraphs 93-104, wherein at least 85% of said exon-including nucleic acid probes further comprise, contiguous to a first end of said fragment, a first intronic and/or intergenic sequence that is identically contiguous to said fragment in the human genome.

111. The single exon nucleic acid microarray of any one of paragraphs 93-104, wherein at least 90% of said exon-including nucleic acid probes further comprise, contiguous to a first end of said fragment, a first intronic and/or intergenic sequence that is identically contiguous to said fragment in the human genome.

112. The single exon nucleic acid microarray of any one of paragraphs 93-104, wherein at least 95% of said exon-including nucleic acid probes further comprise, contiguous to a first end of said fragment, a first intronic and/or intergenic sequence that is identically contiguous to said fragment in the human genome.

113. The single exon nucleic acid microarray of any one of paragraphs 93-104, wherein at least 99% of said exon-including nucleic acid probes further comprise, contiguous to a first end of said fragment, a first intronic and/or intergenic sequence that is identically contiguous to said fragment in the human genome.

114. The single exon nucleic acid microarray of any one of paragraphs 93-104, wherein at least 50% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

115. The single exon nucleic acid microarray of any one of paragraphs 93-104, wherein at least 60% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

116. The single exon nucleic acid microarray of any one of paragraphs 93-104, wherein at least 70% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

117. The single exon nucleic acid microarray of any one of paragraphs 93-104, wherein at least 75% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

118. The single exon nucleic acid microarray of any one of paragraphs 93-104, wherein at least 80% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

119. The single exon nucleic acid microarray of any one of paragraphs 93-104, wherein at least 85% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

120. The single exon nucleic acid microarray of any one of paragraphs 93-104, wherein at least 90% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

121. The single exon nucleic acid microarray of any one of paragraphs 93-104, wherein at least 95% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

122. The single exon nucleic acid microarray of any one of paragraphs 93-104, wherein at least 99% of said exon-including nucleic acid probes comprise, contiguous to a first end of said fragment, a first intronic and/or intergenic sequence that is identically contiguous to said fragment in the human genome, and further comprise, contiguous to a second end of said fragment, a second intronic and/or intergenic sequence that is identically contiguous to said fragment in the human genome.

123. The single exon nucleic acid microarray of any one of paragraphs 93-122, wherein at least 50% of said exon-including nucleic acid probes comprise at least a first priming sequence not found in contiguity with the rest of said probe sequence in the human genome.

124. The single exon nucleic acid microarray of any one of paragraphs 93-122, wherein at least 50% of said exon-including nucleic acid probes comprise at least a first priming sequence not found in contiguity with the rest of said probe sequence in the human genome.

125. The single exon nucleic acid microarray of any one of paragraphs 93-122, wherein at least 60% of said exon-including nucleic acid probes comprise at least a first priming sequence not found in contiguity with the rest of said probe sequence in the human genome.

126. The single exon nucleic acid microarray of any one of paragraphs 93-122, wherein at least 70% of said exon-including nucleic acid probes comprise at least a first priming sequence not found in contiguity with the rest of said probe sequence in the human genome.

127. The single exon nucleic acid microarray of any one of paragraphs 93-122, wherein at least 75% of said exon-including nucleic acid probes comprise at least a first priming sequence not found in contiguity with the rest of said probe sequence in the human genome.

128. The single exon nucleic acid microarray of any one of paragraphs 93-122, wherein at least 80% of said exon-including nucleic acid probes comprise at least a first priming sequence not found in contiguity with the rest of said probe sequence in the human genome.

129. The single exon nucleic acid microarray of any one of paragraphs 93-122, wherein at least 85% of said exon-including nucleic acid probes comprise at least a first priming sequence not found in contiguity with the rest of said probe sequence in the human genome.

130. The single exon nucleic acid microarray of any one of paragraphs 93-122, wherein at least 90% of said exon-including nucleic acid probes comprise at least a first priming sequence not found in contiguity with the rest of said probe sequence in the human genome.

131. The single exon nucleic acid microarray of any one of paragraphs 93-122, wherein at least 95% of said exon-including nucleic acid probes comprise at least a first priming sequence not found in contiguity with the rest of said probe sequence in the human genome.

132. The single exon nucleic acid microarray of any one of paragraphs 93-122, wherein at least 99% of said exon-including nucleic acid probes comprise at least a first priming sequence not found in contiguity with the rest of said probe sequence in the human genome.

133. The single exon nucleic acid microarray of any one of paragraphs 123-132, wherein said first priming sequence is identical among said primer-including single exon probes.

134. The single exon nucleic acid microarray of any one of paragraphs 93-122, wherein at least 50% of said exon-including nucleic acid probes comprise a first priming sequence and a second priming sequence, neither of which priming sequences is found in contiguity with the rest of said probe sequence in the human genome.

135. The single exon nucleic acid microarray of any one of paragraphs 93-122, wherein at least 60% of said exon-including nucleic acid probes comprise a first priming sequence and a second priming sequence, neither of which priming sequences is found in contiguity with the rest of said probe sequence in the human genome.

136. The single exon nucleic acid microarray of any one of paragraphs 93-122, wherein at least 70% of said exon-including nucleic acid probes comprise a first priming sequence and a second priming sequence, neither of which priming sequences is found in contiguity with the rest of said probe sequence in the human genome.

137. The single exon nucleic acid microarray of any one of paragraphs 93-122, wherein at least 75% of said exon-including nucleic acid probes comprise a first priming sequence and a second priming sequence, neither of which priming sequences is found in contiguity with the rest of said probe sequence in the human genome.

138. The single exon nucleic acid microarray of any one of paragraphs 93-122, wherein at least 80% of said exon-including nucleic acid probes comprise a first priming sequence and a second priming sequence, neither of which priming sequences is found in contiguity with the rest of said probe sequence in the human genome.

139. The single exon nucleic acid microarray of any one of paragraphs 93-122, wherein at least 85% of said exon-including nucleic acid probes comprise a first priming sequence and a second priming sequence, neither of which priming sequences is found in contiguity with the rest of said probe sequence in the human genome.

140. The single exon nucleic acid microarray of any one of paragraphs 93-122, wherein at least 90% of said exon-including nucleic acid probes comprise a first priming sequence and a second priming sequence, neither of which priming sequences is found in contiguity with the rest of said probe sequence in the human genome.

141. The single exon nucleic acid microarray of any one of paragraphs 93-122, wherein at least 95% of said exon-including nucleic acid probes comprise a first priming sequence and a second priming sequence, neither of which priming sequences is found in contiguity with the rest of said probe sequence in the human genome.

142. The single exon nucleic acid microarray of any one of paragraphs 93-122, wherein at least 99% of said exon-including nucleic acid probes comprise a first priming sequence and a second priming sequence, neither of which priming sequences is found in contiguity with the rest of said probe sequence in the human genome.

143. The single exon nucleic acid microarray of any one of paragraphs 134-142, wherein said first priming sequence is identical among said primer-including single exon probes, and wherein said second priming sequence is identical among said primer-including single exon probes.

144. The single exon nucleic acid microarray of any one of paragraphs 93-143, wherein said plurality of nucleic acid probes averages at least 200 bp in length.

145. The single exon nucleic acid microarray of any one of paragraphs 93-143, wherein said plurality of nucleic acid probes averages at least 250 bp in length.

146. The single exon nucleic acid microarray of any one of paragraphs 93-143, wherein said plurality of nucleic acid probes averages at least 300 bp in length.

147. The single exon nucleic acid microarray of any one of paragraphs 93-143, wherein said plurality of nucleic acid probes averages at least 400 bp in length.

148. The single exon nucleic acid microarray of any one of paragraphs 93-143, wherein said plurality of nucleic acid probes averages at least 500 bp in length.

149. The single exon nucleic acid microarray of any one of paragraphs 93-148, wherein said plurality of addressably disposed nucleic acid probes consist essentially of DNA.

150. The single exon nucleic acid microarray of paragraph 149, wherein said DNA is single-stranded.

151. The single exon nucleic acid microarray of paragraph 149, wherein said DNA is double-stranded.

152. The single exon nucleic acid microarray of any one of paragraphs 93-148, wherein said plurality of addressably disposed nucleic acid probes consist essentially of RNA.

153. The single exon nucleic acid microarray of any one of paragraphs 93-148, wherein said plurality of addressably disposed nucleic acid probes consist essentially of PNA.

154. The single exon nucleic acid microarray of any one of paragraphs 93-153, wherein at least 50% of said exon-including nucleic acid probes lack prokaryotic and bacteriophage vector sequence.

155. The single exon nucleic acid microarray of any one of paragraphs 93-153, wherein at least 60% of said exon-including nucleic acid probes lack prokaryotic and bacteriophage vector sequence.

156. The single exon nucleic acid microarray of any one of paragraphs 93-153, wherein at least 70% of said exon-including nucleic acid probes lack prokaryotic and bacteriophage vector sequence.

157. The single exon nucleic acid microarray of any one of paragraphs 93-153, wherein at least 75% of said exon-including nucleic acid probes lack prokaryotic and bacteriophage vector sequence.

158. The single exon nucleic acid microarray of any one of paragraphs 93-153, wherein at least 80% of said exon-including nucleic acid probes lack prokaryotic and bacteriophage vector sequence.

159. The single exon nucleic acid microarray of any one of paragraphs 93-153, wherein at least 85% of said exon-including nucleic acid probes lack prokaryotic and bacteriophage vector sequence.

160. The single exon nucleic acid microarray of any one of paragraphs 93-153, wherein at least 90% of said exon-including nucleic acid probes lack prokaryotic and bacteriophage vector sequence.

161. The single exon nucleic acid microarray of any one of paragraphs 93-153, wherein at least 95% of said exon-including nucleic acid probes lack prokaryotic and bacteriophage vector sequence.

162. The single exon nucleic acid microarray of any one of paragraphs 93-153, wherein at least 99% of said exon-including nucleic acid probes lack prokaryotic and bacteriophage vector sequence.

163. The single exon nucleic acid microarray of any one of paragraphs 93-162, wherein at least 50% of said exon-including nucleic acid probes lack homopolymeric stretches of A or T.

164. The single exon nucleic acid microarray of any one of paragraphs 93-162, wherein at least 60% of said exon-including nucleic acid probes lack homopolymeric stretches of A or T.

165. The single exon nucleic acid microarray of any one of paragraphs 93-162, wherein at least 70% of said exon-including nucleic acid probes lack homopolymeric stretches of A or T.

166. The single exon nucleic acid microarray of any one of paragraphs 93-162, wherein at least 75% of said exon-including nucleic acid probes lack homopolymeric stretches of A or T.

167. The single exon nucleic acid microarray of any one of paragraphs 93-162, wherein at least 80% of said exon-including nucleic acid probes lack homopolymeric stretches of A or T.

168. The single exon nucleic acid microarray of any one of paragraphs 93-162, wherein at least 85% of said exon-including nucleic acid probes lack homopolymeric stretches of A or T.

169. The single exon nucleic acid microarray of any one of paragraphs 93-162, wherein at least 90% of said exon-including nucleic acid probes lacks homopolymeric stretches of A or T.

170. The single exon nucleic acid microarray of any one of paragraphs 93-162, wherein at least 95% of said exon-including nucleic acid probes lacks homopolymeric stretches of A or T.

171. The single exon nucleic acid microarray of any one of paragraphs 93-162, wherein at least 99% of said exon-including nucleic acid probes lacks homopolymeric stretches of A or T.

172. The single exon nucleic acid microarray of any one of paragraphs 93-171, wherein said plurality of addressably disposed nucleic acid probes are noncovalently bound to said substrate.

173. The single exon nucleic acid microarray of any one of paragraphs 93-171, wherein said plurality of addressably disposed nucleic acid probes are covalently bound to said substrate.

174. The single exon nucleic acid microarray of any one of paragraphs 93-173, wherein said substrate is selected from the group consisting of glass, amorphous silicon, crystalline silicon, and plastic.

175. The single exon nucleic acid microarray of paragraph 174, wherein said substrate is glass.

176. The single exon nucleic acid microarray of paragraph 174, wherein said substrate is amorphous or crystalline silicon.

177. The single exon nucleic acid microarray of paragraph 174, wherein said substrate is a plastic.

178. The single exon nucleic acid microarray of paragraph 177, wherein said plastic is selected from the group consisting of polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, and mixtures thereof.

179. The single exon nucleic acid microarray of any one of paragraphs 93-178, wherein said eukaryotic genome averages at least two introns per gene.

180. The single exon nucleic acid microarray of any one of paragraphs 93-178, wherein said eukaryotic genome averages at least three introns per gene.

181. The single exon nucleic acid microarray of any one of paragraphs 93-178, wherein said eukaryotic genome averages at least four introns per gene.

182. The single exon nucleic acid microarray of any one of paragraphs 93-178, wherein said eukaryotic genome averages at least five introns per gene.

183. The single exon nucleic acid microarray of any one of paragraphs 93-178, wherein said genome is a human genome.

184. A single exon nucleic acid microarray for measuring human gene expression, comprising:

a plurality of single exon nucleic acid probes according to any one of paragraphs 1-50,

wherein said plurality of probes is addressably disposed upon a substrate.

185. A single exon nucleic acid microarray for measuring gene expression in human brain, comprising:

a plurality of single exon nucleic acid probes according to any one of paragraphs 2, 12-15 as dependent from paragraph 2, 17, and 27-50 as dependent from paragraph 2,

wherein said plurality of probes is addressably disposed upon a substrate.

186. A single exon nucleic acid microarray for measuring gene expression in human heart, comprising:

a plurality of single exon nucleic acid probes according to any one of paragraphs 3, 12-15 as dependent from paragraph 3, 18, and 27-50 as dependent from paragraph 3,

wherein said plurality of probes is addressably disposed upon a substrate.

187. A single exon nucleic acid microarray for measuring gene expression in human liver, comprising:

a plurality of single exon nucleic acid probes according to any one of paragraphs 4, 12-15 as dependent from paragraph 4, 19, and 27-50 as dependent from paragraph 4,

wherein said plurality of probes is addressably disposed upon a substrate.

188. A single exon nucleic acid microarray for measuring gene expression in human fetal liver, comprising:

a plurality of single exon nucleic acid probes according to any one of paragraphs 5, 12-15 as dependent from paragraph 5, 20, and 27-50 as dependent from paragraph 5,

wherein said plurality of probes is addressably disposed upon a substrate.

189. A single exon nucleic acid microarray for measuring gene expression in human placenta, comprising:

a plurality of single exon nucleic acid probes according to any one of paragraphs 6, 12-15 as dependent from paragraph 6, 21, and 27-50 as dependent from paragraph 6,

wherein said plurality of probes is addressably disposed upon a substrate.

190. A single exon nucleic acid microarray for measuring gene expression in human lung, comprising:

a plurality of single exon nucleic acid probes according to any one of paragraphs 7, 12-15 as dependent from paragraph 7, 22, and 27-50 as dependent from paragraph 7,

wherein said plurality of probes is addressably disposed upon a substrate.

191. A single exon nucleic acid microarray for measuring gene expression in human bone marrow, comprising:

a plurality of single exon nucleic acid probes according to any one of paragraphs 8, 12-15 as dependent from paragraph 8, 23, and 27-50 as dependent from paragraph 8,

wherein said plurality of probes is addressably disposed upon a substrate.

192. A single exon nucleic acid microarray for measuring gene expression in HeLa or other human cervical epithelial cells, comprising:

a plurality of single exon nucleic acid probes according to any one of paragraphs 9, 12-15 as dependent from paragraph 9, 24, and 27-50 as dependent from paragraph 9,

wherein said plurality of probes is addressably disposed upon a substrate.

193. A single exon nucleic acid microarray for measuring gene expression in BT 474 or other human mammary epithelial cells, comprising:

a plurality of single exon nucleic acid probes according to any one of paragraphs 10, 12-15 as dependent from paragraph 10, 24, and 27 -50 as dependent from paragraph 10,

wherein said plurality of probes is addressably disposed upon a substrate.

194. A single exon nucleic acid microarray for measuring gene expression in HBL 100 or other human mammary epithelial cells,

a plurality of single exon nucleic acid probes according to any one of paragraphs 11, 12-15 as dependent from paragraph 11, 25, and 27 -50 as dependent from paragraph 11,

wherein said plurality of probes is addressably disposed upon a substrate.

195. The single exon nucleic acid microarray for measuring human gene expression of any one of paragraphs 184-194, wherein said plurality of single exon nucleic acid probes averages at least 200 bp in length.

196. The single exon nucleic acid microarray for measuring human gene expression of any one of paragraphs 184-194, wherein said plurality of single exon nucleic acid probes averages at least 250 bp in length.

197. The single exon nucleic acid microarray for measuring human gene expression of any one of paragraphs 184-194, wherein said plurality of single exon nucleic acid probes averages at least 300 bp in length.

198. The single exon nucleic acid microarray for measuring human gene expression of any one of paragraphs 184-194, wherein said plurality of single exon nucleic acid probes averages at least 400 bp in length.

199. The single exon nucleic acid microarray for measuring human gene expression of any one of paragraphs 184-194, wherein said plurality of single exon nucleic acid probes averages at least 500 bp in length.

200. The single exon nucleic acid microarray for measuring human gene expression of any one of paragraphs 184-199, wherein said plurality of addressably disposed nucleic acid probes are noncovalently bound to said substrate.

201. The single exon nucleic acid microarray for measuring human gene expression of any one of paragraphs 184-199, wherein said plurality of addressably disposed nucleic acid probes are covalently bound to said substrate.

202. The single exon nucleic acid microarray for measuring human gene expression of any one of paragraphs 184-199, wherein said substrate is selected from the group consisting of glass, amorphous silicon, crystalline silicon, and plastic.

203. The single exon nucleic acid microarray of paragraph 202, wherein said substrate is glass.

204. The single exon nucleic acid microarray of paragraph 202, wherein said substrate is amorphous or crystalline silicon.

205. The single exon nucleic acid microarray of paragraph 202, wherein said substrate is a plastic.

206. The single exon nucleic acid microarray of paragraph 205, wherein said plastic is selected from the group consisting of polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, and mixtures thereof.

207. A method of measuring eukaryotic gene expression, comprising:

contacting the single exon microarray of any one of paragraphs 93-206 with a first collection of detectably labeled nucleic acids, said first collection nucleic acids derived from mRNA of at least one eukaryotic tissue or cell type; and then

measuring the label detectably bound to each probe of said microarray.

208. A method of measuring gene expression in human brain, comprising:

contacting the single exon microarray of any one of paragraphs 93-183, 185, and 195-206 as dependent from paragraph 185, with a first collection of detectably labeled nucleic acids, said first collection nucleic acids derived from mRNA of human brain; and then

measuring the label detectably bound to each probe of said microarray.

209. A method of measuring gene expression in human heart, comprising:

contacting the single exon microarray of any one of paragraphs 93-183, 186, and 195-206 as dependent from paragraph 186, with a first collection of detectably labeled nucleic acids, said first collection nucleic acids derived from mRNA of human heart; and then

measuring the label detectably bound to each probe of said microarray.

210. A method of measuring gene expression in human liver, comprising:

contacting the single exon microarray of any one of paragraphs 93-183, 187, and 195-206 as dependent from paragraph 187, with a first collection of detectably labeled nucleic acids, said first collection nucleic acids derived from mRNA of human liver; and then

measuring the label detectably bound to each probe of said microarray.

211. A method of measuring gene expression in human fetal liver, comprising:

contacting the single exon microarray of any one of paragraphs 93-183, 188, and 195-206 as dependent from paragraph 188, with a first collection of detectably labeled nucleic acids, said first collection nucleic acids derived from mRNA of human fetal liver; and then

measuring the label detectably bound to each probe of said microarray.

212. A method of measuring gene expression in human placenta, comprising:

contacting the single exon microarray of any one of paragraphs 93-183, 189, and 195-206 as dependent from paragraph 189, with a first collection of detectably labeled nucleic acids, said first collection nucleic acids derived from mRNA of human placenta; and then

measuring the label detectably bound to each probe of said microarray.

213. A method of measuring gene expression in human lung, comprising:

contacting the single exon microarray of any one of paragraphs 93-183, 190, and 195-206 as dependent from paragraph 190, with a first collection of detectably labeled nucleic acids, said first collection nucleic acids derived from mRNA of human lung; and then

measuring the label detectably bound to each probe of said microarray.

214. A method of measuring gene expression in human bone marrow, comprising:

contacting the single exon microarray of any one of paragraphs 93-183, 191, and 195-206 as dependent from paragraph 191, with a first collection of detectably labeled nucleic acids, said first collection nucleic acids derived from mRNA of human bone marrow; and then

measuring the label detectably bound to each probe of said microarray.

215. A method of measuring gene expression in HeLa or other human cervical epithelial cells, comprising:

contacting the single exon microarray of any one of paragraphs 93-183, 192, and 195-206 as dependent from paragraph 192, with a first collection of detectably labeled nucleic acids, said first collection nucleic acids derived from mRNA of human cervical epithelial cells; and then

measuring the label detectably bound to each probe of said microarray.

216. A method of measuring gene expression in BT474 or other human mammary epithelial cells, comprising:

contacting the single exon microarray of any one of paragraphs 93-183, 193, and 195-206 as dependent from paragraph 193, with a first collection of detectably labeled nucleic acids, said first collection nucleic acids derived from mRNA of human mammary epithelial cells; and then

measuring the label detectably bound to each probe of said microarray.

217. A method of measuring gene expression in HBL 100 or other human mammary epithelial cells, comprising:

contacting the single exon microarray of any one of paragraphs 93-183, 194, and 195-206 as dependent from paragraph 194, with a first collection of detectably labeled nucleic acids, said first collection nucleic acids derived from mRNA of human mammary epithelial cells; and then

measuring the label detectably bound to each probe of said microarray.

218. The method of any one of paragraphs 207-217, further comprising comparing said measurement to a second measurement, said second measurement identically obtained using a second, control, collection of nucleic acids.

219. The method of paragraph 218, wherein said control nucleic acids are derived from a plurality of human tissues and/or cell types.

220. The method of paragraph 218 or 219, wherein said microarray is contacted simultaneously with said first and second collections of detectably labeled nucleic acids, wherein said first and second collection nucleic acids are distinguishably labeled.

221. The method of any one of paragraphs 207-220, wherein said detectable label is a fluorescent label.

222. The method of paragraph 221, wherein said fluorescent label is a cyanine dye.

223. The method of paragraph 222, wherein said cyanine dye is CY3 or CY5.

224. A method of identifying exons in a eukaryotic genome, comprising:

algorithmically predicting at least one exon from genomic sequence of said eukaryote; and then

detecting specific hybridization of detectably labeled nucleic acids to a single exon probe,

wherein said detectably labeled nucleic acids are derived from mRNA of said eukaryote, said probe is a single exon probe having a fragment identical in sequence to, or complementary in sequence to, said predicted exon, said probe is included within a single exon microarray according to any one of paragraphs 93-183, and said fragment is selectively hybridizable at high stringency.

225. The method of paragraph 224, wherein said algorithmic predicting includes use of at least one gene prediction algorithm.

226. The method of paragraph 225, wherein said at least one gene prediction algorithm is that embodied in software selected from the group consisting of: GRAIL, GRAIL II, GENEFINDER, GENSCAN and DICTION.

227. The method of paragraph 226, wherein said software is GRAIL or GRAIL II.

228. The method of paragraph 226, wherein said software is GENEFINDER.

229. The method of paragraph 226, wherein said software is DICTION.

230. The method of paragraph 225, wherein said algorithmic predicting includes finding consensus among at least two of said algorithms.

231. The method of paragraph 224, wherein said algorithmic predicting includes use of comparative sequence analysis algorithms.

232. The method of any one of paragraphs 224-231, wherein said mRNA-derived nucleic acids derive from mRNA from a plurality of tissues and/or cell types.

233. The method of paragraph 232, wherein said eukaryote is a human, and said plurality of tissues and/or cell types is selected from the group consisting of: brain, heart, liver, fetal liver, placenta, lung, bone marrow, HeLa cells, BT 474 cells, or HBL 100 cells.

234. The method of any one of paragraphs 224-233, wherein said detecting involves simultaneous detection of two fluorophores having distinguishable emission spectra.

235. The method of paragraph 234, wherein one of said fluorophores is Cy3.

236. The method of paragraph 234, wherein one of said fluorophores is Cy5.

237. The method of paragraph 234, wherein one of said fluorophores is Cy3 and the other is Cy5.

238. A method of assigning exons to a single gene, comprising:

identifying a plurality of exons from genomic sequence according to the method of any one of paragraphs 224-237; and then

measuring the expression of each of said exons in a plurality of tissues and/or cell types using hybridization to single exon microarrays having a probe with said exon,

wherein a common pattern of expression of said exons in said plurality of tissues and/or cell types indicates that the exons should be assigned to a single gene.

239. A visual display of eukaryotic genomic sequence annotated with information about a predetermined biologic function, comprising:

a first visual element, each point along the length of which first visual element maps linearly and uniquely to a nucleotide of said genomic sequence;

a second visual element, first and second boundaries of which second visual element map linearly to a first and second nucleotide of said genomic sequence, wherein said first and second nucleotides delimit a region of said genomic sequence predicted to have said predetermined function; and

a third visual element, first and second boundaries of which third visual element map linearly to a first and second nucleotide of said genomic sequence, wherein said first and second nucleotides delimit a region of said genomic sequence experimentally confirmed to have said predetermined function.

240. The visual display of paragraph 239, wherein said display is electronic.

241. A method of selling and/or licensing genome-derived single-exon microarrays to a customer desiring to measure gene expression, comprising:

making available for computerized query a database having a record corresponding to each genome-derived single exon microarray available for sale and/or license;

responding to a customer query of the database by returning to the customer at least one record, or an identifier of that record, that best meets the customer query criteria; and

offering for sale or license to the querying customer the genome-derived single exon microarray identified in that at least one record.

242. The method of paragraph 241, wherein said database is resident on a server computer and is available for query by a remotely located client.

243. The method of paragraph 242, wherein said client and said server communicate over the Internet.

244. The method of paragraph 242, wherein said client and said server communicate over an intranet.

245. The method of paragraph 242, wherein said client and said server communicate over a LAN.

246. The method of paragraph 242, wherein said client and said server communicate over a WAN.

247. The method of any one of paragraphs 241-246, further comprising a later step of permitting the customer to place an order for the identified microarray.

248. The method of any one of paragraphs 241-247, wherein said at least one of said genome-derived single exon microarrays available for sale and/or license is a single exon microarray according to any one of paragraphs 93-206.

249. A method of designing and/or manufacturing a genome-derived single exon microarray that has genome-derived single exon probes that have at least one common attribute desired by a customer, the method comprising:

receiving from a customer at least one criterion for common probe attribute;

using the at least one criterion to identify, within a database having records corresponding to available genome-derived single exon probes, those that meet the criterion; and then

disposing such identified probes on a substrate capable of functioning in microarray hybridization experiments.

250. The method of paragraph 249, wherein said common attribute is common expression in a tissue and/or cell type.

251. The method of paragraph 250, wherein said tissue and/or cell type is selected from the group consisting of: human brain, heart, liver, fetal liver, placenta, lung, bone marrow, HeLa cells, BT 474 cells, or HBL 100 cells.

252. The method of paragraph 251, wherein said tissue is human brain, and said single exon probes are selected from single exon nucleic acid probes according to any one of paragraphs 2, 12-15 as dependent from paragraph 2, 17, and 27-50 as dependent from paragraph 2.

253. The method of paragraph 251, wherein said tissue is human heart, and said single exon probes are selected from single exon nucleic acid probes according to any one of paragraphs 3, 12-15 as dependent from paragraph 3, 18, and 27-50 as dependent from paragraph 3.

254. The method of paragraph 251, wherein said tissue is human liver, and said single exon probes are selected from single exon nucleic acid probes according to any one of paragraphs 4, 12-15 as dependent from paragraph 4, 19, and 27-50 as dependent from paragraph 4.

255. The method of paragraph 251, wherein said tissue is human fetal liver, and said single exon probes are selected from single exon nucleic acid probes according to any one of paragraphs 5, 12-15 as dependent from paragraph 5, 20, and 27-50 as dependent from paragraph 5.

256. The method of paragraph 251, wherein said tissue is human placenta, and said single exon probes are selected from single exon nucleic acid probes according to any one of paragraphs 6, 12-15 as dependent from paragraph 6, 21, and 27-50 as dependent from paragraph 6.

257. The method of paragraph 251, wherein said tissue is human lung, and said single exon probes are selected from single exon nucleic acid probes according to any one of paragraphs 7, 12-15 as dependent from paragraph 7, 22, and 27-50 as dependent from paragraph 7.

258. The method of paragraph 251, wherein said tissue is human bone marrow, and said single exon probes are selected from single exon nucleic acid probes according to any one of paragraphs 8, 12-15 as dependent from paragraph 8, 23, and 27-50 as dependent from paragraph 8.

259. The method of paragraph 251, wherein said cell type is HeLa or other human cervical epithelial cells, and said single exon probes are selected from single exon nucleic acid probes according to any one of paragraphs 9, 12-15 as dependent from paragraph 9, 24, and 27-50 as dependent from paragraph 9.

260. The method of paragraph 251, wherein said cell type is BT 474 or other human mammary epithelial cells, and said single exon probes are selected from single exon nucleic acid probes according to any one of paragraphs 10, 12-15 as dependent from paragraph 10, 24, and 27-50 as dependent from paragraph 10.

261. The method of paragraph 251, wherein said cell type is HBL 100 or other human mammary epithelial cells, and said single exon probes are selected from single exon nucleic acid probes according to any one of paragraphs 11, 12-15 as dependent from paragraph 11, 25, and 27-50 as dependent from paragraph 11.

262. The method of any one of paragraphs 249-261, wherein said customer communicates said attribute electronically.

263. A method for making available by subscription expression data obtained from use of the genome-derived single exon probes and genome-derived single exon microarrays customers that desire such information, comprising:

making available to a subscription client for computerized query a database, said database having records containing expression data generated using single exon probes disposed upon microarrays; and then

responding to a customer query of the database by returning to the customer at least one record, or an identifier of the at least one record, that best meets the customer query criteria.

264. The method of paragraph 263, wherein at least one record of said database contains expression data from a single exon probe selected from single exon probes of any one of paragraphs 1-50.

265. The method of paragraph 263 or 264, wherein said customer query of said database is communicated over the Internet.

266. The method of paragraph 263 or 264, wherein said customer query of said database is communicated over an intranet.

267. The method of paragraph 263 or 264, wherein said customer query of said database is communicated over a LAN.

268. The method of paragraph 263 or 264, wherein said customer query of said database is communicated over a WAN.

269. A method of attracting investment to a company that makes and sells microarrays or expression data obtained from microarrays, comprising:

advertising the availability for distribution, sale, or license of genome-derived single exon probes, microarrays, and/or data therefrom; and then

selling stock in the company.

270. The method of paragraph 269, wherein said advertising is performed on an Internet web page, in an Internet chat room, and/or a Usenet newsgroup, and wherein the stock sale is consummated through an electronic brokerage.

271. A nucleic acid microarray, the improvement comprising: disposing upon said microarray genome-derived single exon probes.

272. A protein, polypeptide, or peptide comprising: at least 8 amino acids of the sequence set forth in any one of SEQ ID NOs: 33,300-49,117.

273. A protein, polypeptide, or peptide comprising: at least 15 amino acids of the sequence set forth in any one of SEQ ID NOs: 33,300-49,117.

274. A protein, polypeptide, or peptide comprising: the sequence set forth in any one of SEQ ID NOs: 33,300-49,117.

275. The protein, polypeptide, or peptide of any one of paragraphs 272-274, wherein said protein, polypeptide or peptide is detectably labeled.

276. The protein, polypeptide, or peptide of any one of paragraphs 272-274, fused to a detectable moiety.

277. The protein, polypeptide or peptide of paragraph 276, wherein said detectable moiety is green or blue fluorescent protein.

278. An isolated nucleic acid that encodes the protein, polypeptide, or peptide of any one of paragraphs 272-274.

279. An antibody, wherein said antibody is specific for the protein, polypeptide, or peptide of any one of paragraphs 272-274.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1 illustrates a process for predicting functional regions from genomic sequence, confirming the functional activity of such regions experimentally, and associating and displaying the data so obtained in meaningful and useful relationship to the original sequence data, according to the present invention;

FIG. 2 further elaborates that portion of the process schematized in FIG. 1 for predicting functional regions from genomic sequence, according to the present invention;

FIG. 3 illustrates a visual display according to the present invention, herein denominated a “Mondrian”, in which a single genomic sequence is annotated with predicted and experimentally confirmed functional information;

FIG. 4 presents a Mondrian of a hypothetical annotated genomic sequence, further identifying typical color conventions when the Mondrian is used to annotate genomic sequence with exon-specific expression data, as in FIGS. 9 and 10;

FIG. 5 is a chart that summarizes data from experimental Example 1, showing the size distributions of predicted exon length (dashed line) and actual PCR products (amplicons) (solid line) as obtained from human genomic sequence according to the methods of the present invention;

FIG. 6 is a histogram that summarizes data from experimental Examples 1 and 2, showing the number of tissues in which predicted exons could be shown to be expressed using simultaneous two color hybridization to a genome-derived single exon microarray of the present invention. The graph shows the number of sequence-verified products that were either not expressed in any of the ten tested tissues/cell types (“0”), expressed in one or more but not all tested tissues (“1”-“9”), or expressed in all tissues tested (“10”);

FIG. 7 is a pictorial representation of data from experimental Examples 1 and 2, showing the expression (ratio relative to control) of probes having verified sequences that were expressed with signal intensity greater than 3 in at least one tissue, with:

FIG. 7A showing the expression as measured by microarray hybridization in each of the 10 measured tissues, and the expression as measured “bioinformatically” by query of EST, NR and SwissProt databases; with

FIG. 7B showing the legend for display of physical expression (ratio) in FIG. 7A; and with

FIG. 7C showing the legend for scoring EST hits as depicted in FIG. 7A;

FIG. 8 is a chart of data from experimental Examples 1 and 2, showing a comparison of normalized CY3 signal intensity for arrayed sequences that were identical to sequences in existing EST, NR and SwissProt databases (known) (solid line) or that were dissimilar (unknown) (dashed line), where “known” is defined as having a BLAST expect (“E”) value of less than 1e−30 (1×10⁻³⁰) and “unknown” is defined as having a BLAST expect (“E”) value of greater than 1e−30 (1×10⁻³⁰) (“unknown”);

FIG. 9 presents a visual display, herein termed a “Mondrian”, of BAC AC008172 (bases 25,000 to 130,000), containing the carbamyl phosphate synthetase gene (AF154830.1); and

FIG. 10 is a Mondrian of BAC A049839.

### DETAILED DESCRIPTION OF THE INVENTION

Definitions

As used herein, “nucleic acid” includes polynucleotides having natural nucleotides in native 5′-3′ phosphodiester linkage—e.g., DNA or RNA—as well as polynucleotides that have nonnatural nucleotide analogues, nonnative internucleoside bonds, or both, so long as the nonnatural polynucleotide is capable of sequence-discriminating basepairing under experimentally desired conditions. Unless otherwise specified, the term “nucleic acid” includes any topological conformation; the term thus explicitly comprehends single-stranded, double-stranded, partially duplexed, triplexed, hairpinned, circular, and padlocked conformations.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment.

For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature.

As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

As used herein, an isolated nucleic acid “encodes” a reference polypeptide when at least a portion of the nucleic acid, or its complement, can be directly translated to provide the amino acid sequence of the reference polypeptide, or when the isolated nucleic acid can be used, alone or as part of an expression vector, to express the reference polypeptide in vitro, in a prokaryotic host cell, or in a eukaryotic host cell.

As used herein, the term “exon” refers to a nucleic acid sequence found in genomic DNA that is bioinformatically predicted and/or experimentally confirmed to contribute contiguous sequence to a mature mRNA transcript.

As used herein, the phrase “open reading frame” and the equivalent acronym “ORF” refer to that portion of an exon that can be translated in its entirety into a sequence of contiguous amino acids. As so defined, an ORF is wholly contained within its respective exon and has length, measured in nucleotides, exactly divisible by 3. As so defined, an ORF need not encode the entirety of a natural protein.

As used herein, the phrase “ORF-encoded peptide” refers to the predicted or actual translation of an ORF.

As used herein, the phrase “degenerate variant” of a reference nucleic acid sequence intends all nucleic acid sequences that can be directly translated, using the standard genetic code, to provide an amino acid sequence identical to that translated from the reference nucleic acid sequence.

As used herein, the term “microarray” and the equivalent phrase “nucleic acid microarray” refer to a substrate-bound collection of plural nucleic acids, hybridization to each of the plurality of bound nucleic acids being separately detectable. The substrate can be solid or porous, planar or non-planar, unitary or distributed.

As so defined, the term “microarray” and phrase “nucleic acid microarray” include all the devices so called in Schena (ed.), DNA Microarrays: A Practical Approach (Practical Approach Series), Oxford University Press (1999) (ISBN: 0199637768); Nature Genet. 21(1)(suppl):l -60 (1999); and Schena (ed.), Microarray Biochip: Tools and Technology, Eaton Publishing Company/BioTechniques Books Division (2000) (ISBN: 1881299376), the disclosures of which are incorporated herein by reference in their entireties.

As so defined, the term “microarray” and phrase “nucleic acid microarray” include substrate-bound collections of plural nucleic acids in which the plurality of nucleic acids are distributably disposed on a plurality of beads, rather than on a unitary planar substrate, as is described, inter alia, in Brenner et al., Proc. Natl. Acad. Sci. USA 97(4):166501670 (2000), the disclosure of which is incorporated herein by reference in its entirety; in such case, the term “microarray” and phrase “nucleic acid microarray” refer to the plurality of beads in aggregate.

As used herein with respect to solution phase hybridization, the term “probe”, or equivalently, “nucleic acid probe” or “hybridization probe”, refers to an isolated nucleic acid of known sequence that is, or is intended to be, detectably labeled. As used herein with respect to a nucleic acid microarray, the term “probe” (or equivalently “nucleic acid probe” or “hybridization probe”) refers to the isolated nucleic acid that is, or is intended to be, bound to the substrate. In either such context, the term “target” refers to nucleic acid intended to be bound to probe by sequence complementarity.

As used herein, the expression “probe comprising SEQ ID NO:X”, and variants thereof, intends a nucleic acid probe, at least a portion of which probe has either (i) the sequence directly as given in the referenced SEQ ID NO:X, or (ii) a sequence complementary to the sequence as given in the referenced SEQ ID NO:X, the choice as between sequence directly as given and complement thereof dictated by the requirement that the probe be complementary to the desired target.

As used herein, the phrases “expression of a probe” and “expression of an isolated nucleic acid” and their linguistic equivalents intend that the probe or, respectively, the isolated nucleic acid, can hybridize detectably under high stringency conditions to a sample of nucleic acids that derive from mRNA transcripts from a given source. For example, and by way of illustration only, expression of a probe in “liver” means that the probe can hybridize detectably under high stringency conditions to a sample of nucleic acids that derive from mRNA obtained from liver.

As used herein, “a single exon probe” comprises at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon but include one or more exons that are found adjacent to the reference exon in the genome.

For purposes herein, “high stringency conditions” are defined for solution phase hybridization as aqueous hybridization (i.e., free of formamide) in 6× SSC (where 20× SSC contains 3.0 M NaCl and 0.3 M sodium citrate), 1% SDS at 65° C. for at least 8 hours, followed by one or more washes in 0.2× SSC, 0.1% SDS at 65° C. “Moderate stringency conditions” are defined for solution phase hybridization as aqueous hybridization (i.e., free of formamide) in 6× SSC, 1% SDS at 65° C. for at least 8 hours, followed by one or more washes in 2× SSC, 0.1% SDS at room temperature.

For microarray-based hybridization, standard “high stringency conditions” are defined as hybridization in 50% formamide, 5× SSC, 0.2 μg/μl poly(dA), 0.2 μg/μl human cₒt1 DNA, and 0.5% SDS, in a humid oven at 42° C. overnight, followed by successive washes of the microarray in 1× SSC, 0.2% SDS at 55° C. for 5 minutes, and then 0.1× SSC, 0.2% SDS, at 55° C. for 20 minutes. For microarray-based hybridization, “moderate stringency conditions”, suitable for cross-hybridization to mRNA encoding structurally- and functionally-related proteins, are defined to be the same as those for high stringency conditions but with reduction in temperature for hybridization and washing to room temperature (approximately 25° C.).

As used herein, the terms “protein”, “polypeptide”, and “peptide” are used interchangeably to refer to a naturally-occurring or synthetic polymer of amino acid monomers (residues), irrespective of length, where amino acid monomer here includes naturally-occurring amino acids, naturally-occurring amino acid structural variants, and synthetic non-naturally occurring analogs that are capable of participating in peptide bonds. The terms “protein”, “polypeptide”, and “peptide” explicitly permits of post-translational and post-synthetic modifications, such as glycosylation.

The term “oligopeptide” herein denotes a protein, polypeptide, or peptide having 25 or fewer monomeric subunits.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment.

For example, a protein can be said to be “isolated” when it includes amino acid analogues or derivatives not found in nature, or includes linkages other than standard peptide bonds.

When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

A “purified protein” (equally, a purified polypeptide, peptide, or oligopeptide) is an isolated protein, as above described, present at a concentration of at least 95%, as measured on a mass basis with respect to total protein in a composition. A “substantially purified protein” (equally, a substantially purified polypeptide, peptide, or oligopeptide) is an isolated protein, as above described, present at a concentration of at least 70%, as measured on a mass basis with respect to total protein in a composition.

As used herein, the phrase “protein isoforms” refers to a plurality of proteins having nonidentical primary amino acid sequence but that share amino acid sequence encoded by at least one common exon.

As used herein, the phrase “alternative splicing” and its linguistic equivalents includes all types of RNA processing that lead to expression of plural protein isoforms from a single gene; accordingly, the phrase “splice variant(s)” and its linguistic equivalents embraces mRNAs transcribed from a given gene that, however processed, collectively encode plural protein isoforms. For example, and by way of illustration only, splice variants can include exon insertions, exon extensions, exon truncations, exon deletions, alternatives in the 5′ untranslated region (“5′ UT”) and alternatives in the 3′ untranslated region (“3′ UT”). Such 3′ alternatives include, for example, differences in the site of RNA transcript cleavage and site of poly(A) addition. See, e.g., Gautheret et al., Genome Res. 8:524-530 (1998).

As used herein, “orthologues” are separate occurrences of the same gene in multiple species. The separate occurrences have similar, albeit nonidentical, amino acid sequences, the degree of sequence similarity depending, in part, upon the evolutionary distance of the species from a common ancestor having the same gene.

As used herein, the term “paralogues” indicates separate occurrences of a gene in one species. The separate occurrences have similar, albeit nonidentical, amino acid sequences, the degree of sequence similarity depending, in part, upon the evolutionary distance from the gene duplication event giving rise to the separate occurrences.

As used herein, the term “homologues” is generic to “orthologues” and “paralogues”.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′2, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

As used herein, “antigen” refers to a ligand that can be bound by an antibody; an antigen need not itself be immunogenic. The portions of the antigen that make contact with the antibody are denominated “epitopes”.

“Specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷M, with specific binding reactions of greater specificity typically having affinity or avidity of at least 10⁻⁸ M to at least about 10⁻⁹ M.

As used herein, “molecular binding partners”—and equivalently, “specific binding partners”—refer to pairs of molecules, typically pairs of biomolecules, that exhibit specific binding. Nonlimiting examples are receptor and ligand, antibody and antigen, and biotin to any of avidin, streptavidin, neutrAvidin and captAvidin.

As used herein with respect to the visual display of annotated genomic sequence, the term “rectangle” means any geometric shape that has at least a first and a second border, wherein each of the first and second borders is capable of mapping uniquely to a point of another visual object of the display.

Methods and Apparatus for Generating Single Exon Probes from Genomic Sequence Data

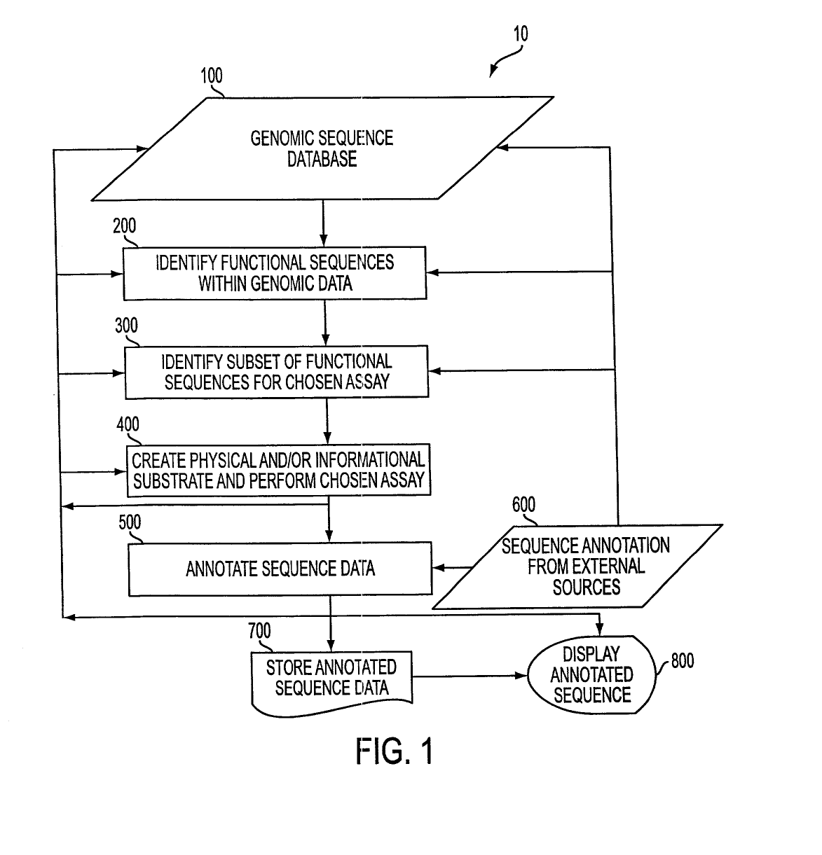

In a first aspect, the present invention provides 16,834 human genome-derived single exon nucleic acid probes useful for gene expression analysis, particularly for gene expression analysis by microarray hybridization. FIG. 1 illustrates in broad outline the process by which these probes were generated.

FIG. 1 illustrates a process for predicting functional regions from genomic sequence, confirming and characterizing the functional activity of such regions experimentally, and then associating and displaying the information so obtained in meaningful and useful relationship to the original genomic sequence data. The process is further described in commonly owned and copending U.S. application Ser. No. 09/774,203, filed Jan. 29, 2001, the disclosure of which is incorporated herein by reference in its entirety, and is detailed additionally in experimental Examples 1-4, infra. To generate single exon nucleic acid probes useful for gene expression analysis, the functional activity to be predicted, confirmed, associated and displayed is the ability of the predicted region to contribute to a mature mRNA transcript.

As shown, the initial input into process 10 is drawn from one or more databases 100 containing genomic sequence data. Because genomic sequence is usually obtained from subgenomic fragments, the sequence data typically will be stored in a series of records corresponding to these subgenomic sequenced fragments. Some fragments will have been catenated to form larger contiguous sequences (“contigs”); others will not. A finite percentage of sequence data in the database will typically be erroneous, consisting inter alia of vector sequence, sequence created from aberrant cloning events, sequence of artificial polylinkers, and sequence that was erroneously read.

Each sequence record in database 100 will minimally contain as annotation a unique sequence identifier (accession number), and will typically be annotated further to identify the date of accession, species of origin, and depositor. Because database 100 can contain nongenomic sequence, each sequence will typically be annotated further to permit query for genomic sequence. Chromosomal origin, optionally with map location, can also be present. Data can be, and over time increasingly will be, further annotated with additional information, in part through use of the present invention, as described below. Annotation can be present within the data records, in information external to database 100 and linked to the records thereto, or through a combination of the two.

Databases useful as genomic sequence database 100 in the present invention include GenBank, and particularly include several divisions thereof, including the htgs (draft), NT (nucleotide, command line), and NR (nonredundant) divisions. GenBank is produced by the National Institutes of Health and is maintained by the National Center for Biotechnology Information (NCBI). Databases of genomic sequence from species other than human, such as mouse, rat, Arabidopsis thaliana, C. elegans, C. brigsii, Drosophila melanogaster, zebra fish, and other higher eukaryotic organisms will also prove useful as genomic sequence database 100.

Genomic sequence obtained by query of genomic sequence database 100 is then input into one or more processes 200 for identification of regions therein that are predicted to have a biological function as specified by the user. Such functions include, but are not limited to, contributing to a mature mRNA transcript, encoding protein, regulating transcription, regulating message transport after transcription, regulating message splicing after transcription, regulating message degradation after transcription, contributing to or controlling chromosomal somatic recombination, contributing to chromosomal stability or movement, contributing to allelic exclusion or X chromosome inactivation, and the like. To generate single exon probes useful for gene expression analysis, the functional activity to be predicted will be the ability to contribute to a mature mRNA transcript and/or protein coding.

The particular genomic sequence to be input into process 200 will depend upon the function for which relevant sequence is to be identified as well as upon the approach chosen for such identification. Process step 200 can be iterated to identify different functions within a given genomic region. In such case, the input often will be different for the several iterations.

Sequences predicted to have the requisite function by process 200 are then input into process 300, where a subset of the input sequences suitable for experimental confirmation is identified. Experimental confirmation can involve physical and/or bioinformatic assay. Where the subsequent experimental assay is bioinformatic, rather than physical, there are fewer constraints on the sequences that can be tested, and in this latter case therefore process 300 can output the entirety of the input sequence.

The subset of sequences output from process 300 is then used in process 400 for experimental verification and characterization of the function predicted in process 200, which experimental verification can, and often will, include both physical and bioinformatic assay.

Process 500 annotates the sequence data with the functional information obtained in the physical and/or bioinformatic assays of process 400. Such annotation can be done using any technique that usefully relates the functional information to the sequence, as, for example, by incorporating the functional data into the sequence data record itself, by linking records in a hierarchical or relational database, by linking to external databases, by a combination thereof, or by other means well known within the database arts. The data can even be submitted for incorporation into databases maintained by others, such as GenBank, which is maintained by NCBI.

As further noted in FIG. 1, additional annotation can be input into process 500 from external sources 600.

The annotated data is then optionally displayed in process 800, either before, concomitantly with, or after optional storage 700 on nontransient media, such as magnetic disk, optical disc, magnetooptical disk, flash memory, or the like.

FIG. 1 shows that the experimental data output from process 400 can be used in each preceding step of process 10: e.g., facilitating identification of functional sequences in process 200, facilitating identification of an experimentally suitable subset thereof in process 300, and facilitating creation of physical and/or informational substrates for, and performance of subsequent assay, of functional sequences in process 400.

Information from each step can be passed directly to the succeeding process, or stored in permanent or interim form prior to passage to the succeeding process. Often, data will be stored after each, or at least a plurality, of such process steps. Any or all process steps can be automated.

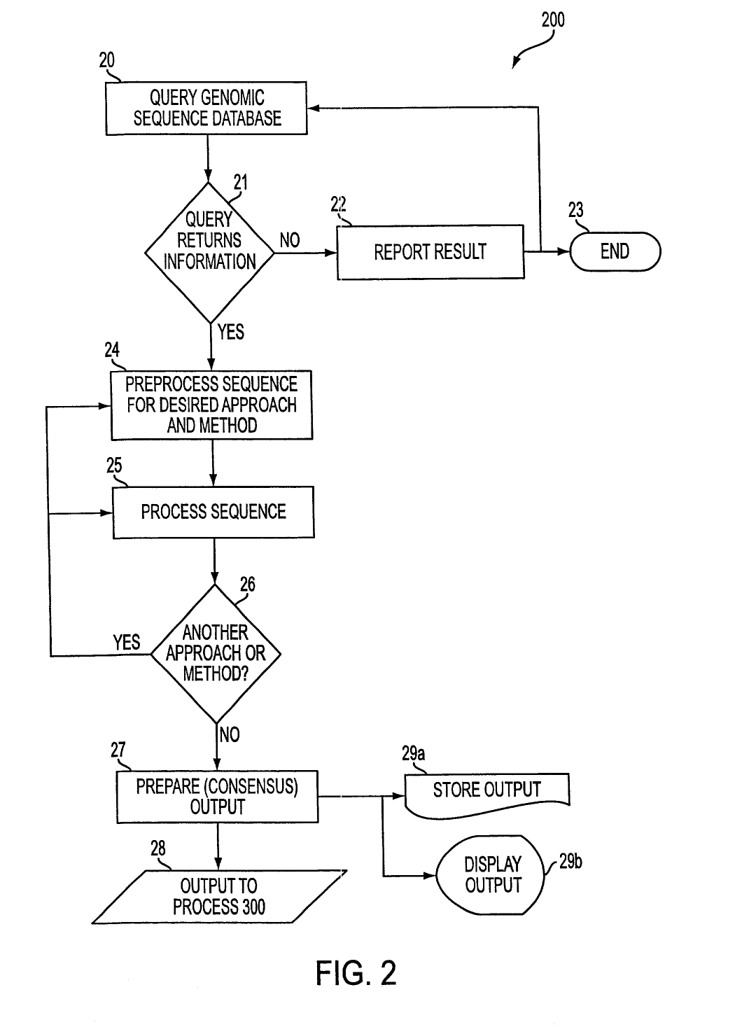

FIG. 2 further elaborates the prediction of functional sequence within genomic sequence according to process 200.

Genomic sequence database 100 is first queried 20 for genomic sequence.

The sequence required to be returned by query 20 will depend, in the first instance, upon the function to be identified.

For example, genomic sequences that function to encode protein can be identified inter alia using gene prediction approaches, comparative sequence analysis approaches, or combinations of the two. In gene prediction analysis, sequence from one genome is input into process 200 where at least one, preferably a plurality, of algorithmic methods are applied to identify putative coding regions. In comparative sequence analysis, by contrast, corresponding, e.g., syntenic, sequence from a plurality of sources, typically a plurality of species, is input into process 200, where at least one, possibly a plurality, of algorithmic methods are applied to compare the sequences and identify regions of least variability.

The exact content of query 20 will also depend upon the database queried. For example, if the database contains both genomic and nongenomic sequence, perhaps derived from multiple species, and the function to be predicted is protein coding in human genomic DNA, the query will accordingly require that the sequence returned be genomic and derived from humans.

Query 20 can also incorporate criteria that compel return of sequence that meets operative requirements of the subsequent analytical method. Alternatively, or in addition, such operative criteria can be enforced in subsequent preprocess step 24.

For example, if the function sought to be identified is protein coding, query 20 can incorporate criteria that return from genomic sequence database 100 only those sequences present within contigs sufficiently long as to have obviated substantial fragmentation of any given exon among a plurality of separate sequence fragments.

Such criteria can, for example, consist of a required minimal individual genomic sequence fragment length, such as 10 kb, more typically 20 kb, 30 kb, 40 kb, and preferably 50 kb or more, as well as an optional further or alternative requirement that sequence from any given clone, such as a bacterial artificial chromosome (“BAC”), be presented in no more than a finite maximal number of fragments, such as no more than 20 separate pieces, more typically no more than 15 fragments, even more typically no more than about 10-12 fragments.

Our results have shown that genomic sequence from bacterial artificial chromosomes (BACs) is sufficient for gene prediction analysis according to the present invention if the sequence is at least 50 kb in length, and if additionally the sequence from any given BAC is presented in fewer than 15, and preferably fewer than 10, fragments. Accordingly, query 20 can incorporate a requirement that data accessioned from BAC sequencing be in fewer than 15, preferably fewer than 10, fragments.

An additional criterion that can be incorporated into the query can be the date, or range of dates, of sequence accession. Although the process has been described above as if genomic sequence database 100 were static, it is of course understood that the genomic sequence databases need not be static, and indeed are typically updated on a frequent, even hourly, basis.

Thus, as further described in experimental Examples 1, 2, and 4, infra, it is possible to query the database for newly added sequence, either newly added after an absolute date or newly added relative to a prior analysis performed using the methods and apparatus of the present invention. In this way, the process herein described can incorporate a dynamic, temporal component.

One utility of such temporal limitation is to identify, from newly accessioned genomic sequence, the presence of novel genes, particularly those not previously identified by EST sequencing (or other sequencing efforts that are similarly based upon gene expression). As further described in Example 1, such an approach has shown that newly accessioned human genomic sequence, when analyzed for sequences that function to encode protein, readily identifies genes that are novel over those in existing EST and other expression databases. In fact, as shown below, fully ⅔ of genes identified in newly accessioned human genomic sequence have not hitherto been identified. This makes the methods of the present invention extremely powerful gene discovery tools. And as would be appreciated, such gene discovery can be performed using genomic sequence from species other than human. Particularly useful species are those used as model systems during drug development, such as rodent, particularly mouse and rat, Arabidopsis thaliana, C. elegans, C. brigsii, Drosophila melanogaster, and zebra fish.

If query 20 incorporates multiple criteria, such as above-described, the multiple criteria can be performed as a series of separate queries or as a single query, depending in part upon the query language, the complexity of the query, and other considerations well known in the database arts.

If query 20 returns no genomic sequence meeting the query criteria, the negative result can be reported by process 22, and process 200 (and indeed, entire process 10) ended 23, as shown. Alternatively, or in addition to report and termination of the initial inquiry, a new query 20 can be generated that takes into account the initial negative result.

When query 20 returns sequence meeting the query criteria, the returned sequence is then passed to optional preprocessing 24, suitable and specific for the desired analytical approach and the particular analytical methods thereof to be used in process 25.

Preprocessing 24 can include processes suitable for many approaches and methods thereof, as well as processes specifically suited for the intended subsequent analysis.

Preprocessing 24 suitable for most approaches and methods will include elimination of sequence irrelevant to, or that would interfere with, the subsequent analysis. Such sequence includes repetitive sequence, such as Alu repeats and LINE elements, vector sequence, artificial sequence, such as artificial polylinkers, and the like. Such removal can readily be performed by identification and subsequent masking of the undesired sequence.

Identification can be effected by comparing the genomic sequence returned by query 20 with public or private databases containing known repetitive sequence, vector sequence, artificial sequence, and other artifactual sequence. Such comparison can readily be done using programs well known in the art, such as CROSS_MATCH or REPEATMASKER, the latter available on-line at

http://ftp.genome.washington.edu/RM/RepeatMasker.html, or by proprietary sequence comparison programs the engineering of which is well within the skill in the art.

Alternatively, or in addition, undesirable, including artifactual, sequence can be identified algorithmically without comparison to external databases and thereafter removed. For example, synthetic polylinker sequence can be identified by an algorithm that identifies a significantly higher than average density of known restriction sites. As another example, vector sequence can be identified by algorithms that identify nucleotide or codon usage at variance with that of the bulk of the genomic sequence.

Once identified, undesired sequence can be removed. Removal can usefully be done by masking the undesired sequence as, for example, by converting the specific nucleotide references to one that is unrecognized by the subsequent bioinformatic algorithms, such as “X”. Alternatively, but at present less preferred, the undesired sequence can be excised from the returned genomic sequence, leaving gaps.

Preprocessing 24 can further include selection from among duplicative sequences of that one sequence of highest quality. Higher quality can be measured as a lower percentage of, fewest number of, or least densely clustered occurrence of ambiguous nucleotides, defined as those nucleotides that are identified in the genomic sequence using symbols indicating ambiguity. Higher quality can also or alternatively be valued by presence in the longest contig.

Preprocessing 24 can, and often will, also include formatting of the data as specifically appropriate for passage to the analytical algorithms of process 25. Such formatting can and typically will include, inter alia, addition of a unique sequence identifier, either derived from the original accession number in genomic sequence database 100, or newly applied, and can further include additional annotation. Formatting can include conversion from one to another sequence listing standard, such as conversion to or from FASTA or the like, depending upon the input expected by the subsequent process.

Preprocessing, which can be optional depending upon the function desired to be identified and the informational requirements of the methods for effecting such identification, is followed by sequence processing 25, where sequences with the desired function are identified within the genomic sequence.

As mentioned above, such functions can include, but are not limited to, encoding protein, regulating transcription, regulating message transport after transcription, regulating message splicing after transcription, regulating message degradation after transcription, contributing to or controlling chromosomal somatic recombination, contributing to chromosomal stability or movement, contributing to allelic exclusion or X chromosome inactivation, and the like.

Where the function specified is protein coding, the above-described process can be used rapidly and efficienty to identify individual exons in genomic sequence. As discussed below, we have used the methods and apparatus of the present invention to identify 16,465 exons in human genomic sequence whose expression we have confirmed in at least one human tissue or cell type. Fully two-thirds of the exons initially characterized belong to genes that were not at the time of our discovery represented in existing public expression (EST, cDNA) databases, making the methods and apparatus of the present invention extremely powerful tools for novel gene discovery.

And as further mentioned below and described in detail in commonly owned and copending U.S. patent application Ser. No. 09/632,366, filed Aug. 3, 2000, the disclosure of which is incorporated herein by reference in its entirety, the genome-derived single exon probes and microarrays of the present invention prove exceedingly useful in the high throughput identification of a large variety of alternative splice events in eukaryotic cells and tissues.

To generate such probes, process 25 is used to identify putative coding regions. Two exemplary approaches useful in process 25 for identifying sequence that encodes putative genes are gene prediction and comparative sequence analysis.

Gene prediction can be performed using any of a number of algorithmic methods, embodied in one or more software programs, that identify open reading frames (ORFs) using a variety of heuristics, such as GRAIL, DICTION, GENSCAN, and GENEFINDER.

Comparative sequence analysis similarly can be performed using any of a variety of known programs that identify regions with lower sequence variability.

An advantage of comparative sequence analysis is that genomic sequence can be input into process 200 that is less comprehensive and/or of lesser quality than that required by gene prediction programs.

We have, for example, recently used comparative sequence analysis to identify sequences that are orthologous as between human and mouse genomes, and output the mouse sequences so identified (“similons”) into process 300; this approach has permitted us to identify, and then to confirm and measure expression of, novel mouse exons and genes. As is well known in the pharmaceutical arts, genes identified in model systems permit targets of pharmaceutical intervention to be validated in a model system; validated targets facilitate screening for potential therapeutic lead agents.

As further described in Example 1, below, gene prediction software programs yield a range of results. For the newly accessioned human genomic sequence input in Example 1, for example, GRAIL identified the greatest percentage of genomic sequence as putative coding region, 2% of the data analyzed; GENEFINDER was second, calling 1%; and DICTION yielded the least putative coding region, with 0.8% of genomic sequence called as coding region.

Increased reliability can be obtained when consensus is required among several such methods. Although discussed herein particularly with respect to exon calling, consensus among methods will in general increase reliability of predicting other functions as well.

Thus, as indicated by query 26, sequence processing 25, optionally with preprocessing 24, can be repeated with a different method, with consensus among such iterations determined and reported in process 27.

Process 27 compares the several outputs for a given input genomic sequence and identifies consensus among the separately reported results. The consensus itself, as well as the sequence meeting that consensus, is then stored in process 29a, displayed in process 29b, and/or output to process 300 for subsequent identification of a subset thereof suitable for assay.

Multiple levels of consensus can be calculated and reported by process 27.

For example, as further described in Example 1, infra, process 27 can report consensus as between all specific pairs of methods of gene prediction, as consensus among any one or more of the pairs of methods of gene prediction, or as among all of the gene prediction algorithms used. Thus, in Example 1, process 27 reported that GRAIL and GENEFINDER programs agreed on 0.7% of genomic sequence, that GPAIL and DICTION agreed on 0.5% of genomic sequence, and that the three programs together agreed on 0.25% of the data analyzed. Put another way, 0.25% of the genomic sequence was identified by all three of the programs as containing putative coding region.

As another example, three of the four gene prediction algorithms that we presently use—GENEFINDER, GENSCAN, and GRAIL—predict frame information in addition to the position of exons. If there is overlap in position and frame of the predicted exons, even if not complete identity, the predicted exons are merged in process 27 to generate the largest possible consensus coding region. The process is iterated until all possible overlaps have been merged. This approach reduces the mean number of exons present in each amplicon, and is preferred in generating exon-specific probes useful for detecting exon elongation and exon truncation alternative splice events.

Furthermore, consensus can be required among different approaches to identifying a chosen function.

For example, if the function desired to be identified is coding of protein sequence, and a first used approach to exon calling is gene prediction, the process can be repeated on the same input sequence, or subset thereof, with another approach, such as comparative sequence analysis. In such a case, where comparative sequence analysis follows gene prediction, the comparison can be performed not only on genomic nucleic acid sequence, but additionally or alternatively can be performed on the predicted amino acid sequence translated from exons prior-identified by the gene prediction approach.

Although shown as an iterative process, the multiple analyses required to achieve consensus can be done in series, in parallel, or some combination thereof.

Predicted functional sequence, optionally representing a consensus among a plurality of methods and approaches for determination thereof, is passed to process 300 for identification of a subset thereof for functional assay.

Where the function sought to be identified is protein coding, process 300 is used to identify a subset thereof suitable for experimental verification by physical and/or bioinformatic approaches.

Where the goal is the identification and confirmation of expression of only a single exon of gene, putative exons identified in process 200 can be classified, or binned, bioinformatically into putative genes. This binning can be based inter alia upon consideration of the average number of exons/gene in the species chosen for analysis, upon density of exons that have been called on the genomic sequence, and other empirical rules; the putative gene structure is also provided by various of these gene prediction programs. Thereafter, one or more among the exons can be chosen for subsequent use in gene expression assay.

Where the goal is, instead, the identification and confirmation of expression of all, or of a plurality, of the exons of a gene—as is typically desired for detection of alternative splice events, as further described in commonly owned and copending U.S. patent application Ser. No. 09/632,366, filed Aug. 3, 2000, the disclosure of which is incorporated herein by reference in its entirety—putative exons identified in process 200 can be classified, or binned, bioinformatically into putative genes; thereafter, all of the exons of the gene can be chosen for subsequent confirmation in gene expression assay.

Where such subsequent gene expression assay uses amplified nucleic acid, considerations such as desired amplicon length, primer synthesis requirements, putative exon length, sequence GC content, existence of possible secondary structure, and the like can be used to identify and select those exons that appear most likely successfully to amplify.

Where subsequent gene expression assay relies upon nucleic acid hybridization, whether or not using amplified product, further considerations involving hybridization stringency can be applied to identify that subset of sequences that will most readily permit sequence-specific discrimination at a chosen hybridization and wash stringency. One particular such consideration is avoidance of putative exons that span repetitive sequence; such sequence can hybridize spuriously to nonspecific message, reducing specific signal in the hybridization.

For bioinformatic assay, there are fewer constraints on the sequences that can be tested experimentally, and in this latter case, therefore, process 300 can output the entirety of the input sequence.

The subset of sequences identified by process 300 as suitable for use in assay is then used in process 400 to create the physical and/or informational substrate for experimental verification of the predictions made in process 200, and thereafter to assay those substrates.

Where the goal is to generate single exon nucleic acid probes useful for gene expression analysis, as by microarray hybridization, process 400 is used to confirm expression of the predicted exon. In a particularly useful approach, used herein to confirm expression of the 16,834 genome-derived single exon probes of the present invention, expression is confirmed and measured using genome-derived single exon nucleic acid microarrays, as follows.

Predicted exons are amplified from genomic DNA. Amplification can be performed using the polymerase chain reaction (PCR). Although PCR is conveniently used, other amplification approaches, such as rolling circle amplification, can also be used.

Amplification schemes can be designed to capture the entirety of each predicted exon in an amplicon with minimal additional (that is, flanking intronic or intergenic) sequence. Because exons predicted from genomic sequence using the methods of the present invention differ in length, such an approach results in amplicons of varying length.

However, we have found that most exons predicted from human genomic sequence are shorter than 500 bp in length. Although amplicons of at least about 75 base pairs, more preferably at least about 100 base pairs, even more preferably at least about 200 base pairs can be immobilized as probes on nucleic acid microarrays, our early experimental results using the methods of the present invention suggested that longer amplicons, at least about 400 base pairs, more preferably about 500 base pairs, are more effectively immobilized on glass slides or other prepared surfaces.

Although we had suspected that the intronic and intergenic material flanking putative exons in such longer amplicons might cause interference with exon-specific hybridization during microarray experiments, we have found instead, to our surprise, that the ratio of expression of any such probe as between an experimental tissue (or cell type) and a control tissue is not significantly affected by the presence in the probes of sequence that does not contribute to hybridization to message or cDNA.

Equally surprising, the art had suggested that single exon probes would not provide sufficient signal intensity for high stringency hybridization analyses. Although low stringency hybridization conditions have been designed that permit informative hybridization to highly redundant oligonucleotide-based microarrays, it was believed that the high stringency hybridization conditions typically used for EST-based microarrays would not be usable with single exon probes. We have found, surprisingly, that single-exon probes provide adequate signal at high stringency.

As a result, we have found that we are readily able to use genome-derived amplification products having a single exon flanked by intergenic and/or intronic sequence to confirm the expression of bioinformatically predicted exons.

To the extent that chemical synthesis methods permit oligonucleotides to be generated of sufficient length to encompass an exon, such oligonucleotides can be used as probes in lieu of amplified material. At present, however, amplified products can be generated that exceed the reasonable size limit of chemically synthesized oligonucleotides; amplification thus more readily permits probes to be generated that have single exons flanked by intronic and/or intergenic sequence.

Probes having flanking intergenic and/or intronic sequence permit a wider range of alternative splice events to be detected than do probes that contain only exonic sequence. For example, exon extension would be detectable with such probes as an increase in signal intensity: we have found a near-linear relationship between signal intensity and length of hybridizing sequence. And when used to assay heteronucloear, i.e., immature mRNA, probes having intronic and/or intergenic flanking sequence permit a wider variety of events to be assessed.

Furthermore, certain advantages derive from application to the microarray of amplicons of defined size.

Therefore, amplification schemes can alternatively, and preferably, be designed to amplify regions of defined size, preferably at least about 300 bp, more preferably at least about 400 bp, most preferably about 500 bp, centered about each predicted exon. Such an approach results in a population of amplicons of limited size diversity, but that typically contain intronic and/or intergenic nucleic acid in addition to, and flanking, the putative exon.

Conversely, somewhat fewer than 10% of exons predicted from human genomic sequence according to the methods of the present invention exceed 500 bp in length. Portions of such longer exons, preferably at least about 300 bp, more preferably at least about 400 bp, most preferably about 500 bp, can be amplified. However, in our early experiments we found that the percentage success at amplifying pieces of such exons is low, and that such putative exons are more effectively amplified when larger fragments, at least about 1000 bp, typically at least about 1500 bp, and even as large as 2000 bp are amplified. Further routine optimization of the PCR reaction would permit 500 bp portions of the longer exons to be amplified.

For amplification, the putative exons selected in process 300 are input into one or more primer design programs, such as PRIMER3 (available online for use at

http://www-genome.wi.mit.edu/cgi-bin/primer/),

with a goal of amplifying at least about 500 base pairs of genomic sequence centered within or about exons predicted to be no more than about 500 bp (or at least about 1000-1500 bp of genomic sequence for exons predicted to exceed 500 bp in length) and the primers synthesized by standard techniques. Primers with the requisite sequences can be purchased commercially or synthesized by standard techniques.

Conveniently, a first predetermined sequence can be added commonly to each exon-specific 5′ primer and a second, typically different, predetermined sequence commonly added to each 3′ exon-unique primer. This serves to immortalize the amplicon; that is, it serves to permit further amplification of any amplicon using a single set of primers complementary respectively to the common 5′ and common 3′ sequence elements. The presence of these “universal” priming sequences further facilitates later sequence verification, providing a sequence common to all amplicons at which to prime sequencing reactions. The common 5′ and 3′ sequences can further serve to add a cloning site should any of the exons warrant further study.

Such predetermined sequence is usefully at least about 10 nt in length, typically at least about 12 nt, more typically about 15 nt in length, and usually does not exceed about 25 nt in length. The “universal” priming sequences used in the examples presented infra were each 16 nt long and are further described in commonly owned and copending U.S. patent application Ser. No. 09/608,408, filed Jun. 30, 2000, the disclosure of which is incorporated herein by reference in its entirety. To conserve space, the 5′ and 3′ universal primer sequences are omitted from the probe sequences set forth in the Sequence Listing, the disclosure of which is incorporated herein by reference in its entirety.

The genomic DNA to be used as substrate for amplification will come from the eukaryotic species from which the genomic sequence data had originally been obtained, or a closely related species, and can conveniently be prepared by well known techniques from somatic or germline tissue or cultured cells of the organism. See, e.g., Short Protocols in Molecular Biology: A Compendium of Methods from Current Protocols in Molecular Biology, Ausubel et al. (eds.), 4^{th} edition (April 1999), John Wiley & Sons (ISBN: 047132938X) and Maniatis et al., Molecular Cloning: A Laboratory Manual, 2^{nd} edition (December 1989), Cold Spring Harbor Laboratory Press (ISBN: 0879693096), the disclosures of which are incorporated herein by reference in their entireties. Many such prepared genomic DNAs are available commercially, with the human genomic DNAs typically also having certification of donor informed consent.

After partial purification, as by size exclusion spin column or adsorption to glass, with or without confirmation as to amplicon quality as by gel electrophoresis, each amplicon (single exon probe) is disposed in an array upon a support substrate.

Methods for creating microarrays by deposition and fixation of nucleic acids onto support substrates are well known in the art. Reviewed in Schena (ed.), DNA Microarrays: A Practical Approach (Practical Approach Series), Oxford University Press (1999) (ISBN: 0199637768); Nature Genet. 21(1)(suppl):1-60 (1999); Schena (ed.), Microarray Biochip: Tools and Technology, Eaton Publishing Company/BioTechniques Books Division (2000) (ISBN: 1881299376), the disclosures of which are incorporated herein by reference in their entireties.

Typically, the support substrate can be glass, although other materials, such as amorphous silicon, crystalline silicon, or plastics, can be used. Plastics usefully can include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures or copolymers thereof.

Typically, the support can be rectangular, although other shapes, particularly circular disks and even spheres, present certain advantages. Particularly advantageous alternatives to glass slides as support substrates for array of nucleic acids are optical discs, as described in Demers, “Spatially Addressable Combinatorial Chemical Arrays in CD-ROM Format,” international patent publication WO 98/12559, incorporated herein by reference in its entirety.

The amplified nucleic acids can be attached covalently to a surface of the support substrate or, more typically, applied to a derivatized surface in a chaotropic agent that facilitates denaturation and adherence by presumed noncovalent interactions, or some combination thereof.

Robotic spotting devices useful for arraying nucleic acids on support substrates can be constructed using public domain specifications (The MGuide, version 2.0, http://cmgm.stanford.edu/pbrown/mguide/index.html), or can conveniently be purchased from commercial sources, such as the Molecular Dynamics MicroArray Generation III Array Spotter, which is available from Amersham Pharmacia Biotech (Piscataway, N.J., USA). Spotting can also be effected by printing methods, including those using ink jet technology.

As is well known in the art, microarrays typically also contain immobilized control nucleic acids. For controls useful in providing measurements of background signal for the genome-derived single exon microarrays of the present invention, a plurality of E. coli genes can readily be used. As further described in Example 1, 16 or 32 E. coli genes suffice to provide a robust measure of nonspecific hybridization in such microarrays.

As is well known in the art, the amplified product disposed in arrays on a support substrate to create a nucleic acid microarray can consist entirely of natural nucleotides linked by phosphodiester bonds, or alternatively can include either nonnative nucleotides, alternative internucleoside linkages, or both, so long as complementary binding can be obtained in the hybridization reaction. If enzymatic amplification is used to produce the immobilized probes, the amplifying enzyme will impose certain further constraints upon the types of nucleic acid analogs that can be generated.

Although particularly described herein as using high density microarrays constructed on planar substrates, the methods of the present invention for confirming the expression of exons predicted from genomic sequence can use any of the known types of microarrays, as herein defined, including microarrays on nonplanar, nonunitary, distributed substrates, such as the nonplanar, bead-based microarrays as are described in Brenner et al., Proc. Natl. Acad. Sci. USA 97(4):166501670 (2000); U.S. Pat. Nos. 6,057,107; and 5,736,330, the disclosures of which are incorporated herein by reference in their entireties. In theory, a packed collection of such beads provides in aggregate a higher density of nucleic acid probe than can be achieved with spotting or lithography techniques on a single planar substrate.

In addition, gene expression can be confirmed using hybridization to lower density arrays, such as those constructed on membranes, such as nitrocellulose, nylon, and positively-charged derivatized nylon membranes.

Planar microarrays on solid substrates, however, provide certain useful advantages, including compatibility with existing readers. For example, each standard microscope slide can include at least 1000, typically at least 2000, preferably 5000 or more, and up to 19,000 or more nucleic acid probes of discrete sequence.

Each putative gene can be represented in the array by a single predicted exon or by a plurality of exons predicted to belong to the same gene. And as is well known in the art, each probe of defined sequence, representing a single predicted exon, can be deposited in a plurality of locations on a single microarray to provide redundancy of signal.

The genome-derived single exon microarrays described above differ in several fundamental and advantageous ways from microarrays presently used in the gene expression art, including (1) those created by deposition of mRNA-derived nucleic acids, (2) those created by in situ synthesis of oligonucleotide probes, and (3) those constructed from yeast genomic DNA.

Most nucleic acid microarrays that are in use for study of eukaryotic gene expression have as immobilized probes nucleic acids that are derived—either directly or indirectly—from expressed message. It is common, for example, for such microarrays to be derived from cDNA/EST libraries, either from those previously described in the literature, such as those from the I.M.A.G.E. consortium, Lennon et al., “The I.M.A.G.E. Consortium: an Integrated Molecular Analysis of Genomes and Their Expression, Genomics 33(1):151-2 (1996), or from the de novo construction of “problem specific” libraries targeted at a particular biological question, R. S. Thomas et al., Toxicologist 54:68-69 (2000), incorporated herein by reference in their entireties. Such microarrays are herein collectively denominated “EST microarrays”.

Such EST microarrays by definition can measure expression only of those genes found in EST libraries, which we show herein (see infra) to represent only a fraction of expressed genes. Thus, as further discussed in Example 1, infra, fully ⅔ of genes identified from newly-accessioned human genomic sequence data by the methods of the present invention—for which expression was subsequently confirmed—do not appear in EST or other expression databases, and could not, therefore, have been represented as probes on an EST microarray.

Furthermore, EST and cDNA libraries—and thus microarrays based thereupon—are biased by the tissue or cell type of message origin.

In addition, representation of a message in an EST and/or CDNA library depends upon the successful reverse transcription, optionally but typically with subsequent successful cloning, of the message. This introduces substantial bias into the population of probes available for arraying in EST microarrays. For example, as we show in the Examples, infra, the subset of genes identified from genomic sequence by the methods of the present invention that had previously been accessioned in EST or other expression databases are biased toward genes with higher expression levels.

In contrast, neither reverse transcription nor cloning is required to produce the probes arrayed on the genome-derived single exon microarrays of the present invention. And although the ultimate deposition of a probe on the genome-derived single exon microarray of the present invention depends upon a successful amplification from genomic material, a priori knowledge of the sequence of the desired amplicon affords greater opportunity to recover any given probe sequence recalcitrant to amplification than is afforded by the requirement for successful reverse transcription and cloning of unknown message in EST approaches. Furthermore, if the sequence cannot be amplified, the sequence can at times be chemically synthesized in its entirety for use in the present invention.

Thus, the genome-derived single exon microarrays of the present invention present a far greater diversity of probes for measuring gene expression, with far less bias, than do EST microarrays presently used in the art.

As a further consequence of their ultimate origin from expressed message, the probes in EST microarrays often contain poly-A (or complementary poly-T) stretches derived from the poly-A tail of mature mRNA. These homopolymeric stretches contribute to cross-hybridization, that is, to a spurious signal occasioned by hybridization to the homopolymeric tail of a labeled cDNA that lacks sequence homology to the gene-specific portion of the probe.

In contrast, the probes arrayed in the genome-derived single exon microarrays of the present invention lack homopolymeric stretches derived from message polyadenylation, and thus can provide more specific signal. Typically, at least about 50% of the probes on the genome-derived single exon microarrays of the present invention lack homopolymeric regions consisting of A or T, where a homopolymeric region is defined for purposes herein as stretches of 25 or more, typically 30 or more, identical nucleotides. More typically, at least about 60%, even more typically at least about 75%, of probes on the genome-derived single exon microarrays of the present invention lack such homopolymeric stretches.

A further distinction, which also affects the specificity of hybridization, is occasioned by the typical derivation of EST microarray probes from cloned material. Because much of the probe material disposed as probes on EST microarrays is excised or amplified from plasmid, phage, or phagemid vectors, EST microarrays typically include a fair amount of vector sequence, more so when the probes are amplified, rather than excised, from the vector.

In contrast, the vast majority of probes in the genome-derived single exon microarrays of the present invention contain no prokaryotic or bacteriophage vector sequence, having been amplified directly or indirectly from genomic DNA. Typically, therefore, at least about 50%, more typically at least about 60%, 70%, and even 80% or more of individual exon-including probes disposed on a genome-derived single exon microarray of the present invention lack vector sequence, and particularly lack sequences drawn from plasmids and bacteriophage. Preferably, at least about 85%, more preferably at least about 90%, most preferably more than 90% of exon-including probes in the genome-derived single exon microarray of the present invention lack vector sequence. With attention to removal of vector sequences through preprocessing 24, percentages of vector-free exon-including probes can be as high as 95-99%. The substantial absence of vector sequence from the genome-derived single exon microarrays of the present invention results in greater specificity during hybridization, since spurious cross-hybridization to a probe vector sequence is reduced.

As a further consequence of excision or amplification of probes from vectors in construction of EST microarrays, the probes arrayed thereon often contain artificial sequence, derived from vector polylinker multiple cloning sites, at both 5′ and 3′ ends. The probes disposed upon the genome-derived single exon microarrays need have no such artificial sequence appended thereto.

As mentioned above, however, the exon-specific primers used to amplify putative exons can include artificial sequences, typically 5′ to the exon-specific primer sequence, useful for “universal” (that is, independent of exon sequence) priming of subsequent amplification or sequencing reactions. When such “universal” 5′ and/or 3′ priming sequences are appended to the amplification primers, the probes disposed upon the genome-derived single exon microarray will include artificial sequence similar to that found in EST microarrays. However, the genome-derived single exon microarray of the present invention can be made without such sequences, and if so constructed, presents an even smaller amount of nonspecific sequence that would contribute to nonspecific hybridization.

Yet another consequence of typical use of cloned material as probes in EST microarrays is that such microarrays contain probes that result from cloning artifacts, such as chimeric molecules containing coding region of two separate genes. Derived from genomic material, typically not thereafter cloned, the probes of the genome-derived single exon microarrays of the present invention lack such cloning artifacts, and thus provide greater specificity of signal in gene expression measurements.

A further consequence of the cloned origin of probes on many EST microarrays is that the individual probes often have disparate sizes, which can cause the optimal hybridization stringency to vary among probes on a single microarray. In contrast, as discussed above, the probes arrayed on the genome-derived single exon microarrays of the present invention can readily be designed to have a narrow distribution in sizes, with the range of probe sizes no greater than about 10% of the average size, typically no greater than about 5% of the average probe size.

Because of their origin from fully- or partially-spliced message, probes disposed upon EST arrays will often include multiple exons. The percentage of such exon-spanning probes in an EST microarray can be calculated, on average, based upon the predicted number of exons/gene for the given species and the average length of the immobilized probes. For human genes, the near-complete sequence of human chromosome 22, Dunham et al., Nature 402(6761):489-95 (1999), predicts that human genes average 5.5 exons/gene. Even with probes of 200-500 bp, the vast majority of human EST microarray probes include more than one exon.

In contrast, by virtue of their origin from algorithmically identified exons in genomic sequence, the probes in the genome-derived single exon microarrays of the present invention can comprise individual exons, which provides the ability, as further discussed in commonly owned and copending U.S. patent application Ser. No. 09/632,366, filed Aug. 3, 2000, incorporated herein by reference in its entirety, to detect and to characterize the expression of splice variants.

Although the presence of multiexon probes will not interfere with the ability to confirm expression of predicted exons in a first level screen, it is preferred that at least about 50%, typically at least about 60%, even more typically at least about 70% of probes disposed on the genome-derived microarray of the present invention consist of, or include, no more than one exon. In preferred embodiments, at least about 75%, more preferably at least about 80%, 85%, 90%, 95%, and even 99% of probes in the genome-derived microarrays of the present invention consist of, or include, no more than one exon.

Although, in the most preferred embodiments, at least about 95%, and even at least about 99% of probes in the genome-derived microarray consist of, or include, no more than one exon, we have found that our early bioinformatic parameters typically produce, at this stage of analysis, about 10% of probes that potentially contain two exons. We expect that some fraction of these probes will prove to encode only a single exon, and that further optimization of our bioinformatic approach will reduce the percentage of probes having more than one potential exon. Nonetheless, we exclude such probes from the appended tables and Sequence Listing, incorporated herein by reference in their entireties.

Further distinguishing the genome-derived single exon microarrays of the present invention from the EST arrays in the art, the exons that are represented in EST microarrays are often biased toward the 3′ or 5′ end of their respective genes, since sequencing strategies used for EST identification are so biased. In contrast, no such 3′ or 5′ bias necessarily inheres in the selection of exons for disposition on the genome-derived single exon microarrays of the present invention.

Conversely, the probes provided on the genome-derived single exon microarrays of the present invention typically, but need not necessarily, include intronic and/or intergenic sequence that is absent from EST microarrays, which are derived from mature mRNA. As above-mentioned, such inclusion, although not mandatory, is advantageous, particularly in use of the probes for detection of alternative splice events. Typically, therefore, at least about 50%, more typically at least about 60%, and even more typically at least about 70% of the exon-including probes on the genome-derived single exon microarrays of the present invention include sequence drawn from noncoding regions. In some embodiments, at least about 80%, more typically at least about 85%, 90%, 91%, 92%, 93%, 94%, 95%, 96%, 97%, 98%, and even 99% or more of exon-including probes on the genome-derived single exon microarrays of the present invention will include sequence drawn from noncoding regions.

The genome-derived single exon microarrays of the present invention are also quite different from in situ synthesis microarrays, where probe size is severely constrained by limitations of the photolithographic or other in situ synthesis processes.

Typically, probes arrayed on in situ synthesis microarrays are limited to a maximum of about 25 bp. As a well known consequence, hybridization to such chips must be performed at low stringency. In order, therefore, to achieve unambiguous sequence-specific hybridization results, the in situ synthesis microarray requires substantial redundancy, with concomitant programmed arraying for each probe of probe analogues with altered (i.e., mismatched) sequence.

In contrast, the longer probe length of the genome-derived single exon microarrays of the present invention allows much higher stringency hybridization and wash. Typically, therefore, exon-including probes on the genome-derived single exon microarrays of the present invention average at least about 100 bp, more typically at least about 200 bp, preferably at least about 250 bp, even more preferably about 300 bp, 400 bp, or in preferred embodiments, at least about 500 bp in length. By obviating the need for substantial probe redundancy, this approach permits a higher density of probes for discrete exons or genes to be arrayed on the microarrays of the present invention than can be achieved for in situ synthesis microarrays.

A further distinction is that the probes in in situ synthesis microarrays typically are covalently linked to the substrate surface. In contrast, the probes disposed on the genome-derived microarray of the present invention typically are, but need not necessarily be, bound noncovalently to the substrate.

Furthermore, the short probe size on in situ microarrays causes large percentage differences in the melting temperature of probes hybridized to their complementary target sequence, and thus causes large percentage differences in the theoretically optimum stringency across the array as a whole.

In contrast, the larger probe size in the microarrays of the present invention create lower percentage differences in melting temperature across the range of arrayed probes.

A further significant advantage of the microarrays of the present invention over in situ synthesized arrays is that the quality of each individual probe can be confirmed before deposition. In contrast, the quality of probes cannot be assessed on a probe-by-probe basis for the in situ synthesized microarrays presently being used.

The genome-derived single exon microarrays of the present invention are also distinguished over, and present substantial benefits over, the genome-derived microarrays from lower eukaryotes such as yeast. See, e.g., Lashkari et al., Proc. Natl. Acad. Sci. USA 94:13057-13062 (1997).

Only about 220-250 of the 6100 or so nuclear genes in Saccharomyces cerevisiae—that is, only about 4 to 5%—have standard, spliceosomal, introns, Lopez et al., Nucl. Acids Res. 28:85-86 (2000); Spingola et al., RNA 5(2):221-34 (1999), permitting the ready amplification and disposition of single-exon amplicons on such microarray without the requirement for antecedent use of gene prediction and/or comparative sequence analyses.

A significant aspect of the present invention is the ability to identify and to confirm expression of predicted coding regions in genomic sequence drawn from eukaryotic organisms that have a higher percentage of genes having introns than do yeast such as Saccharomyces cerevisiae, particularly in genomic sequence drawn from eukaryotes in which at least about 10%, typically at least about 20%, more typically at least about 50% of protein-encoding genes have introns. In preferred embodiments, the methods and apparatus of the present invention are used to identify and confirm expression of exons of novel genes from genomic sequence of eukaryotes in which the average number of introns per gene is at least about one, more typically at least about two, even more typically at least about three or more.

After the physical substrate is prepared, experimental verification of predicted function is performed. To verify the expression of the 16,834 single exon probes of the present invention, the single exon microarrays were used in simultaneous two-color hybridization reactions, as follows.

Expression is conveniently measured and reported for each probe in the microarray both as a signal intensity and as a ratio of the expression measured relative to a control, according to techniques well known in the microarray art, reviewed in Schena (ed.), DNA Microarrays: A Practical Approach (Practical Approach Series), Oxford University Press (1999) (ISBN: 0199637768); Nature Genet. 21(1)(suppl):1-60 (1999); Schena (ed.), Microarray Biochip: Tools and Technology, Eaton Publishing Company/BioTechniques Books Division (2000) (ISBN: 1881299376), the disclosures of which are incorporated herein by reference in their entireties. See also Example 2, infra. The mRNA source for the reference (control) used to calculate expression ratios can be heterogeneous, as from a pool of multiple tissues and/or cell types or, alternatively, can be drawn from a homogeneous mRNA source, such as a single cultured cell-type.

In Examples 1 and 2, infra, we used a pool of 10 tissues/cell types as control. We have since observed that almost every probe that demonstrates expression in the control pool can readily be shown to be expressed in HeLa cells. Since use of a pooled control might mask subtle alternative splice events, we have used HeLa as the source of control message in more recent experiments, such as those reported in Example 4, infra.

mRNA can be prepared by standard techniques, Short Protocols in Molecular Biology: A Compendium of Methods from Current Protocols in Molecular Biology, Ausubel et al. (eds.), 4th edition (April 1999), John Wiley & Sons (ISBN: 047132938X) and Maniatis et al., Molecular Cloning: A Laboratory Manual, 2^{nd} edition (December 1989), Cold Spring Harbor Laboratory Press (ISBN: 0879693096), the disclosures of which are incorporated herein by reference in their entireties, or purchased commercially. The mRNA is then typically reverse-transcribed in the presence of labeled nucleotides: the index source (that in which expression is desired to be measured) is reverse transcribed in the presence of nucleotides labeled with a first label, typically a fluorophore (equivalently denominated fluorochrome; fluor; fluorescent dye); the reference source is reverse transcribed in the presence of a second label, typically a fluorophore, typically fluorometrically-distinguishable from the first label.

As further described in Example 2, infra, Cy3 and Cy5 dyes prove particularly useful in these methods. After partial purification of the index and reference targets, hybridization to the probe array is conducted according to standard techniques, typically under a coverslip or in an automatic slide processing unit.

After wash, microarrays are conveniently scanned using a commercial microarray scanning device, such as a Molecular Dynamics Generation III Array Scanner (Amersham Pharmacia Biotech, Piscataway, N.J., USA) or Molecular Dynamics Avalanche Scanner (Amersham Pharmacia Biotech, Piscataway, N.J., USA). Data on expression is then passed, with or without interim storage, to process 500 , where the results for each probe are related to the original sequence.

Although the use of high density genome-derived microarrays on solid planar substrates is presently a preferred approach for the physical confirmation and characterization of the expression of sequences predicted to encode protein, other types of microarrays, as well as lower density macro arrays, can also be used.

Experimental verification in process 400 of the function predicted from genomic sequence in process 200 can be bioinformatic, rather than, or additional to, physical verification. Where the function desired to be identified is protein coding, as in the generation of the 16,834 single exon probes of the present invention, the predicted exons can be compared bioinformatically to sequences known or suspected of being expressed.

Thus, the sequences output from process 300 (or process 200), can be used to query expression databases, such as EST databases, SNP (“single nucleotide polymorphism”) databases, known cDNA and mRNA sequences, SAGE (“serial analysis of gene expression”) databases, and more generalized sequence databases that allow query for expressed sequences. Such query can be done by any sequence query algorithm, such as BLAST (“basic local alignment search tool”). The results of such query—including information on identical sequences and information on nonidentical sequences that have diffuse or focal regions of sequence homology to the query sequence—can then be passed directly to process 500, or used to inform analyses subsequently undertaken in process 200, process 300, or process 400.

Experimental data, whether obtained by physical or bioinformatic assay in process 400, is passed to process 500 where it is usefully related to the sequence data itself, a process colloquially termed “annotation”. Such annotation can be done using any technique that usefully relates the functional information to the sequence, as, for example, by incorporating the functional data into the record itself, by linking records in a hierarchical or relational database, by linking to external databases, or by a combination thereof. Such database techniques are well within the skill in the art.

The annotated sequence data can be stored locally, uploaded to genomic sequence database 100, and/or displayed 800.

Display of Annotated Genomic Sequence

The methods and apparatus of the present invention rapidly produce functional information from genomic sequence. As is further discussed below, we have, for example, used the methods and apparatus of the present invention to identify 16,465 exons (in 16,834 separate single exon probes) in human genomic sequence whose expression we have confirmed in at least one human tissue or cell type. Fully two-thirds of the exons belong to genes that were not then represented in existing public expression (EST, cDNA) databases. We have also used these single exon probes to identify alternative splice events in novel genes.

Coupled with the escalating pace at which sequence now accumulates, the ability rapidly to identify and confirm the function of regions of genomic DNA provided by the present invention produces a need for methods of displaying the information in meaningful ways. It is, therefore, another aspect of the present invention to provide means for displaying annotated sequence, and in particular for displaying sequence annotated according to the methods and apparatus of the present invention. Further, such display can be used as a preferred graphical user interface for electronic search, query, and analysis of such annotated sequence.

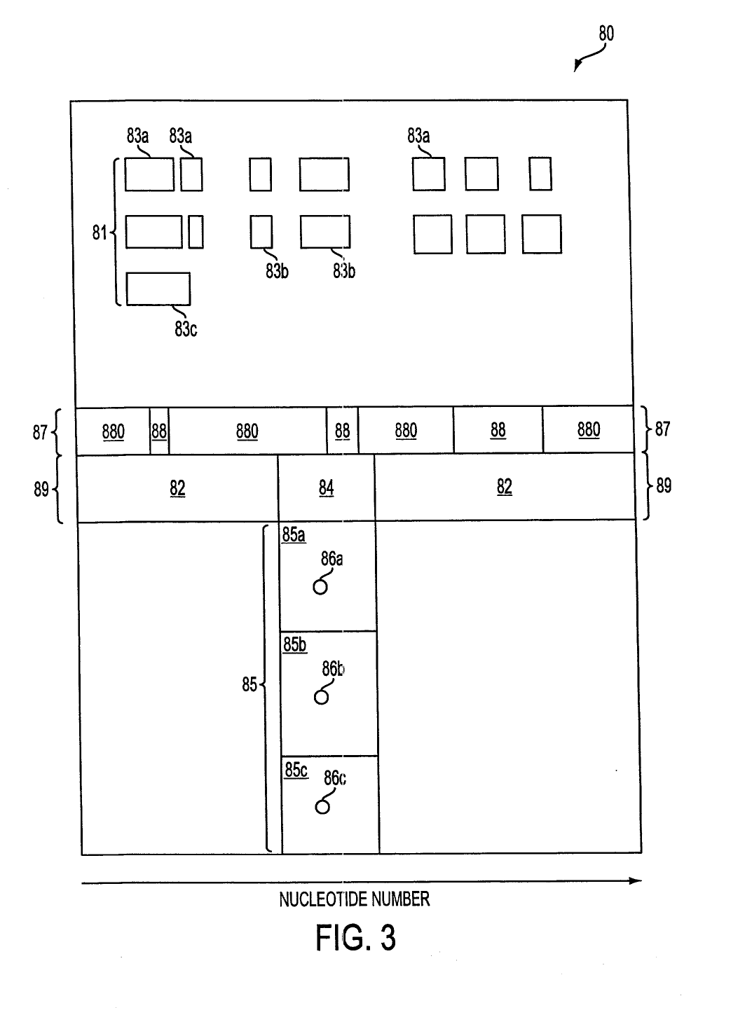

FIG. 3 schematizes visual display 80 presenting a single genomic sequence annotated according to the present invention. Because of its nominal resemblance to artistic works of Piet Mondrian, visual display 80 is alternatively described herein as a “Mondrian”.

Each of the visual elements of display 80 is aligned with respect to the genomic sequence being annotated (the “annotated sequence”). Given the number of nucleotides typically represented in an annotated sequence, representation of individual nucleotides would rarely be readable in hard copy output of display 80. Typically, therefore, the annotated sequence is schematized as rectangle 89, extending from the left border of display 80 to its right border. By convention herein, the left border of rectangle 89 represents the first nucleotide of the sequence and the right border of rectangle 89 represents the last nucleotide of the sequence.

As further discussed below, however, the Mondrian visual display of annotated sequence can serve as a convenient graphical user interface for computerized representation, analysis, and query of information stored electronically. For such use, the individual nucleotides can conveniently be linked to the X axis coordinate of rectangle 89. This permits the annotated sequence at any point within rectangle 89 readily to be viewed, either automatically—for example, by time-delayed appearance of a small overlaid window (“tool tip”) upon movement of a cursor or other pointer over rectangle 89—or through user intervention, as by clicking a mouse or other pointing device at a point in rectangle 89.

Visual display 80 is generated after user specification of the genomic sequence to be displayed. Such specification can consist of or include an accession number for a single clone (e.g., a single BAC accessioned into GenBank), wherein the starting and stopping nucleotides are thus absolutely identified, or alternatively can consist of or include an anchor or fulcrum point about which a chosen range of sequence is anchored, thus providing relative endpoints for the sequence to be displayed. For example, the user can anchor such a range about a given chromosomal map location, gene name, or even a sequence returned by query for similarity or identity to an input query sequence. When visual display 80 is used as a graphical user interface to computerized data, additional control over the first and last displayed nucleotide will typically be dynamically selectable, as by use of standard zooming and/or selection tools.

Field 81 of visual display 80 is used to present the output from process 200, that is, to present the bioinformatic prediction of those sequences having the desired function within the genomic sequence. Functional sequences are typically indicated by at least one rectangle 83 (83a, 83b, 83c), the left and right borders of which respectively indicate, by their X-axis coordinates, the starting and ending nucleotides of the region predicted to have function.

Where a single bioinformatic method or approach identifies a plurality of regions having the desired function, a plurality of rectangles 83 is disposed horizontally in field 81. Where multiple methods and/or approaches are used to identify function, each such method and/or approach can be represented by its own series of horizontally disposed rectangles 83, each such horizontally disposed series of rectangles offset vertically from those representing the results of the other methods and approaches.

Thus, rectangles 83a in FIG. 3 represent the functional predictions of a first method of a first approach for predicting function, rectangles 83b represent the functional predictions of a second method and/or second approach for predicting that function, and rectangles 83c represent the predictions of a third method and/or approach.

Where the function desired to be identified is protein coding, field 81 is used to present the bioinformatic prediction of sequences encoding protein. For example, rectangles 83a can represent the results from GRAIL or GRAIL II, rectangles 83b can represent the results from GENEFINDER, and rectangles 83c can represent the results from DICTION.

Optionally, and preferably, rectangles 83 collectively representing predictions of a single method and/or approach are identically colored and/or textured, and are distinguishable from the color and/or texture used for a different method and/or approach.

Alternatively, or in addition, the color, hue, density, or texture of rectangles 83 can be used further to report a measure of the bioinformatic reliability of the prediction. For example, many gene prediction programs will report a measure of the reliability of prediction. Thus, increasing degrees of such reliability can be indicated, e.g., by increasing density of shading. Where display 80 is used as a graphical user interface, such measures of reliability, and indeed all other results output by the program, can additionally or alternatively be made accessible through linkage from individual rectangles 83, as by time-delayed window (“tool tip” window), or by pointer (e.g., mouse)-activated link.

As described above, increased predictive reliability can be achieved by requiring consensus among methods and/or approaches to determining function. Thus, field 81 can include a horizontal series of rectangles 83 that indicate one or more degrees of consensus in predictions of function, including the combined length of the separately predicted exons that overlap in frame.

Although FIG. 3 shows three series of horizontally disposed rectangles in field 81, display 80 can include as few as one such series of rectangles and as many as can discriminably be displayed, depending upon the number of methods and/or approaches used to predict a given function. For example, addition of a fourth gene prediction program, such as GENSCAN

(http://genes.mit.edu/GENSCANinfo.html),

to the three gene prediction programs used in our first experiments (GRAIL, GENEFINDER, DICTION) would be accommodated by a fourth series of rectangles disposed horizontally in field 81, but offset vertically from rectangles 81a, 81b, and 81c.

Furthermore, field 81 can be used to show predictions of a plurality of different functions. However, the increased visual complexity occasioned by such display makes more useful the ability of the user to select a single function for display. When display 80 is used as a graphical user interface for computer query and analysis, such function can usefully be indicated and user-selectable, as by a series of graphical buttons or tabs (not shown in FIG. 3).

Rectangle 89 is shown in FIG. 3 as including interposed rectangle 84. Rectangle 84 represents the portion of annotated sequence for which predicted functional information has been assayed physically, with the starting and ending nucleotides of the assayed material indicated by the X axis coordinates of the left and right borders of rectangle 84. Rectangle 85, with optional inclusive circles 86 (86a, 86b, and 86c) displays the results of such physical assay.

Although a single rectangle 84 is shown in FIG. 3, physical assay is not limited to just one region of annotated genomic sequence. It is expected that an increasing percentage of regions predicted to have function by process 200 will be assayed physically, and that display 80 will accordingly, for any given genomic sequence, have an increasing number of rectangles 84 and 85, representing an increased density of sequence annotation. For example, for purposes of generating exon-specific probes for alternative splice detection, it is preferred that a plurality of exons, preferably all of the exons, that commonly belong to a single gene will be assayed experimentally for expression; accordingly, display 80 will have, for the genomic sequence encompassing such exons, a series of rectangles 84 and 85 for each of the assayed exons.

Where the function desired to be identified is protein coding, rectangle 84 identifies the sequence of the probe used to measure expression. In embodiments of the present invention where expression is measured using genome-derived single exon microarrays, rectangle 84 identifies the sequence included within the probe immobilized on the solid support surface of the microarray. As noted supra, such probe will often include a small amount of additional, synthetic, material incorporated during amplification and designed to permit reamplification of the probe, which sequence is typically not shown in display 80.

Rectangle 87 is used to present the results of bioinformatic assay of the genomic sequence. For example, where the function desired to be identified is protein coding, process 400 can include bioinformatic query of expression databases with the sequences predicted in process 200 to encode exons. And as discussed above, because bioinformatic assay presents fewer constraints than does physical assay, often the entire output of process 200 can be used for such assay, without further subsetting thereof by process 300. Therefore, rectangle 87 typically need not have separate indicators therein of regions submitted for bioinformatic assay; that is, rectangle 87 typically need not have regions therein analogous to rectangles 84 within rectangle 89.

Rectangle 87 as shown in FIG. 3 includes smaller rectangles 880 and 88. Rectangles 880 indicate regions that returned a positive result in the bioinformatic assay, with rectangles 88 representing regions that did not return such positive results. Where the function desired to be predicted and displayed is protein coding, rectangles 880 indicate regions of the predicted exons that identify sequence with significant similarity in expression databases, such as EST, SNP, SAGE databases, with rectangles 88 indicating genes novel over those identified in existing expression data bases.

Rectangles 880 can further indicate, through color, shading, texture, or the like, additional information obtained from bioinformatic assay.

For example, where the function assayed and displayed is protein coding, the degree of shading of rectangles 880 can be used to represent the degree of sequence similarity found upon query of expression databases. The number of levels of discrimination can be as few as two (identity, and similarity, where similarity has a user-selectable lower threshold).

Alternatively, as many different levels of discrimination can be indicated as can visually be discriminated.

Where display 80 is used as a graphical user interface, rectangles 880 can additionally provide links directly to the sequences identified by the query of expression databases, and/or statistical summaries thereof. As with each of the precedingly-discussed uses of display 80 as a graphical user interface, it should be understood that the information accessed via display 80 need not be resident on the computer presenting such display, which often will be serving as a client, with the linked information resident on one or more remotely located servers.

Rectangle 85 displays the results of physical assay of the sequence delimited by its left and right borders.

Rectangle 85 can consist of a single rectangle, thus indicating a single assay, or alternatively, and increasingly typically, will consist of a series of rectangles (85a, 85b, 85c) indicating separate physical assays of the same sequence.

Where the function assayed is gene expression, and where gene expression is assayed as herein described using simultaneous two-color fluorescent detection of hybridization to genome-derived single exon microarrays, individual rectangles 85 can be colored to indicate the degree of expression relative to control. Conveniently, shades of green can be used to depict expression in the sample over control values, and shades of red used to depict expression less than control, corresponding to the spectra of the Cy3 and Cy5 dyes conventionally used for respective labeling thereof.

Additional functional information can be provided in the form of circles 86 (86a, 86b, 86c), where the diameter of the circle can be used to indicate a parameter different from that set forth in rectangle 85. For example, where the annotated functions are the distribution of expression of the one or more predicted exons, rectangle 85 can report expression relative to control and circle 86 can be used to report signal intensity. As discussed infra, such relative expression (expression ratio) and absolute expression (signal intensity) can be expressed using normalized values.

Where display 80 is used as a graphical user interface, rectangle 85 can be used as a link to further information about the assay. For example, where the assay is one for gene expression, each rectangle 85 can be used to link to information about the source of the hybridized mRNA, the identity of the control, raw or processed data from the microarray scan, or the like.

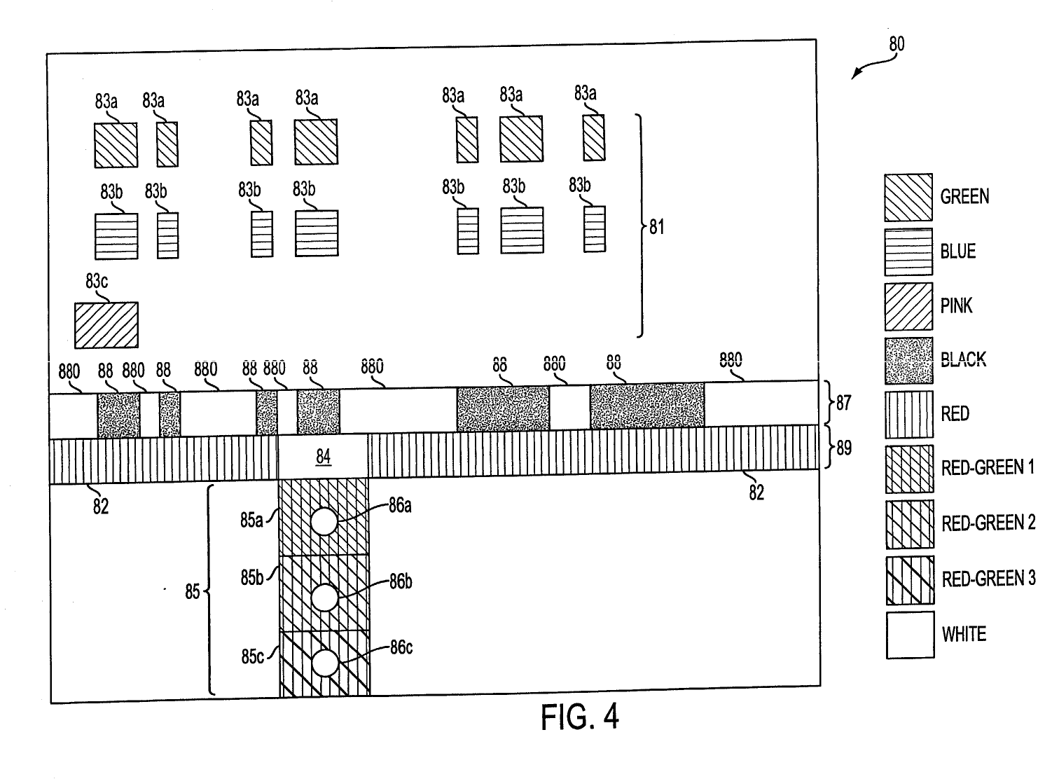

For purposes of illustration only, FIG. 4 shows an embodiment of display 80 showing typical color conventions when hypothetical genomic sequence is annotated with exon-specific expression data. As would of course readily be understood, the color choice is arbitrary, and alternative colors can be used.

In this typical presentation, BAC sequence (“Chip seq.”) 89 is presented in red, with the physically assayed region thereof (corresponding to rectangle 84 in FIG. 3) shown in white. Algorithmic gene predictions are shown in field 81, with predictions by GRAIL shown in green, predictions by GENEFINDER shown in blue, and predictions by DICTION shown in pink. Within rectangle 87, regions of sequence that, when used to query expression databases, return identical or similar sequences (“EST hit”) are shown as white rectangles (corresponding to rectangles 880 in FIG. 3), gray indicates low homology, and black indicates unknowns (where black and gray would correspond to rectangles 88 in FIG. 3).

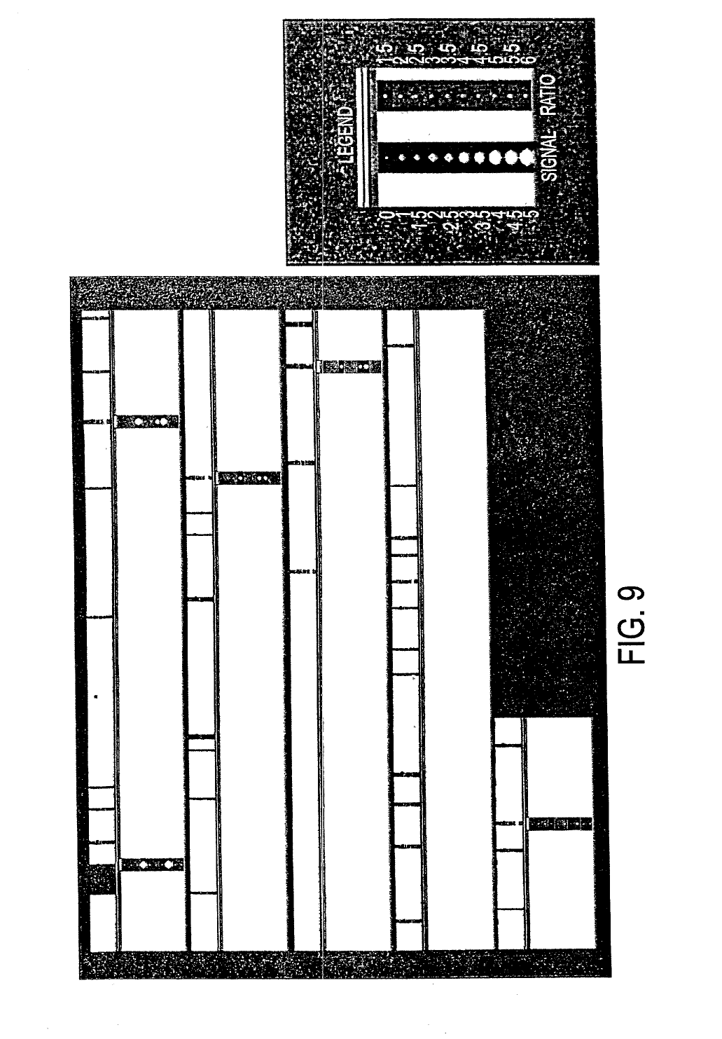

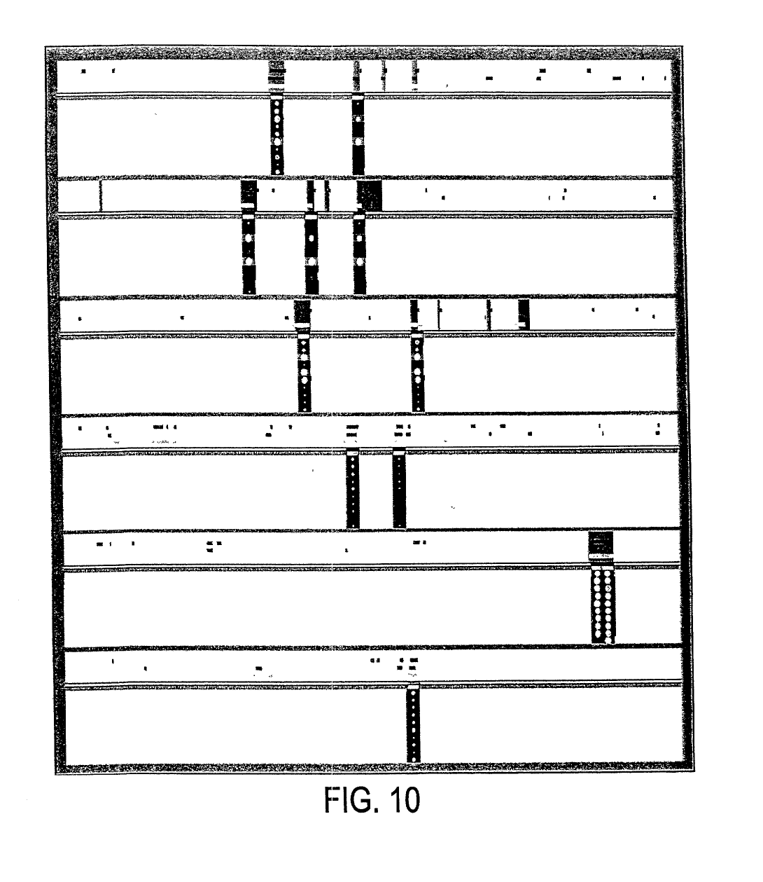

Although FIGS. 3 and 4 show a single stretch of sequence, uninterrupted from left to right, longer sequences are usefully represented by vertical stacking of such individual Mondrians, as shown in FIGS. 9 and 10.

Using our visual display tool, the Mondrian, we have found that consensus in the pattern of expression of individual exons is a powerful means for identifying exons that commonly belong to a single gene. It is, therefore, another aspect of the present invention to provide methods, including methods based upon visual display, for associating exons that commonly belong to a single gene using, as the criterion for association, consensus in their patterns of expression in a plurality of tissues and/or cell types.

As further discussed in Example 3, FIG. 9 presents a Mondrian of BAC AC008172 (bases 25,000 to 130,000 shown), containing the carbamyl phosphate synthetase gene (AF154830.1), the sequence and structure of which has previously been reported. Purple background within the region shown as field 81 in FIG. 3 indicates all 37 known exons for this gene.

As can be seen, GRAIL II successfully identified 27 of the known exons (73%), GENEFINDER successfully identified 37 of the known exons (100%), while DICTION identified 7 of the known exons (19%).

Seven of the predicted exons were selected for physical assay, of which 5 successfully amplified by PCR and were sequenced. These five exons were all found to be from the same gene, the carbamyl phosphate synthetase gene (AF154830.1).

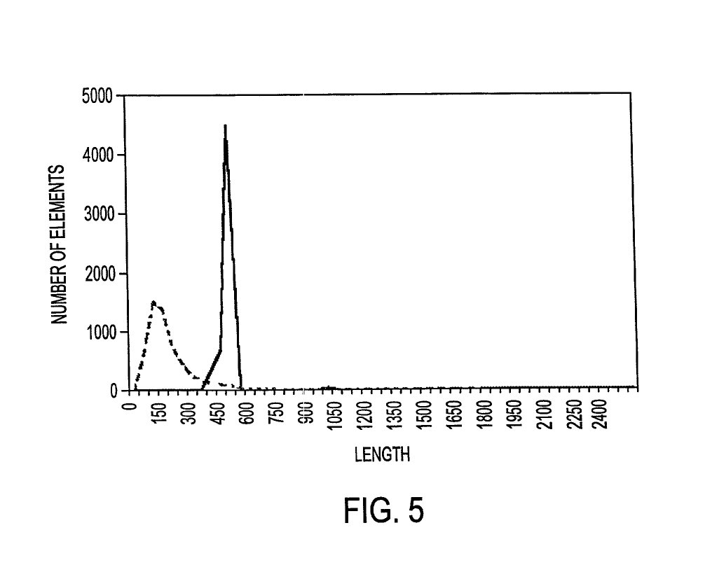

The five exons were arrayed and gene expression measured across 10 tissues. As is readily seen by visual inspection of the resulting Mondrian (FIG. 5), the five single-exon probes report identical expression ratio patterns: each exon is expressed above control (i.e., in green) in the tissues represented by the fourth, seventh, and eighth rectangles (corresponding to rectangles 85 in FIG. 3) and is expressed at or below control in the remaining tissues.

Of course, an exon that is removed or truncated by alternatively splicing in one of the assayed tissues would produce a variant expression pattern. For purposes of associating exons as belonging commonly to a single gene, however, a consensus among assayed tissues would still identify the exon as presumptively belonging to the same gene.

The methods of this aspect of the invention can, and typically will, be automated. For example, WO 99/58720, incorporated herein by reference in its entirety, describes algorithms for ordering the relatedness of a plurality of multidimensional expression data sets. The methods set forth therein can readily be adapted to ordering the relatedness of data sets, wherein each data set comprises expression ratios of an individual exon across a plurality of tissues and cell types, permitting exons with related, but not necessarily identical, patterns of expression to be classified as belonging to a common gene.

Utility of Genome-Derived Single Exon Probes and Microarrays

The methods and apparatus above-described rapidly produce functional information from genomic sequence. When the functions to be identified are protein coding and the distribution of expression of the predicted exon, the methods and apparatus of the present invention rapidly identify and confirm the expression of portions of genomic sequence that function to encode protein. Using these methods and apparatus, we have, for example, generated 16,834 single exon probes that demonstrate significant expression in one or more of ten tested human tissues or cell types.

As would immediately be appreciated by one of skill in the art, each single exon probe having demonstrable expression in one or more tissues is currently available for use in measuring the level of its exon's expression in each of the tissues in which expression has been confirmed. The utility is specific to the probe; under high stringency hybridization conditions, each probe reports the level of expression of message specifically containing that exon.

Accordingly, each single exon probe of the present invention, each of which has been shown to have demonstrable expression in one or more tissues or cell types, is currently available as a tool for specifically measuring the expression of the probe's exon in at least one tissue or cell type. Sequences of the probes and respective expression data can be found in the appended Sequence Listing and Tables 4-13, incorporated herein by reference.

Measuring tools are well known in many arts, not just in molecular biology, and are known to possess credible, specific, and substantial utility. For example, U.S. Pat. No. 6,016,191 describes and claims a tool for measuring characteristics of fluid flow in a hydrocarbon well; U.S. Pat. No. 6,042,549 describes and claims a device for measuring exercise intensity; U.S. Pat. No. 5,889,351 describes and claims a device for measuring viscosity and for measuring characteristics of a fluid; U.S. Pat. No. 5,570,694 describes and claims a device for measuring blood pressure; U.S. Pat. No. 5,930,143 describes and claims a device for measuring the dimensions of machine tools; U.S. Pat. No. 5,279,044 describes and claims a measuring device for determining an absolute position of a movable element; U.S. Pat. No. 5,186,042 describes and claims a device for measuring action force of a wheel, and U.S. Pat. No. 4,246,774 describes and claims a device for measuring the draft of smoking articles such as cigarettes.

As for those genome-derived single exon probes identified by the methods of the present invention, for which expression has not yet been demonstrated (and that are, on that basis, excluded from the appended Sequence Listing), such probes are currently available as tools for surveying tissues to detect the presence of expressed messages that contain their specific exon.

Survey tools—i.e., tools for determining the presence and/or location of a desired object by search of an area—are well known in many arts, not just in molecular biology, and are known to possess credible, specific, and substantial utility. For example, U.S. Pat. No. 6,046,800 describes and claims a device for surveying an area for objects that move; for example, U.S. Pat. No. 6,025,201 describes and claims an apparatus for locating and discriminating platelets from non-platelet particles or cells on a cell-by-cell basis in a whole blood sample; U.S. Pat. No. 5,990,689 describes and claims a device for detecting and locating anomalies in the electromagnetic protection of a system, and U.S. Pat. No. 5,984,175 describes and claims a device for detecting and identifying wearable user identification units.

It will be appreciated, of course, that those probes for which expression has been demonstrated are useful for both measurement in the tissues for which expression has been confirmed and for survey in other tissues.

It will also be appreciated that the inability to demonstrate expression of certain of the probes predicted by the methods of the present invention, making such probes more useful as survey, than as measurement, tools, confers upon these probes some of their useful advantages over prior nucleic acid probes.

Significant among such advantages is the presence of probes for novel genes.

As mentioned above and further detailed in Examples 1 and 2, infra, fully ⅔ of the exons identified using the methods of the present invention are not present in existing expression databases. And the fewer the number of tissues in which the exon can be shown to be expressed, the more likely the exon will prove to be part of a novel gene: as further discussed in Example 2, exons whose expression was measurable in only a single of the tested tissues were represented in existing expression databases at a rate of only 11%, whereas 36% of exons whose expression was measurable in 9 tissues were present in existing expression databases, and fully 45% of those exons expressed in all ten tested tissues were present in existing expressed sequence databases.

Thus, the genome-derived single exon probes of the present invention that have not yet been shown to be expressed in one of our ten standard tissues will likely contain the largest percentage of unknown genes.

And it should further be appreciated that the genome-derived single exon probes of the present invention for which significant expression has not yet been shown, and that are thus useful principally as survey tools, will later be demonstrated to be expressed in other tissues, cell types, and/or developmental stages, and will thus assume substantial additional utility as tools for measuring specific gene expression.

Either as tools for measuring gene expression or tools for surveying gene expression, the genome-derived single exon probes of the present invention have significant advantages over the cDNA or EST-based probes that are currently available for achieving these utilities.

As mentioned earlier with respect to the genome-derived single exon microarrays used to confirm expression of predicted exons, which discussion is incorporated herein by reference in its entirety, the genome-derived single exon probes of the present invention advantageously lack the biases inherent in probes drawn from EST libraries, such as expression level bias, 3′ or 5′ end-bias, bias imposed by reverse transcription, bias imposed by cloning limitations; advantageously lack the homopolymeric stretches often seen in probes derived from expressed message; advantageously lack the prokaryotic or bacteriophage vector sequence often seen in probes derived from expressed message; advantageously lack cloning artifacts; and provide a much enhanced ability to monitor the expression of individual exons, and thus to detect alternative splicing events.

The genome-derived single exon probes of the present invention are useful in constructing genome-derived single exon microarrays; the genome-derived single exon microarrays, in turn, are saleable products of manufacture that are useful for measuring and for surveying gene expression.

When included on a microarray, each genome-derived single exon probe of the present invention makes the microarray specifically useful for detecting the probe's specific exon, thus imparting upon the microarray device the ability to detect a signal where, absent such probe, it would have reported no signal. This utility makes each individual probe on such microarray akin to an antenna, circuit, firmware or software element included in an electronic apparatus, where the antenna, circuit, firmware or software element imparts upon the apparatus the ability newly and additionally to detect signal in a portion of the radio-frequency spectrum where previously it could detect no signal; such devices are known to have specific, substantial, and credible utility.

Gene expression analysis using microarrays—conventionally using microarrays having probes derived from expressed message—is well-established as useful.

For example, Kaminski et al., “Global Analysis of Gene Expression in Pulmonary Fibrosis Reveals Distinct Programs Regulating Lung Inflammation and Fibrosis,” Proc. Natl. Acad. Sci. USA 97(4):1778-83 (2000), incorporated herein by reference, describe the use of microarrays to analyze the gene expression programs that underlie pulmonary fibrosis in a mouse model; the analysis identified two distinct groups of genes involved in the time-dependent inflammatory and fibrotic responses to the drug bleomycin.

For example, Livesey et al., “Microarray Analysis of the Transcriptional Network Controlled by the Photoreceptor Homeobox Gene Crx,” Curr. Biol. 10(6):301-310 (2000), incorporated herein by reference, used microarrays to study the transcriptional network of the Crx transcription factor, which is preferentially expressed in the retina, and mutation of which results in congenital blindness or photoreceptor degeneration in humans; the analysis identified genes differentially expressed as between mutated mice which completely lacked the mouse version of the Crx gene and normal mice.

For example, Geiss et al., “Large-scale Monitoring of Host Cell Gene Expression During HIV-1 Infection Using cDNA Microarrays,” Virology 266(1):8-16 (2000), incorporated herein by reference, used nucleic acid microarrays to monitor the expression of approximately 1500 cellular cDNAs in infected CD4⁺ T-cells: twenty cellular genes were identified as differentially expressed at 3 days post infection, and thus promising targets for development of anti-HIV drugs.

For example, Alon et al., “Broad Patterns of Gene Expression Revealed by Clustering Analysis of Tumor and Normal Colon Tissues Probed by Oligonucleotide Arrays,” Proc. Natl. Acad. Sci. USA 96(12):6745-50 (1999), incorporated herein by reference, describe use of microarrays to distinguish colon cancer samples from samples of normal colon tissue based on subtle differences in gene expression patterns, even when expression of individual genes varied only slightly between the tissues.

For example, Scherf et al., “A Gene Expression Database for the Molecular Pharmacology of Cancer,” Nat. Genet. 24(3):236-44 (2000), incorporated herein by reference, used microarrays to assess gene expression profiles in 60 human cancer cell lines used in drug discovery screens by the National Cancer Institute; the expression data were then correlated to drug responsiveness to identify particular genes, the expression of which may predict drug sensitivity and resistance.

For example, Alizadeh et al., “Distinct types of diffuse large B-cell lymphoma identified by gene expression profiling,” Nature 403(6769):503-11 (2000), incorporated herein by reference, used microarrays to stratify patients having diffuse large B-cell lymphoma according to expression patterns indicative of different stages of B-cell differentiation, providing improved diagnostic resolution.

For example, Perou et al., “Distinctive Gene Expression Patterns in Human Mammary Epithelial Cells and Breast Cancers, Proc. Natl. Acad. Sci. USA 96(16):9212-7 (1999), incorporated herein by reference, used microarrays to identify patterns of gene expression in human mammary epithelial cells growing in culture and in primary human breast tumors. Two such clusters were found to have patterns that correlated with variation in cell proliferation rates and with activation of the IFN-regulated signal transduction pathway, respectively. This analysis demonstrates that variation in gene expression patterns in human cancers can serve as a means to classify solid tumors.

For example, Sgroi et al., “In vivo Gene Expression Profile Analysis of Human Breast Cancer Progression,” Cancer Res. 59(22):5656-61 (1999), incorporated herein by reference, used cDNA microarrays to monitor in vivo gene expression levels in purified normal, invasive, and metastatic breast cell populations from a single patient; the analysis demonstrated that microarrays can be used to track gene expression changes associated with progression of cancer, with diagnostic, prognostic, and therapeutic implications.

For example, Wang et al., “Identification of Genes Differentially Over-expressed in Lung Squamous Cell Carcinoma Using Combination of cDNA Subtraction and Microarray Analysis,” Oncogene 19(12):1519-28 (2000), incorporated herein by reference, used a combination of cDNA library subtraction and microarrays to identify seventeen genes preferentially over-expressed in lung squamous cell carcinoma, including four novel genes; the analysis demonstrated that microarrays can be used to identify tumor-specific genes useful for diagnosis and therapy.

For example, Whitney et al., “Analysis of Gene Expression in Multiple Sclerosis Lesions Using cDNA Microarrays,” Ann. Neurol. 46(3):425-8 (1999), incorporated herein by reference, used microarrays to monitor the expression pattern of over 5,000 genes and compare the gene expression profile of normal white matter to that found in acute lesions from the brain of a single patient with multiple sclerosis (MS); sixty-two differentially expressed genes were identified.

For example, Shelton et al., “Microarray Analysis of Replicative Senescence,” Curr. Biol. 9(17):939-45 (1999), incorporated herein by reference, used microarrays to study gene expression associated with cell aging, or senescence, an arrested state in which cells remain viable but stop replicating and display an altered pattern of gene and protein expression, in dermal fibroblasts, retinal pigment epithelial cells, and vascular endothelial cells. Comparison of early- and late-passage cells stimulated with serum showed specific deficits in the early and mid G1 response of senescent cells, demonstrating that microarrays can be used to identify genes involved in the cellular aging process.

For example, Voehringer et al., “Gene Microarray Identification of Redox and Mitochondrial Elements That Control Resistance or Sensitivity to Apoptosis,” Proc. Natl. Acad. Sci. USA 97(6):2680-5 (2000), incorporated herein by reference, used microarrays to study gene expression patterns in an apoptosis-sensitive and apoptosis-resistant murine B cell lymphoma model system before and after irradiation; the analysis demonstrated that microarrays can be used to explore a fundamental cell biological process, programmed cell death, that figures prominently in the ability of cancer cells to survive exposure to chemotherapeutic agents and tumor killing radiation.

For example, Heller et al., “Discovery and Analysis of Inflammatory Disease-related Genes Using cDNA Microarrays,” Proc. Natl. Acad. Sci. USA 94(6):2150-5 (1997), incorporated herein by reference, used cDNA microarrays to profile gene expression associated with rheumatoid arthritis and inflammatory bowel disease; the cytokine IL-3, the chemokine Gro alpha, and the metalloproteinase matrix metallo-elastase were implicated in both diseases, whereas tissue inhibitor of metalloproteinase 1, ferritin light chain, and manganese superoxide dismutase genes were identified as differentially expressed between the two diseases.

For example, Nuwaysir et al., “Microarrays and Toxicology: the Advent of Toxicogenomics,” Mol. Carcinog. 24(3):153-9 (1999), incorporated herein by reference, used cDNA microarrays to measure gene expression as a means to identify compounds potentially toxic to humans and the environment, and to determine their mechanism of action.

For example, Bartosiewicz et al., “Development of a Toxicological Gene Array and Quantitative Assessment of This Technology,” Arch. Biochem. Biophys. 376(1):66-73 (2000), incorporated herein by reference, used microarrays containing expressed sequence tags for xenobiotic metabolizing enzymes, proteins associated with glutathione regulation, DNA repair enzymes, heat shock proteins, and housekeeping genes to examine gene expression in mouse liver in response to administration of beta-naphthoflavone (beta-NF); the analysis demonstrated that microarrays can be used to observe the effects of toxic compounds on gene expression in organs, such as liver, responsible for their metabolism.

In such toxicologic screens, and analogously in microarray-based gene expression screens of pharmacologic drug candidates upon cells, each probe provides specific useful data. In particular, it should be appreciated that even those probes that show no change in expression are as informative as those that do change, serving, in essence, as negative controls.

For example, where gene expression analysis is used to assess toxicity of chemical agents on cells, the failure of the agent to change a gene's expression level is evidence that the drug likely does not affect the pathway of which the gene's expressed protein is a part. Analogously, where gene expression analysis is used to assess side effects of pharmacologic agents—whether in lead compound discovery or in subsequent screening of lead compound derivatives—the inability of the agent to alter a gene's expression level is evidence that the drug does not affect the pathway of which the gene's expressed protein is a part.

WO 99/58720, incorporated herein by reference in its entirety, provides methods for quantifying the relatedness of a first and second gene expression profile and for ordering the relatedness of a plurality of gene expression profiles. The methods so described permit useful information to be extracted from a greater percentage of the individual gene expression measurements from a microarray than methods previously used in the art. Furthermore, the function of the genes need not be known for the level of expression to provide useful information.

Other uses of microarrays are described in Gerhold et al., “DNA Chips: Promising Toys Have Become Powerful Tools,” Trends Biochem. Sci. 24(5):168-173 (1999) and Zweiger, “Knowledge Discovery in Gene-expression-microarray Data: Mining the Information Output of the Genome,” Trends Biotechnol. 17(11):429-436 (1999); Schena (ed.), DNA Microarrays: A Practical Approach (Practical Approach Series), Oxford University Press (1999) (ISBN: 0199637768); Nature Genet. 21(1) (suppl):1-60 (1999); Schena (ed.), Microarray Biochip: Tools and Technology, Eaton Publishing Company/BioTechniques Books Division (2000) (ISBN: 1881299376), the disclosures of each of which is incorporated herein by reference in its entirety.

The genome-derived single exon microarrays of the present invention have each of the above-described well-established utilities in gene expression analysis.

In addition, the genome-derived single exon microarrays of the present invention present substantial and useful advantages in the conduct of such analyses over the devices conventional in the art. Those advantages include each of the advantages over EST-based and in situ synthesized oligo-based microarrays that were discussed hereinabove with respect to confirmation of expression of predicted exons, which discussion is incorporated herein by reference in its entirety, including, inter alia: inclusion of novel probes; absence of the biases inherent in EST libraries, such as expression level bias, 3′ or 5′ end-bias, bias imposed by reverse transcription or by cloning limitations; the absence from the probes of homopolymeric stretches; the absence from the probes of prokaryotic or bacteriophage vector sequence; the absence from the probes of cloning artifacts; the ability to monitor the expression of individual exons; and the uniformity in size and in duplex melting temperature of the arrayed probes.

Gene expression analysis by microarray hybridization is, of course, principally a laboratory-based art. Devices and apparatus used principally in laboratories to facilitate laboratory research are well-established to possess specific, substantial, and credible utility. For example, U.S. Pat. No. 6,001,233 describes and claims a gel electrophoresis apparatus having a cam-activated clamp; for example, U.S. Pat. No. 6,051,831 describes and claims a high mass detector for use in time-of-flight mass spectrometers; for example, U.S. Pat. No. 5,824,269 describes and claims a flow cytometer—as is well known, few gel electrophoresis apparatuses, TOF-MS devices, or flow cytometers are sold for home use.

Indeed, and in particular, nucleic acid microarrays, as devices intended for laboratory use in measuring gene expression, are well-established to have specific, substantial and credible utility. Thus, the genome-derived single exon microarrays of the present invention have at least the specific, substantial and credible utilities of the microarrays claimed as devices and articles of manufacture in the following U.S. patents, the disclosures of each of which is incorporated herein by reference: U.S. Pat. Nos. 5,445,934 (“Array of oligonucleotides on a solid substrate”); No. 5,744,305 (“Arrays of materials attached to a substrate”); and No. 6,004,752 (“Solid support with attached molecules”)

The genome-derived single exon nucleic acid microarrays of the present invention have further utility as products of manufacture that are specifically useful in methods of doing business.

For example, a first such method comprises selling and/or licensing genome-derived single-exon microarrays to a customer desiring to measure gene expression, in consideration of fees paid by such customer.

Such methods can usefully be implemented on a computer. In such computerized embodiments, the method comprises: making available for computerized query a database having a record corresponding to each genome-derived single exon microarray available for sale and/or license; responding to a customer query of the database by returning to the customer at least one record, or an identifier of that record, that best meets the customer query criteria; and offering for sale or license to the querying customer the genome-derived single exon microarray identified in that at least one record.

This method can usefully be implemented by making the database resident on a server computer and available for query by a remotely located client, for example by query over the Internet, an intranet, LAN or WAN. The method can usefully include a later step of permitting the customer to place an order for the identified microarray. This later step can usefully employ the one-click method described and claimed in U.S. Pat. No. 5,960,411, incorporated herein by reference in its entirety.

The genome-derived single exon microarrays of the present invention, as final products of manufacture, also have utility in a second method of doing business, the method comprising designing and/or manufacturing a genome-derived single exon microarray, in consideration of fees paid by those desiring a custom nucleic acid microarray, that has genome-derived single exon probes sharing at least one common attribute.

Such method can usefully be implemented on a computer. In such computerized embodiments, the method comprises: receiving from a customer at least one criterion for common probe attribute, such as tissue of expression; using the at least one criterion to identify within a database having records corresponding to available genome-derived single exon probes those that meet the criterion; and then disposing such identified probes on a support substrate capable of functioning in microarray hybridization experiments.

One useful common probe attribute is expression in a given tissue or cell type. Each of Examples 5-14, infra, presents (in Tables 4-13, respectively) genome-derived single exon probes demonstrated to have significant expression in the identified tissue or cell type; the probes presented therein are thus specifically useful in such a method.

The genome-derived single exon microarrays of the present invention are further useful in a third method of doing business, wherein expression data obtained from use of the genome-derived single exon probes and genome-derived single exon microarrays of the present invention are made available by subscription to customers that desire such information, in consideration for fees.

This aspect of the invention can usefully be implemented on a computer. In such computerized embodiments, the method comprises: making available to a subscription client for computerized query a database having records containing expression data generated using genome-derived single exon probes disposed upon microarrays; and responding to a customer query of the database by returning to the customer at least one record, or an identifier of the at least one record, that best meets the customer query criteria. Each of examples 5-14 presents records from such a database, organized by tissue and/or cell type, and collectively containing expression data for 16,834 single exon probes having unique exons.

The response to the customer in this method can usefully return only the amount of information minimally required to permit the customer to decide whether to pay for further information from the identified record or records. The method can also usefully include a later step of permitting the customer to place an order for the identified microarray. This later step can usefully employ the one-click method described and claimed in U.S. Pat. No. 5,960,411, incorporated herein by reference in its entirety. The third method of doing business, in each of its embodiments, can usefully be performed by making the database resident on a server computer and available for query by a remotely located client, for example by query over the Internet, an intranet, a LAN or WAN.

Each single exon probe has specific utility in such a method, providing information not hitherto present therein. Furthermore, the specific utility is amplified for each successive addition, it being well-established in the database and network arts that each new record or node added to the database or network increases the value of all preceding records or nodes.

The genome-derived single exon nucleic acid probes and microarrays of the present invention have the further real world use in a fourth method of doing business, comprising attracting investment to a company that makes and sells the same. One of ordinary skill in the art would immediately appreciate from the fact that the market capitalization of Affymetrix, Inc. (Sunnyvale, Calif.) on May 14, 2001 was $1.692 billion, although earnings per share were $−1.05, and from the fact that the market capitalization of Incyte Genomics, Inc. (Palo Alto, Calif.) on May 14, 2001 was $1.182 billion, with an earnings/share of −$0.61, that nucleic acid microarrays attract investor dollars without expectation of their sale generating profits.

Thus, the genome-derived single exon nucleic acid microarrays of the present invention are further useful in a method of doing business, the method comprising: advertising the availability for distribution, sale, or license of genome-derived single exon probes, microarrays, and/or data therefrom; and then selling stock in the company.

This method can usefully be implemented on a computer, wherein the advertising is performed on an Internet web page, in an Internet chat room, and/or a Usenet newsgroup; and wherein the stock sale is consummated through an electronic brokerage.

The genome-derived single exon probes and microarrays of the present invention have the further credible, specific, and substantial real world utility of providing income to the drafter of the patent specification in which the probes and microarrays are specifically described and claimed. Specificity of this utility derives from the requirements of 35 U.S.C. § § 102(a), (b), (f) and (g); given the present structure of legal compensation in the United States, the income, and thus utility, is substantial; one or ordinary skill in the art would find the compensation not incredible.

The genome-derived single exon probes of the present invention have additional utilities.

For example, as mentioned above, the single exon probes are particularly useful for identifying and characterizing alternative splicing events, as further described in commonly owned and copending U.S. patent application Ser. No. 09/632,366, filed Aug. 3, 2000, the disclosure of which is incorporated herein by reference in its entirety.

As another example, the single exon probes of the present invention can be used as hybridization probes to detect, characterize, and quantify copy number of their included exon in samples of genomic nucleic acid.

Thus, the probes of the present invention can be used to detect and characterize gross alterations in the genomic locus that includes their exon, such as deletions, insertions, translocations, and duplications through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999) (ISBN: 0471013455), the disclosure of which is incorporated herein by reference in its entirety.

The probes of the present invention can be used to assess smaller genomic alterations using, e.g., Southern blot detection of restriction fragment length polymorphisms.

The probes can also be used to isolate nucleic acids that include their exon, both from transcript-derived nucleic acid samples, such as pools or libraries of cDNA, and from genomic DNA-derived nucleic acid samples.

The probes of the present invention can also be used to prime synthesis of nucleic acid, for purpose of either analysis or isolation, using mRNA, cDNA, or genomic DNA as template.

For use as primers, at least 17 contiguous nucleotides of the probe will typically be used. Often, at least 18, 19, or 20 contiguous nucleotides of the probe will be used, and on occasion at least 20, 22, 24, or 25 contiguous nucleotides of exon will be used, and even 30 nucleotides or more of the probe can be used to prime specific synthesis.

The nucleic acid primers of the present invention can be used, for example, to prime first strand cDNA synthesis on an mRNA template. In such case, the primer will typically be drawn from the exonic portion of the single exon probe.

Such primer extension can be done directly to analyze the message. Alternatively, synthesis on an mRNA template can be done to produce first strand cDNA. The first strand cDNA can thereafter be used, inter alia, directly as a single-stranded probe, as a template for sequencing—permitting identification of alterations, including deletions, insertions, and substitutions, both normal allelic variants and mutations associated with abnormal phenotypes- or as a template, either for second strand cDNA synthesis (e.g., as an antecedent to insertion into a cloning or expression vector), or for amplification.

The nucleic acid primers of the present invention can also be used, for example, to prime single base extension (SBE) for SNP detection (see, e.g., U.S. Pat. No. 6,004,744, the disclosure of which is incorporated herein by reference in its entirety).

The nucleic acid primers of the present invention can also be used to prime specific amplification of the probe, or portions thereof, using transcript-derived or genomic DNA as template.

Primer-directed amplification methods are now well-established in the art. Methods for performing the polymerase chain reaction (PCR) are compiled, inter alia, in McPherson, PCR (Basics: From Background to Bench), Springer Verlag (2000) (ISBN: 0387916008); Innis et al. (eds.), PCR Applications: Protocols for Functional Genomics, Academic Press (1999) (ISBN: 0123721857); Gelfand et al. (eds.), PCR Strategies, Academic Press (1998) (ISBN: 0123721822); Newton et al., PCR, Springer-Verlag New York (1997) (ISBN: 0387915060); Burke (ed.), PCR: Essential Techniques, John Wiley & Son Ltd (1996) (ISBN: 047195697X); White (ed.), PCR Cloning Protocols: From Molecular Cloning to Genetic Engineering, Vol. 67, Humana Press (1996) (ISBN: 0896033430); McPherson et al. (eds.), PCR 2: A Practical Approach, Oxford University Press, Inc. (1995) (ISBN: 0199634254), the disclosures of which are incorporated herein by reference in their entireties.

Methods for performing RT-PCR are collected, e.g., in Siebert et al. (eds.), Gene Cloning and Analysis by RT-PCR, Eaton Publishing Company/Bio Techniques Books Division, 1998 (ISBN: 1881299147); Siebert (ed.), PCR Technique:RT-PCR, Eaton Publishing Company/BioTechniques Books (1995) (ISBN:1881299139), the disclosure of which is incorporated herein by reference in its entirety.

Isothermal amplification approaches, such as rolling circle amplification, are also now well-described. See, e.g., Schweitzer et al., Curr. Opin. Biotechnol. 12(1):21-7 (2001); U.S. Pat. Nos. 5,854,033 and 5,714,320 and international patent publications WO 97/19193 and WO 00/15779, the disclosures of which are incorporated herein by reference in their entireties. Rolling circle amplification can be combined with other techniques to facilitate SNP detection. See, e.g., Lizardi et al., Nature Genet. 19(3):225-32 (1998).

Nucleic acids of the present invention, inserted into vectors that flank the nucleic acid insert with a phage promoter, such as T7, T3, or SP6 promoter, can be used to drive in vitro expression of RNA complementary to either strand of the nucleic acids of the present invention. The RNA can be used, inter alia, as a single-stranded probe, to effect subtraction, or for in vitro translation.

Additionally, the nucleic acids of the present invnetion can be used to express the ORF-encoded peptide, or fragments thereof, either alone, or as part of fusion proteins.

Protein expression can be effected in eukaryotic or in prokaryotic cells using vectors, host cells, and methods well known in the art.

Expressed in vitro, the ORF-encoded peptide or fusion thereof can thereafter be isolated, to be used, inter alia, as a standard in immunoassays specific for the ORF-encoded peptide; to be used as a therapeutic agent, e.g., to be administered as passive replacement therapy in individuals deficient in the protein of which the ORF-encoded peptide is an active moiety or to be administered as a vaccine; to be used for in vitro production of specific antibody, the antibody thereafter to be used, e.g., as an analytical reagent for detection and quantitation of proteins containing the ORF-encoded peptide or to be used as an immunotherapeutic agent.

The isolated nucleic acids of the present invention (genome-derived single exon probes) can also be used to drive in vivo expression of the ORF-encoded peptides of the present invention. In vivo expression can be driven from a vector—typically a viral vector, often a vector based upon a replication incompetent retrovirus, an adenovirus, or an adeno-associated virus (AAV)—for purpose of gene therapy. In vivo expression can also be driven from plasmid vectors, including for purpose of “naked” nucleic acid vaccination, as further described in U.S. Pat. Nos. 5,589,466; 5,679,647; 5,804,566; 5,830,877; 5,843,913; 5,880,104; 5,958,891; 5,985,847; 6,017,897; 6,110,898; 6,204,250, the disclosures of which are incorporated herein by reference in their entireties.

The nucleic acids of the present invention can also be used for antisense inhibition of translation. See Phillips (ed.), Antisense Technology, Part B, Methods in Enzymology Vol. 314, Academic Press, Inc. (1999) (ISBN: 012182215X); Phillips (ed.), Antisense Technology, Part A, Methods in Enzymology Vol. 313, Academic Press, Inc. (1999) (ISBN: 0121822141); Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Agrawal et al. (eds.), Antisense Research and Application, Springer-Verlag New York, Inc. (1998) (ISBN: 3540638334); Lichtenstein et al. (eds.), Antisense Technology: A Practical Approach, Vol. 185, Oxford University Press, INC. (1998) (ISBN: 0199635838); Gibson (ed.), Antisense and Ribozyme Methodology: Laboratory Companion, Chapman & Hall (1997) (ISBN: 3826100794); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties.

The single exon probes of the present invention, and fragments thereof, can be integrated non-homologously into the genome of somatic cells, e.g. CHO cells, COS cells, or 293 cells, with or without amplification of the insertional locus, in order, e.g., to create stable cell lines capable of producing the ORF-encoded peptides of the present invention.

The single exon probes of the present invention, and fragments thereof, can also be used for targeted gene correction or alteration, possibly by cellular mechanisms different from those engaged during homologous recombination.

For example, partially duplexed RNA/DNA chimeras have been shown to have utility in targeted gene correction, U.S. Pat. Nos. 5,945,339, 5,888,983, 5,871,984, 5,795,972, 5,780,296, 5,760,012, 5,756,325, 5,731,181, the disclosures of which are incorporated herein by reference in their entireties. So too have small oligonucleotides fused to triplexing domains have been shown to have utility in targeted gene correction, Culver et al., “Correction of chromosomal point mutations in human cells with bifunctional oligonucleotides,” Nature Biotechnol. 17(10):989-93 (1999), as have oligonucleotides having modified terminal bases or modified terminal internucleoside bonds, Gamper et al., Nucl. Acids Res. 28(21):4332-9 (2000), the disclosures of which are incorporated herein by reference.

Genome-Derived Single Exon Probes

In light of the above-described credible, specific, and substantial utilities, it is an aspect of the present invention to provide genome-derived single exon nucleic acid probes. The invention particularly provides genome-derived single-exon probes known to be expressed in one or more tissues. In particular embodiments, the present invention provides human single-exon probes that include specifically-hybridizable fragments of the exons presented in SEQ ID NOs: 16,835-33,299, wherein the fragment hybridizes at high stringency (under high stringency conditions) to an expressed human gene. In particular embodiments, the invention provides single exon probes having SEQ ID NOs: 1-16,834.

It will be appreciated that the Sequence Listing appended hereto and incorporated herein by reference presents, by convention, only that strand of the probe and exon sequences that can be directly translated, reading from 5′ to 3′ end. As would be well understood by one of skill in the art, single stranded probes must be complementary in sequence to the target; it is well within the skill in the art to determine such complementary sequence. It will further be understood that double stranded probes can be used in both solution-phase hybridization and microarray-based hybridization if suitably denatured.

Thus, it is an aspect of the present invention to provide single-stranded nucleic acid probes that have sequence complementary to those described herein above and below, and double-stranded probes one strand of which has sequence complementary to the probes described herein.

Unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Unless otherwise indicated, nucleotide sequences of the isolated nucleic acids of the present invention were determined by sequencing a DNA molecule that had resulted, directly or indirectly, from at least one enzymatic polymerization reaction (e.g., reverse transcription and/or polymerase chain reaction) using an automated sequencer (such as the MegaBACE™ 1000, Molecular Dynamics, Sunnyvale, Calif., USA), or by reliance upon such sequence or upon genomic sequence prior-accessioned into a public database. Unless otherwise indicated, all amino acid sequences of the polypeptides of the present invention were predicted by translation from the nucleic acid sequences so determined.

As a consequence, any nucleic acid sequence presented herein may contain errors introduced by erroneous incorporation of nucleotides during polymerization, by erroneous base calling by the automated sequencer (although such sequencing errors have been minimized for the nucleic acids directly determined herein, unless otherwise indicated, by the sequencing of each of the complementary strands of a duplex DNA), or by similar errors accessioned into the public database.

Furthermore, single nucleotide polymorphisms (SNPs) occur frequently in eukaryotic genomes—more than 1.4 million SNPs have already identified in the human genome, International Human Genome Sequencing Consortium, Nature 409:860-921 (2001)—and the sequence determined from one individual of a species may differ from other allelic forms present within the population. Additionally, small deletions and insertions, rather than single nucleotide polymorphisms, are not uncommon in the general population, and often do not alter the function of the protein.

Accordingly, it is an aspect of the present invention to provide nucleic acids not only identical in sequence to those described with particularity herein, but also to provide isolated nucleic acids at least about 90% identical in sequence to those described with particularity herein, typically at least about 91%, 92%, 93%, 94%, or 95% identical in sequence to those decribed with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention, as by random or directed mutagenesis.

For purposes herein, percent identity of two nucleic acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at

http://www.ncbi.nlm.nih.gov/blast/bl2seq/bl2.html.

To assess percent identity of nucleic acids, the BLASTN module of BLAST 2 SEQUENCES is used with default values of (i) reward for a match: 1; (ii) penalty for a mismatch: −2; (iii) open gap 5 and extension gap 2 penalties; (iv) gap X_dropoff 50 expect 10 word size 11 filter, and both sequences are entered in their entireties.

As is well known, the genetic code is degenerate, with each amino acid except methionine translated from a plurality of codons, thus permitting a plurality of nucleic acids of disparate sequence to encode the identical protein. As is also well known, codon choice for optimal expression varies from species to species. The isolated nucleic acids of the present invention being useful for expression of ORFs encoded by the exons present within the probes (ORF-encoded peptides), it is, therefore, another aspect of the present invention to provide isolated isolated nucleic acids that encode the ORF-encoded peptides, and portions thereof, not only identical in sequence to those described with particularity herein, but degenerate variants thereof as well.

As is also well known, amino acid substitutions occur frequently among natural allelic variants, with conservative substitutions often occasioning only de minimis change in protein function.

Accordingly, it is an aspect of the present invention to provide nucleic acids not only identical in sequence to those described with particularity herein, but also to provide isolated nucleic acids that encode the ORF-encoded peptides, and fragments thereof, having conservative amino acid substitutions. It is a further aspect to provide nucleic acids that encode the ORF-encoded peptides having moderately conservative amino acid substitutions.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 9 | 9 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | 2 | 4 | −1 | 0 | −3 | −3 | 1 | −2 | −4 | 0 | 0 | 0 | −4 | −3 | −2 | |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −1 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

As is also well known in the art, relatedness of nucleic acids can also be characterized using a functional test, the ability of the two nucleic acids to base-pair to one another at defined hybridization stringencies.

It is, therefore, another aspect of the invention to provide isolated nucleic acids not only identical in sequence to those described with particularity herein, but also to provide isolated nucleic acids (“cross-hybridizing nucleic acids”) that hybridize under high stringency conditions (as defined herein) to all or to a portion of the nucleic acids of the present invention, as set forth in the appended Sequence Listing (“reference nucleic acids”). It is a further aspect of the invention to provide isolated nucleic acids that hybridize under moderate stringency conditions to all or to a protion of the nucleic acids of the present invention, as set forth in the appended Sequence Listing (“reference nucleic acids”).

Such cross-hybridizing nucleic acids are useful, inter alia, as probes for, and to drive expression of, proteins related to the ORF-encoded peptides of the present invention as alternative isoforms, homologues, paralogues, and orthologues. Particularly preferred orthologues are those from other primate species, such as chimpanzee, rhesus macaque, baboon, and gorilla, from rodents, such as rats, mice, guinea pigs, and from livestock, such as cow, pig, sheep, horse, goat.

The hybridizing portion of the reference nucleic acid is typically at least 15 nucleotides in length, often at least 17 nucleotides in length. Often, however, the hybridizing portion of the reference nucleic acid is at least 20 nucleotides in length, 25 nucleotides in length, and even 30 nucleotides, 35 nucleotides, 40 nucleotides, and 50 nucleotides in length. Of course, cross-hybridizing nucleic acids that hybridize to a larger portion of the reference nucleic acid—for example, to a portion of at least 50 nt, at least 100 nt, at least 150 nt, 200 nt, 250 nt, 300 nt, 350 nt, 400 nt, 450 nt, or 500 nt or more—or even to the entire length of the reference nucleic acid, are also useful.

The hybridizing portion of the cross-hybridizing nucleic acid is at least 75% identical in sequence to at least a portion of the reference nucleic acid. Typically, the hybridizing portion of the cross-hybridizing nucleic acid is at least 80%, often at least 85%, 86%, 87%, 88%, 89% or even at least 90% identical in sequence to at least a portion of the reference nucleic acid. Often, the hybridizing portion of the cross-hybridizing nucleic acid will be at least 91%, 92%, 93%, 94%, 95%, 96%, 97%, 98%, or 99% identical in sequence to at least a portion of the reference nucleic acid sequence. At times, the hybridizing portion of the cross-hybridizing nucleic acid will be at least 99.5% identical in sequence to at least a portion of the reference nucleic acid.

The invention also provides fragments of various of the nucleic acids set forth in the appended Sequence Listing (“reference nucleic acids”).

By “fragments” of a reference nucleic acid is here intended isolated nucleic acids, however obtained, that have a nucleotide sequence identical to a portion of the reference nucleic acid sequence, which portion is at least 17 nucleotides and less than the entirety of the reference nucleic acid. As so defined, “fragments” need not be obtained by physical fragmentation of the reference nucleic acid, although such provenance is not thereby precluded.

In theory, an oligonucleotide of 17 nucleotides is of sufficient length as to occur at random less frequently than once in the three gigabase human genome, and thus to provide a nucleic acid probe that can uniquely identify the reference sequence in a nucleic acid mixture of genomic complexity. As is well known, further specificity can be obtained by probing nucleic acid samples of subgenomic complexity, and/or by using plural fragments as short as 17 nucleotides in length collectively to prime amplification of nucleic acids, as, e.g., by polymerase chain reaction (PCR).

As is well known in the art, nucleic acid fragments that encode at least 6 contiguous amino acids (i.e., fragments of 18 nucleotides or more) are useful in directing the expression or the synthesis of peptides that have utility in mapping the epitopes of the protein encoded by the reference nucleic acid. See, e.g., Geysen et al., “Use of peptide synthesis to probe viral antigens for epitopes to a resolution of a single amino acid,” Proc. Natl. Acad. Sci. USA 81:3998-4002 (1984); and U.S. Pat. Nos. 4,708,871 and 5,595,915, the disclosures of which are incorporated herein by reference in their entireties.

As is also well known, fragments that encode at least 8 contiguous amino acids (i.e., fragments of 24 nucleotides or more) are useful in directing the expression or the synthesis of peptides that have utility as immunogens. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties.

The nucleic acid fragment of the present invention is thus at least 17 nucleotides in length, typically at least 18 nucleotides in length, and often at least 24 nucleotides in length. Often, the nucleic acid of the present invention is at least 25 nucleotides in length, and even 30 nucleotides, 35 nucleotides, 40 nucleotides, or 45 nucleotides in length. Of course, larger fragments having at least 50 nt, at least 100 nt, at least 150 nt, 200 nt, 250 nt, 300 nt, 350 nt, 400 nt, 450 nt, or 500 nt or more are also useful, and at times preferred.

The isolated nucleic acids of the present invention—including single exon probes and fragments thereof—can be composed of natural nucleotides in native 5′-3′ phosphodiester internucleoside linkage—e.g., DNA or RNA—or can contain any or all of nonnatural nucleotide analogues, nonnative internucleoside bonds, or post-synthesis modifications, either throughout the length of the nucleic acid or localized to one or more portions thereof. As is well known in the art, when the isolated nucleic acid is used as a hybridization probe, the range of such nonnatural analogues, nonnative internucleoside bonds, or post-synthesis modifications will be limited to those that permit sequence-discriminating basepairing of the resulting nucleic acid. When used to direct expression or RNA or protein in vitro or in vivo, the range of such nonnatural analogues, nonnative internucleoside bonds, or post-synthesis modifications will be limited to those that permit the nucleic acid to function properly as a polymerization substrate. When the isolated nucleic acid is used as a therapeutic agent, the range of such changes will be limited to those that do not confer toxicity upon the isolated nucleic acid.

For example, when desired to be used as probes, the isolated nucleic acids of the present invention can usefully include nucleotide analogues that incorporate labels that are directly detectable, such as radiolabels or fluorophores, or nucleotide analogues that incorporate labels that can be visualized in a subsequent reaction, such as biotin or various haptens.

Common radiolabeled analogues include those labeled with ³³P, ³²P, and ³⁵S, such as α-³²P-dATP, α-₃₂P-dCTP, α-³²P-dGTP, α-³²P-dTTP, α-³²P-3′dATP, α-³²P-ATP, α-³²P-CTP, α-³²P-GTP, α-³²P-UTP, α-³⁵S-DATP, γ-³⁵S-GTP, γ-33P-dATP, and the like.

Commercially available fluorescent nucleotide analogues readily incorporated into the nucleic acids of the present invention include Cy3-dCTP, Cy3-dUTP, Cy5-dCTP, Cy3-dUTP (Amersham Pharmacia Biotech, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY® TMR-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP, Alexa Fluor® 546-14-UTP (Molecular Probes, Inc. Eugene, Oreg., USA).

Protocols are available for custom synthesis of nucleotides having other fluorophores. Henegariu et al., “Custom Fluorecent-Nucleotide Synthesis as an Alternative Method for Nucleic Acid Labeling,” Nature Biotechnol. 18:345-348 (2000), the disclosure of which is incorporated herein by reference in its entirety.

Haptens that are commonly conjugated to nucleotides for subsequent labeling include biotin (biotin-11-dUTP, Molecular Probes, Inc., Eugene, Oreg., USA; biotin-21-UTP, biotin-21-dUTP, Clontech Laboratories, Inc., Palo Alto, Calif., USA), digoxigenin (DIG-11-dUTP, alkali labile, DIG-11-UTP, Roche Diagnostics Corp., Indianapolis, Ind., USA), and dinitrophenyl (dinitrophenyl-11-dUTP, Molecular Probes, Inc., Eugene, Oreg., USA).

As another example, when desired to be used for antisense inhibition of translation, the isolated nucleic acids of the present invention can usefully include altered, often nuclease-resistant, internucleoside bonds. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797), or for targeted gene correction, Gamper et al., Nucl. Acids Res. 28(21):4332-9 (2000), the disclosures of which are incorporated herein by reference in their entireties.

Modified oligonucleotide backbones often preferred when the nucleic acid is to be used for antisense purposes are, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties.

Preferred modified oligonucleotide backbones for antisense use that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the disclosures of which are incorporated herein by reference in their entireties.

In other preferred oligonucleotide mimetics, both the sugar and the internucleoside linkage are replaced with novel groups, such as peptide nucleic acids (PNA).

In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages.

The uncharged nature of the PNA backbone provides PNA/DNA and PNA/RNA duplexes with a higher thermal stability than is found in DNA/DNA and DNA/RNA duplexes, resulting from the lack of charge repulsion between the PNA and DNA or RNA strand. In general, the Tm of a PNA/DNA or PNA/RNA duplex is 1° C. higher per base pair than the Tm of the corresponding DNA/DNA or DNA/RNA duplex (in 100 mM NaCl).

The neutral backbone also allows PNA to form stable DNA duplexes largely independent of salt concentration. At low ionic strength, PNA can be hybridized to a target sequence at temperatures that make DNA hybridization problematic or impossible. And unlike DNA/DNA duplex formation, PNA hybridization is possible in the absence of magnesium. Adjusting the ionic strength, therefore, is useful if competing DNA or RNA is present in the sample, or if the nucleic acid being probed contains a high level of secondary structure.

PNA also demonstrates greater specificity in binding to complementary DNA. A PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater.

Additionally, nucleases and proteases do not recognize the PNA polyamide backbone with nucleobase sidechains. As a result, PNA oligomers are resistant to degradation by enzymes, and the lifetime of these compounds is extended both in vivo and in vitro. In addition, PNA is stable over a wide pH range.

Because its backbone is formed from amide bonds, PNA can be synthesized using a modified peptide synthesis protocol. FNA oligomers can be synthesized by both Fmoc and tBoc methods. Representative U.S. patents that teach the preparation of PNA compounds include, but are not limited to, U.S. Pat. Nos. 5,539,082; 5,714,331; and 5,719,262, each of which is herein incorporated by reference; automated PNA synthesis is readily achievable on commercial synthesizers (see, e.g., “PNA User's Guide,” Rev. 2, February 1998, Perseptive Biosystems Part No. 60138, Applied Biosystems, Inc., Foster City, Calif.).

PNA chemistry and applications are reviewed, inter alia, in Ray et al., FASEB J. 14(9):1041-60 (2000); Nielsen et al., Pharmacol Toxicol. 86(1):3-7 (2000); Larsen et al., Biochim Biophys Acta. 1489(1):159-66 (1999); Nielsen, Curr. Opin. Struct. Biol. 9(3):353-7 (1999), and Nielsen, Curr. Opin. Biotechnol. 10(l):71-5 (1999), the disclosures of which are incorporated herein by reference in their entireties.

Differences from nucleic acid compositions found in nature—e.g., nonnative bases, altered internucleoside linkages, post-synthesis modification—can be present throughout the length of the nucleic acid or can, instead, usefully be localized to discrete portions thereof. As an example of the latter, chimeric nucleic acids can be synthesized that have discrete DNA and RNA domains and demonstrated utility for targeted gene repair, as further described in U.S. Pat. Nos. 5,760,012 and 5,731,181, the disclosures of which are incorporated herein by reference in their entireties. As another example, chimeric nucleic acids comprising both DNA and PNA have been demonstrated to have utility in modified PCR reactions. See Misra et al., Biochem. 37: 1917-1925 (1998); see also Finn et al., Nucl. Acids Res. 24: 3357-3363 (1996), incorporated herein by reference.

Unless otherwise specified, nucleic acids of the present invention can include any topological conformation appropriate to the desired use; the term thus explicitly comprehends, among others, single-stranded, double-stranded, triplexed, quadruplexed, partially double-stranded, partially-triplexed, partially-quadruplexed, branched, hairpinned, circular, and padlocked conformations. Padlock conformations and their utility are further described in Baner et al., Curr. Opin. Biotechnol. 12:11-15 (2001); Escude et al., Proc. Natl. Acad. Sci. USA 14;96(19):10603-7 (1999); Nilsson et al., Science 265(5181):2085-8 (1994), the disclosures of which are incorporated herein by reference in their entireties. Triplex and quadruplex conformations, and their utility, are reviewed in Praseuth et al., Biochim. Biophys. Acta. 1489(1):181-206 (1999); Fox, Curr. Med. Chem. 7(1):17-37 (2000); Kochetkova et al., Methods Mol. Biol. 130:189-201 (2000); Chan et al., J. Mol. Med. 75(4):267-82 (1997), the disclosures of which are incorporated herein by reference in their entireties.

The nucleic acids of the present invention can be detectably labeled. Commonly-used labels include radionuclides, such as ³²P, 33P, ³⁵S, ³H (and for nmr detection, ¹³C and ¹⁵N), haptens that can be detected by specific antibody or high affinity binding partner (such as avidin), and fluorophores.

As noted above, detectable labels can be incorporated by inclusion of labeled nucleotide analogues in the nucleic acid. Such analogues can be incorporated by enzymatic polymerization, such as by nick translation, random priming, polymerase chain reaction (PCR), terminal transferase tailing, and end-filling of overhangs, for DNA molecules, and in vitro transcription driven, e.g., from phage promoters, such as T7, T3, and SP6, for RNA molecules. Commercial kits are readily available for each such labeling approach.

Analogues can also be incorporated during automated solid phase chemical synthesis.

As is well known, labels can also be incorporated after nucleic acid synthesis, with the 5′ phosphate and 3′ hydroxyl providing convenient sites for post-synthetic covalent attachment of detectable labels.

Various other post-synthetic approaches permit internal labeling of nucleic acids.

For example, fluorophores can be attached using a cisplatin reagent that reacts with the N7 of guanine residues (and, to a lesser extent, adenine bases) in DNA, RNA, and PNA to provide a stable coordination complex between the nucleic acid and fluorophore label (Universal Linkage System) (available from Molecular Probes, Inc., Eugene, Oreg., USA and Amersham Pharmacia Biotech, Piscataway, N.J., USA); see Alers et al., Genes, Chromosomes & Cancer, Vol. 25, pp. 301-305 (1999); Jelsma et al., J. NIH Res. 5:82 (1994); Van Belkum et al., BioTechniques 16:148-153 (1994), incorporated herein by reference. As another example, nucleic acids can be labeled using a disulfide-containing linker (FastTag™ Reagent, Vector Laboratories, Inc., Burlingame, Calif., USA) that is photo- or thermally coupled to the target nucleic acid using aryl azide chemistry; after reduction, a free thiol is available for coupling to a hapten, fluorophore, sugar, affinity ligand, or other marker.

Multiple independent or interacting labels can be incorporated into the nucleic acids of the present invention. For example, both a fluorophore and a moiety that in proximity thereto acts to quench fluorescence can be included to report specific hybridization through release of fluorescence quenching, Tyagi et al., Nature Biotechnol. 14: 303-308 (1996); Tyagi et al., Nature Biotechnol. 16, 49-53 (1998); Sokol et al., Proc. Natl. Acad. Sci. USA 95: 11538-11543 (1998); Kostrikis et al., Science 279:1228-1229 (1998); Marras et al., Genet. Anal. 14: 151-156 (1999); U.S. Pat. Nos. 5,846,726, 5,925,517, 5925517, or to report exonucleotidic excision, U.S. Pat. No. 5,538,848; Holland et al., Proc. Natl. Acad. Sci. USA 88:7276-7280 (1991); Heid et al., Genome Res. 6(10):986-94 (1996); Kuimelis et al., Nucleic Acids Symp Ser. (37):255-6 (1997); U.S. Pat. No. 5,723,591, the disclosures of which are incorporated herein by reference in their entireties.

In one aspect, the invention provides the individual single exon probes of the present invention as isolated nucleic acids.

In one series of embodiments of this aspect of the invention, the probe is provided in quantity sufficient to perform a hybridization reaction.

When provided in quantity sufficient to perform a hybridization reaction, the probe can be in any form directly hybridizable to the target that contains the probe's exon or ORF (or their complement), such as double stranded DNA, single-stranded DNA complementary to the target, single-stranded RNA complementary to the target, or chimeric DNA/RNA molecules so hybridizable.

Usefully, however, such probes are instead provided in a form and quantity suitable for amplification. Typically, such probes are provided in a form and quantity suitable for amplification by PCR or by other well known amplification technique. One such technique additional to PCR is rolling circle amplification, as is described, inter alia, in U.S. Pat. Nos. 5,854,033 and 5,714,320 and international patent publications WO 97/19193 and WO 00/15779, the disclosures of which are incorporated herein by reference in their entireties. As is well understood, where the probes are to be provided in a form suitable for amplification, the range of nucleic acid analogues and/or internucleoside linkages will be constrained by the requirements and nature of the amplification enzyme.

Where the probe is to be provided in form suitable for amplification, the quantity need not be sufficient for direct hybridization, and need be sufficient only to function as an amplification template, typically at least about 1 pg, more typically at least about 10 pg, and usually at least about 100 pg or more.

Each discrete amplifiable probe can also be packaged with amplification primers, either in a single composition that comprises probe template and primers, or in a kit that comprises such primers separately packaged therefrom. As mentioned above, which discussion is incorporated here by reference, the exon-specific 5′ primers used for genomic amplification can have a first common sequence added thereto, and the exon-specific 31 primers used for genomic amplification can have a second, different, common sequence added thereto, thus permitting, in this embodiment, the use of a single set of 5′ and 3′ primers to amplify any one of the probes. The probe composition and/or kit can also include buffers, enzyme, etc., required to effect amplification.

In another embodiment, only amplification primers are provided. The primers are sufficient to permit generation of the single exon probe by amplification from genomic DNA, which can be provided by the user.

As mentioned above, when intended for use on a genome-derived single exon microarray of the present invention, the genome-derived single exon probes of the present invention will typically average at least about 50-100 bp, more typically at least about 200 bp, preferably at least about 250 bp, even more preferably about 300 bp, 400 bp, or in preferred embodiments, at least about 500 bp in length, including (and typically, but not necessarily centered about) the exon. Furthermore, when intended for use on a genome-derived single exon microarray of the present invention, the genome-derived single exon probes of the present invention will typically not contain a detectable label.

When intended for use in solution phase hybridization, however—that is, for use in a hybridization reaction in which the probe is not first bound to a support substrate (although the target may indeed be so bound)—length constraints that are imposed in microarray-based hybridization approaches will be relaxed, and such probes will typically be labeled.

In such case, the only functional constraint that dictates the minimum size of such probe is that each such probe must be capable of specific hybridization. In theory, a probe of as little as 17 nucleotides is capable of uniquely identifying its cognate sequence in the human genome. For hybridization to expressed message—a subset of target sequence that is much reduced in complexity as compared to genomic sequence—even fewer nucleotides are required for specificity.

Therefore, the probes of the present invention can include as few as 20 bp of exon, typically at least about 25 bp of exon, more typically at least about 50 bp or exon, or more. The minimum amount of exon required to be included in the probe of the present invention in order to provide specific signal in either solution phase or microarray-based hybridizations can readily be determined by routine experimentation using standard high stringency conditions.

When intended for use in solution phase hybridization, the maximum size of the single exon probes of the present invention is dictated by the proximity of other expressed exons in genomic DNA: although each single exon probe can include intergenic and/or intronic material contiguous to the exon in the human genome, each probe of the present invention will typically include portions of only one expressed exon.

Thus, each single exon probe will include no more than about 25 kb of contiguous genomic sequence, more typically no more than about 20 kb of contiguous genomic sequence, more usually no more than about 15 kb, even more usually no more than about 10 kb. Usually, probes that are maximally about 5 kb will be used, more typically no more than about 3 kb.

And when intended for use in solution hybridization, the probes of the present invention can usefully have detectable labels.

As mentioned above, which discussion is incorporated here by reference, the probes can, but need not, contain intergenic and/or intronic material that flanks the exon, on one or both sides, in the same linear relationship to the exon that the intergenic and/or intronic material bears to the exon in genomic DNA. The probes typically do not, however, contain nucleic acid derived from more than one expressed exon; that is, they do not contain as contiguous sequence nucleic acids that are expressed from, but present discontinuously in, the genome.

The probes, either in quantity sufficient for hybridization or sufficient for amplification, can be provided in individual vials or containers, and can be provided dry (e.g., lyophilized), or solvated. If solvated, the solution can usefully include buffers and salts as desired for hybridization and/or amplification. Furthermore, if desired to be spotted on a microarray, the probes can usefully be provided in a solution of chaotropic agent to facilitate adherence to the microarray support substrate.

Alternatively, such probes can usefully be packaged as a plurality of such individual genome-derived single exon probes.

In one embodiment of this aspect, a small quantity of each probe is disposed, typically without attachment to substrate, in a spatially-addressable ordered set, typically one per well of a microtiter dish. Although a 96 well microtiter plate can be used, greater efficiency is obtained using higher density arrays, such as are provided by microtiter plates having 384, 864, 1536, 3456, 6144, or 9600 wells. And although microtiter plates having physical depressions (wells) are conveniently used, any device that permits addressable withdrawal of reagent from fluidly-noncommunicating areas can be used.

Each of the probes of the ordered set can be provided in any of the forms that are described above with respect to the probes as individually packaged.

As mentioned above, the exon-specific 5′ primers used for genomic amplification can have a first common sequence added thereto, and the exon-specific 3′ primers used for genomic amplification can have a second, different, common sequence added thereto, thus permitting, in certain embodiments, the use of a single set of 5′ and 3′ primers to amplify any one of the probes from the amplifiable ordered set.

Such collections of genome-derived single exon probes can usefully include a plurality of probes chosen for a common attribute, such as common expression in a given tissue, cell type, developmental stage, disease state, or the like.

In such defined subsets, typically at least 50% of the probes will have the common attribute, such as expression in the defined tissue or cell type. More typically, at least about 60% of the probes will be expressed in the defined tissue, even more typically at least about 75%, and preferably at least about 80%, 85%, or, in preferred embodiments, at least about 90%, and even 95% or more of the probes will have the common attribute, such as expression in the defined tissue or cell type.

In exemplified embodiments of this aspect of the invention, the present invention particularly provides subsets of probes having SEQ ID NOs: 1-16,834, the plurality of probes in each subset chosen for their common expression in one of human brain, heart, liver, fetal liver, placenta, lung, bone marrow, HeLa cells, BT 474 cells, or HBL 100 cells. Examples 5-14, infra, present such subsets in Tables 4-13, respectively; probes that have been shown to be expressed in a plurality of the tested tissues appear in the respective plurality of examples and tables.

Genome-Derived Single Exon Microarrays

In light of the above-described credible, specific, and substantial utilities, it is another aspect of the present invention to provide genome-derived single exon nucleic acid microarrays useful for gene expression analysis.

In a first series of embodiments of this aspect of the invention, the genome-derived single exon nucleic acid microarrays are as described hereinabove (“Methods and Apparatus for Generating Single Exon Probes from Genomic Sequence Data”), which discussion is incorporated here by reference in its entirety.

In preferred embodiments, the microarrays include a plurality of probes known to be expressed in one or more tissues. In particular embodiments, the genome-derived single exon microarrays of the present invention include a plurality of probes, each of which plurality includes a nucleotide sequence as set forth in any one of SEQ ID NOs: 16,835-33,299, the complement thereof, or a fragment of the referenced SEQ ID NO: or complement thereof, wherein the probe hybridizes at high stringency (i.e., under high stringency conditions) to a nucleic acid expressed in human cells, and wherein the probe includes portions of no more than one human exon.

Among these embodiments are microarrays that include a plurality of probes, each of which plurality comprises a nucleotide sequence as set forth in SEQ ID NOs. 1-16,834 or the complement thereof. It would be understood that although SEQ ID NOs: 1-16,834 exclude the 51 and 31 universal primer sequences above-described, such sequences can, and often will, be present in the probes as disposed on the microarray substrate.

Single exon microarrays of the present invention can usefully include a plurality of genome-derived single exon probes chosen for a common attribute, such as common expression in a given tissue, cell type, developmental stage, disease state, or the like.

These “subset-defined” genome-derived single exon microarrays can be distinguished by the percentage of probes thereon that are known to have a common attribute, such as expression in a defined tissue or cell type. On such “subset-defined” microarrays, typically at least 50% of the probes will have the common attribute, typically expression in the defined tissue or cell type. More typically, at least about 60% of the probes will be expressed in the defined tissue, even more typically at least about 75%, and preferably at least about 80%, 85%, or, in preferred embodiments, at least about 90%, and even 95% or more of the probes will have the common attribute, such as expression in the defined tissue or cell type.

The invention particularly provides defined subset genome-derived single exon microarrays in which the common attribute is expression respectively in human brain, heart, liver, fetal liver, placenta, lung, bone marrow, HeLa cells, BT 474 cells, or HBL 100 cells.

When used for gene expression analysis, the “defined subset” genome-derived single exon microarrays provide greater physical informational density than do the genome-derived single exon microarrays that have lower percentages of probes known to be expressed commonly in the tested tissue. At a fixed probe density, for example, a given microarray surface area of the defined subset genome-derived single exon microarray can yield a greater number of expression measurements. Alternatively, at a given probe density, the same number of expression measurements can be obtained from a smaller substrate surface area. Alternatively, at a fixed probe density and fixed surface area, probes can be provided redundantly, providing greater reliability in signal measurement for any given probe. Furthermore, with a higher percentage of probes known to be expressed in the assayed tissue, the dynamic range of the detection means can be adjusted to reveal finer levels discrimination among the levels of expression.

The present invention particularly provides defined subset genome-derived single exon microarrays having subsets of the probes having SEQ ID NOs: 1-16,834, the plurality of probes in each subset chosen for their common expression in one of human brain, heart, liver, fetal liver, placenta, lung, bone marrow, HeLa cells, BT 474 cells, or HBL 100 cells. Examples 5-14, infra, present such subsets in Tables 4-13, presented herewith in electronic format and incorporated by reference in the respective examples.

In another aspect of the present invention, a genome-derived single-exon microarray is packaged together with an addressable set of individual probes, the set of individual probes including at least a subset of the probes on the microarray. In alternative embodiments, the ordered set of amplifiable probes is packaged separately from the genome-derived single exon microarray.

In some embodiments, the microarray and/or ordered probe set are further packaged with recorded media that provide probe identification and addressing information, and that can additionally contain annotation information, such as gene expression data. Such recordable media can be packaged with the microarray, with the ordered probe set, or with both.

If the microarray is constructed on a substrate that incorporates recorded media, such as is described in international patent application No. WO 98/12559, entitled “Spatially addressable combinatorial chemical arrays in CD-ROM format,” incorporated herein by reference in its entirety, then separate packaging of the genome-derived single exon microarray and the bioinformatic information is not required.

Vectors and Host Cells

In another aspect, the present invention provides vectors that comprise one or more of the isolated nucleic acids of the present invention, and host cells in which such vectors have been introduced.

The vectors can be used, inter alia, for propagating the nucleic acids of the present invention in host cells (cloning vectors), for shuttling the nucleic acids of the present invention between host cells derived from disparate organisms (shuttle vectors), for inserting the nucleic acids of the present invention into host cell chromosomes (insertion vectors), for expressing sense or antisense RNA transcripts of the nucleic acids of the present invention in vitro or within a host cell, and for expressing polypeptides encoded by the nucleic acids of the present invention, alone or as fusions to heterologous polypeptides. Vectors of the present invention will often be suitable for several such uses.

Vectors are by now well-known in the art, and are described, inter alia, in Jones et al. (eds.), Vectors: Cloning Applications: Essential Techniques (Essential Techniques Series), John Wiley & Son Ltd 1998 (ISBN: 047196266X); Jones et al. (eds.), Vectors: Expression Systems: Essential Techniques (Essential Techniques Series), John Wiley & Son Ltd, 1998 (ISBN:0471962678); Gacesa et al., Vectors: Essential Data, John Wiley & Sons, 1995 (ISBN: 0471948411); Cid-Arregui (eds.), Viral Vectors: Basic Science and Gene Therapy, Eaton Publishing Co., 2000 (ISBN: 188129935X); Sambrook et al., Molecular Cloning: A Laboratory Manual (3^{rd} ed.), Cold Spring Harbor Laboratory Press, 2001 (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X), the disclosures of which are incorporated herein by reference in their entireties. Furthermore, an enormous variety of vectors are available commercially. Use of existing vectors and modifications thereof being well within the skill in the art, only basic features need be described here.

Typically, vectors are derived from virus, plasmid, prokaryotic or eukaryotic chromosomal elements, or some combination thereof, and include at least one origin of replication, at least one site for insertion of heterologous nucleic acid, typically in the form of a polylinker with multiple, tightly clustered, single cutting restriction sites, and at least one selectable marker, although some integrative vectors will lack an origin that is functional in the host to be chromosomally modified, and some vectors will lack selectable markers. Vectors of the present invention will further include at least one nucleic acid of the present invention inserted into the vector in at least one location.

Where present, the origin of replication and selectable markers are chosen based upon the desired host cell or host cells; the host cells, in turn, are selected based upon the desired application.

For example, prokaryotic cells, typically E. coli, are typically chosen for cloning. In such case, vector replication is predicated on the replication strategies of coliform-infecting phage—such as phage lambda, M13, T7, T3 and P1—or on the replication origin of autonomously replicating episomes, notably the ColE1 plasmid and later derivatives, including pBR322 and the pUC series plasmids. Where E. coli is used as host, selectable markers are, analogously, chosen for selectivity in gram negative bacteria: e.g., typical markers confer resistance to antibiotics, such as ampicillin, tetracycline, chlorampenicol, kanamycin, streptomycin, zeocin; auxotrophic markers can also be used.

As another example, yeast cells, typically S. cerevisiae, are chosen, inter alia, for eukaryotic genetic studies, due to the ease of targeting genetic changes by homologous recombination and to the ready ability to complement genetic defects using recombinantly expressed proteins, for identification of interacting protein components, e.g. through use of a two-hybrid system, and for protein expression. Vectors of the present invention for use in yeast will typically, but not invariably, contain an origin of replication suitable for use in yeast and a selectable marker that is functional in yeast.

Integrative YIp vectors do not replicate autonomously, but integrate, typically in single copy, into the yeast genome at low frequencies and thus replicate as part of the host cell chromosome; these vectors lack an origin of replication that is functional in yeast, although they typically have at least one origin of replication suitable for progation of the vector in bacterial cells. YEp vectors, in contrast, replicate episomally and autonomously due to presence of the yeast 2 micron plasmid origin (2 μm ori). The YCp yeast centromere plasmid vectors are autonomously replicating vectors containing centromere sequences, CEN, and autonomously replicating sequences, ARS; the ARS sequences are believed to correspond to the natural replication origins of yeast chromosomes. YACs are based on yeast linear plasmids, denoted YLp, containing homologous or heterologous DNA sequences that function as telomeres (TEL) in vivo, as well as containing yeast ARS (origins of replication) and CEN (centromeres) segments.

Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trpl-D1 and lys2-201. The URA3 and LYS2 yeast genes further permit negative selection based on specific inhibitors, 5-fluoro-orotic acid (FOA) and a-aminoadipic acid (AA), respectively, that prevent growth of the prototrophic strains but allows growth of the ura3 and lys2 mutants, respectively. Other selectable markers confer resistance to, e.g., zeocin.

As yet another example, insect cells are often chosen for high efficiency protein expression. Where the host cells are from Spodoptera frugiperda—e.g., Sf9 and Sf21 cell lines, and expresSF™ cells (Protein Sciences Corp., Meriden, Conn., USA)—the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following cotransfection with AcMNPV DNA, a homologous recombination event occurs between these sequences resulting in a recombinant virus carrying the gene of interest and the polyhedrin or p10 promoter. Selection can be based upon visual screening for lacZ fusion activity.

As yet another example, mammalian cells are often chosen for expression of proteins intended as pharmaceutical agents, and are also chosen as host cells for screening of potential agonist and antagonists of a protein or a physiological pathway.

Where mammalian cells are chosen as host cells, vectors intended for autonomous extrachromosomal replication will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus ElA). Vectors intended for integration, and thus replication as part of the mammalian chromosome, can, but need not, include an origin of replication functional in mammalian cells, such as the SV40 origin. Vectors based upon viruses, such as adenovirus, adeno-associated virus, vaccinia virus, and various mammalian retroviruses, will typically replicate according to the viral replicative strategy.

Selectable markers for use in mammalian cells include resistance to neomycin (G418), blasticidin, hygromycin and to zeocin, and selection based upon the purine salvage pathway using HAT medium.

Vectors of the present invention will also often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

Expression vectors of the present invention—that is, those vectors that will drive expression of polypeptides from the inserted heterologous nucleic acid—will often include a variety of other genetic elements operatively linked to the protein-encoding heterologous nucleic acid insert, typically genetic elements that drive transcription, such as promoters and enhancer elements, those that facilitate RNA processing, such as transcription termination and/or polyadenylation signals, and those that facilitate translation, such as ribosomal consensus sequences.

For example, vectors for expressing proteins of the present invention in prokaryotic cells, typically E. coli, will include a promoter, often a phage promoter, such as phage lambda pL promoter, the trc promoter, a hybrid derived from the trp and lac promoters, the bacteriophage T7 promoter (in E. coli cells engineered to express the T7 polymerase), or the araBAD operon. Often, such prokaryotic expression vectors will further include transcription terminators, such as the aspA terminator, and elements that facilitate translation, such as a consensus ribosome binding site and translation termination codon, Schomer et al., Proc. Natl. Acad. Sci. USA 83:8506-8510 (1986).

As another example, vectors for expressing proteins of the present invention in yeast cells, typically S. cerevisiae, will include a yeast promoter, such as the CYC1 promoter, the GAL1 promoter, ADH1 promoter, or the GPD promoter, and will typically have elements that facilitate transcription termination, such as the transcription termination signals from the CYC1 or ADH1 gene.

As another example, vectors for expressing proteins of the present invention in mammalian cells will include a promoter active in mammalian cells. Such promoters are often drawn from mammalian viruses—such as the enhancer-promoter sequences from the immediate early gene of the human cytomegalovirus (CMV), the enhancer-promoter sequences from the Rous sarcoma virus long terminal repeat (RSV LTR), and the enhancer-promoter from SV40. Often, expression is enhanced by incorporation of polyadenylation sites, such as the late SV40 polyadenylation site and the polyadenylation signal and transcription termination sequences from the bovine growth hormone (BGH) gene, and ribosome binding sites. Furthermore, vectors can include introns, such as intron II of rabbit β-globin gene and the SV40 splice elements.

Vector-drive protein expression can be constitutive or inducible.

Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters. Examples of inducible synthetic promoters are the hybrid Plac/ara-1 promoter and the PLtetO-1 promoter. The PltetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline.

As another example of inducible elements, hormone response elements, such as the glucocorticoid response element (GRE) and the estrogen response element (ERE), can confer hormone inducibility where vectors are used for expression in cells having the respective hormone receptors. To reduce background levels of expression, elements responsive to ecdysone, an insect hormone, can be used instead, with coexpression of the ecdysone receptor.

Expression vectors can be designed to fuse the expressed polypeptide to small protein tags that facilitate purification and/or visualization.

For example, proteins of the present invention can be expressed with a polyhistidine tag that facilitates purification of the fusion protein by immobilized metal affinity chromatography, for example using NiNTA resin (Qiagen Inc., Valencia, Calif., USA) or TALON resin (cobalt immobilized affinity chromatography medium, Clontech Labs, Palo Alto, Calif., USA). As another example, the fusion protein can include a chitin-binding tag and self-excising intein, permitting chitin-based purification with self-removal of the fused tag (IMPACT™ system, New England Biolabs, Inc., Beverley, Mass., USA). Alternatively, the fusion protein can include a calmodulin-binding peptide tag, permitting purification by calmodulin affinity resin (stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA).

Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-VS antibody (Invitrogen, Carlsbad, Calif., USA), FLAG® epitope, detectable by anti-FLAG® antibody (Stratagene, La Jolla, Calif., USA), and the HA epitope.

For secretion of expressed proteins, vectors can include appropriate sequences that encode secretion signals, such as leader peptides. For example, the pSecTag2 vectors (Invitrogen, Carlsbad, Calif., USA) are 5.2 kb mammalian expression vectors that carry the secretion signal from the V-J2-C region of the mouse Ig kappa-chain for efficient secretion of recombinant proteins from a variety of mammalian cell lines.

Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as green fluorescent protein (GFP), fusions to the IgG Fc region, and fusions for use in two hybrid systems.

Vectors for phage display fuse the encoded polypeptide to, e.g., the gene III protein (pIII) or gene VIII protein (pVIII) for display on the surface of filamentous phage, such as M13. See Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, San Diego: Academic Press, Inc., 1996; Abelson et al. (eds.), Combinatorial Chemistry, Methods in Enzymology vol. 267, Academic Press (May 1996).

Vectors for yeast display, e.g. the pYD1 yeast display vector (Invitrogen, Carlsbad, Calif., USA), use the a-agglutinin yeast adhesion receptor to display recombinant protein on the surface of S. cerevisiae. Vectors for mammalian display, e.g., the pDisplay™ vector (Invitrogen, Carlsbad, Calif., USA), target recombinant proteins using an N-terminal cell surface targeting signal and a C-terminal transmembrane anchoring domain of platelet derived growth factor receptor.

A wide variety of vectors now exist that fuse proteins encoded by heterologous nucleic acids to the chromophore of the substrate-independent, intrinsically fluorescent green fluorescent protein from Aequorea Victoria (“GFP”) and its variants. These proteins are intrinsically fluorescent: the GFP-like chromophore is entirely encoded by its amino acid sequence and can fluoresce without requirement for cofactor or substrate.

Structurally, the GFP-like chromophore comprises an 11-stranded β-barrel (β-can) with a central α-helix, the central α-helix having a conjugated n-resonance system that includes two aromatic ring systems and the bridge between them. The n-resonance system is created by autocatalytic cyclization among amino acids; cyclization proceeds through an imidazolinone intermediate, with subsequent dehydrogenation by molecular oxygen at the Cα-Cβ bond of a participating tyrosine.

The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al., “Deletions of the Aequorea victoria Green Fluorescent Protein Define the Minimal Domain Required for Fluorescence,” J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation.

The methods for engineering such modified GFP-like chromophores and testing them for fluorescence activity, both alone and as part of protein fusions, are well-known in the art. Early results of these efforts are reviewed in Heim et al., Curr. Biol. 6:178-182 (1996), incorporated herein by reference in its entirety; a more recent review, with tabulation of useful mutations, is found in Palm et al., “Spectral Variants of Green Fluorescent Protein,” in Green Fluorescent Proteins, Conn (ed.), Methods Enzymol. vol. 302, pp. 378-394 (1999), incorporated herein by reference in its entirety. A variety of such modified chromophores are now commercially available and can readily be used in the fusion proteins of the present invention.

For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins of the present invention. A variety of EGFP vectors, both plasmid and viral, are available commercially (Clontech Labs, Palo Alto, Calif., USA), including vectors for bacterial expression, vectors for N-terminal protein fusion expression, vectors for expression of C-terminal protein fusions, and for bicistronic expression.

Toward the other end of the emission spectrum, EBFP (“enhanced blue fluorescent protein”) and BFP2 contain four amino acid substitutions that shift the emission from green to blue, enhance the brightness of fluorescence and improve solubility of the protein, Heim et al., Curr. Biol. 6:178-182 (1996); Cormack et al., Gene 173:33-38 (1996). EBFP is optimized for expression in mammalian cells whereas BFP2, which retains the original jellyfish codons, can be expressed in bacteria; as is further discussed below, the host cell of production does not affect the utility of the resulting fusion protein. The GFP-like chromophores from EBFP and BFP2 can usefully be included in the fusion proteins of the present invention, and vectors containing these blue-shifted variants are available from Clontech Labs (Palo Alto, Calif., USA).

Analogously, EYFP (“enhanced yellow fluorescent protein”), also available from Clontech Labs, contains four amino acid substitutions, different from EBFP, Ormo et al., Science 273:1392-1395 (1996), that shift the emission from green to yellowish-green. Citrine, an improved yellow fluorescent protein mutant, is described in Heikal et al., Proc. Natl. Acad. Sci. USA 97:11996-12001 (2000). ECFP (“enhanced cyan fluorescent protein”) (Clontech Labs, Palo Alto, Calif., USA) contains six amino acid substitutions, one of which shifts the emission spectrum from green to cyan. Heim et al., Curr. Biol. 6:178-182 (1996); Miyawaki et al., Nature 388:882-887 (1997). The GFP-like chromophore of each of these GFP variants can usefully be included in the fusion proteins of the present invention.

The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties. See also Conn (ed.), Green Fluorescent Protein, Methods in

Fusions to the IgG Fc region increase serum half life of protein pharmaceutical products through interaction with the FcRn receptor (also denominated the FcRp receptor and the Brambell receptor, FcRb), further described in international patent application nos. WO 97/43316, WO 97/34631, WO 96/32478, WO 96/18412.

The present invention further includes host cells comprising the vectors of the present invention, either present episomally within the cell or integrated, in whole or in part, into the host cell chromosome.

As noted earlier, host cells can be prokaryotic or eukaryotic. Representative examples of appropriate host cells include, but are not limited to, bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda—e.g., Sf9 and Sf21 cell lines, and expresSF™ cells (Protein Sciences Corp., Meriden, Conn., USA)—Drosophila S2 cells, and Trichoplusia ni High Five® Cells (Invitrogen, Carlsbad, Calif., USA); and mammalian cells. Typical mammalian cells include COS1 and COS7 cells, chinese hamster ovary (CHO) cells, NIH 3T3 cells, 293 cells, HEPG2 cells, HeLa cells, L cells, murine ES cell lines (e.g., from strains 129/SV, C57/BL6, DBA-1, 129/SVJ), K562, Jurkat cells, and BW5147. Other mammalian cell lines are well known and readily available from the American Type Culture Collection (ATCC) (Manassas, Va., USA) and the National Institute of General medical Sciences (NIGMS) Human Genetic Cell Repository at the Coriell Cell Repositories (Camden, N.J., USA).

Methods for introducing the vectors and nucleic acids of the present invention into the host cells are well known in the art; the choice of technique will depend primarily upon the specific vector to be introduced and the host cell chosen.

For example, phage lambda vectors will typically be packaged using a packaging extract (e.g., Gigapack® packaging extract, Stratagene, La Jolla, Calif., USA), and the packaged virus used to infect E. coli. Plasmid vectors will typically be introduced into chemically competent or electrocompetent bacterial cells.

E. coli cells can be rendered chemically competent by treatment, e.g., with CaCl₂, or a solution of Mg²⁺, Mn²⁺, Ca²⁺, Rb⁺ or K³⁰ , dimethyl sulfoxide, dithiothreitol, and hexamine cobalt (III), Hanahan, J. Mol. Biol. 166(4):557-80 (1983), and vectors introduced by heat shock. A wide variety of chemically competent strains are also available commercially (e.g., Epicurian Coli® XL10-Gold® Ultracompetent Cells (Stratagene, La Jolla, Calif., USA); DH5A competent cells (Clontech Laboratories, Palo Alto, Calif., USA); TOP10 Chemically Competent E. coli Kit (Invitrogen, Carlsbad, Calif., USA)).

Bacterial cells can be rendered electrocompetent—that is, competent to take up exogenous DNA by electroporation—by various pre-pulse treatments; vectors are introduced by electroporation followed by subsequent outgrowth in selected media. An extensive series of protocols is provided online in Electroprotocols (BioRad, Richmond, Calif., USA)

(http://www.bio-rad.com/LifeScience/pdf/New_Gene_Pulser.pdf).

Vectors can be introduced into yeast cells by spheroplasting, treatment with lithium salts, electroporation, or protoplast fusion.

Spheroplasts are prepared by the action of hydrolytic enzymes—a snail-gut extract, usually denoted Glusulase, or Zymolyase, an enzyme from Arthrobacter luteus—to remove portions of the cell wall in the presence of osmotic stabilizers, typically 1 M sorbitol. DNA is added to the spheroplasts, and the mixture is co-precipitated with a solution of polyethylene glycol (PEG) and Ca²⁺. Subsequently, the cells are resuspended in a solution of sorbitol, mixed with molten agar and then layered on the surface of a selective plate containing sorbitol.

For lithium-mediated transformation, yeast cells are treated with lithium acetate, which apparently permeabilizes the cell wall, DNA is added and the cells are co-precipitated with PEG. The cells are exposed to a brief heat shock, washed free of PEG and lithium acetate, and subsequently spread on plates containing ordinary selective medium. Increased frequencies of transformation are obtained by using specially-prepared single-stranded carrier DNA and certain organic solvents. Schiestl et al., Curr. Genet. 16(5-6):339-46 (1989).

For electroporation, freshly-grown yeast cultures are typically washed, suspended in an osmotic protectant, such as sorbitol, mixed with DNA, and the cell suspension pulsed in an electroporation device. Subsequently, the cells are spread on the surface of plates containing selective media. Becker et al., Methods Enzymol. 194:182-7 (1991). The efficiency of transformation by electroporation can be increased over 100-fold by using PEG, single-stranded carrier DNA and cells that are in late log-phase of growth. Larger constructs, such as YACs, can be introduced by protoplast fusion.

Mammalian and insect cells can be directly infected by packaged viral vectors, or transfected by chemical or electrical means.

For chemical transfection, DNA can be coprecipitated with CaPO₄ or introduced using liposomal and nonliposomal lipid-based agents. Commercial kits are available for CaPO₄ transfection (CalPhos™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN® Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene, PolyFect Superfect® (Qiagen, Inc., Valencia, Calif., USA).

Protocols for electroporating mammalian cells can be found online in Electroprotocols (Bio-Rad, Richmond, Calif., USA) (http://www.bio-rad.com/LifeScience/pdf/New_Gene_Pulser.pdf). See also, Norton et al. (eds.), Gene Transfer Methods: Introducinq DNA into Living Cells and Organisms, BioTechiques Books, Eaton Publishing Co. (2000) (ISBN 1-881299-34-1), incorporated herein by reference in its entirety.

ORF-encoded Peptides

It is another aspect of the present invention to provide peptides encoded by the ORFs presented in SEQ ID NOs: 1-33,299; the sequences of the ORF-encoded peptides are set forth in SEQ ID NOs: 33,300-49,117. The concordance among probe, exon, and ORF-encoded peptide SEQ ID NOs: is set forth in Tables 4-13, infra. It is another aspect to provide fragments and fusions of the ORF-encoded peptides of the present invention.

Unless otherwise indicated, amino acid sequences of the proteins of the present invention were determined as a predicted translation from a nucleic acid sequence. Accordingly, any amino acid sequence presented herein may contain errors due to errors in the nucleic acid sequence, as described in detail above. Furthermore, single nucleotide polymorphisms (SNPs) occur frequently in eukaryotic genomes—more than 1.4 million SNPs have already identified in the human genome, International Human Genome Sequencing Consortium, Nature 409:860-921 (2001)—and the sequence determined from one individual of a species may differ from other allelic forms present within the population. Small deletions and insertions can often be found that do not alter the function of the protein.

Accordingly, it is an aspect of the present invention to provide proteins not only identical in sequence to those described with particularity herein, but also to provide, for each ORF-encoded peptide described herein, isolated proteins at least about 90% identical in sequence, typically at least about 91%, 92%, 93%, 94%, or 95% identical in sequence to those decribed with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention by way of random or directed mutagenesis.

For purposes herein, percent identity of two amino acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at

http://www.ncbi.nlm.nih.gov/blast/b12seq/b12.html,

To assess percent identity of amino acid sequences, the BLASTP module of BLAST 2 SEQUENCES is used with default values of (i) BLOSUM62 matrix, Henikoff et al., Proc. Natl. Acad. Sci USA 89(22):10915-9 (1992); (ii) open gap 11 and extension gap 1 penalties; and (iii) gap x_dropoff 50 expect 10 word size 3 filter, and both sequences are entered in their entireties.

As is well known, amino acid substitutions occur frequently among natural allelic variants, with conservative substitutions often occasioning only de minimis change in protein function.

Accordingly, it is an aspect of the present invention to provide proteins not only identical in sequence to those described with particularity herein, but also to provide isolated proteins having the sequence of the ORF-encoded peptides, or portions thereof, with conservative amino acid substitutions. It is a further aspect to provide isolated proteins having the sequence of ORF-encoded peptides, and portions thereof, with moderately conservative amino acid substitutions. These conservatively-sustituted or moderately conservatively-substituted variants can be naturally occurring or can result from human intervention.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 9 | 9 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | 2 | 4 | −1 | 0 | −3 | −3 | 1 | −2 | −4 | 0 | 0 | 0 | −4 | −3 | −2 | |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −1 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

As is also well known in the art, relatedness of proteins can also be characterized using a functional test, the ability of the encoding nucleic acids to base-pair to one another at defined hybridization stringencies.

It is, therefore, another aspect of the invention to provide isolated proteins not only identical in sequence to those described with particularity herein, but also to provide, for each ORF-encoded peptide of the present invention, isolated proteins (“hybridization related proteins”) that are encoded by nucleic acids that hybridize under high stringency conditions (as defined herein above) to all or to a portion of the ORF-encoded peptide's encoding exon (“reference nucleic acids”). It is a further aspect of the invention to provide, for each ORF-encoded peptide described with particularity herein, isolated proteins (“hybridization related proteins”) that are encoded by nucleic acids that hybridize under moderate sringency conditions (as defined herein above) to all or to a portion of each of the encoding exon of the respective ORF-encoded peptide.

The hybridization related proteins can be alternative isoforms, homologues, paralogues, and orthologues of the ORF-encoded peptides of the present invention. Particularly preferred orthologues are those from other primate species, such as chimpanzee, rhesus macaque, baboon, and gorilla, from rodents, such as rats, mice, guinea pigs, and from livestock, such as cow, pig, sheep, horse, goat.

Relatedness of proteins can also be characterized using a second functional test, the ability of a first protein competitively to inhibit the binding of a second protein to an antibody.

It is, therefore, another aspect of the present invention to provide isolated proteins not only identical in sequence to the ORF-encoded peptides described with particularity herein, but also to provide, for each ORF-encoded peptide, isolated proteins (“cross-reactive proteins”) that competitively inhibit the binding of antibodies to all or to a portion of the respective ORF-encoded peptide of the present invention (“reference proteins”). Such competitive inhibition can readily be determined using immunoassays well known in the art.

As further described below, the isolated proteins of the present invention can readily be used as specific immunogens to raise antibodies that specifically recognize the ORF-encoded peptides, their isoforms, homologues, paralogues, and/or orthologues. The antibodies, in turn, can be used, inter alia, specifically to assay for the respective ORF-encoded peptides of the present invention—e.g. by ELISA for detection of protein fluid samples, such as serum, by immunohistochemistry or laser scanning cytometry, for detection of protein in tissue samples, or by flow cytometry, for detection of intracellular protein in cell suspensions—for specific antibody-mediated isolation and/or purification of ORF-encoded peptides of the present invention, as for example by immunoprecipitation, and for use as specific agonists or antagonists of ORF-encoded peptide action.

The isolated proteins of the present invention are also immediately available for use as specific standards in assays used specifically to determine the concentration and/or amount of the respective ORF-encoded peptide (and proteins comprising the same) of the present invention. For example, ELISA kits for detection and quantitation of protein analytes include purified protein of known concentration for use as a measurement standard (e.g., the human interferon-γ OptEIA kit, catalog no. 555142, Pharmingen, San Diego, Calif., USA includes human recombinant gamma interferon, baculovirus produced).

The isolated proteins of the present invention are also immediately available for use as specific biomolecule capture probes for surface-enhanced laser desorption ionization (SELDI) detection of protein-protein interactions, WO 98/59362; WO 98/59360; WO 98/59361; and Merchant et al., Electrophoresis 21(6):1164-77 (2000), the disclosures of which are incorporated herein by reference in their entireties. The isolated proteins of the present invention are also immediately available for use as specific biomolecule capture probes on BIACORE surface plasmon resonance probes.

The isolated proteins of the present invention are also useful as a therapeutic supplement in patients having a specific deficiency in ORF-encoded peptide production.

In another aspect, the invention also provides fragments of various of the proteins of the present invention. The protein fragments are useful, inter alia, as antigenic and immunogenic fragments of the ORF-encoded peptide.

By “fragments” of a protein is here intended isolated proteins (equally, polypeptides, peptides, oligopeptides), however obtained, that have an amino acid sequence identical to a portion of the reference amino acid sequence, which portion is at least 6 amino acids and less than the entirety of the reference nucleic acid. As so defined, “fragments” need not be obtained by physical fragmentation of the reference protein, although such provenance is not thereby precluded.

Fragments of at least 6 contiguous amino acids are useful in mapping B cell and T cell epitopes of the reference protein. See, e.g., Geysen et al., “Use of peptide synthesis to probe viral antigens for epitopes to a resolution of a single amino acid,” Proc. Natl. Acad. Sci. USA 81:3998-4002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of the proteins of the present invention have utility in such a study.

Fragments of at least 8 contiguous amino acids, often at least 15 contiguous amino acids, have utility as immunogens for raising antibodies that recognize the proteins of the present invention. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties. As further described in the above-cited references, virtually all 8-mers, conjugated to a carrier, such as a protein, prove immunogenic—that is, prove capable of eliciting antibody for the conjugated peptide; accordingly, all fragments of at least 8 amino acids of the proteins of the present invention have utility as immunogens.

Fragments of at least 8, 9, 10 or 12 contiguous amino acids are also useful as competitive inhibitors of binding of the entire protein, or a portion thereof, to antibodies (as in epitope mapping), and to natural binding partners, such as subunits in a multerimic complex or to receptors or ligands of the subject protein; this competitive inhibition permits identification and separation of molecules that bind specifically to the protein of interest, U.S. Pat. Nos. 5,539,084 and 5,783,674, incorporated herein by reference in their entireties.

The protein, or protein fragment, of the present invention is thus at least 6 amino acids in length, typically at least 8, 9, 10 or 12 amino acids in length, and often at least 15 amino acids in length. Often, the protein or the present invention, or fragment thereof, is at least 20 amino acids in length, even 25 amino acids, 30 amino acids, 35 amino acids, or 50 amino acids or more in length. Of course, larger fragments having at least 75 amino acids, 100 amino acids, or even 150 amino acids are also useful, and at times preferred.

The present invention further provides fusions of each of the proteins and protein fragments of the present invention to heterologous polypeptides.

By fusion is here intended that the protein or protein fragment of the present invention is linearly contiguous to the heterologous polypeptide in a peptide-bonded polymer of amino acids or amino acid analogues; by “heterologous polypeptide” is here intended a polypeptide that does not naturally occur in contiguity with the protein or protein fragment of the present invention. As so defined, the fusion can consist entirely of a plurality of fragments of the ORF-encoded peptide in altered arrangement; in such case, any of the ORF-encoded peptide fragments can be considered heterologous to the other ORF-encoded peptide fragments in the fusion protein. More typically, however, the heterologous polypeptide is not drawn from the ORF-encoded peptide itself.

The fusion proteins of the present invention will include at least one fragment of the protein of the present invention, which fragment is at least 6, typically at least 8, often at least 15, and usefully at least 16, 17, 18, 19, or 20 amino acids long. The fragment of the protein of the present to be included in the fusion can usefully be at least 25 amino acids long, at least 50 amino acids long, and can be at least 75, 100, or even 150 amino acids long. Fusions that include the entirety of the ORF-encoded peptide of the present invention have particular utility.

The heterologous polypeptide included within the fusion protein of the present invention is at least 6 amino acids in length, often at least 8 amino acids in length, and usefully at least 15, 20, and 25 amino acids in length. Fusions that include larger polypeptides, such as the IgG Fc region, and even entire proteins (such as GFP chromophore-containing proteins), have particular utility.

As described above in the description of vectors and expression vectors of the present invention, which discussion is incorporated here by reference in its entirety, heterologous polypeptides to be included in the fusion proteins of the present invention can usefully include those designed to facilitate purification and/or visualization of recombinantly-expressed proteins. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of the presence of the respective ORF-encoded peptide.

As also discussed above, heterologous polypeptides to be included in the fusion proteins of the present invention can usefully include those that facilitate secretion of recombinantly expressed proteins—into the periplasmic space or extracellular milieu for prokaryotic hosts, into the culture medium for eukaryotic cells—through incorporation of secretion signals and/or leader sequences.

Other useful protein fusions of the present invention include those that permit use of the protein of the present invention as bait in a yeast two-hybrid system. See Bartel et al. (eds.), The Yeast Two-Hybrid System, Oxford University Press (1997) (ISBN: 0195109384); Zhu et al., Yeast Hybrid Technologies, Eaton Publishing, (2000) (ISBN 1-881299-15-5); Fields et al., Trends Genet. 10(8):286-92 (1994); Mendelsohn et al., Curr. Opin. Biotechnol. 5(5):482-6 (1994); Luban et al., Curr. Opin. Biotechnol. 6(1):59-64 (1995); Allen et al., Trends Biochem. Sci. 20(12):511-6 (1995); Drees, Curr. Opin. Chem. Biol. 3(1):64-70 (1999); Topcu et al., Pharm. Res. 17(9):1049-55 (2000); Fashena et al., Gene 250(1-2):1-14 (2000), the disclosures of which are incorporated herein by reference in their entireties. Typically, such fusion is to either E. coli LexA or yeast GAL4 DNA binding domains. Related bait plasmids are available that express the bait fused to a nuclear localization signal.

Other useful protein fusions include those that permit display of the encoded protein on the surface of a phage or cell, fusions to intrinsically fluorescent proteins, such as green fluorescent protein (GFP), and fusions to the IgG Fc region.

The proteins and protein fragments of the present invention can also usefully be fused to protein toxins, such as Pseudomonas exotoxin A, diphtheria toxin, shiga toxin A, anthrax toxin lethal factor, ricin, in order to effect ablation of cells that bind or take up the proteins of the present invention.

The isolated proteins, protein fragments, and protein fusions of the present invention can be composed of natural amino acids linked by native peptide bonds, or can contain any or all of nonnatural amino acid analogues, nonnative bonds, and post-synthetic (post translational) modifications, either throughout the length of the protein or localized to one or more portions thereof.

As is well known in the art, when the isolated protein is used, e.g., for epitope mapping, the range of such nonnatural analogues, nonnative inter-residue bonds, or post-synthesis modifications will be limited to those that permit binding of the peptide to antibodies. When used as an immunogen for the preparation of antibodies in a non-human host, such as a mouse, the range of such nonnatural analogues, nonnative inter-residue bonds, or post-synthesis modifications will be limited to those that do not interfere with the immunogenicity of the protein. When the isolated protein is used as a therapeutic agent, such as a vaccine or for replacement therapy, the range of such changes will be limited to those that do not confer toxicity upon the isolated protein.

Non-natural amino acids can be incorporated during solid phase chemical synthesis or by recombinant techniques, although the former is typically more common.

Solid phase chemical synthesis of peptides is well established in the art. Procedures are described, inter alia, in Chan et al. (eds.), Fmoc Solid Phase Peptide Synthesis: A Practical Approach (Practical Approach Series, (Paper)), Oxford Univ. Press (March 2000) (ISBN: 0199637245); Jones, Amino Acid and Peptide Synthesis (Oxford Chemistry Primers, No 7), Oxford Univ. Press (August 1992) (ISBN: 0198556683); and Bodanszky, Principles of Peptide Synthesis (Springer Laboratory), Springer Verlag (December 1993) (ISBN: 0387564314), the disclosures of which are incorporated herein by reference in their entireties.

Non-natural amino acids can be incorporated during solid phase chemical synthesis. For example, D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., Biochem. Biophys. Res. Com. 209:817-821 (1995)), and various halogenated phenylalanine derivatives.

Amino acid analogues having detectable labels are also usefully incorporated during synthesis to provide a labeled polypeptide.

Biotin, for example, can be added using biotinoyl—(9-fluorenylmethoxycarbonyl)-L-lysine (FMOC biocytin) (Molecular Probes, Eugene, Oreg., USA). The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS—FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)—TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, including, e.g., Fmoc-2-aminobicyclo[2.2.1]heptane-2-carboxylic acid, Fmoc-3-endo-aminobicyclo[2.2.1]heptane-2-endo-carboxylic acid, Fmoc-3-exo-aminobicyclo[2.2.1]heptane-2-exo-carboxylic acid, Fmoc-3-endo-amino-bicyclo[2.2.1]hept-5-ene-2-endo-carboxylic acid, Fmoc-3-exo-amino-bicyclo[2.2.l]hept-5-ene-2-exo-carboxylic acid, Fmoc-cis-2-amino-1-cyclohexanecarboxylic acid, Fmoc-trans-2-amino-1-cyclohexanecarboxylic acid, Fmoc-1-amino-1-cyclopentanecarboxylic acid, Fmoc-cis-2-amino-1-cyclopentanecarboxylic acid, Fmoc-1-amino-1-cyclopropanecarboxylic acid, Fmoc-D-2-amino-4-(ethylthio)butyric acid, Fmoc-L-2-amino-4-(ethylthio)butyric acid, Fmoc-L-buthionine, Fmoc-S-methyl-L-Cysteine, Fmoc-2-aminobenzoic acid (anthranillic acid), Fmoc-3-aminobenzoic acid, Fmoc-4-aminobenzoic acid, Fmoc-2-aminobenzophenone-2′-carboxylic acid, Fmoc-N-(4-aminobenzoyl)-b-alanine, Fmoc-2-amino-4,5-dimethoxybenzoic acid, Fmoc-4-aminohippuric acid, Fmoc-2-amino-3-hydroxybenzoic acid, Fmoc-2-amino-5-hydroxybenzoic acid, Fmoc-3-amino-4-hydroxybenzoic acid, Fmoc-4-amino-3-hydroxybenzoic acid, Fmoc-4-amino-2-hydroxybenzoic acid, Fmoc-5-amino-2-hydroxybenzoic acid, Fmoc-2-amino-3-methoxybenzoic acid, Fmoc-4-amino-3-methoxybenzoic acid, Fmoc-2-amino-3-methylbenzoic acid, Fmoc-2-amino-5-methylbenzoic acid, Fmoc-2-amino-6-methylbenzoic acid, Fmoc-3-amino-2-methylbenzoic acid, Fmoc-3-amino-4-methylbenzoic acid, Fmoc-4-amino-3-methylbenzoic acid, Fmoc-3-amino-2-naphtoic acid, Fmoc-D,L-3-amino-3-phenylpropionic acid, Fmoc-L-Methyldopa, Fmoc-2-amino-4,6-dimethyl-3-pyridinecarboxylic acid, Fmoc-D,L-?-amino-2-thiophenacetic acid, Fmoc-4-(carboxymethyl)piperazine, Fmoc-4-carboxypiperazine, Fmoc-4-(carboxymethyl)homopiperazine, Fmoc-4-phenyl-4-piperidinecarboxylic acid, Fmoc-L-1,2,3,4-tetrahydronorharman-3-carboxylic acid, Fmoc-L-thiazolidine-4-carboxylic acid, all available from The Peptide Laboratory (Richmond, Calif., USA).

Non-natural residues can also be added biosynthetically by engineering a suppressor tRNA, typically one that recognizes the UAG stop codon, by chemical aminoacylation with the desired unnatural amino acid and. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl Acad. Sci. USA 96(9):4780-5 (1999).

The isolated proteins, protein fragments and fusion proteins of the present invention can also include nonnative inter-residue bonds, including bonds that lead to circular and branched forms.

The isolated proteins and protein fragments of the present invention can also include post-translational and post-synthetic modifications, either throughout the length of the protein or localized to one or more portions thereof.

For example, when produced by recombinant expression in eukaryotic cells, the isolated proteins, fragments, and fusion proteins of the present invention will typically include N-linked and/or O-linked glycosylation, the pattern of which will reflect both the availability of glycosylation sites on the protein sequence and the identity of the host cell. Further modification of glycosylation pattern can be performed enzymatically.

As another example, recombinant polypeptides of the invention may also include an initial modified methionine residue, in some cases resulting from host-mediated processes.

When the proteins, protein fragments, and protein fusions of the present invention are produced by chemical synthesis, post-synthetic modification can be performed before deprotection and cleavage from the resin or after deprotection and cleavage. Modification before deprotection and cleavage of the synthesized protein often allows greater control, e.g. by allowing targeting of the modifying moiety to the N-terminus of a resin-bound synthetic peptide.

Useful post-synthetic (and post-translational) modifications include conjugation to detectable labels, such as fluorophores.

A wide variety of amine-reactive and thiol-reactive fluorophore derivatives have been synthesized that react under nondenaturating conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X.

A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yello, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

The polypeptides of the present invention can also be conjugated to fluorophores, other proteins, and other macromolecules, using bifunctional linking reagents.

Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

The proteins, protein fragments, and protein fusions of the present invention can be conjugated, using such cross-linking reagents, to fluorophores that are not amine- or thiol-reactive.

Other labels that usefully can be conjugated to the proteins, protein fragments, and fusion proteins of the present invention include radioactive labels, echosonographic contrast reagents, and MRI contrast agents.

The proteins, protein fragments, and protein fusions of the present invention can also usefully be conjugated using cross-linking agents to carrier proteins, such as KLH, bovine thyroglobulin, and even bovine serum albumin (BSA), to increase immunogenicity for raising antibodies that recognize the ORF-encoded peptide immunogen.

The proteins, protein fragments, and protein fusions of the present invention can also usefully be conjugated to polyethylene glycol (PEG); PEGylation increases the serum half life of proteins administered intravenously for replacement therapy. Delgado et al., Crit. Rev. Ther. Drug Carrier Syst. 9(3-4):249-304 (1992); Scott et al., Curr. Pharm. Des. 4(6):423-38 (1998); DeSantis et al., Curr. Opin. Biotechnol. 10(4):324-30 (1999), incorporated herein by reference in their entireties. PEG monomers can be attached to the protein directly or through a linker, with PEGylation using PEG monomers activated with tresyl chloride (2,2, 2-trifluoroethanesulphonyl chloride) permitting direct attachment under mild conditions.

The isolated proteins of the present invention, including fusions thereof, can be produced by recombinant expression, typically using the expression vectors of the present invention as above-described or, if fewer than about 100 amino acids, by chemical synthesis (typically, solid phase synthesis), and, on occasion, by in vitro translation.

Production of the isolated proteins of the present invention can optionally be followed by purification.

Purification of recombinantly expressed proteins is now well within the skill in the art. See, e.g., Thorner et al. (eds.), Applications of Chimeric Genes and Hybrid Proteins, Part A: Gene Expression and Protein Purification (Methods in Enzymology, Volume 326), Academic Press (2000), (ISBN: 0121822273); Harbin (ed.), Cloning, Gene Expression and Protein Purification: Experimental Procedures and Process Rationale, Oxford Univ. Press (2001) (ISBN: 0195132947); Marshak et al., Strategies for Protein Purification and Characterization: A Laboratory Course Manual, Cold Spring Harbor Laboratory Press (1996) (ISBN: 0-87969-385-1); and Roe (ed.), Protein Purification Applications, Oxford University Press (2001), the disclosures of which are incorporated herein by reference in their entireties, and thus need not be detailed here.

Briefly, however, if purification tags have been fused through use of an expression vector that appends such tag, purification can be effected, at least in part, by means appropriate to the tag, such as use of immobilized metal affinity chromatography for polyhistidine tags. Other techniques common in the art include ammonium sulfate fractionation, immunoprecipitation, fast protein liquid chromatography (FPLC), high performance liquid chromatography (HPLC), and preparative gel electrophoresis.

Purification of chemically-synthesized peptides can readily be effected, e.g., by HPLC.

Accordingly, it is an aspect of the present invention to provide the isolated proteins of the present invention in pure or substantially pure form.

A purified protein of the present invention is an isolated protein, as above described, that is present at a concentration of at least 95%, as measured on a mass basis with respect to total protein in a composition. Such purities can often be obtained during chemical synthesis without further purification, as, e.g., by HPLC. Purified proteins of the present invention can be present at a concentration (measured on a mass basis with respect to total protein in a composition) of 96%, 97%, 98%, and even 99%. The proteins of the present invention can even be present at levels of 99.5%, 99.6%, and even 99.7%, 99.8%, or even 99.9% following purification, as by HPLC.

Although high levels of purity are preferred when the isolated proteins of the present invention are used as therapeutic agents—such as vaccines, or for replacement therapy—the isolated proteins of the present invention are also useful at lower purity. For example, partially purified proteins of the present invention can be used as immunogens to raise antibodies in laboratory animals.

Thus, in another aspect, the present invention provides the isolated proteins of the present invention in substantially purified form. A “substantially purified protein” of the present invention is an isolated protein, as above described, present at a concentration of at least 70%, measured on a mass basis with respect to total protein in a composition. Usefully, the substantially purified protein is present at a concentration, measured on a mass basis with respect to total protein in a composition, of at least 75%, 80%, or even at least 85%, 90%, 91%, 92%, 93%, 94%, 94.5% or even at least 94.9%.

In preferred embodiments, the purified and substantially purified proteins of the present invention are in compositions that lack detectable ampholytes, acrylamide monomers, bis-acrylamide monomers, and polyacrylamide.

The proteins, fragments, and fusions of the present invention can usefully be attached to a substrate. The substrate can porous or solid, planar or non-planar; the bond can be covalent or noncovalent.

For example, the proteins, fragments, and fusions of the present invention can usefully be bound to a porous substrate, commonly a membrane, typically comprising nitrocellulose, polyvinylidene fluoride (PVDF), or cationically derivatized, hydrophilic PVDF; so bound, the proteins, fragments, and fusions of the present invention can be used to detect and quantify antibodies, e.g. in serum, that bind specifically to the immobilized protein of the present invention.

As another example, the proteins, fragments, and fusions of the present invention can usefully be bound to a substantially nonporous substrate, such as plastic, to detect and quantify antibodies, e.g. in serum, that bind specifically to the immobilized protein of the present invention. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof; when the assay is performed in standard microtiter dish, the plastic is typically polystyrene.

The proteins, fragments, and fusions of the present invention can also be attached to a substrate suitable for use as a surface enhanced laser desorption ionization source; so attached, the protein, fragment, or fusion of the present invention is useful for binding and then detecting secondary proteins that bind with sufficient affinity or avidity to the surface-bound protein to indicate biologic interaction therebetween. The proteins, fragments, and fusions of the present invention can also be attached to a substrate suitable for use in surface plasmon resonance detection; so attached, the protein, fragment, or fusion of the present ivnention is useful for binding and then detecting secondary proteins that bind with sufficient affinity or avidity to the surface-bound protein to indicate biological interaction therebetween.

Antibodies and Antibody-Producing Cells

In another aspect, the invention provides antibodies, including fragments and derivatives thereof, that bind specifically to the ORF-encoded peptides, and fragments thereof, of the present invention, or that bind to one or more of the proteins and protein fragments encoded by the isolated nucleic acids of the present invention. The antibodies of the present invention specifically recognize any or all of linear epitopes, discontinuous epitopes, or conformational epitopes of such proteins or protein fragments, either as present on the protein in its native conformation or, in some cases, as present on the proteins as denatured, as, e.g., by solubilization in SDS.

In other embodiments, the invention provides antibodies, including fragments and derivatives thereof, the binding of which can be competitively inhibited by one or more of the ORF-encoded peptides and peptide fragments of the present invention, or by one or more of the proteins and protein fragments encoded by the isolated nucleic acids of the present invention.

As used herein, the term “antibody” refers to a polypeptide, at least a portion of which is encoded by at least one immunoglobulin gene, which can bind specifically to a first molecular species, and to fragments or derivatives thereof that remain capable of such specific binding.

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies of the present invention will discriminate over adventitious binding to proteins other than the ORF-encoded peptide by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the protein of the present invention in human serum.

Typically, the affinity or avidity of an antibody (or antibody multimer, as in the case of an IgM pentamer) of the present invention for a protein or protein fragment of the present invention will be at least about 1×10⁻⁶ molar (M), typically at least about 5×10⁻⁷ M, usefully at least about 1×10⁻⁷ M, with affinities and avidities of at least 1×10⁻⁸ M, 5×10⁻⁹ M, and 1×10⁻¹⁰ M proving especially useful.

The antibodies of the present invention can be naturally-occurring forms, such as IgG, IgM, IgD, IgE, and IgA, from any mammalian species.

Human antibodies can, but will infrequently, be drawn directly from human donors or human cells. In such case, antibodies to the proteins of the present invention will typically have resulted from fortuitous immunization, such as autoimmune immunization, with the protein or protein fragments of the present invention. Such antibodies will typically, but will not invariably, be polyclonal.

Human antibodies are more frequently obtained using transgenic animals that express human immunoglobulin genes, which transgenic animals can be affirmatively immunized with the protein immunogen of the present invention. Human Ig-transgenic mice capable of producing human antibodies and methods of producing human antibodies therefrom upon specific immunization are described, inter alia, in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE and IgA antibodies of the present invention are also usefully obtained from other mammalian species, including rodents—typically mouse, but also rat, guinea pig, and hamster—lagomorphs, typically rabbits, and also larger mammals, such as sheep, goats, cows, and horses. In such cases, as with the transgenic human-antibody-producing non-human mammals, fortuitous immunization is not required, and the non-human mammal is typically affirmatively immunized, according to standard immunization protocols, with the protein or protein fragment of the present invention.

As discussed above, virtually all fragments of 8 or more contiguous amino acids of the proteins of the present invention can be used effectively as immunogens when conjugated to a carrier, typically a protein such as bovine thryoglobulin, keyhole limpet hemocyanin, or bovine serum albumin, conveniently using a bifunctional linker such as those described elsewhere above, which discussion is incorporated by reference here.

Immunogenicity can also be conferred by fusion of the proteins and protein fragments of the present invention to other moieties.

For example, peptides of the present invention can be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85:5409-5413 (1988); Posnett et al., J. Biol. Chem. 263, 1719-1725 (1988).

Protocols for immunizing non-human mammals are well-established in the art, Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference, and often include multiple immunizations, either with or without adjuvants such as Freund's complete adjuvant and Freund's incomplete adjuvant.

Antibodies from nonhuman mammals can be polyclonal or monoclonal, with polyclonal antibodies having certain advantages in immunohistochemical detection of the proteins of the present invention and monoclonal antibodies having advantages in identifying and distinguishing particular epitopes of the proteins of the present invention.

Following immunization, the antibodies of the present invention can be produced using any art-accepted technique. Such techniques are well known in the art, Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907); Howard et al. (eds.), Basic Methods in Antibody Production and Characterization, CRC Press (2000) (ISBN: 0849394457); Harlow et al. (eds.), Antibodies: A Laboratory Manual, Cold Spring Harbor Laboratory (1998) (ISBN: 0879693142); Davis (ed.), Monoclonal Antibody Protocols, Vol. 45, Humana Press (1995) (ISBN: 0896033082); Delves (ed.), Antibody Production: Essential Techniques, John Wiley & Son Ltd (1997) (ISBN: 0471970107); Kenney, Antibody Solution: An Antibody Methods Manual, Chapman & Hall (1997) (ISBN: 0412141914), incorporated herein by reference in their entireties, and thus need not be detailed here.

Briefly, however, such techniques include, inter alia, production of monoclonal antibodies by hybridomas and expression of antibodies or fragments or derivatives thereof from host cells engineered to express immunoglobulin genes or fragments thereof. These two methods of production are not mutually exclusive: genes encoding antibodies specific for the proteins or protein fragments of the present invention can be cloned from hybridomas and thereafter expressed in other host cells. Nor need the two necessarily be performed together: e.g., genes encoding antibodies specific for the proteins and protein fragments of the present invention can be cloned directly from B cells known to be specific for the desired protein, as further described in U.S. Pat. No. 5,627,052, the disclosure of which is incorporated herein by reference in its entirety, or from antibody-displaying phage.

Recombinant expression in host cells is particularly useful when fragments or derivatives of the antibodies of the present invention are desired.

Host cells for recombinant antibody production—either whole antibodies, antibody fragments, or antibody derivatives—can be prokaryotic or eukaryotic.

Prokaryotic hosts are particularly useful for producing phage displayed antibodies of the present invention. The technology of phage-displayed antibodies, in which antibody variable region fragments are fused, for example, to the gene III protein (pIII) or gene VIII protein (pVIII) for display on the surface of filamentous phage, such as M13, is by now well-established, Sidhu, Curr. Opin. Biotechnol. 11(6):610-6 (2000); Griffiths et al., Curr. Opin. Biotechnol. 9(1):102-8 (1998); Hoogenboom et al., Immunotechnology, 4(1):1-20 (1998); Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433-455 (1994), and techniques and protocols required to generate, propagate, screen (pan), and use the antibody fragments from such libraries have recently been compiled, Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, Academic Press, Inc. (1996); Abelson et al. (eds.), Combinatorial Chemistry, Methods in Enzymology vol. 267, Academic Press (May 1996), the disclosures of which are incorporated herein by reference in their entireties.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention.

For example, antibody fragments of the present invention can be produced in Pichia pastoris, Takahashi et al., Biosci. Biotechnol. Biochem. 64(10):2138-44 (2000); Freyre et al., J. Biotechnol. 76(2-3):157-63 (2000); Fischer et al., Biotechnol. Appl. Biochem. 30 (Pt 2):117-20 (1999); Pennell et al., Res. Immunol. 149(6):599-603 (1998); Eldin et al., J. Immunol. Methods. 201(1):67-75 (1997); and in Saccharomyces cerevisiae, Frenken et al., Res. Immunol. 149(6):589-99 (1998); Shusta et al., Nature Biotechnol. 16(8):773-7 (1998), the disclosures of which are incorporated herein by reference in their entireties.

Antibodies, including antibody fragments and derivatives, of the present invention can also be produced in insect cells, Li et al., Protein Expr. Purif. 21(1):121-8 (2001); Ailor et al., Biotechnol. Bioeng. 58(2-3):196-203 (1998); Hsu et al., Biotechnol. Prog. 13(l):96-104 (1997); Edelman et al., Immunology 91(1):13-9 (1997); and Nesbit et al., J. Immunol. Methods. 151(1-2):201-8 (1992), the disclosures of which are incorporated herein by reference in their entireties.

Antibodies and fragments and derivatives thereof of the present invention can also be produced in plant cells, Giddings et al., Nature Biotechnol. 18(11):1151-5 (2000); Gavilondo et al., Biotechniques 29(1):128-38 (2000); Fischer et al., J. Biol. Regul. Homeost. Agents 14(2):83-92 (2000); Fischer et al., Biotechnol. Appl. Biochem. 30 (Pt 2):113-6 (1999); Fischer et al., Biol. Chem. 380(7-8):825-39 (1999); Russell, Curr. Top. Microbiol. Immunol. 240:119-38 (1999); and Ma et al., Plant Physiol. 109(2):341-6 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells.

Verma et al., J. Immunol. Methods 216(1-2):165-81 (1998), review and compare bacterial, yeast, insect and mammalian expression systems for expression of antibodies.

Antibodies of the present invention can also be prepared by cell free translation, as further described in Merk et al., J. Biochem. (Tokyo). 125(2):328-33 (1999) and Ryabova et al., Nature Biotechnol. 15(1):79-84 (1997), and in the milk of transgenic animals, as further described in Pollock et al., J. Immunol. Methods 231(1-2):147-57 (1999), the disclosures of which are incorporated herein by reference in their entireties.

The invention further provides antibody fragments that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful fragments are Fab, Fab′, Fv, F(ab)′₂, and single chain Fv (scFv) fragments. Other useful fragments are described in Hudson, Curr. Opin. Biotechnol. 9(4):395-402 (1998).

It is also an aspect of the present invention to provide antibody derivatives that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful derivatives are chimeric, primatized, and humanized antibodies; such derivatives are less immunogenic in human beings, and thus more suitable for in vivo administration, than are unmodified antibodies from non-human mammalian species.

Chimeric antibodies typically include heavy and/or light chain variable regions (including both CDR and framework residues) of immunoglobulins of one species, typically mouse, fused to constant regions of another species, typically human. See, e.g., U.S. Pat. No. 5,807,715; Morrison et al., Proc. Natl. Acad. Sci USA.81(21):6851-5 (1984); Sharon et al., Nature 309(5966):364-7 (1984); Takeda et al., Nature 314(6010):452-4 (1985), the disclosures of which are incorporated herein by reference in their entireties. Primatized and humanized antibodies typically include heavy and/or light chain CDRs from a murine antibody grafted into a non-human primate or human antibody V region framework, usually further comprising a human constant region, Riechmann et al., Nature 332(6162):323-7 (1988); Co et al., Nature 351(6326):501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

The antibodies of the present invention, including fragments and derivatives thereof, can usefully be labeled. It is, therefore, another aspect of the present invention to provide labeled antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

The choice of label depends, in part, upon the desired use.

For example, when the antibodies of the present invention are used for immunohistochemical staining of tissue samples, the label can usefully be an enzyme that catalyzes production and local deposition of a detectable product.

Enzymes typically conjugated to antibodies to permit their immunohistochemical visualization are well known, and include alkaline phosphatase, β-galactosidase, glucose oxidase, horseradish peroxidase (HRP), and urease. Typical substrates for production and deposition of visually detectable products include o-Nitrophenyl-beta-D-galactopyranoside (ONPG); o-Phenylenediamine Dihydrochloride (OPD); p-Nitrophenyl Phosphate (PNPP); p-Nitrophenyl-beta-D-galactopryanoside (PNPG); 3′,3′Diaminobenzidine (DAB); 3-Amino-9-ethylcarbazole (AEC); 4-Chloro-l-naphthol (CN); 5-Bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133:331-53 (1986); Kricka et al., J. Immunoassay 17(1):67-83 (1996); and Lundqvist et al., J. Biolumin. Chemilumin. 10(6):353-9 (1995), the disclosures of which are incorporated herein by reference in their entireties. Kits for such enhanced chemiluminescent detection (ECL) are available commercially.

The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores.

There are a wide variety of fluorophore labels that can usefully be attached to the antibodies of the present invention.

For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yello, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention.

For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as 33P, 32P, 35S, ³H, and ¹²⁵I.

As another example, when the antibodies of the present invention are used for radioimmunotherapy, the label can usefully be ²²⁸Th, ²²⁷Ac, ²²⁵Ac, ²²³Ra, ²¹³Bi, ²¹²Pb, ²¹²Bi, ²¹¹At, ²⁰³Pb, ¹⁹⁴Os, ¹⁸⁸Re, ¹⁵³Re. ¹⁵³Sm, ¹⁴⁹Tb, ¹³¹I, ¹²⁵I, ¹¹¹In, ¹⁰⁵Rh, ^{99m}mTc, ⁹⁷Ru, ⁹⁰Y, ⁹⁰Sr, ⁸⁸Y, ⁷²Se, ⁶⁷Cu, or ⁴⁷Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2):529-38 (1998), or by radioisotopic labeling As would be understood, use of the labels described above is not restricted to the application as for which they were mentioned.

The antibodies of the present invention, including fragments and derivatives thereof, can also be conjugated to toxins, in order to target the toxin's ablative action to cells that display and/or express the proteins of the present invention. Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975), the disclosures of which are incorporated herein by reference in their entireties, for review.

The antibodies of the present invention can usefully be attached to a substrate, and it is, therefore, another aspect of the invention to provide antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention, attached to a substrate.

Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

For example, the antibodies of the present invention can usefully be attached to paramagnetic microspheres, typically by biotin-streptavidin interaction, which microsphere can then be used for isolation of cells that express or display the proteins of the present invention. As another example, the antibodies of the present invention can usefully be attached to the surface of a microtiter plate for ELISA.

As noted above, the antibodies of the present invention can be produced in prokaryotic and eukaryotic cells. It is, therefore, another aspect of the present invention to provide cells that express the antibodies of the present invention, including hybridoma cells, B cells, plasma cells, and host cells recombinantly modified to express the antibodies of the present invention.

In yet a further aspect, the present invention provides aptamers evolved to bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Business Methods

As mentioned above, the genome-derived single exon probes and microarrays of the present invention are useful in methods of doing business. In another aspect, therefore, the present invention provides such methods of doing business.

A first such method comprises: selling and/or licensing genome-derived single-exon microarrays to a customer desiring to measure gene expression, in consideration of fees paid by such customer.

Such methods can usefully be implemented on a computer. In such computerized embodiments, the method comprises: making available for computerized query a database having a record corresponding to each genome-derived single exon microarray available for sale and/or license; responding to a customer query of the database by returning to the customer at least one record, or an identifier of that record, that best meets the customer query criteria; and offering for sale or license to the querying customer the genome-derived single exon microarray identified in that at least one record.

This method can usefully be implemented by making the database resident on a server computer and available for query by a remotely located client, for example by query over the Internet, an intranet, LAN or WAN. The method can usefully include a later step of permitting the customer to place an order for the identified microarray. This later step can usefully employ the one-click method described and claimed in U.S. Pat. No. 5,960,411, incorporated herein by reference in its entirety.

The genome-derived single exon microarrays of the present invention, as final products, have utility in a second method of doing business, the method comprising designing and/or manufacturing a genome-derived single exon microarray, in consideration of fees paid by those desiring a custom nucleic acid microarray, that has genome-derived single exon probes sharing at least one common attribute.

Such method can usefully be implemented on a computer. In such computerized embodiments, the method comprises: receiving from a customer at least one criterion for common probe attribute, such as tissue of expression; using the at least one criterion to identify within a database having records corresponding to available genome-derived single exon probes those that meet the criterion; and then disposing such identified probes on a support substrate capable of functioning in microarray hybridization experiments. Examples 5-14, infra, identify the level of expression in one or more of ten tissues or cell types of 16,834 human genome-derived single exon probes, each of which is specifically useful in such a method.

The genome-derived single exon microarrays of the present invention are further useful in a third method of doing business, wherein expression data obtained from use of the genome-derived single exon probes and genome-derived single exon microarrays of the present invention are made available by subscription to customers that desire such information, in consideration for fees.

This aspect of the invention can usefully be implemented on a computer. In such computerized embodiments, the method comprises: making available to a subscription client for computerized query a database having records containing expression data generated using genome-derived single exon probes disposed upon microarrays; and responding to a customer query of the database by returning to the customer at least one record, or an identifier of the at least one record, that best meets the customer query criteria. Each of examples 5-14 presents records from such a database, organized by tissue and/or cell type, and collectively containing expression data for 16,834 single exon probes collectively including 16,465 individual exons.

The response to the customer in this method can usefully return only the amount of information minimally required to permit the customer to decide whether to pay for further information from the identified record or records. The method can also usefully include a later step of permitting the customer to place an order for the identified microarray. This later step can usefully employ the one-click method described and claimed in U.S. Pat. No. 5,960,411, incorporated herein by reference in its entirety. The third method of doing business, in each of its embodiments, can usefully be performed by making the database resident on a server computer and available for query by a remotely located client, for example by query over the Internet, an intranet, a LAN or WAN.

Thus, the genome-derived single exon nucleic acid microarrays of the present invention are further useful in a method of doing business, the method comprising: advertising the availability for distribution, sale, or license of genome-derived single exon probes, microarrays, and/or data therefrom; and then selling stock in the company. This method can usefully be implemented on a computer, wherein the advertising is performed on an Internet web page, in an Internet chat room, and/or a Usenet newsgroup; and wherein the stock sale is consummated through an electronic brokerage.

The following examples are offered by way of illustration and not by way of limitation.

### EXAMPLE 1

### Preparation of Single Exon Microarrays from Exons Predicted in Human Genomic Sequence

Bioinformatics Results

All human BAC sequences in fewer than 10 pieces that had been accessioned in a five month period immediately preceding this study were downloaded from GenBank. This corresponds to ≈2200 clones, totaling ≈350 MB of sequence, or approximately 10% of the human genome.

After masking repetitive elements using the program CROSS_MATCH, the sequence was analyzed for open reading frames using three separate gene finding programs. The three programs predict genes using independent algorithmic methods developed on independent training sets: GRAIL uses a neural network, GENEFINDER uses a hidden Markoff model, and DICTION, a program proprietary to Genetics Institute, operates according to a different heuristic. The results of all three programs were used to create a prediction matrix across the segment of genomic DNA.

The three gene finding programs yielded a range of results. GRAIL identified the greatest percentage of genomic sequence as putative coding region, 2% of the data analyzed. GENEFINDER was second, calling 1%, and DICTION yielded the least putative coding region, with 0.8% of genomic sequence called as coding region.

The consensus data were as follows. GRAIL and GENEFINDER agreed on 0.7% of genomic sequence, GRAIL and DICTION agreed on 0.5% of genomic sequence, and the three programs together agreed on 0.25% of the data analyzed. That is, 0.25% of the genomic sequence was identified by all three of the programs as containing putative coding region.

Exons predicted by any two of the three programs (“consensus exons”) were assorted into “gene bins” using two criteria: (1) any 7 consecutive exons within a 25 kb window were placed together in a bin as likely contributing to a single gene, and (2) all exons within a 25 kb window were placed together in a bin as likely contributing to a single gene if fewer than 7 exons were found within the 25 kb window. PCR

The largest exon from each gene bin that did not span repetitive sequence was then chosen for amplification, as were all consensus exons longer than 500 bp. This method approximated one exon per gene; however, a number of genes were found to be represented by multiple elements.

Previously, we had determined that DNA fragments fewer than 250 bp in length do not bind well to the amino-modified glass surface of the slides used as support substrate for construction of microarrays; therefore, amplicons were designed in the present experiments to approximate 500 bp in length.

Accordingly, after selecting the largest exon per gene bin, a 500 bp fragment of sequence centered on the exon was passed to the primer picking software, PRIMER3 (available online for use at http://www-genome.wi.mit.edu/cgi-bin/primer/). A first additional sequence was commonly added to each exon-unique 5′ primer, and a second, different, additional sequence was commonly added to each exon-unique 3′ primer, to permit subsequent reamplification of the amplicon using a single set of “universal” 5′ and 3′ primers, thus immortalizing the amplicon. The addition of universal priming sequences also facilitates sequence verification, and can be used to add a cloning site should some exons be found to warrant further study.

The exons were then PCR amplified from genomic DNA, verified on agarose gels, and sequenced using the universal primers to validate the identity of the amplicon to be spotted in the microarray.

Primers were supplied by Operon Technologies (Alameda, Calif.). PCR amplification was performed by standard techniques using human genomic DNA (Clontech, Palo Alto, Calif.) as template. Each PCR product was verified by SYBR® green (Molecular Probes, Inc., Eugene, Oreg.) staining of agarose gels, with subsequent imaging by Fluorimager (Molecular Dynamics, Inc., Sunnyvale, Calif.). PCR amplification was classified as successful if a single band appeared.

The success rate for amplifying exons of interest directly from genomic DNA using PCR was approximately 75%. FIG. 5 graphs the distribution of predicted exon length and distribution of amplified PCR products, with exon length shown by dashed line and PCR product length shown by solid line. Although the range of exon sizes is readily seen to extend to beyond 900 bp, the mean predicted exon size was only 229 bp, with a median size of 150 bp (n=9498). With an average amplicon size of 475±25 bp, approximately 50% of the average PCR amplification product contained predicted coding region, with the remaining 50% of the amplicon containing either intron, intergenic sequence, or both.

Using a strategy predicated on amplifying about 500 bp, it was found that long exons had a higher PCR failure rate. To address this, the bioinformatics process was adjusted to amplify 1000, 1500 or 2000 bp fragments from exons larger than 500 bp. This improved the rate of successful amplification of exons exceeding 500 bp, constituting about 9.2% of the exons predicted by the gene finding algorithms.

Approximately 75% of the probes disposed on the array (90% of those that successfully PCR amplified) were sequence-verified by sequencing in both the forward and reverse direction using MegaBACE sequencer (Molecular Dynamics, Inc., Sunnyvale, Calif.), universal primers, and standard protocols.

Some genomic clones (BACs) yielded very poor PCR and sequencing results. The reasons for this are unclear, but may be related to the quality of early draft sequence or the inclusion of vector and host contamination in some submitted sequence data.

Although the intronic and intergenic material flanking coding regions could theoretically interfere with hybridization during microarray experiments, subsequent empirical results demonstrated that differential expression ratios were not significantly affected by the presence of noncoding sequence. The variation in exon size was similarly found not to affect differential expression ratios significantly; however, variation in exon size was observed to affect the absolute signal intensity (data not shown).

The 350 MB of genomic DNA was, by the above-described process, reduced to 9750 discrete probes, which were spotted in duplicate onto glass slides using commercially available instrumentation (MicroArray GenII Spotter and/or MicroArray GenIII Spotter, Molecular Dynamics, Inc., Sunnyvale, Calif.). Each slide additionally included either 16 or 32 E. coli genes, the average hybridization signal of which was used as a measure of background biological noise.

Each of the probe sequences was BLASTed against the human EST data set, the NR data set, and SwissProt GenBank (May 7, 1999 release 2.0.9).

One third of the probe sequences (as amplified) produced an exact match (BLAST Expect (“E”) values less than 1 e-100) (i.e., 1×10⁻¹⁰⁰) to either an EST (20% of sequences) or a known mRNA (13% of sequences). A further 22% of the probe sequences showed some homology to a known EST or mRNA (BLAST E values from 1e−5 to 1e−99) (1×10⁻⁵ to 1×10⁻⁹⁹). The remaining 45% of the probe sequences showed no significant sequence homology to any expressed, or potentially expressed, sequences present in public databases.

All of the probe sequences (as amplified) were then analyzed for protein similarities with the SwissProt database using BLASTX, Gish et al., Nature Genet. 3:266 (1993). The predicted functional breakdowns of the ⅔ of probes identical or homologous to known sequences are presented in Table 1.

TABLE 1
Function of Predicted Exons As Deduced From
Comparative Sequence Analysis

| | | | Function Predicted from |
|---|---|---|---|
| | | | Comparative Sequence |
| Total | V6 chip | V7 chip | Analysis |
| 211 | 96 | 115 | Receptor |
| 120 | 43 | 77 | Zinc Finger |
| 30 | 11 | 19 | Homeobox |
| 25 | 9 | 16 | Transcription Factor |
| 17 | 11 | 7 | Transcription |
| 118 | 57 | 61 | Structural |
| 95 | 39 | 56 | Kinase |
| 36 | 18 | 18 | Phosphatase |
| 83 | 31 | 52 | Ribosomal |
| 45 | 19 | 26 | Transport |
| 21 | 7 | 14 | Growth Factor |
| 17 | 12 | 5 | Cytochrome |
| 50 | 33 | 17 | Channel |

As can be seen, the two most common types of genes were transcription factors and receptors, making up 2.2% and 1.8% of the arrayed elements, respectively.

### EXAMPLE 2

### Gene Expression Measurements From Genome-derived Single Exon Microarrays

The two genome-derived single exon microarrays prepared according to Example 1 were hybridized in a series of simultaneous two-color fluorescence experiments to (1) Cy3-labeled cDNA synthesized from message drawn individually from each of brain, heart, liver, fetal liver, placenta, lung, bone marrow, HeLa, BT 474, or HBL 100 cells, and (2) Cy5-labeled cDNA prepared from message pooled from all ten tissues and cell types, as a control in each of the measurements. Hybridization and scanning were carried out using standard protocols and Molecular Dynamics equipment.

Briefly, MRNA samples were bought from commercial sources (Clontech, Palo Alto, Calif. and Amersham Pharmacia Biotech (APB)). Cy3-dCTP and Cy5-dCTP (both from APB) were incorporated during separate reverse transcriptions of 1 μg of polyA⁺ mRNA performed using 1 μg oligo(dT)12-18 primer and 2 μg random 9mer primers as follows. After heating to 70° C., the RNA:primer mixture was snap cooled on ice. After snap cooling on ice, added to the RNA to the stated final concentration was: 1× Superscript II buffer, 0.01 M DTT, 100μM DATP, 100 μM dGTP, 100 μM dTTP, 50 μM dCTP, 50 μM Cy3-dCTP or Cy5-dCTP 50 μM, and 200 U Superscript II enzyme. The reaction was incubated for 2 hours at 42° C. After 2 hours, the first strand cDNA was isolated by adding 1 U Ribonuclease H, and incubating for 30 minutes at 37° C. The reaction was then purified using a Qiagen PCR cleanup column, increasing the number of ethanol washes to 5. Probe was eluted using 10 mM Tris pH 8.5.

Using a spectrophotometer, probes were measured for dye incorporation. Volumes of both Cy3 and Cy5 cDNA corresponding to 50 pmoles of each dye were then dried in a Speedvac, resuspended in 30 μl hybridization solution containing 50% formamide, 5× SSC, 0.2 μg/μl poly(dA), 0.2 μg/μl human cₒt1 DNA, and 0.5% SDS.

Hybridizations were carried out under a coverslip, with the array placed in a humid oven at 42° C. overnight. Before scanning, slides were washed in 1× SSC, 0.2% SDS at 55° C. for 5 minutes, followed by 0.1× SSC, 0.2% SDS, at 55° C. for 20 minutes. Slides were briefly dipped in water and dried thoroughly under a gentle stream of nitrogen.

Slides were scanned using a Molecular Dynamics Gen3 scanner, as described. Schena (ed.), Microarray Biochip: Tools and Technology, Eaton Publishing Company/BioTechniques Books Division (2000) (ISBN: 1881299376).

Although the use of pooled cDNA as a reference permitted the survey of a large number of tissues, it attenuates the measurement of relative gene expression, since every highly expressed gene in the tissue/cell type-specific fluorescence channel will be present to a level of at least 10% in the control channel. Because of this fact, both signal and expression ratios (the latter hereinafter, “expression” or “relative expression”) for each probe were normalized using the average ratio or average signal, respectively, as measured across the whole slide.

Data were accepted for further analysis only when signal was at least three times greater than biological noise, the latter defined by the average signal produced by the E. coli control genes.

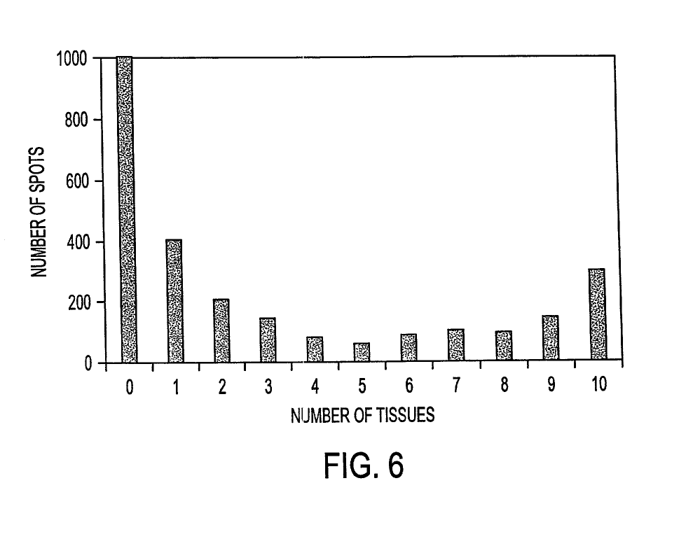

The relative expression signal for these probes was then plotted as a function of tissue or cell type, and is presented in FIG. 6.

FIG. 6 shows the distribution of expression across a panel of ten tissues. The graph shows the number of sequence-verified products that were either not expressed (“0”), expressed in one or more but not all tested tissues (“1” - “9”), and expressed in all tissues tested (“10”).

Of 9999 arrayed elements on the two microarrays (including positive and negative controls and “failed” products), 2353 (51%) were expressed in at least one tissue or cell type. Of the gene elements showing significant signal—where expression was scored as “significant” if the normalized Cy3 signal was greater than 1, representing signal 5-fold over biological noise (0.2)—39% (991) were expressed in all 10 tissues. The next most common class (15%) consisted of gene elements expressed in only a single tissue.

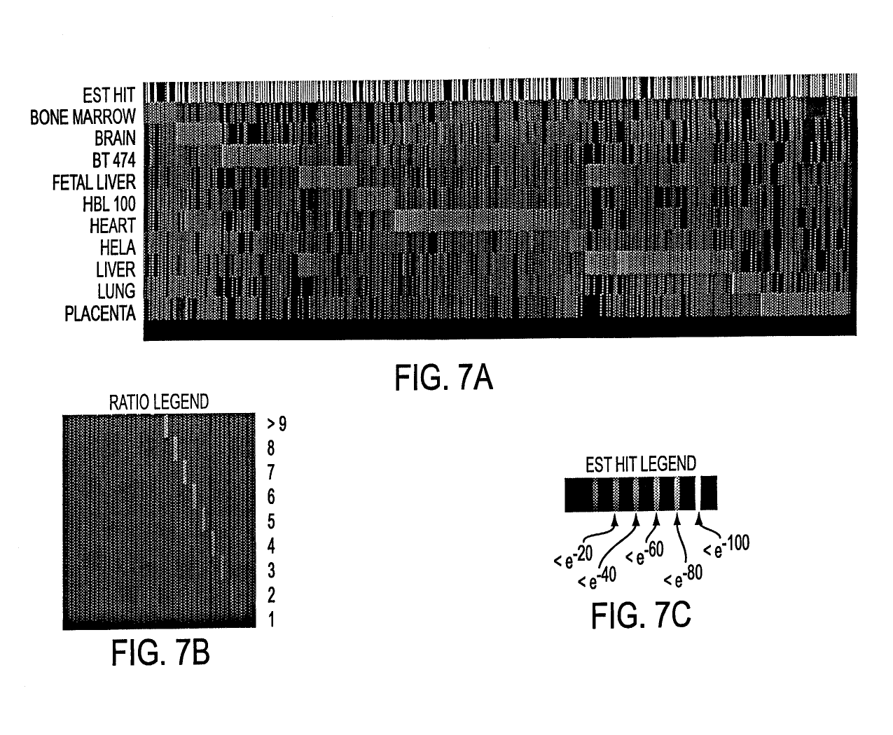

The genes expressed in a single tissue were further analyzed, and the results of the analyses are compiled in FIG. 7.

FIG. 7A is a matrix presenting the expression of all verified sequences that showed signal intensity greater than 3 in at least one tissue. Each clone is represented by a column in the matrix. Each of the 10 tissues assayed is represented by a separate row in the matrix, and relative expression (expression ratio) of a clone in that tissue is indicated at the respective node by intensity of green shading, with the intensity legend shown in panel B. The top row of the matrix (“EST Hit”) contains “bioinformatic” rather than “physical” expression data—that is, presents the results returned by query of EST, NR and SwissProt databases using the probe sequence. The legend for “bioinformatic expression” (i.e., degree of homology returned) is presented in panel C. Briefly, white is known, black is novel, with gray depicting nonidentical with significant homology. “E”xpect values are: white—E values<1e−100) (i.e., <1×10⁻¹⁰⁰); gray—E values from 1e−05 to 1e−99 (i.e., 1×10⁻⁵ to 1×10⁻⁹⁹); black—E values>1e−05 (i.e., >1×10⁻⁵).

As FIG. 7 readily shows, heart and brain were demonstrated to have the greatest numbers of genes that were shown to be uniquely expressed in the respective tissue. In brain, 200 uniquely expressed genes were identified; in heart, 150. The remaining tissues gave the following figures for uniquely expressed genes: liver, 100; lung, 70; fetal liver, 150; bone marrow, 75; placenta, 100; HeLa, 50; HBL, 100; and BT474, 50.

It was further observed that there were many more “novel” genes among those that were up-regulated in only one tissue, as compared with those that were down-regulated in only one tissue. In fact, it was found that exons whose expression was measurable in only a single of the tested tissues were represented in sequencing databases at a rate of only 11%, whereas 36% of the exons whose expression was measurable in 9 of the tissues were present in public databases. As for those exons expressed in all ten tissues, fully 45% were present in existing expressed sequence databases. These results are not unexpected, since genes expressed in a greater number of tissues have a higher likelihood of being, and thus of having been, discovered by EST approaches.

Comparison of Signal from Known and Unknown Genes

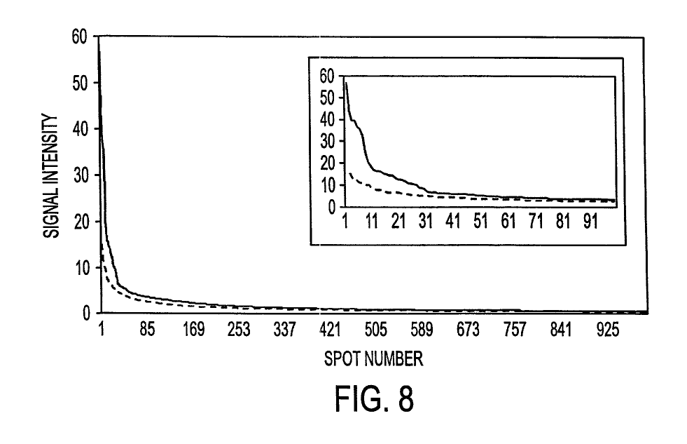

The normalized signal of the genes found to have high homology to genes present in the GenBank human EST database were compared to the normalized signal of those genes not found in the GenBank human EST database. The data are shown in FIG. 8.

FIG. 8 shows in dashed line the normalized Cy3 signal intensity for all sequence-verified products with a BLAST Expect (“E”) value of greater than 1e−30 (designated “unknown”) upon query of existing EST, NR and SwissProt databases, and shows in blue the normalized Cy3 signal intensity for all sequence-verified products with a BLAST Expect value of less than 1e−30 (“known”). Note that biological background noise has an averaged normalized Cy3 signal intensity of 0.2.

As expected, the most highly expressed of the exons were “known” genes. This is not surprising, since very high signal intensity correlates with very commonly-expressed genes, which have a higher likelihood of being found by EST sequence.

However, a significant point is that a large number of even the high expressers were “unknown”. Since the genomic approach used to identify genes and to confirm their expression does not bias exons toward either the 3′ or 5′ end of a gene, many of these high expression genes will not have been detected in an end-sequenced cDNA library.

The significant point is that presence of the gene in an EST database is not a prerequisite for incorporation into a genome-derived microarray, and further, that arraying such “unknown” exons can help to assign function to as-yet undiscovered genes.

Verification of Gene Expression

To ascertain the validity of the approach described above to identify genes from raw genomic sequence, expression of two of the probes was assayed using reverse transcriptase polymerase chain reaction (RT PCR) and northern blot analysis.

Two microarray probes were selected on the basis of exon size, prior sequencing success, and tissue-specific gene expression patterns as measured by the microarray experiments. The primers originally used to amplify the two respective exons from genomic DNA were used in RT PCR against a panel of tissue-specific cDNAs (Rapid-Scan gene expression panel 24 human cDNAs) (OriGene Technologies, Inc., Rockville, Md.).

Sequence AL079300_{—}1 was shown by microarray hybridization to be present in cardiac tissue, and sequence AL031734_{—}1 was shown by microarray experiment to be present in placental tissue (data not shown). RT-PCR on these two sequences confirmed the tissue-specific gene expression as measured by microarrays, as ascertained by the presence of a correctly sized PCR product from the respective tissue type cDNAs.

Clearly, all microarray results cannot, and indeed should not, be confirmed by independent assay methods, or the high throughput, highly parallel advantages of microarray hybridization assays will be lost. However, in addition to the two RT-PCR results presented above, the observation that ⅓ of the arrayed genes exist in expression databases provides powerful confirmation of the power of our methodology—which combines bioinformatic prediction with expression confirmation using genome-derived single exon microarrays—to identify novel genes from raw genomic data.

To verify that the approach further provides correct characterization of the expression patterns of the identified genes, a detailed analysis was performed of the microarrayed sequences that showed high signal in brain.

For this latter analysis, sequences that showed high (normalized) signal in brain, but which showed very low (normalized) signal (less than 0.5, determined to be biological noise) in all other tissues, were further studied. There were 82 sequences that fit these criteria, approximately 2% of the arrayed elements. The 10 sequences showing the highest signal in brain in microarray hybridizations are detailed in Table 2, along with assigned function, if known or reasonably predicted.

TABLE 2
Function of the Most Highly
Expressed Genes Expressed Only in Brain

| | | | Homology | |
|---|---|---|---|---|
| | | | to EST | |
| Microarray | Normal- | Expres- | present | Gene Function |
| Sequence | ized | sion | in | as described |
| Name | Signal | Ratio | GenBank | by GenBank |
| AP000217-1 | 5.2 | +7.7 | High | S-100 protein, |
| | | | | b-chain, Ca²⁺ |
| | | | | binding |
| | | | | protein |
| | | | | expressed in |
| | | | | central |
| | | | | nervous system |
| AP000047-1 | 2.3 | | High | Unknown |
| | | | | Function |
| AC006548-9 | 1.7 | | High | Similar to |
| | | | | mouse membrane |
| | | | | glyco-protein |
| | | | | M6, expressed |
| | | | | in central |
| | | | | nervous system |
| AC007245-5 | 1.5 | | High | Similar to |
| | | | | amphiphysin, a |
| | | | | synaptic |
| | | | | vesicle- |
| | | | | associated |
| | | | | protein. Ref |
| | | | | 21 |
| L44140-4 | 1.2 | +2.0 | High | Endothelial |
| | | | | actin-binding |
| | | | | protein found |
| | | | | in nonmuscle |
| | | | | filamin |
| AC004689-9 | 1.2 | +3.5 | High | Protein |
| | | | | Phosphatase |
| | | | | PP2A, |
| | | | | neuronal/ |
| | | | | downregulates |
| | | | | activated |
| | | | | protein |
| | | | | kinases |
| AL031657-1 | 1.2 | +3.0 | High | Unknown |
| | | | | function/ |
| | | | | Contains the |
| | | | | anhyrin motif, |
| | | | | a common |
| | | | | protein |
| | | | | sequence motif |
| AC009266-2 | 1.1 | +3.7 | Low | Low homology |
| | | | | to the |
| | | | | Synaptotagmin |
| | | | | I protein in |
| | | | | rat/present at |
| | | | | low levels |
| | | | | throughout rat |
| | | | | brain |
| AP000086-1 | 1.0 | +2.7 | Low | Unknown, very |
| | | | | poor homology |
| | | | | to collagen |
| AC004689-3 | 1.0 | | High | Protein |
| | | | | Phosphatase |
| | | | | PP2A, |
| | | | | neuronal/ |
| | | | | downregulates |
| | | | | activated |
| | | | | protein |
| | | | | kinases |

Of the ten sequences studied by these latter confirmatory approaches, eight were previously known. Of these eight, six had previously been reported to be important in the central nervous system or brain. The exon giving the highest signal (AP00217-1) was found to be the gene encoding an S100B Ca²⁺ binding protein, reported in the literature to be highly and uniquely expressed in the central nervous system. Heizmann, Neurochem. Res. 9:1097 (1997).

A number of the brain-specific probe sequences (including AC006548-9, AC009266-2) did not have homology to any known human cDNAs in GenBank but did show homology to rat and mouse cDNAs. Sequences AC004689-9 and AC004689-3 were both found to be phosphatases present in neurons (Millward et al., Trends Biochem. Sci. 24(5):186-191 (1999)). Two microarray sequences, AP000047-1 and AP000086-1 have unknown function, with AP000086-1 being absent from GenBank. Functionality can now be narrowed down to a role in the central nervous system for both of these genes, showing the power of designing microarrays in this fashion.

Next, the function of the chip sequences with the highest (normalized) signal intensity in brain, regardless of expression in other tissues, was assessed. In this latter analysis, we found expression of many more common genes, since the sequences were not limited to those expressed only in brain. For example, looking at the 20 highest signal intensity spots in brain, 4 were similar to tubulin (AC00807905; AF146191-2; AC007664-4; AF14191-2), 2 were similar to actin (AL035701-2; AL034402-1), and 6 were found to be homologous to glyceraldehyde-3-phosphate dehydrogenase (GAPDH) (AL035604-1; Z86090-1; AC006064-L, AC006064-K; AC035604-3; AC006064-L). These genes are often used as controls or housekeeping genes in microarray experiments of all types.

Other interesting genes highly expressed in brain were a ferritin heavy chain protein, which is reported in the literature to be found in brain and liver (Joshi et al., J. Neurol. Sci. 134(Suppl):52-56 (1995)), a result confirmed with the array. Other highly expressed chip sequences included a translation elongation factor 1α (AC007564-4), a DEAD-box homolog (AL023804-4), and a Y-chromosome RNA-binding motif (Chai et al., Genomics 49(2):283-89 (1998))(AC007320-3). A low homology analog (AP00123-½) to a gene, DSCR1, thought to be involved in trisomy 21 (Down's syndrome), showed high expression in both brain and heart, in agreement with the literature (Fuentes et al., Mol. Genet. 4(10):1935-44 (1995)).

As a further validation of the approach, we selected the BAC AC006064 to be included on the array. This BAC was known to contain the GAPDH gene, and thus could be used as a control for the exon selection process. The gene finding and exon selection algorithms resulted in choosing 25 exons from BAC AC006064 for spotting onto the array, of which four were drawn from the GAPDH gene. Table 3 shows the comparison of the average expression ratio for the 4 exons from BAC006064 compared with the average expression ratio for 5 different dilutions of a commercially available GAPDH cDNA (Clontech).

TABLE 3
Comparison of Expression Ratio, for each tissue, of
GAPDH

| | AC006064 (n = 4) | Control ( n = 5) |
|---|---|---|
| Bone Marrow | −1.81 ± 0.11 | −1.85 ± 0.08 |
| Brain | −1.41 ± 0.11 | −1.17 ± 0.05 |
| BT474 | 1.85 ± 0.09 | 1.66 ± 0.12 |
| Fetal Liver | −1.62 ± 0.07 | −1.41 ± 0.05 |
| HBL100 | 1.32 ± 0.05 | 2.64 ± 0.12 |
| Heart | 1.16 ± 0.09 | 1.56 ± 0.10 |
| HeLa | 1.11 ± 0.06 | 1.30 ± 0.15 |
| Liver | −1.62 ± 0.22 | −2.07 ± |
| Lung | −4.95 ± 0.93 | −3.75 ± 0.21 |
| Placenta | −3.56 ± 0.25 | −3.52 ± 0.43 |

Each tissue shows excellent agreement between the experimentally chosen exons and the control, again demonstrating the validity of the present exon mining approach. In addition, the data also show the variability of expression of GAPDH within tissues, calling into question its classification as a housekeeping gene and utility as a housekeeping control in microarray experiments.

### EXAMPLE 3

### Representation of Sequence and Expression Data as a “Mondrian ”

For each genomic clone processed for microarray as above-described, a plethora of information was accumulated, including full clone sequence, probe sequence within the clone, results of each of the three gene finding programs, EST information associated with the probe sequences, and microarray signal and expression for multiple tissues, challenging our ability to display the information.

Accordingly, we devised a new tool for visual display of the sequence with its attendant annotation which, in deference to its visual similarity to the paintings of Piet Mondrian, is hereinafter termed a “Mondrian”. FIGS. 3 and 4 present the key to the information presented on a Mondrian.

FIG. 9 presents a Mondrian of BAC AC008172 (bases 25,000 to 130,000 shown), containing the carbamyl phosphate synthetase gene (AF154830.1). Purple background within the region shown as field 81 in FIG. 3 indicates all 37 known exons for this gene.

As can be seen, GRAIL II successfully identified 27 of the known exons (73%), GENEFINDER successfully identified 37 of the known exons (100%), while DICTION identified 7 of the known exons (19%).

Seven of the predicted exons were selected for physical assay, of which 5 successfully amplified by PCR and were sequenced. These five exons were all found to be from the same gene, the carbamyl phosphate synthetase gene (AF154830.1).

The five exons were arrayed, and gene expression measured across 10 tissues. As is readily seen in the Mondrian, the five chip sequences on the array show identical expression patterns, elegantly demonstrating the reproducibility of the system.

FIG. 10 is a Mondrian of BAC AL049839. We selected 12 exons from this BAC, of which 10 successfully sequenced, which were found to form between 5 and 6 genes. Interestingly, 4 of the genes on this BAC are protease inhibitors. Again, these data elegantly show that exons selected from the same gene show the same expression patterns, depicted below the red line. From this figure, it is clear that our ability to find known genes is very good. A novel gene is also found from 86.6 kb to 88.6 kb, upon which all the exon finding programs agree. We are confident we have two exons from a single gene since they show the same expression patterns and the exons are proximal to each other. Backgrounds in the following colors indicate a known gene (top to bottom):

red=kallistatin protease inhibitor (P29622);

purple=plasma serine protease inhibitor (P05154);

turquoise=al anti-chymotrypsin (P01011);

mauve=40S ribosomal protein (P08865).

Note that chip sequence 8 and 12 did not sequence verify.

### EXAMPLE 4

### Genome-derived Single Exon Probes Useful for Measuring Human Gene Expression

The protocols set forth in Examples 1 and 2, supra, were applied with some modification to additional human genomic sequence as it became newly available in GenBank. From the collective efforts of these and the experiments reported in Example 2, we generated 16,834 unique human genome-derived single exon probes that could be shown to be expressed at significant levels in one or more of ten tested tissues.

Modifications to the protocols for bioinformatic prediction of exons set forth in Examples 1 and 2 were as follows.

First, we added a fourth gene prediction program, GENSCAN, to the three originally used, DICTION, GENEFINDER, and GRAIL.

Second, we increased the resolution of our exon predictions, as follows.

In the experiments reported in Examples 1 and 2, we applied a 25 bp window in scanning genomic sequence: exons were called when any two of the three gene prediction programs identified an exon anywhere within the window. In the more recent experiments, we looked for consensus on a nucleotide by nucleotide basis: when any two or more of the four programs identified the nucleotide as falling within an exon, the nucleotide was called as belonging to an exon. This had the additional benefit of merging overlapping predicted exons.

Finally, we applied a lower size threshold of 75 contiguous nucleotides to each consensus exon.

Each probe was completely sequenced on both strands prior to its use on a genome-derived single exon microarray; sequencing confirmed the exact chemical structure of each probe. An added benefit of sequencing is that it placed us in possession of a set of single base-incremented fragments of the sequenced nucleic acid, starting from the sequencing primer 3′ OH. (Since the single exon probes were first obtained by PCR amplification from genomic DNA, we were of course additionally in possession of an even larger set of single base incremented fragments of each of the 16,834 single exon probes, each fragment corresponding to an extension product from one of the two amplification primers.)

Hybridization analysis was conducted essentially as set forth in Examples 1 and 2, with one modification.

In Examples 1 and 2, we used a pool of 10 tissues/cell types as control. We have since observed that every probe that demonstrates expression in the control pool can readily be shown to be expressed in HeLa cells, and have used HeLa as the source of control message in the more recent experiments.

In the analysis of hybridization results, the uniform absolute signal intensity threshold used in Examples 1 and 2 to identify signals large enough to be considered biologically significant (0.5, representing a level roughly 10 times greater than the average of all E. coli control spots on a first iteration chip) was replaced with a statistical threshold determined for each channel and each hybridization as follows.

Starting typically with 32 E. coli sequences, spotted in duplicate (left and right side) for a total of 64 control spots per microarray, control spots were eliminated if we observed more than a five-fold difference between the left and right side raw (unnormalized) signals for the probe.

The median of the normalized signal from the remaining control spots was calculated (see infra for normalization routine).

Control spots were eliminated as outliers if they had signal intensity greater than the median of the normalized signals plus 2.4 (where 2.4 is roughly 12 times the observed standard deviation of control spot populations) and normalization was performed as set forth below.

The mean and standard deviation of the normalized signal intensity from the remaining control spots were calculated, and the mean plus three standard deviations of the controls was then applied as a minimum intensity threshold for the particular hybridization experiment, giving a 99% confidence that expression is significant.

Signal normalization was accomplished as follows. For each hybridization (each microarray, separately for each of the two colors), the median value of all of the spots was determined. For each probe, the normalized signal value is the arithmetic mean of the probe's duplicate intensities (each DNA probe, including controls, is spotted twice per slide) divided by the population median.

Using this threshold, we identified a total of 16,834 single exon probes that produce significant signal in one or more of ten tested tissues/cell types. The structures of these 16,834 single exon probes are clearly presented in the Sequence Listing appended hereto as SEQ ID NOs: 1-16,834. The 16 nt 5′ primer universal primer sequence and 16 nt 3′ universal primer sequence present on the amplicon are not included in the sequence listing, and may be found in commonly owned and copending U.S. patent application Ser. No. 09/608,408, filed Jun. 30, 2000, the disclosure of which is incorporated herein by reference in its entirety.

The sequences of the exons present within each of the single exon probes is presented in the Sequence Listing as SEQ ID NOs: 16,835-33,299. Certain of the exons appear in a plurality of probes, accounting for the slight discrepancy in the number of probe sequences versus the number of exon sequences.

We also predicted the sequence of the ORF within the exon of each of the 16,834 probes, where ORF was defined as that portion of an exon that can be translated in its entirety into a sequence of contiguous amino acids.

To predict the ORF, we first looked for consensus as between any two or more of the four gene prediction programs. Consensus was required in two parameters: (1) as with prediction of the exon, each nucleotide must have been identified by two or more programs as falling within an exon; and, additionally, (2) the programs relied upon to establish that consensus must have agreed on the frame. Presence of a stop codon disqualified the predicted ORF. ORFs shorter than 50 nt were also disregarded.

Absent consensus as to nucleotide and frame, each of the six frames of the predicted exon were examined individually for stop codons and the longest open reading frame of at least 51 nt selected as the exon's likely ORF. Certain of the exons have no ORF as defined by either set of criteria.

We then translated the predicted ORFs using the standard genetic code. Translations of the ORF-encoded peptides are presented in the Sequence Listing as SEQ ID NOs: 33,300-49,117.

Concordance as among PROBE SEQ ID NO:, EXON SEQ ID NO:, and ORF-encoded peptide SEQ ID NO: is presented collectively in Tables 4-13, presented in electronic format and incorporated by reference respectively in Examples 5-14, infra. The tables also provide the expression data for each probe.

Each example, with its respective table, presents the subset of probes that is significantly expressed in the respective tissue and/or cell type: human brain, heart, liver, fetal liver, placenta, lung, bone marrow, HeLa cells, BT 474 cells, or HBL 100 cells; each example thus presents the subset of probes that was recognized to be useful for measuring expression of their cognate genes in the respective tissue.

The sequence of each of the probes, exons, and ORF-encoded peptides was used as a query to identify the most similar sequence in each of dbEST, GenBank NR, and SWISSPROT. The query programs used were BLAST (nucleic acid sequence query of dbEST and NR), BLASTX (nucleic acid sequence query of SWISSPROT), TBLASTX (peptide sequence query of dbEST and NR), and BLASTP (peptide sequence query of SWISSPROT). Because the query sequences are themselves derived from genomic sequence in GenBank, only nongenomic hits from NR were scored.

The attached Sequence Listing reports, for each SEQ ID NO:, the accession number of the entry from each of the three queried databases that gave the highest absolute expect (“E”) value (the “top hit”), along with the “E” value itself. The Sequence Listing is incorporated herein by reference in its entirety.

Tables 4-13 provide a subset of these query results.

The smallest in value of the expect (“E”) scores for each exon query sequence across the three database divisions was used as a measure of the “expression novelty” of the probe's exon. Each of Tables 4-13 is sorted in descending order based on this measure, reported as “Most Similar (top) Hit BLAST E Value”.

As sorted, each table thus lists its respective probes from least similar to sequences known to be expressed (i.e., highest BLAST E value), at the beginning of each table, to most similar to sequences known to be expressed (i.e., lowest BLAST E value), at the bottom of each table.

Each table further provides, for each listed probe, the accession number of the database sequence that yielded the “Most Similar (top) Hit BLAST E Value” (when queried with exon sequence), along with the name of the database in which the database sequence is found (“Top Hit Database Source”), and a portion of the descriptor for the top hit (“Top Hit Descriptor”) as provided in the sequence database. For those exons that are similar in sequence, but nonidentical to known sequences (e.g., those with BLAST E values between about 1e−05 and 1e−100), the descriptor reveals the likely function of the protein encoded by the probe's exon.

Using BLAST E value cutoffs of 1e−05 (i.e., 1×10⁻⁵) and 1e−100 (i.e., 1×10⁻¹⁰⁰) as evidence of similarity to sequences known to be expressed is of course arbitrary: in Example 2, supra, a BLAST E value of 1e−30 was used as the boundary when only two classes were to be defined for analysis (unknown, >1e−30; known <1e−30) (see also FIG. 8). Furthermore, even when the “Most Similar (Top) Hit BLAST E Value” is low, e.g., less than about 1e−100—which is probative evidence that the query sequence has previously been shown to be expressed—the top hit is highly unlikely exactly to match the probe sequence.

First, such expression entries typically will not have the intronic and/or intergenic sequence present within the single exon probes listed in the Tables. Second, even the exon itself is unlikely in such cases to be present identically in the databases, since most of the EST and mRNA clones in existing databases include multiple exons, without any indication of the location of exon boundaries.

As noted, the data presented in tables 4-13 represent a subset of the data present within the appended Sequence Listing, which is incorporated herein by reference in its entirety. For each probe (SEQ ID NOs: 1-16,834), exon (SEQ ID NOs: 16,835-33,299, respectively), and ORF-encoded peptide (SEQ ID NOs: 33,300-49,117, the sequence listing further provides, through iterated annotation fields <220> and <223> (not necessarily presented in the Sequence Listing LISTING in this order):

(a) the accession number of the BAC or contig from which the sequence was derived (“MAP TO”), thus providing a link to the chromosomal map location and other information about the genomic milieu of the probe sequence;

(b) the name of each tissue or cell type in which significant expression could be demonstrated, with the expression signal in the respective tissue;

(c) the most similar sequence provided by BLAST query of the EST database, with accession number and BLAST E value for the “hit”;

(d) the most similar sequence provided by BLAST query of the GenBank NR/NT database, with accession number and BLAST E value for the “hit”; and

(e) the most similar sequence provided by BLASTX query of the SWISSPROT database, with accession number and BLAST E value for the “hit”.

### EXAMPLE 5

### Genome-derived Single Exon Probes Useful for Measuring Expression of Genes in Human Brain

Diseases of the brain and nervous system are a significant cause of human morbidity and mortality. Increasingly, genetic factors are being found that contribute to predisposition, onset, and/or aggressiveness of most, if not all, of these diseases. Although mutations in single genes have been identified as causative for some diseases of the brain and nervous system, for the most part these disorders are believed to have polygenic etiologies.

For example, over the past few decades Alzheimer's disease (AD), once considered a rare disorder, has become recognized as a major public health problem; over 4,000,000 people in the United States are now estimated to suffer with various stages of this progressive, degenerative brain disorder.

Although there is no agreement on the exact incidence or prevalence of Alzheimer's disease, in part due to varying diagnostic criteria and difficulties of differential diagnosis among dementias, the studies are consistent in pointing to an exponential rise in prevalence of this disease with age. After age 65, the percentage of affected people approximately doubles with every decade of life, regardless of definition. Among people age 85 or older, studies suggest that 25 to 35 percent have dementia, including Alzheimer's disease; one study reports that 47.2 percent of people over age 85 have Alzheimer's disease, exclusive of other dementias.

Alzheimer's disease progressively destroys memory, reason, judgment, language, and, eventually, the ability to carry out even the simplest of tasks. Anatomic changes associated with Alzheimer's disease begin in the entorhinal cortex, proceed to the hippocampus, and then gradually spread to other regions, particularly the cerebral cortex. Chief among such anatomic changes are the presence of characteristic extracellular plaques and internal neurofibrillary tangles.

Alzheimer's disease has been suspected to have a multifactorial genetic etiological component for almost half a century. Sjogren et al., Acta Psychiat. Neurol. Scand. 82(suppl.): 1-152 (1952).

At least four genes have been identified to date that contribute to development of Alzheimer's disease: AD1 is caused by mutations in the amyloid precursor gene (APP); AD2 is associated with the APOE4 allele on chromosome 19; AD3 is caused by mutation in a chromosome 14 gene encoding a 7-transmembrane domain protein, presenilin-1 (PSEN1), and AD4 is caused by mutation in a gene on chromosome 1 that encodes a similar 7-transmembrane domain protein, presenilin-2 (PSEN2).

There is strong evidence, however, for additional, as yet uncharacterized, AD loci on other chromosomes.

For example, Daw et al., Am. J. Hum. Genet. 66: 196-204 (2000), estimated the number of additional quantitative trait loci (QTLs) and their contribution to the variance in age at onset of AD, and reported that 4 loci make a contribution to the variance in age at onset of late-onset AD similar to or greater in magnitude than that made by apoE, with one locus making a contribution several times greater than that of apoE. These results suggest that several genes not yet localized may play a larger role than does apoE in late-onset AD.

In accord, three groups recently announced the possible existence of an AD susceptibility gene on chromosome 10. Bertram et al., Science 290(5500):2302-2303 (2000); Ertekin-Taner et al., Science 290(5500):2303-2304 (2000); and Myers et al., Science 290(5500):2304-23055 (2000).

As another example, multiple sclerosis (MS) affects about 350,000 Americans, with approximately 200 new cases diagnosed each week, with an estimated annual monetary cost in the U.S. alone of $2.5 billion.

Clinically, MS is an unpredictable disorder, with symptoms, presentation and course falling broadly into one of several clinical patterns. In relapsing-remitting (RR) MS, the disease first manifests as a series of attacks followed by complete or partial remissions, with symptoms returning later after a period of stability. In primary-progressive (PP) MS, there is a gradual clinical decline with no distinct remissions, although there may be temporary plateaus or minor relief from symptoms. Secondary-progressive (SP) MS begins with a relapsing-remitting course followed by a later primary-progressive course. Rarely, patients may have a progressive-relapsing (PR) course in which the disease takes a progressive path punctuated by acute attacks. PP, SP, and PR MS are sometimes lumped together and called chronic progressive MS. The waxing and waning course characteristic of RR, SP and PR MS makes differential diagnosis difficult.

Anatomically, MS attacks are associated with focal inflammation in areas of the white matter of the central nervous system (CNS), accompanied or followed by demyelination in these areas, termed plaques. Destruction of the myelin sheath slows or blocks neurological transmission, leading to diminished or lost function. Clinical manifestations depend upon the location of the plaques and severity of demyelination, and range from fatigue, the most common symptom of MS, to visual impairment, due to inflammation of the optic nerve, termed optic neuritis, to numbness and paresthesias, to focal muscular weakness, ataxia, and bladder incontinence.

Increasing evidence suggests that genotype contributes to susceptibility to MS.

As early as 1965, McAlpine, in Multiple Sclerosis: A Reappraisal (McAlpine, ed.), Williams and Wilkins Co. pp. 61-74 (1965), concluded that the risk to a first-degree relative of a patient with multiple sclerosis is at least 15 times that for a member of the general population, but could discern no definite genetic pattern of inheritance.

Subsequently, many studies associated MS with HLA (MHC) haplotype. Haines et al., Hum. Molec. Genet. 7:1229-1234 (1998), studying a data set of 98 multiplex MS families, confirmed earlier reports that genetic linkage to the MHC can be explained by association with the HLA-DR2 allele, but suggested that MHC association explains only between 17% and 62% of the genetic etiology of MS.

From a review of genomic screens, Dyment et al., Hum. Molec. Genet. 6: 1693-1698 (1997), concluded that a number of genes with interacting effects are likely and that no single region has a major influence on familial risk. Chataway et al., Brain 121: 1869-1887 (1998), reporting a follow-up on U.K. studies using a systematic genome screen to determine the genetic basis of MS, stated that a gene of major effect had been excluded from 95% of the genome and one with a moderate role from 65%, results thus suggesting that multiple sclerosis depends on independent or epistatic effects of several genes, each with small individual effects, rather than a very few genes of major biologic importance.

As a yet further example, schizophrenia has long been recognized to have complex, likely polygenic, genetic contributions.

Schizophrenia is a common psychiatric disorder, occurring in 1 to 1.5 percent of the population worldwide, and is characterized by variable constellations of symptoms drawn from a universe of behavioral abnormalities. Although there are accepted alternative diagnostic criteria, primary criteria for diagnosis require two or more of the following, each present for a significant portion of time during a 1-month period (or less if successfully treated): (1) delusions; (2) hallucinations; (3) disorganized speech (e.g., frequent derailment or incoherence); (4) grossly disorganized or catatonic behavior; (5) negative symptoms, i.e., affective flattening, alogia, or avolition. See Diagnostic and Statistic Manual of Mental Disorders DSM-IV-TR, American Psychiatric Association (2000).

Only one such symptom is required if delusions are bizarre or hallucinations consist of a voice keeping up a running commentary on the person's behavior or thoughts, or consist of two or more voices conversing with each other.

Three-quarters of persons with schizophrenia develop the disease between 16 and 25 years of age: onset is uncommon after age 30, rare after age 40. In the 16 to 25 year old age group, schizophrenia affects more men than women; in the 25-30 year old group, the incidence is higher in women than in men. Studies have shown that some persons with schizophrenia recover completely, and many others improve to the point where they can live independently, often with the maintenance of drug therapy. However, approximately 15 percent of people with schizophrenia respond only moderately to medication and require extensive support throughout their lives, while another 15 percent simply do not respond to existing treatment.

Schizophrenia has long been known to have a significant genetic component. Studies have consistently demonstrated that the risk to relatives of a proband with schizophrenia is higher than the risk to relatives of controls. Moldin, in Genetics and Mental Disorders: Report of the NIMH Genetics Workgroup (NIH publication 98-4268, (1998), reviewed family and twin studies published between 1920 and 1987 and found the recurrence risk ratios to be 48 for monozygotic twins, 11 for first-degree relatives, 4.25 for second-degree relatives, and 2 for third-degree relatives. He also found that concordance rates for monozygotic twins averaged 46%, even when reared in different families, whereas the concordance rates for dizygotic twins averaged only 14%. The prevalence of schizophrenia is known to be higher in biologic than in adoptive relatives of schizophrenic adoptees.

The mode of inheritance is unclear, however. Susceptibility has been mapped to many loci, including chromosomes 1q21-q22, 5, 6p23, 8p22-p21, 11q, 13q14-q21, 13q32, 15q15, 15q14, 18p, and 22q11. Chromosome 19 has also been implicated in schizophrenia, at 2 different sites, as have sites on the X chromosome. Wei et al., Nature Genet. 25:376-377 (2000) report more specifically that the NOTCH4 locus is associated with susceptibility to schizophrenia.

In general, however, it is believed that development of schizophrenia involves multiple loci.

For example, Williams et al., Hum. Molec. Genet. 8: 1729-1739 (1999) undertook a systematic search for linkage in 196 affected sib pairs (ASPs) with schizophrenia. Using 229 microsatellite markers at an average intermarker distance of 17.26 cM, followed in a second stage by a further 54 markers allowing the regions identified in stage 1 to be typed at an average spacing of 5.15 cM, Williams et al. considered results on chromosomes 4p, 18q, and Xcen as suggestive; however, given the scores, Williams et al. interpreted their results as suggesting that common genes of major effect (susceptibility ratio more than 3) are unlikely to exist for schizophrenia.

Similarly, Shaw et al., Am. J. Med. Genet. 81(5):364-76 (1998), in a genome-wide search for schizophrenia susceptibility genes, found that twelve chromosomes (1, 2, 4, 5, 8, 10, 11, 12, 13, 14, 16, and 22) had at least one region with a nominal P value <0.05, that two of these chromosomes had a nominal P value<0.01 (chromosomes 13 and 16), and that five chromosomes (1, 2, 4, 11, and 13) had at least one marker with a lod score>2.0, suggesting the existence of multiple loci that contribute to schizophrenia susceptibility.

As yet another example, multiple genes are thought to predispose to epilepsy.

Epilepsy is characterized by recurrent, paroxysmal disorders of cerebral function (seizures); that is, by sudden, brief attacks of altered consciousness, motor activity, sensory phenomena, or inappropriate behavior. The risk of developing epilepsy is 1% in the period from birth to age 20, and 3% at age 75.

Epilepsy is caused by excessive discharge of cerebral neurons. Clinical manifestations depend on the type and location of discharge. In partial seizures, for example, the excess neuronal discharge is contained within one region of the cerebral cortex. Simple partial seizures consist of motor, sensory, or psychomotor phenomena without loss of consciousness; the specific phenomenon reflects the affected area of the brain. In generalized seizures, the discharge bilaterally and diffusely involves the entire cortex. Sometimes a focal lesion of one part of a hemisphere activates the entire cerebrum bilaterally so rapidly that it produces a generalized tonic-clonic seizure before a focal sign appears.

Epilepsy is a family of disorders. Those that are idiopathic are believed to have multiple genetic contributions. For example, idiopathic generalized epilepsy (IGE) is characterized by recurring generalized seizures in the absence of detectable brain lesions and/or metabolic abnormalities. Twin and family studies suggest that genetic factors play a key part in its etiology. Although a mutation in the CACNB4 gene can cause the disorder, linkage to 8q24, Zara et al., Hum. Molec. Genet. 4: 1201-1207(1995), 3q26 and 14q23, Sander et al., Hum. Molec. Genet. 9:1465-1472 (2000), and 2q36 has been also demonstrated, with a multilocus model appearing to fit best the observed familial patterns.

Polygenic contributions to the etiology of various neurologic cancers have similarly been described.

For example, gliomas account for 45% of intracranial tumors, and multiple loci have been implicated in its development, with losses of chromosome 17p, increase in copy number of chromosome 7, structural abnormalities of chromosomes 9p and 19q, and genes on chromosome 10 among the suspects.

Other significant diseases of brain and nervous tissue are also believed to have a genetic, typically polygenic, etiologic component. These diseases include, for example, Parkinson's disease, dementia with Lewy bodies, frontotemporal dementia, corticobasal ganglionic degeneration, progressive supranuclear palsy, prion diseases (Creutzfeld-Jakob, Gerstmann-Strausller-Shenker, familial fatal insomnia), Tourette's Syndrome, corticobasal degeneration, multiple system atrophy, striatonigral degeneration, Shy-Drager syndrome, olivopontocerebellar atrophy, spinocerebellar ataxia, Friedreich ataxia, ataxia-telangiectasia, amyotrophic lateral sclerosis, bulbospinal atrophy (Kennedy's syndrome), spinal muscular atrophy, neuronal storage diseases (sphingolipid, mucopolysaccharide, mucolipid), leukodystrophy, Krabbe disease, metachromic leukodystrophy, adrenoleukodystrophy, Pelizaeus-Merzbacher disease, Canavan disease, mitochondrial encephalomyopathy, Leigh disease, neurofibromatosis (Type 1 and Type II), tuberous sclerosis, paraneoplastic syndrome, subacute cerebellar degeneration, subacute sensory neuropathy, opsoclonus/myoclonus, retinal degeneration, stiff-man syndrome and Von Hippel-Lindau disease.

Many neurologic cancers other than gliomas have also been shown or suspected to have genetic bases or contributions. Among these cancers are astrocytoma, fibrillary astrocytoma, pilocytic astrocytoma, pleomorphic xanthoastrocytoma, oligodendroglioma, ependymoma, gangliocytoma, ganglioglioma, medulloblastoma, primary brain germ cell tumor, pineocytoma, pineoblastoma, and meningioma.

Other disorders of brain and central nervous system that likely have genetic components include the various forms of neural deafness, catatonia, depression, bipolar (manic-depressive) disorder, Wilson's Disease, Pick disease, neuromyelitis optica (Devic disease), central pontine myelinolysis, Marchiafava-Bignami disease, Guillain-Barre syndrome, sleep disorders (insomnia, myoclonus, narcolepsy, cataplexy, sleep apnea), amnesia, aphasias (including Broca's aphasia and Wernicke's aphasia), cortical blindness, visual agnosia, auditory agnosia, and Kluver-Bucy syndrome.

The human genome-derived single exon nucleic acid probes and microarrays of the present invention are useful for predicting, diagnosing, grading, staging, monitoring and prognosing diseases of human brain, particularly those diseases with polygenic etiology. With each of the single exon probes described in this Example having been shown to be expressed at detectable levels in human brain, and with about ⅔ of the probes identifying novel genes, single exon microarrays of the present invention that include such probes provide exceptionally high informational content for such studies.

For example, diagnosis (including differential diagnosis among clinically indistinguishable disorders), staging, and/or grading of a disease can be based upon the quantitative relatedness of a patient gene expression profile to one or more reference expression profiles known to be characteristic of a given neurologic disease, or to specific grades or stages thereof.

In one embodiment, the patient gene expression profile is generated by hybridizing nucleic acids obtained directly or indirectly from transcripts expressed in the patient's brain (or other CNS tissues, including cultured tissues) to the genome-derived single exon microarray of the present invention. Reference profiles are obtained similarly by hybridizing nucleic acids from individuals with known disease. Methods for quantitatively relating gene expression profiles, without regard to the function of the protein encoded by the gene, are disclosed in WO 99/58720, incorporated herein by reference in its entirety.

In another approach, the genome-derived single exon probes and microarrays of the present invention can be used to interrogate genomic DNA, rather than pools of expressed message; this latter approach permits predisposition to and/or prognosis of neurologic disease to be assessed through the massively parallel determination of altered copy number, deletion, or mutation in the patient's genome of exons known to be expressed in human brain. The algorithms set forth in WO 99/58720 can be applied to such genomic profiles without regard to the function of the protein encoded by the interrogated gene.

Table 4, presented herewith in electronic format and incorporated herein by reference in its entirety, presents expression, homology, and functional information for the genome-derived single exon probes of the present invention that are expressed significantly in human brain.

### EXAMPLE 6

Genome-derived Single Exon Probes Useful for Measuring Expression of Genes in Heart

Diseases of the heart and vascular system are a significant cause of human morbidity and mortality. Increasingly, genetic factors are being found that contribute to predisposition, onset, and/or aggressiveness of most, if not all, of these diseases. Although mutations in single genes have on occasion been identified as causative, these disorders are for the most part believed to have polygenic etiologies.

For example, cardiovascular disease (CVD), which includes coronary heart disease, stroke, and peripheral arterial vascular disease, is the leading cause of death in the United States and other developed countries. In developing regions, coronary heart disease and stroke are ranked second and third, respectively, as causes of mortality. In the United States alone, about 1 million deaths (about 42% of total deaths per year) result from CVD each year. CVD is also a significant cause of morbidity, with about 1.5 million people suffering myocardial infarction, and about 500,000 suffering strokes in the United States each year. With risk for CVD increasing with age, and an increasingly aging population, CVD will continue to be a major health problem into the future.

CVD is caused by arterial lesions that begin as fatty streaks, which consist of lipid-laden foam cells, and develop into fibrous plaques. The atherosclerotic plaque may grow slowly, and over several decades may produce a severe stenosis or result in arterial occlusion. Some plaques are stable, but other, more unstable, ones may rupture and induce thrombosis. The thrombi may embolize, rapidly occluding the lumen and leading to myocardial infarction or acute ischemic syndrome.

Risk factors for CVD include age and gender. In addition, a family history of CVD significantly increases risk, indicating a genetic basis for development of this disease complex. Obesity, especially truncal obesity, the cause of which is suspected to be genetic, is yet another risk factor for CVD. Familial disorders such as hyperlipidemia, hypoalphalipoproteinemia, hypertriglyceridemia, hypercholesterolemia, hyperinsulinemia, homocystinuria, and dysbetalipoproteinemia, all of which lead to lipid or lipoprotein abnormalities, can predispose one to the development of CVD. Both insulin-dependent and non-insulin-dependent diabetes mellitus, both of which have genetic components, have been also linked to the development of atherosclerosis.

The literature is replete with evidence for genetic causes of cardiovascular diseases. For example, studies by Allayee et al., Am. J. Hum. Genet. 63:577-585(1998), indicated a genetic association between familial combined hyperlipidemia (FCHL) and small dense LDL particles. The studies also concluded that the genetic determinants for LDL particle size are shared, at least in part, among FCHL families and the more general population at risk for CVD. Juo et al., Am. J. Hum. Genet. 63: 586-594 (1998) demonstrated that small, dense LDL particles and elevated apolipoprotein B levels, both of which are commonly found in members of FCHL families, share a common major gene plus individual polygenic components. The common major gene was estimated to explain 37% of the variants of adjusted LDL particle size and 23% of the variants of adjusted apoB levels.

The atherogenic lipoprotein phenotype (ALP) is a common heritable trait, symptoms of which include a prevalence of small, dense LDL particles, increased levels of triglyceride-rich lipoproteins, reduced levels of high density lipoprotein, and increased risk of CVD, particularly myocardial infarction. Both Nishina et al., Proc. Nat. Acad. Sci. 89: 708-712 (1992) and Rotter et al., Am. J. Hum. Genet. 58: 585-594(1996) demonstrated linkage between ALP and the LDLR locus. Rotter et al., supra, also reported linkage to the CETP locus on chromosome 16 and to the SOD1 locus on chromosome 6, and possibly also to the APOA1/APOC3/APOA4 cluster on chromosome 11.

Mutations in genes identified as components of lipid metabolism, e.g., apolipoprotein E (apoE) and LDL receptor (LDLR), have been shown to be associated with predisposition to the development of CVD. For example, several apoE variants had been found to be associated with familial dysbetalipoproteinemia, characterized by elevated plasma cholesterol and triglyceride levels and an increased risk for atherosclerosis (de Knijff et al., Mutat 4: 178-194 (1994)). Mutations in the LDLR gene have been associated with the familial hypercholesterolemia, an autosomal dominant disorder characterized by elevation of serum cholesterol bound to low density lipoprotein (LDL), that can lead to increased susceptibility to CVD.

To date, mutations in numerous genes have been shown to be associated with increased CVD susceptibility. However, the identified genetic associations are believed not to account for all genetic contributions to CVD.

As yet another example, hypertension is a major health problem because of its high prevalence and its association with increased risk of CVD. Approximately 25% of all adults and over 60% of persons older than 60 years in the United States have high blood pressure.

Arterial or systemic hypertension is diagnosed when the average of two or more diastolic BP measurements on at least two subsequent visits is 90 mm Hg or more, or when the average of multiple systolic BP readings on two or more subsequent visits is consistently greater than 140 mm Hg. Pulmonary hypertension is defined as pressure within the pulmonary arterial system elevated above the normal range; pulmonary hypertension may lead to right ventricle (RV) failure.

Hypertension, together with other cardiovascular risk factors, leads to atherosclerosis and other forms of CVD, primarily by damaging the vascular endothelium. In more than 40% of the U.S. population, hypertension is accompanied by hyperlipidemia and leads to the development of atherosclerotic plaques. In the absence of hyperlipidemia, intimal thickening occurs. Non-atherosclerotic hypertension-induced vascular damage can lead to stroke or heart failure.

Familial diseases associated with secondary hypertension include familial renal disease, polycystic kidney disease, medullary thyroid cancer, pheochromocytoma, and hyperparathyroidism. Hypertension is also twice as common in patients with diabetes mellitus.

More than 95% of all hypertension cases are essential hypertension, that is, lack identifiable antecedent clinical cause. Essential hypertension shows clustering in families and can result from a variety of genetic diseases. In most cases, high blood pressure results from a complex interaction of factors with both genetic and environmental components. The recent search for genes that contribute to the development of essential hypertension has shown that the disorder is polygenic in origin. However, with several exceptions (such as angiotensinogen, angiotensin receptor-1, beta-3 subunit of guanine nucleotide-binding protein, tumor necrosis factor receptor-2, and α-adducin), the particular genes involved are still being sought.

Susceptibility loci for essential hypertension have been mapped to chromosomes 17 and 15q. Hasstedt et al., Am. J. Hum. Genet. 43: 14-22 (1988) measured red cell sodium in 1,800 normotensive members of 16 Utah pedigrees ascertained through hypertensive or normotensive probands, siblings with early stroke death, or brothers with early coronary disease, and suggested that red blood cell sodium was determined by 4 alleles at a single locus. This major locus was thought to explain 29% of the variance in red cell sodium, and polygenic inheritance explained another 54.6%. A higher frequency of the high red blood cell sodium genotype in pedigrees in which the proband was hypertensive rather than normotensive provided evidence that this major locus increases susceptibility to hypertension.

From a study of systolic blood pressure in 278 pedigrees, Perusse et al., Am. J. Hum. Genet. 49: 94-105 (1991) reported that variability in systolic blood pressure is likely influenced by allelic variation of a single gene, with gender and age dependence. They also suggested that a single gene may be associated with a steeper increase of blood pressure with age among males and females.

There is strong evidence, however, for additional as yet uncharacterized, hypertension-associated loci on other chromosomes.

For example, Xu et al., Am. J. Hum. Genet. 64: 1694-1701 (1999) carried out a systematic search for chromosomal regions containing genes that regulate blood pressure by scanning the entire autosomal genome using 367 polymorphic markers. Because of the sampling design, the number of sib pairs, and the availability of genotyped parents, this study represented one of the most powerful of its kind. Although no regions achieved a 5% genomewide significance level, maximum lod scores were greater than 2.0 for regions of chromosomes 3, 11, 15, 16, and 17.

As another example, cardiac arrhythmias account for several thousand deaths each year. Arrhythmias such as ventricular fibrillation, which causes more than 300,000 sudden deaths annually in the United States alone, encompass a multitude of disorders. Another type of arrhythmia, idiopathic dilated cardiomyopathy, of which familial dilated cardiomyopathy accounts for 20-25%, is responsible for more than 10,000 deaths in the United States annually and is the predominant indication for cardiac transplantation.

Cardiac arrhythmias can be divided into bradyarrhythmias (slowed rhythms) or tachyarrhythmias (speeded rhythms). Bradyarrhythmias result from abnormalities of intrinsic automatic behavior or conduction, primarily within the atrioventricular node and the His-Purkinje's network. Tachyarrhythmias are caused by altered automaticity, reentry, or triggered automaticity.

Bradyarrhythmias arising from suspected polygenic disorders include Long QT syndrome 4, atrioventricular block, familial sinus node disease, progressive cardiac conduction defect, and familial cardiomyopathy. Tachyarrhythmias with possible underlying polygenic causes include familial ventricular tachycardia, Wolff-Parkinson-White syndrome, familial arrhythmogenic right ventricular dysplasia, heart-hand syndrome V, Mal de Meleda, familial ventricular fibrillation, and familial noncompaction of left ventricular myocardium.

For some of the arrhythmias, one or more of the causative genes have been identified.

For example, atrioventricular block has been associated with mutations in the SCN5A gene, as well as mutations in a locus mapped to 19q13. Studies have shown linkage of familial sinus node disease to a marker on 10q22-q24. Familial ventricular tachycardia has been linked to mutations in genes encoding the G protein subunit alpha-i2 (GNAI1), and/or related genes. Examination of families with Wolff-Parkinson-White syndrome suggest an autosomal dominant pattern of inheritance and evidence of linkage of the disorder to DNA markers on band 7q3. Linkage analysis shows strong evidence for localization of a gene for Mal de Meleda disease on 8qter. Familial ventricular fibrillation can be caused by mutations in the cardiac sodium channel gene SCN5A. Familial noncompaction of left ventricular myocardium has been linked to mutations in the gene encoding tafazzin (TAZ), or in the FK506-binding protein 1A gene (FKBPlA).

Familial dilated cardiomyopathy is characterized by an autosomal dominant pattern of inheritance with age-related penetrance. The linkage of familial dilated cardiomyopathy to several loci indicate that it is polygenic. These loci include CMD1A on 1p11-q11, CMD1B on 9q13, CMD1C on 10q21, CMD1D on 1q32, CMD1E on 3p, CMD1F on 6q, CMD1G on 2q31, CMD1H on 2q14-q22, and CMD1I, which results from mutation in the DES gene on 2q35. In addition, cardiomyopathy can also be caused by mutations in the ACTC gene, the cardiac beta-myosin heavy chain gene (MYH7), or the cardiac troponin T gene.

Familial arrhythmogenic right ventricular dysplasia is inherited as an autosomal dominant with reduced penetrance and is one of the major genetic causes of juvenile sudden death. It is estimated that the prevalence of familial arrhythmogenic right ventricular dysplasia ranges from 6 per 10,000 in the general population to 4.4 per 1,000 in some areas. Several loci for familial arrhythmogenic right ventricular dysplasia have been mapped indicating that this disease is also polygenic in nature. These loci include ARVD1 on 14q23-q24, ARVD2 on 1q42-q43, ARVD3 on 14q12-q22, ARVD4 on 2q32.1-q32.3, ARVD5 on 3p23, and ARVD6 on 10p14-p12.

Progressive cardiac conduction defect (PCCD), also called Lenegre-Lev disease, is one of the most common cardiac conduction diseases. It is characterized by progressive alteration of cardiac conduction through the His-Purkinje system with right or left bundle branch block and widening of QRS complexes, leading to complete atrioventricular block and ultimately causing syncope and sudden death. It represents the major cause of pacemaker implantation in the world (0.15 implantations per 1,000 inhabitants per year in developed countries). The cause of PCCD is unknown but familial cases with right bundle branch block have been reported suggesting that at least some cases are of genetic origin. Reports have linked PCCD to HB1 on 19q13.3, and to mutations in the SCN5A gene (Schott et al., Nature Genet. 23: 20-21 (1999)).

As yet a further example, congenital heart disease occurs at a rate of 8 per 1000 live births, which corresponds to approximately 32,000 infants with newly diagnosed congenital heart disease each year in the United States. Twenty percent of infants with congenital heart disease die within the first year of life. Approximately 80% of the first-year survivors live to reach adulthood. Congenital heart disease also has economic impact due to the estimated 20,000 surgical procedures performed to correct circulatory defects in these patients. The estimated number of adults with congenital heart disease in the United States is currently about 900,000.

In 90% of patients, congenital heart disease is attributable to multifactorial inheritance. Only 5-10% of malformations are due to primary genetic factors, which are either chromosomal or a result of a single mutant gene.

The most common congenital heart disease found in adults is bicuspid aortic valve. This defect occurs in 2% of the general population and accounts for approximately 50% of operated cases of aortic stenosis in adults. Atrial septal defect is responsible for 30-40% of congenital heart disease seen in adults. The most common congenital cardiac defect observed in the pediatric population is ventricular septal defect, which accounts for 15-20% of all congenital lesions. Tetralogy of Fallot is the most common cyanotic congenital anomaly observed in adults. Other congenital heart diseases include Eisenmenger's syndrome, patent ductus arteriosus, pulmonary stenosis, coarctation of the aorta, transposition of the great arteries, tricuspid atresia, univentricular heart, Ebstein's anomaly, and double-outlet right ventricle.

A number of studies have identified putative genetic loci associated with one or more congenital heart diseases.

Congenital heart disease affects more than 40% of all Down syndrome patients. The candidate chromosomal region containing the putative gene or genes for congenital heart disease associated with Down syndrome is 21q22.2-q22.3, between ETS2 and MX1.

DiGeorge syndrome (DGS) is characterized by several symptoms including outflow tract defects of the heart such as teratology of Fallot. Most cases result from a deletion of chromosome 22q11.2 (the DiGeorge syndrome chromosome region, or DGCR). The 22q11 deletion is the second most common cause of congenital heart disease after Down syndrome. Several genes are lost in this deletion including the putative transcription factor TUPLE1. This deletion is associated with a variety of phenotypes, e.g., Shprintzen syndrome; conotruncal anomaly face (or Takao syndrome); and isolated outflow tract defects of the heart including Tetralogy of Fallot, truncus arteriosus, and interrupted aortic arch.

Whereas 90% of cases of DGS may now be attributed to a 22qll deletion, other associated chromosome defects have been identified. For example, Greenberg et al., Am. J. Hum. Genet. 43:605-611 (1988), reported 1 case of DGS with del10p13 and one with a 18q21.33 deletion. Fukushima et al., Am. J. Hum. Genet. 51 (suppl.):A80 (1992) reported linkage with a deletion of 4q21.3-q25. Gottlieb et al., Am. J. Hum. Genet. 62: 495-498 (1998) concluded that the deletion of more than 1 region on 10p could be associated with the DGS phenotype. The association of the DiGeorge syndrome with at least 2 and possibly more chromosomal locations suggests strongly the involvement of several genes in this disease.

Digilio et al., J. Med. Genet. 34: 188-190 (1997), calculated empiric risk figures for recurrence of isolated Tetralogy of Fallot in families after exclusion of del(22q11), and concluded that gene(s) different from those located on 22q11 must be involved in causing familial aggregation of nonsyndromic Tetralogy of Fallot. Johnson et al., Am. J. Med. Genet. (1997) conducted a cytogenetic evaluation of 159 cases of Tetralogy of Fallot. They reported that a del(22q11) was identified in 14% who underwent fluorescence in situ hybridization (FISH) testing with the N25 cosmid probe.

Other congenital heart disease are also suspected to be of polygenic origin. For example, Holmes et al., Birth Defects Orig. Art. Ser. X(4): 228-230 (1974) described familial clustering of hypoplastic left heart syndrome in siblings consistent with multifactorial causation.

Other significant diseases of the heart and vascular system are also believed to have a genetic, typically polygenic, etiological component. These diseases include, for example, hypoplastic left heart syndrome, cardiac valvular dysplasia, Pfeiffer cardiocranial syndrome, oculofaciocardiodental syndrome, Kapur-Toriello syndrome, Sonoda syndrome, Ohdo Blepharophimosis syndrome, heart-hand syndrome, Pierre-Robin syndrome, Hirschsprung disease, Kousseff syndrome, Grange occlusive arterial syndrome, Kearns-Sayre syndrome, Kartagener syndrome, Alagille syndrome, Ritscher-Schinzel syndrome, Ivemark syndrome, Young-Simpson syndrome, hemochromatosis, Holzgreve syndrome, Barth syndrome, Smith-Lemli-Opitz syndrome, glycogen storage disease, Gaucher-like disease, Fabry disease, Lowry-Maclean syndrome, Rett syndrome, opitz syndrome, Marfan syndrome, Miller-Dieker lissencephaly syndrome, mucopolysaccharidosis, Bruada syndrome, humerospinal dysostosis, Phaver syndrome, McDonough syndrome, Marfanoid hypermobility syndrome, atransferrinemia, Cornelia de Lange syndrome, Leopard syndrome, Diamond-Blackfan anemia, Steinfeld syndrome, progeria, and Williams-Beuren syndrome.

The human genome-derived single exon nucleic acid probes and microarrays of the present invention are useful for predicting, diagnosing, grading, staging, monitoring and prognosing diseases of human heart and vascular system, particularly those diseases with polygenic etiology. With each of the single exon probes described in this Example shown to be expressed at detectable levels in human heart, and with about ⅔ of the probes identifying novel genes, single exon microarrays of the present invention that include such probes provide exceptionally high informational content for such studies.

For example, diagnosis (including differential diagnosis among clinically indistinguishable disorders), staging, and/or grading of a disease can be based upon the quantitative relatedness of a patient gene expression profile to one or more reference expression profiles known to be characteristic of a given heart or vascular disease, or to specific grades or stages thereof.

In one embodiment, the patient gene expression profile is generated by hybridizing nucleic acids obtained directly or indirectly from transcripts expressed in the patient's heart or vascular tissues to the genome-derived single exon microarray of the present invention. Reference profiles are obtained similarly by hybridizing nucleic acids obtained directly or indirectly from transcripts expressed in heart or vascular tissue of individuals with known disease. Methods for quantitatively relating gene expression profiles, without regard to the function of the protein encoded by the gene, are disclosed in WO 99/58720, incorporated herein by reference in its entirety.

In another approach, the genome-derived single exon probes and microarrays of the present invention can be used to interrogate genomic DNA, rather than pools of expressed message; this latter approach permits predisposition to and/or prognosis of heart or vascular disease to be assessed through the massively parallel determination of altered copy number, deletion, or mutation in the patient's genome of exons known to be expressed in human heart. The algorithms set forth in WO 99/58720 can be applied to such genomic profiles without regard to the function of the protein encoded by the interrogated gene.

Table 5, presented herewith in electronic format and incorporated herein by reference in its entirety, presents expression, homology, and functional information for the genome-derived single exon probes of the present invention that are expressed significantly in human heart.

### EXAMPLE 7

### Genome-derived Single Exon Probes Useful for Measuring Expression of Genes in Liver

Diseases of the liver are a significant cause of human morbidity and mortality. Increasingly, genetic factors are being found that contribute to predisposition, onset, and/or aggressiveness of most, if not all, of these diseases; although causative mutations in single genes have been identified for some, these disorders are believed for the most part to have polygenic etiologies.

For example, cirrhosis is a major public health problem. In the industrialized world, it is among the top ten causes of death; among patients aged 45 to 65, it is the third leading cause of death. The high prevalence is largely the result of alcohol abuse, but other major contributors include chronic hepatitis, biliary disease and iron overload. Approximately 10-15% are cryptogenic.

Cirrhosis is a broad description encompassing the common end stage of many forms of liver injury. Many patients with cirrhosis will remain asymptomatic for years, while others show generalized weakness, anorexia, malaise, and weight loss or, occasionally, more severe symptoms.

The progression from fibrosis, an early consequence of liver disease, to cirrhosis, and the specific histologic morphology that characterizes cirrhosis depend on the extent of injury, the presence of continuing damage, and the response of the liver to damage. The liver may be injured acutely and severely (e.g. necrosis with hepatitis), moderately over months or years (e.g. biliary tract obstruction and chronic active hepatitis), or modestly but continuously (e.g. alcohol abuse).

During the repair process, new vessels connecting the hepatic artery and portal vein to the hepatic venules form within the fibrous sheath that surrounds the surviving nodules of liver cells. These vessels restore the intrahepatic circulatory pathway, but provide relatively low-volume, high-pressure drainage that is less efficient than normal and results in increased portal vein pressure (portal hypertension). Thus, cirrhosis is not static and its features depend on the disease activity and stage.

As cirrhosis is the end stage of many forms of liver disease, many genes have been identified that can contribute to the development of cirrhosis. These include, e.g., the genes responsible for Wilson disease (Online Mendelian Inheritance of Man (“OMIM”) 277900), type IV glycogen storage disease (OMIM 232500), galactosemia (OMIM 230400), and a deficiency of alpha-1-antitrypsin (OMIM 107400). There is substantial evidence, however, for as yet uncharacterized loci which cause cirrhosis.

For example, Iber and Maddrey, Prog. Liver Dis. 2: 290-302 (1965), reviewed 13 previously reported families and 8 new to this study, each with 2 or more affected members. They pointed out that, with a single exception, the multiple cases were in the same generation. Within a given family, the age of onset, clinical course, and biopsy findings were very similar, but there were wide differences between families.

Kalra et al., Hum. Hered. 32:170-175 (1982) studied the families of 220 cases of Indian childhood cirrhosis and 70 families of age-matched controls. The hypotheses of autosomal recessive, partial sex-linkage, and doubly recessive inheritance were found untenable and the authors concluded that multifactorial inheritance was most plausible. Lefkowitch et al., New Eng. J. Med. 307:271-277 (1982) described 4 white American sibs who died between ages 4.5 and 6 years of cirrhosis that closely resembled that of the childhood cirrhosis of Asiatic Indians.

Another example of uncharacterized loci which cause cirrhosis are those related to the risk of alcoholism.

Cloninger, Science 236:410-416 (1987), defined two separate types of alcoholism. According to these definitions, type 1 alcohol abuse has its usual onset after the age of 25 years and is characterized by severe psychological dependence and guilt. Type 1 occurs in both men and women and requires both genetic and environmental factors to become manifest. By contrast, type 2 alcohol abuse has its onset before the age of 25; persons with this type of alcoholism are characterized by their inability to abstain from alcohol and by frequent aggressive and antisocial behavior. Type 2 alcoholism is rarely found in women and is much more heritable.

Despite considerable effort to identify genes related to the risk of alcoholism, relatively few genes have been identified. Some of this work has suggested a relationship between the metabolism of dopamine and alcoholism. Blum et al., J.A.M.A. 263:2055-2060 (1990) and Bolos et al., J.A.M.A. 264:3156-3160 (1990) investigated the relationship of the dopamine D2 receptor (DRD2; OMIM 126450) to alcoholism, but the sample size was small and their results were inconclusive. However, Tiihonen et al., Molec. Psychiat. 4, 286-289 (1999), found a markedly higher frequency in a population of type 1 alcoholics of the low activity allele of the enzyme catechol-O-methyltransferase (COMT, OMIM 116790), which has a crucial role in the metabolism of dopamine, suggesting a role for dopamine metabolism in increased risk of alcoholism. For a brief review of recent progress toward the identification of genes related to risk for alcoholism see Buck, Genome 9:927-928 (1998).

As another example, multiple genes have been shown to predispose to hyperlipoproteinemia or hyperlipidemia. Much attention has been focused on these disorders because there is a strong association of hyperlipidemia, especially hypercholesterolemia, with development of coronary artery disease. Coronary artery disease accounts for at least 25% of all deaths in the United States. Coronary artery disease results when the arteries supplying the heart muscle become occluded by plaques composed of lipids like cholesterol, blood clotting components and blood cells.

The major plasma lipids circulate bound to proteins as macromolecular complexes called lipoproteins. Although closely interrelated, the major lipoprotein classes—chylomicron, very-low-density lipoprotein (VLDL), low-density lipoprotein (LDL), and high-density lipoprotein (HDL)—are usually classified in terms of physicochemical properties (e.g., density after centrifugation). Chylomicrons, the largest lipoproteins, carry exogenous triglyceride from the intestine via the thoracic duct to the venous system and into peripheral sites. VLDL carries endogenous triglyceride primarily from the liver to the same peripheral sites for storage or use. Lipases quickly degrade the triglyceride in VLDL to produce intermediate density lipoproteins (IDL) and within 2 to 6 h, IDL is degraded further to generate LDL, which has a plasma half-life of 2 to 3 days. While the overall fate of LDL is unclear, the liver is responsible for removing approximately 70% and active receptor sites have been found on the surfaces of hepatocytes.

Several monogenic conditions that lead to elevated levels of one or more serum lipoproteins have been defined and the responsible gene identified, including, e.g., hyperlipoproteinemia type I (OMIM 238600), familial hypercholesterolemia (OMIM 143890), and familial defective apolipoprotein B (OMIM 107730). However, in many cases the etiology is unknown and there is strong evidence for additional uncharacterized loci.

For example, Zuliani et al., Arterioscler. Thromb. Vasc. Biol. 19:802-809 (1999) identified a Sardinian family with a recessive form of hypercholesterolemia with the clinical features of familial hypercholesterolemia (OMIM 603813), and found that previously identified genes were not responsible for this disorder. They proposed that in this new lipid disorder, a recessive defect causes a selective impairment of the LDL receptor function in the liver. Ciccarese et al., Am. J. Hum. Genet. 66:453-460 (2000) recently mapped this novel disease locus.

Another example is designated familial combined hyperlipidemia (OMIM 144250) which affects approximately 1-2% of the population in the Western world. This disorder can have its basis in mutation in several novel genes, two of which have been mapped to chromosome 1 (Pajukanta et al., Nature Genet. 18:369-373 (1998)) and chromosome 11 (Aouizerat et al., Am. J. Hum. Genet. 65, 397-412 (1999)). The high frequency of this disorder suggests that most, if not all, hyperlipidemias are of multifactorial genetic etiology.

As yet a further example, primary schlerosing cholangitis (PSC) is a disorder characterized by a patchy obliterative inflammatory fibrosis of the large bile ducts. Chronic inflammation leads to extensive bile duct strictures, cholestasis, and gradual progression to biliary cirrhosis. PSC occurs most often in young men and is commonly associated with inflammatory bowel disease, especially ulcerative colitis. The onset is usually insidious, with gradual, progressive fatigue, pruritus, and jaundice. There is no specific therapy for sclerosing cholangitis, and liver transplantation is the only apparent cure.

The etiology of PSC is not known, but both genetic and immunologic abnormalities have been implicated. However, the frequency of HLA-B8 and HLA-DT2, which are associated with a number of autoimmune diseases, is higher in PSC than normal individuals. Prochazka et al., New Eng. J. Med. 322:1842-1844 (1990) found that 100% of 29 patients with primary sclerosing cholangitis carried the HLA-DRw52a antigen, which is normally present in 35% of the population.

As a still further example, sarcoidosis is a disease of unknown cause characterized by non-caseating granulomas in one or more organ systems. These granulomas may resolve completely or proceed to fibrosis. The disorder is systemic, but the liver is affected in approximately 75% of cases. Sarcoidosis occurs mainly in persons aged 20 to 40 yr and is most common in Northern Europeans and American blacks. The lifetime risk of developing sarcoidosis is particularly high among Swedish men (1.15%), Swedish women (1.6%), and African Americans (2.4%).

The much greater frequency in African Americans relative to the United States population overall suggests a genetic contribution to etiology. Early research studying familial aggregation indicated that the disease may have a nongenetic basis because the family pattern did not conform to a simple Mendelian mode of inheritance (Allison, Sth. Med. J. 57: 27-32 (1964)). However, Headings et al., Ann. N.Y. Acad. Sci. 278:377-385 (1976) favored multifactorial genetic inheritance of susceptibility. Nowack et al., Arch. Intern. Med. 147:481-483 (1987), found an unusually high frequency of HLA-DRS in a study of 440 patients with sarcoidosis in Marburg, Germany. They also concluded that the role of an environmental or infectious agent triggering sarcoidosis cannot be envisaged without considering genetically linked cofactors.

Other significant diseases of liver are also believed to have a genetic, typically polygenic, etiologic component. These diseases include, e.g., primary biliary cirrhosis, Zellweger syndrome, cholestasis-lymphedema syndrome, Alstrom syndrome, primary pulmonary hypertension, Berardinelli-Seip congenital lipodystrophy, iron overload in Africa, neonatal cholestatic hepatitis, autosomal recessive KID syndrome, familial hypotransferrinemia, type I congenital dyserythropoietic anemia, porphyria variegata, Finnish lactic acidosis with hepatic hemosiderosis, Rotor syndrome, essential hypertension, ARC syndrome, type II conjugated hyperbilirubinemia, Lambert syndrome, ichthyosis congenita with biliary atresia, Kabuki make-up syndrome, Meckel syndrome, cerebral aneurysm-cirrhosis syndrome, glycogen storage diseases, polycystic kidney and hepatic disease, isolated Caroli disease, trisomy 18-like syndrome, Osler-Rendu-Weber syndrome 3, fatal intrahepatic cholestasis, Coach syndrome, type C Niemann-Pick disease, and hepatocellular cancer.

Altered responses to a variety of infectious agents that target the liver, especially acute viral hepatitis, have also been shown or are suspected to have genetic bases or contributions. In addition to differential susceptibility to primary infectious agents, these altered responses include predisposition to complicating conditions following contact with particular infectious agents. These include, e.g., development of hepatocellular carcinoma 2 correlated with Hepatitis B infection, and severe hepatic fibrosis following Schistosoma mansoni infection.

The central role of the liver in drug metabolism results in exposure of this organ to a large variety of potentially toxic chemical agents and metabolites. These include naturally occurring plant alkaloids and mycotoxins, industrial chemicals, and, additionally, pharmacologic agents used in treating disease. The range of manifestations of toxin- and drug-induced liver disease are virtually as broad as the range of acute and chronic disorders and have also been shown or suspected to have genetic bases or contributions.

Such interactions between drugs and genotype have been shown in the response, e.g., to the anticonvulsant phenytoin, which can cause severe hepatitis-like disease in individuals who are impaired in the ability to detoxify a metabolite of phenytoin in the liver, and in the response to the drug sodium valproate, which can produce severe hepatotoxicity in certain individuals. The abnormal responses to both of these drugs are believed to be influenced by underlying genetic factors.

The human genome-derived single exon nucleic acid probes and microarrays of the present invention are useful for predicting, diagnosing, grading, staging, monitoring and prognosing diseases of human liver, particularly those diseases with polygenic etiology. With each of the single exon probes described in this Example shown to be expressed at detectable levels in human liver, and with about ⅔ of the probes identifying novel genes, single exon microarrays of the present invention that include such probes provide exceptionally high informational content for such studies.

For example, diagnosis (including differential diagnosis among clinically indistinguishable disorders, such as cirrhosis), staging, and/or grading of a disease can be based upon the quantitative relatedness of a patient gene expression profile to one or more reference expression profiles known to be characteristic of a given liver disease, or to specific grades or stages thereof.

In one embodiment, the patient gene expression profile is generated by hybridizing nucleic acids obtained directly or indirectly from transcripts expressed in the patient's liver to the genome-derived single exon microarray of the present invention. Reference profiles are obtained similarly, using nucleic acids obtained directly or indirectly from transcripts expressed by liver of individuals with known liver disease. Methods for quantitatively relating gene expression profiles, without regard to the function of the protein encoded by the gene, are disclosed in WO 99/58720, incorporated herein by reference in its entirety.

In another approach, the genome-derived single exon probes and microarrays of the present invention can be used to interrogate genomic DNA, rather than pools of expressed message; this latter approach permits predisposition to and/or prognosis of liver disease to be assessed through the massively parallel determination of altered copy number, deletion, or mutation in the patient's genome of exons known to be expressed in human liver. The algorithms set forth in WO 99/58720 can be applied to such genomic profiles without regard to the function of the protein encoded by the interrogated gene.

Table 6, presented herewith in electronic format and incorporated herein by reference in its entirety, presents expression, homology, and functional information for the genome-derived single exon probes of the present invention that are expressed significantly in human liver.

### EXAMPLE 8

### Genome-derived Single Exon Probes Useful for Measuring Expression of Genes in Fetal Liver

Table 7, presented herewith in electronic format and incorporated herein by reference in its entirety, presents expression, homology, and functional information for the genome-derived single exon probes of the present invention that are expressed significantly in human fetal liver.

### EXAMPLE 9

### Genome-derived Single Exon Probes Useful for Measuring Expression of Genes in Placenta

Given the substantial impact on human morbidity and mortality of diseases directly caused by genetic defect, and given the profound influence of genetic factors on the predisposition, onset, and/or aggressiveness of most, if not all human diseases, there has long been interest in efficient and safe means for early detection of gene defects and polymorphisms that cause, are associated with, or are implicated in development of disease.

Classically, such antenatal diagnosis was effected during second trimester by metaphase karyotyping of fetal cells that had shed spontaneously into amniotic fluid.

More recently, techniques have been developed that permit direct sampling of placenta earlier in pregnancy.

One technique in current clinical practice is chorionic villus sampling, which can be used to detect gene defects or polymorphisms in cells from the developing fetus, usually between 10 and 12 weeks of pregnancy. In chorionic villus sampling, a small sample of chorionic villi, which are tiny projections that make up part of the placenta, a fetal-derived tissue, is removed through the mother's cervix or the abdominal wall. Placental chromosomal DNA is then isolated from the chorionic villus cells and analyzed to detect a small number of known genetic defects. Such defects range from gross karyotypic changes, such as triploidy, to discrete point mutations known to cause diseases having significant morbidity or mortality.

Although only a few diseases are at present diagnosed by antenatal analysis of human placenta, a far higher number of human diseases and disorders have been catalogued in which dysfunction or misregulation of one or more genes contributes to the disease phenotype. At one end of the spectrum of genetic diseases are those, such as sickle cell trait, in which a single point mutation is responsible for the disease phenotype. At the other end of the spectrum lie disorders such as Down syndrome wherein the presence of a supernumerary chromosome manifests itself in variety of phenotypic defects that vary in severity among affected individuals. For most, possibly all genetic diseases, the precise phenotypic manifestation and its severity is a function of a complex interaction between the definable genetic lesion and the action of many other genes and environmental factors.

Although the incidence of many genetic diseases is low, a sufficient number of such genetic diseases affect a sufficiently large population that they impact the national health economy. For example, cystic fibrosis, caused by mutations in a gene encoding a chloride ion channel and resulting in lung and other disorders, occurs at a rate of about 1 in 3000 births among Caucasians and costs over $1 billion annually for direct medical treatment in the U.S. alone. Furthermore, it is increasingly thought that for many diseases where no clear-cut genetic lesion appears responsible, possession by individuals of particular gene alleles naturally occurring within certain populations places such individuals at increased risk for developing those diseases. Examples include heart disease, neurogenerative disorders, diabetes, cancer and autoimmune disorders.

For yet other diseases, especially cancer, the etiology is truly polygenic in that defects in multiple genes must cooincide in the same individual or even the same cell for the disease to develop and/or progress.

The human genome-derived single exon nucleic acid probes and microarrays of the present invention are useful for antenatal diagnosis of human genetic disorders. With each of the single exon probes described in this Example shown to be expressed at detectable levels in human placenta, and with about ⅔ of the probes identifying novel genes, single exon microarrays of the present invention that include such probes provide exceptionally high informational content for such studies.

For example, antenatal diagnosis can be based upon the quantitative relatedness of a placental gene expression profile to one or more reference expression profiles known to be characteristic of a given disease, or to specific grades or stages thereof.

In one embodiment, the gene expression profile is generated by hybridizing nucleic acids obtained directly or indirectly from placenta, typically through chorionic villus sampling, to the genome-derived single exon microarray of the present invention. Reference profiles are obtained similarly by hybridizing nucleic acids from individuals with known disease. Methods for quantitatively relating gene expression profiles, without regard to the function of the protein encoded by the gene, are disclosed in WO 99/58720, incorporated herein by reference in its entirety.

In another approach, the genome-derived single exon probes and microarrays of the present invention can be used to interrogate genomic DNA, rather than pools of expressed message; this latter approach permits presence and/or predisposition to disease to be assessed through the massively parallel determination of altered copy number, deletion, or mutation of exons known to be expressed in human placenta. The algorithms set forth in WO 99/58720 can be applied to such genomic profiles without regard to the function of the protein encoded by the interrogated gene.

Table 8, presented herewith in electronic format and incorporated herein by reference in its entirety, presents expression, homology, and functional information for the genome-derived single exon probes of the present invention that are expressed significantly in human placenta.

### EXAMPLE 10

Genome-derived Single Exon Probes Useful for Measuring Expression of Genes in Lung

Diseases of the lung are a significant cause of human morbidity and mortality. Increasingly, genetic factors are being found that contribute to predisposition, onset, and/or aggressiveness of most, if not all, of these diseases; although causative mutations in single genes have been identified for some, these disorders are, for the most part, believed to have polygenic etiologies.

For example, asthma affects about 5% of the adult population in the United States, making it the seventh-ranking chronic condition. The worldwide prevalence of asthma has increased more than 30% since the late 1970s, mostly in areas of increased industrialization. The yearly economic costs (including both direct and indirect costs) are estimated at almost $12 billion dollars. Asthma is also one of the most common reasons to seek medical treatment, with over 1.5 million emergency room visits, 500,000 hospitalizations and over 5,500 deaths each year. Outpatient visits are estimated at 15 million per year.

Patients with asthma suffer shortness of breath accompanied by cough, wheezing, and anxiety. Common features of acute asthma attacks include a rapid respiratory rate, tachycardia, and pulsus paradoxus. Acute attacks can be triggered by environmental factors such as allergens, changes in temperature, and exercise; other acute exacerbations have no discernible precipitating cause. If asthma is not treated, it can be life-threatening.

It is now well known that genetic factors predispose to asthma, but the exact nature of this genetic component is still imprecise.

A 1986 human genetic study supported polygenic inheritance, Townley, et. al., J. Allergy Clin. Immun. 77: 101-107 (1986), and more recent studies have suggested that predisposing factors for asthma, if not the disease itself, are heritable. Slutsky, J. Clin. Pharmacol. 39: 246-51 (1999).

In one approach to elaborating the polygenic contributions to asthma, candidate genes have been suggested based upon presumed involvement in the physiologic processes known to contribute to the asthmatic state. Huss et al., Nurs. Clin. North Am. 35: 695-705 (2000).

In other studies, linkages and/or associations of genetic markers with atopy, bronchial hyperresponsiveness and/or asthma have been reported in candidate regions, including the 6p region, which includes both the HLA complex and the Tumor Necrosis Factor a gene (TNF-a), the 11q region which includes the gene coding for the b sub-unit of the high-affinity IgE receptor (FcE R1), the T-cell receptor a gene on chromosome 14, the 5q region bearing numerous candidate genes among which are the interleukin (IL-3, 4, 5, 9, 13) cluster and the b₂-adrenergic receptor gene, the 12q region containing the genes for interferon-gamma (IFNg), a mast cell growth factor (MGF), and an insulin-like growth factor (IGF1). The strongest of these linkages are associated with chromosomes 5 and 11. Other linkage regions have been reported on chromosomes 6, 7, 11, 12 and 13. Demenais, The European Network For Understanding Mechanisms of Severe Asthma, BIOMED 2 Program—European Commission (1998). Linkage regions have also been suggested on chromosomes 3, 16 and 14. Duffy, D., “Review of Molecular Genetics of Asthma and Allergy”,

(http://www2.qimr.edu.au/davidD/asthma6.html).

As another example, chronic obstructive pulmonary disease (COPD) is the fourth most common cause of death in the United States. Although cigarette smoking is the most common cause of COPD, with smokers having a rate 10 to 30 times higher for developing emphysema than non-smokers, genetic factors are thought to play a significant role in susceptibility to COPD; indeed, only 15-20% of long-term cigarette smokers will develop COPD, suggesting that genetic factors strongly affect outcome.

COPD includes both chronic bronchitis and emphysema, which share similar symptoms and frequently coexist. More than 16 million Americans have COPD at a cost currently estimated at $30 billion dollars each year. Chronic obstructive lung disease is characterized by a decline in lung function resulting in difficulty in breathing and physiological changes. In severe COPD, patients breathe at very high lung volumes, having lost the lung's normal elastic recoil. Because COPD does not affect the lung uniformly, ventilation and perfusion distribution is impaired. In areas of the lung with low ventilation-perfusion ratios, arterial hypoxia results. This can further lead to pulmonary hypertension, right ventricular failure, and, ultimately, tissue ischemia, such as coronary artery disease.

The only confirmed genetic risk factor for COPD is the inherited deficiency of alpha 1-proteinase inhibitor (familial emphysema). Familial emphysema accounts for less than 5 percent of all cases of COPD, however, and familial clustering of lung function and COPD suggest the presence of other genetic risk factors. Luisetti et al., Mondaldi Arch. Chest Dis. 50:28-32 (1995); Khoury et. al., Genet Epidemiol. 2: 155-66 (1985).

Among such additional genetic factors are the presence of the GC2 allele, which appears to exert a protective effect against COPD. Horne et. al., Hum. Hered. 40: 173-76 (1990). Other suspected genetic involvement includes genes coding for alpha1-antichymotrypsin, alpha2-macroglobulin, vitamin D-binding protein and blood group antigens. Sandford et. al., Eur. Respir. J. 10: 1380-91 (1997). Finally, the form of the enzyme microsomal epoxide hydrolase is correlated to susceptibility to COPD. Smith et al., The Lancet 350: 630-33 (1997). It remains uncertain, however, whether other loci contribute to predisposition and aggressiveness of COPD.

As yet a further example, lung cancer is the leading cause of cancer death in both men and women in the United States. Although smoking is the primary risk factor, genetics plays a known role in susceptibility to these bronchogenic carcinomas.

The most common of the bronchogenic carcinomas is non-small cell lung cancer (NSCLC), which accounts for 75% of all primary lung cancers. NSCLCs are divided into adenocarcinomas, squamous cell carcinomas, and large cell carcinomas. Small cell lung cancer (SCLC) comprises 20% of primary lung cancers, and carcinoids make up 5%. Other rare forms of lung cancer (all totaling less than 1%) include lymphoma, carcinosarcoma, mucoepidermoid carcinoma, malignant fibrous histiocytoma, melanoma, sarcoma, and blastoma. Lung cancer is generally not associated with clinical symptoms until late in the course of the disease; this late diagnosis is likely to contribute to the poor 5-year survival rate of 14%.

Premalignant changes are thought to include a number of successive mutations in various growth regulation genes. A chromosome 3p deletion, chromosome 9p deletion, and p53 gene mutations have been identified in premalignant lesions. Chromosomal abnormalities identified in both SCLC and NSCLC include deletions involving chromosomes 3p, 5q, 9p, 11p, 13q, and 17p. Weston et. al., Proc. Nat. Acad. Sci. 86: S5099-5103 (1989). For most of these regions, suspected loci are tumor suppressor genes. Additionally, transforming oncogenes such as Ki-ras, H-ras, N-ras, myc, her2neu, c-kit, bcl-2 and cyclin D1 (prad) have also been shown to be activated in certain types of bronchogenic carcinomas. Perucho et. al., Cell 27: 467-76 (1981); Cecil Textbook of Medicine, 21^{st} ed. (2000).

Other contributing genetic loci have been identified, including a deletion of the phosphatase and tensin homolog (PTEN) at 10q23.3. Overexpression of PTEN can inhibit invasion in lung cancer cells, and appears to downregulate integrin alpha(6), laminin beta(3), heparin-binding epidermal growth factor-like growth factor, urokinase-type plasminogen activator, myb protein B, and Akt2. Hong et. al., Am. J. Respir. Cell Mol. Biol 23: 355-63 (2000). In a recent study assessing the risk of lung cancer from environmental tobacco smoke (ETS), women who were homozygous null for glutathione S-transferase (GST)-1 (GSTM1) had a statistically significant greater risk of developing lung cancer from ETS. Bennett et. al., J. Nat. Cancer. Inst. 91: 2009-2014 (1999). The identified genetic factors are believed to be only a subset, however, of loci that contribute to disease.

As a still further example, the interstitial lung diseases (ILDs) share certain pathogenic mechanisms and histopathologic features. ILDs comprise more than 100 disorders characterized by diffuse inflammation and scarring of the lung interstitium, derangement of the alveolar walls and loss of functional alveolar capillary units. Symptoms include breathlessness, exercise intolerance, and progressive respiratory insufficiency. ILD is estimated to account for 100,000 hospital admissions each year.

Genetic factors are known to contribute to the development of some types of ILD. Examples are familial idiopathic pulmonary fibrosis, neurofibromatosis, tuberous sclerosis, Gaucher's disease, Niemann-Pick disease and Hermansky-Pudlak syndrome. ILDs with unknown etiology include, e.g., sarcoidosis, pulmonary hemosiderosis, pulmonary histiocytosis, lymphangioleiomyomatosis, pulmonary alveolar proteinosis, and nonspecific interstitial lung disease.

As an example of still undefined polygenic basis, the etiology of sarcoidosis remains enigmatic, but has long been suspected to have a genetic component. Ethnic preponderance, familial clustering and multigenerational involvement all point towards hereditary susceptibility. Rybicki et. al., Clin. Chest Med. 18: 707-717 (1997). Some studies have shown an association between susceptibility to sarcoidosis and HLA type. Nowack et al., Arch. Intern. Med. 147: 481-83 (1987); Ishihara et. al., Tissue Antigens 50: 650-53 (1997).

Other significant diseases of the lung are also believed to have a genetic, typically polygenic, etiologic component. These diseases include, for example, Kartagener syndrome, fibrocystic pulmonary dysplasia, primary ciliary dyskinesis, pulmonary hypertension, and hyaline membrane disease.

The human genome-derived single exon nucleic acid probes and microarrays of the present invention are useful for predicting, diagnosing, grading, staging, monitoring and prognosing diseases of human lung, particularly those diseases with polygenic etiology. With each of the single exon probes described in this Example shown to be expressed at detectable levels in human lung, and with about ⅔ of the probes identifying novel genes, single exon microarrays of the present invention that include such probes provide exceptionally high informational content for such studies.

For example, diagnosis (including differential diagnosis among clinically indistinguishable disorders, such as the ILDs), staging, and/or grading of a disease can be based upon the quantitative relatedness of a patient gene expression profile to one or more reference expression profiles known to be characteristic of a given lung disease, or to specific grades or stages thereof.

In one embodiment, the patient gene expression profile is generated by hybridizing nucleic acids obtained directly or indirectly from transcripts expressed in the patient's lung to the genome-derived single exon microarray of the present invention. Reference profiles are obtained similarly by hybridizing nucleic acids from individuals with known disease. Methods for quantitatively relating gene expression profiles, without regard to the function of the protein encoded by the gene, are disclosed in WO 99/58720, incorporated herein by reference in its entirety.

In another approach, the genome-derived single exon probes and microarrays of the present invention can be used to interrogate genomic DNA, rather than pools of expressed message; this latter approach permits predisposition to and/or prognosis of lung disease to be assessed through the massively parallel determination of altered copy number, deletion, or mutation in the patient's genome of exons known to be expressed in human lung. The algorithms set forth in WO 99/58720 can be applied to such genomic profiles without regard to the function of the protein encoded by the interrogated gene.

Table 9, presented herewith in electronic format and incorporated herein by reference in its entirety, presents expression, homology, and functional information for the genome-derived single exon probes of the present invention that are expressed significantly in human lung.

### EXAMPLE 11

### Genome-derived Single Exon Probes Useful for Measuring Expression of Genes in Bone Marrow

Because bone marrow is the tissue in which blood cells originate, diseases of the bone marrow are a significant cause of human morbidity and mortality. Increasingly, genetic factors are being found that contribute to predisposition, onset, and/or aggressiveness of most, if not all, of these diseases. Although mutations in single genes have in some cases been identified as causal—notably in the thalassemias and sickle cell anemia—disorders of the bone marrow are, for the most part, believed to have polygenic etiologies.

For example, cancers that originate in the bone marrow and lymphatic tissues such as the lymphomas, leukemias, and myeloma have been recognized as a major health concern. An estimated 632,000 Americans are presently living with lymphoma, leukemia or myeloma, and over 110,000 new cases are anticipated each year. The new cases alone account for 11% of all cancer cases reported in the United States.

Lymphoma is a general term for a group of cancers of lymphocytes that manifest in the tissues of the lymphatic system. Eventually, monoclonal proliferation crowds out healthy cells and creates tumors which enlarge lymph nodes. Approximately 450,000 members of the U.S. population are living with lymphoma: 160,000 with Hodgkin disease (HD) and 290,000 with non-Hodgkin lymphoma.

Hodgkin disease (HD) is a specialized form of lymphoma, and represent about 8% of all lymphomas. HD can be distinguish in tissues by the presence of an abnormal cell called the Reed-Sternberg cell. Incidence rates of HD are higher in adolescents and young adults, but HD is considered to be one of the most curable forms of cancer. Symptoms of HD include painless welling of lymph glands, fatigue, recurrent high fever, sweating at night, skin irritations and loss of weight.

Although an infectious etiology has been proposed to account for the disproportionate incidence of HD among siblings reared together—particularly an association with Epstein Barr Virus (EBV)—multiple genetic contributions have also been suggested.

As early as 1986, linkage to HLA was suggested, with Klitz et al., Am. J. Hum. Genet. 54: 497-505 (1994) reporting an overall association of the nodular sclerosing (NSHD) group with the HLA class II region. Results of the study suggested that susceptibility to NSHD is influenced by more than 1 locus within the class II region. Through a literature search, Shugart and Collins (2000), Europ. J. Hum. Genet. 8: 460-463 (2000), performed a combined segregation and linkage analysis on 59 nuclear families with HD and concluded that HD is most likely determined by both an HLA-associated major gene and other non-HLA genetic factors, in conjunction with environmental effects.

Non-Hodgkin lymphoma (NHL) is a malignant monoclonal proliferation of the lymphoid cells in the immune system, including bone marrow, spleen, liver and GI tract. The pathologic classification of NHL continues to evolve, reflecting new insights into the cells of origin and the biologic bases of these heterogeneous diseases. The course of NHL varies from indolent and initially well tolerated to rapidly fatal. Furthermore, common clinical symptoms of NHL, but rare in HD, are congestion and edema of the face and neck and ureteral compression.

Non-Hodgkin lymphoma (NHL) has been linked to a variety of specific genetic defects, including 26 mutated genes and at least 9 identified chromosomal translocations. Among the mutated genes are: ALK (2p23); API2 (MIHC, cIAP2) (11q22-q23); API4 (survivin, SVV)(17q25(?)); ATM (ATA, ATC) (11q22.3); BCL1 (11q13.3); BCL10 (CLAP, CIPER)(1p22); BCL2 (18q21.3); BCL6 (LAZ3,ZNF51) (3q27); BLYM (1p32); BMI1 (10p13); CCND1 (D11S287E, Cyclin D,PRAD1)(11q13); CD44 (MDU3, HA, MDU2)(11pter-p13); FRAT1 (10q23-q24(?)); FRAT2 (GBP)(10(?)); IL6 (IFNB2)(7p21); IRF4 (MUM1, LSIRF) (6p25-p23); LCP1 (PLS2)(13q14.1-q14.3); MALT1 (MLT)(18q21); MUC1 (PUM,PEM)(1q21); MYBL1 (AMYB, A-MYB)(8q22); MYC (CMYC, C-MYC )(8q24.12-q24.13); NBS1(8q21); NPM1 (B23)(5q35); PCNA (20pl2); TIAM1 (21q22.1); and TP53 (p53, P53)(17q13.1).

Among the chromosomal abnormalities are: t(1;14) (p22;q32); t(14;18)(q32;q21); t(3;14)(q27;q32); t(6;14)(p25,q32); t(11;18)(q21;q21); t(1;14)(q21;q32); t(2;5)(p23;q35); add(14q32)/dup(14p32); and t(ll;14) (ql3;q32).

Additional genetic loci, as yet undiscovered, are believed to account for other occurrences of NHL.

As another example, acute leukemia is a malignant disease of blood-forming tissues such as the bone marrow. It is characterized by the uncontrolled growth of white blood cells. As a result, immature myeloid cells (in acute myelogenous leukemia (AML)) or lymphoid cells (in acute lymphocytic leukemia (ALL)) rapidly accumulate and progressively replace the bone marrow; diminished production of normal red cells, white cells, and platelets ensues. This loss of normal marrow function in turn gives rise to the typical clinical complications of leukemia: anemia, infection, and bleeding.

If untreated, ALL is rapidly fatal; most patients die within several months of diagnosis. With appropriate therapy, many patients can be cured. The survival rate for patients diagnosed with AML or ALL is 14% and 58% respectively. However, the incidences of AML is expected to be greater than ALL: an estimated 10,000 new cases of AML, predominantly in older adults, is anticipated in the U.S. alone, whereas 3,100 new cases of ALL are expected, with 1,500 of these new cases occurring among children. The etiology of acute leukemia is not known.

Although human T-cell lymphotropic virus type I (HTLV-I), a causative agent of adult T-cell leukemia, and HTLV-II, obtained from several patients with a syndrome resembling hairy cell leukemia, have been isolated, the etiologic link between HTLV and malignancy is uncertain. There is, however, evidence which suggests a genetic predisposition to incidences of acute leukemia.

For example, genetic disorders such as Fanconi anemia and Down syndrome appear to increase risk of acute leukemia, specifically, AML. Evidence supporting a chromosome 21 locus for acute myelogenous leukemia (AML) includes the finding of linkage to 21q22.1-q22.2 in a family with a platelet disorder and propensity to develop AML (Ho et al., Blood 87: 5218-5224 (1996), an increased incidence of leukemia in Down syndrome, and frequent somatic translocation in leukemia involving the CBFA gene on 21q22.3. In addition, Horwitz et al., Am. J. Hum. Genet. 61:873-881 (1997), suggest that a gene on 16q22 may be a second cause of acute myelogenous leukemia. Nonparametric linkage analysis gave a P-value of 0.00098 for the conditional probability of linkage. Mutational analysis excluded expansion of the AT-rich minisatellite repeat FRA16B fragile site and the CAG trinucleotide repeat in the E2F-4 transcription factor. Large CAG repeat expansion was excluded as a cause of leukemia in this family.

Similarly, acute lymphoblastic leukemia (ALL) has been suggested to have a genetic predisposition. In particular, linkage to chromosome 9p has been reported by a number of groups. Chilcote et al., New Eng. J. Med. 313: 286-291 (1985), found that 6 of 8 patients with clinical features of lymphomatous ALL (LALL), a distinct category of ALL of T-cell lineage, had karyotypic abnormalities leading to loss of bands 9p22-p21. The mechanisms varied and included deletions, unbalanced translocations, and loss of the entire chromosome; only 1 of 57 patients without LALL had an abnormality of chromosome 9 at diagnosis. Kowalczyk et al., Cancer Genet. Cytogenet. 9:383-385 (1981), had earlier found changes in 9p in a subgroup of ALL cases. Chilcote et al. (1985) pointed out that there is a fragile site at 9p21 and raised the question of familial predisposition on this basis. This fragile site is the breakpoint in the translocation t(9;11)(p21-22;q23), which is associated with acute nonlymphocytic leukemia with monocytic features, ANLL-AMoL-M5a. In a large series, Murphy et al., New Eng. J. Med. 313:1611 (1985), confirmed an abnormality of 9p in 10 to 11% of cases (33 out of more than 300) of acute lymphoblastic leukemia. The breakpoints in 9p clustered in the p22-p21 region. They could not, however, corroborate the specific association with T-cell origin or so-called lymphomatous clinical features. In addition, Taki et al., Proc. Natl. Acad. Sci. USA 96:14535 (1999), recently identified AF5q31, a new AF4-related gene, fused to MLL in infant ALL with ins(5;11)(q31;ql3q23), and suspects that AF5q31 and AF4 might define a new family particularly involved in the pathogenesis of 11q23-associated-ALL.

As yet a further example of a disease affecting bone marrow with likely polygenic etiology is multiple myeloma (MM).

MM is a cancer of plasma cells, the final differentiated stage of B lymphocyte maturation. The malignant clone proliferates in the bone marrow and frequently invades the adjacent bone, producing extensive skeletal destruction that results in bone pain and fractures. Anemia, hypercalcemia, and renal failure are some clinical manifestations associated with MM.

MM causes 1% of all cancer deaths in Western countries. A genetic component to its etiology is suggested by disparate incidence among various groups in the country. Its incidence is higher in men than in women, in people of African descent relative to the U.S. population at large, and in older adults as compared to the young. It has been estimated that 14,000 new cases of myeloma will be diagnosed in the U.S., and over 11,000 persons will die from MM within the year.

Although, Kaposi's sarcoma-associated herpes virus has been associated with MM (Retig et al., Science 276:1851 (1997)), there is evidence that chromosomal abnormalities, such as the deletion of 13ql4 and rearrangements of 14q increase the proliferation of myeloma cells.

Up to 30% of patients who suffer with MM have a balanced translocation, t(4;14)(p16.3;q32), that places the fibroblast growth factor receptor 3 (FGFR3) gene under the control of IgH promoter elements (Chesi et al., Nat. Genet. 16:260 (1997)). This results in increased expression of FGFR3, a member of a family of tyrosine kinase receptors implicated in control of cellular proliferation.

According to Zoger et al., Blood 95:1925 (2000), monoallelic deletions of the retinoblastoma-1 (rb-1) gene and the D13S319 locus were observed in 48 of 104 patients (46.2%) and in 28 of 72 (38.9%) patients, respectively, with newly diagnosed MM. Fluorescence in situ hybridization (FISH) studies found that 13q14 was deleted in all 17 patients with karyotypic evidence of monosomy 13 or deletion of 13q but also in 9 of 19 patients with apparently normal karyotypes. Patients with a 13q14 deletion were more likely to have higher serum levels of beta(2)-microglobulin (P=0.059) and a higher percentage of bone marrow plasma cells (P=0.085) than patients with a normal 13q14 status on FISH analysis. In patients with a deletion of 13q14, myeloma cell proliferation was markedly increased. The presence of a 13q14 deletion on FISH analysis was associated with a significantly lower rate of response to conventional-dose chemotherapy (40.8% compared with 78.6%; P=0.009) and a shorter overall survival (24.2 months compared with >60 months; P<0.005) than in patients without the deletion.

There are numerous other mutated genes and chromosomal abnormalities that may predispose to MM. Examples of such genes are: B2M (15q21-q22); CCND1 (D11S287E, Cyclin D, PRAD1)(11q13); CD19 (16p11.2); HGF (HPTA)(7q21.1); IL6 (IFNB2)(7p21); IRF4 (MUM1, LSIRF)(6p25-p23); LTA (TNFB, LT)(6p21.3); SDC1 (2p24.1); and TNF (TNFA, TNFSF2, DIF)(6p21.3). Examples of chromosomal abnormalities include:

t(6;14)(p25;q32) and t(11;14)(q13;q32).

Other significant diseases or disorders of the bone marrow are also believed, or likely to have, a genetic, typically polygenic, etiologic component. These diseases include, for example, chronic myeloid leukemia, chronic lymphoid leukemia, polycythemia vera, myelofibrosis, primary thrombocythemia, myelodysplastic syndromes, Wiskott-Aldrich, lymphoproliferative syndrome, aplastic anemia, Fanconi anemia, Down syndrome, sickle cell disease, thalassemia, granulocyte disorders, Kostmann syndrome, chronic granulomatous disease, Chediak-Higashi syndrome, platelet disorders, Glanzmann thrombasthenia, Bernard-Soulier syndrome, metabolic storage diseases, osteopetrosis, congenital hemophagocytic syndrome.

The human genome-derived single exon nucleic acid probes and microarrays of the present invention are useful for predicting, diagnosing, grading, staging, monitoring and prognosing diseases of human bone marrow, particularly those diseases with polygenic etiology. With each of the single exon probes described in this Example shown to be expressed at detectable levels in human bone marrow, and with about ⅔ of the probes identifying novel genes, single exon microarrays of the present invention that include such probes provide exceptionally high informational content for such studies.

For example, diagnosis, grading, and/or staging of a disease can be based upon the quantitative relatedness of a patient gene expression profile to one or more reference expression profiles known to be characteristic of a given bone marrow disease, or to specific grades or stages thereof.

In one embodiment, the patient gene expression profile is generated by hybridizing nucleic acids obtained directly or indirectly from transcripts expressed in the patient's bone marrow (or cells cultured therefrom) to the genome-derived single exon microarray of the present invention. Reference profiles are obtained similarly by hybridizing nucleic acids obtained directly or indirectly from transcripts expressed in the bone marrow of individuals with known disease. Methods for quantitatively relating gene expression profiles, without regard to the function of the protein encoded by the gene, are disclosed in Wo 99/58720, incorporated herein by reference in its entirety.

In another approach, the genome-derived single exon probes and microarrays of the present invention can be used to interrogate genomic DNA, rather than pools of expressed message; this latter approach permits predisposition to and/or prognosis of diseases of bone marrow to be assessed through the massively parallel determination of altered copy number, deletion, or mutation in the patient's genome of exons known to be expressed in human bone marrow. The algorithms set forth in WO 99/58720 can be applied to such genomic profiles without regard to the function of the protein encoded by the interrogated gene.

Table 10, presented herewith in electronic format and incorporated herein by reference in its entirety, presents expression, homology, and functional information for the genome-derived single exon probes of the present invention that are expressed significantly in human bone marrow.

### EXAMPLE 12

### Genome-derived Single Exon Probes Useful for Measuring Expression of Genes in HeLa Cells

Table 11, presented herewith in electronic format and incorporated herein by reference in its entirety, presents expression, homology, and functional information for the genome-derived single exon probes of the present invention that are expressed significantly in HeLa cells.

### EXAMPLE 13

### Genome-derived Single Exon Probes Useful for Measuring Expression of Genes in BT 474 Cells

Diseases of the breast are a significant cause of human morbidity and mortality. Increasingly, genetic factors are being found that contribute to predisposition, onset, and/or aggressiveness of most, if not all, of these diseases. Although mutations in single genes have been identified as causative for some diseases of the breast, for the most part these disorders are believed to have polygenic etiologies.

For example, according to the American Cancer Society (ACS), carcinoma of the breast is the second most common cancer in women and, after lung cancer, is the second deadliest. The ACS estimates that in the U.S. there occurred 182,800 new cases of malignant breast cancer in 2000, and about 40,800 deaths from the disease. Although incidence of breast cancer is said to have declined, the disease clearly continues to represent a serious risk to the health and life of American women. Indeed, about one in nine U.S. women will develop breast cancer in her lifetime, and at present mortality rates, about a third of such women will eventually die from the disease.

A variety of factors are known to increase the risk of breast carcinoma. Sex is one: breast cancer in men is rare. Age is another: as women age, their risk for developing breast cancer increases, a 70 year old woman having three times the risk of developing cancer and five times the risk of dying from the disease as compared to a 40 year old woman. Most breast cancers occur after age 50, although in women with a genetic susceptibility, breast cancer tends to occur at an earlier age than in sporadic cases. Reproductive and menstrual history are also known to affect risk, with risk increasing with early menarche and late menopause, and is reduced by early first full term pregnancy. Additional risk factors, oft-times termed “lifestyle factors”, include weight gain, obesity, fat intake, alcohol consumption, and level of physical activity.

That genetic factors underlie the etiology of breast cancer is suggested by the approximately two-fold increased risk for development of breast cancer by women with a first-degree relative who has also developed breast cancer. After gender and age, a positive family history is the strongest known predictive risk factor for breast cancer. Genetic linkage analysis in families with high rates of inherited cancer have facilitated the identification of several genes in which mutations can be shown to contribute substantially to the development and progression of breast cancer, including BRCA1, BRCA2, p53, and PTEN/MMAC1. Further study has made clear, however, that these genes are not alone sufficient to explain all genetic contributions to breast cancer.

For example, BRCA1 appears to be responsible for disease in up to 90% of families with both breast and ovarian cancer, but in only 45% of families with multiple cases of breast cancer without occurrence of ovarian cancer. And mutations in BRCA2, localized to the long arm of chromosome 13, are thought to account for only approximately 35% of multiple case breast cancer families. Furthermore, despite the strong correlation between germline mutations in BRCA1 or BRCA2 and development of breast cancer, only weak connections have been made between these genes and sporadic breast cancer.

Epistatic effects of BRCA1 and BRCA2 mutations on other genetic loci, only some of which have been identified, have been postulated to account for some of the deleterious effects of mutations in these two genes.

Thus, mutations in p53 seem to be much more frequent in BRCAl breast cancers (20/26) and somewhat more frequent in BRCA2-associated breast cancers (10/22) than in grade-matched sporadic cancers (7/20). BRCA mutation-associated cancers contain p53 mutations not typically found in sporadic breast cancer, and 12 individual hereditary breast cancers have been shown to contain more than a single p53 mutation. Mutations of BRCA1 and BRCA2 may thus confer a “mutator” phenotype permitting the accumulation of genetic abnormalities, with p53 inactivation selected during tumor progression.

Additionally, genome-wide screening for chromosomal gains or losses in breast cancers harboring BRCA1 or BRCA2 mutations demonstrated more regions that were amplified or deleted compared to controls, suggesting a generalized increase in large-scale genomic instability. Chromosomes 5q, 4q, and 4p had very frequent loss of heterozygosity in BRCA1 tumors, while BRCA2 tumors were characterized by losses at 13q (near the BRCA2 locus itself) and 6q, and chromosomal gains at 17q (outside of the HER2/neu locus) and 20q.

Mutations of other genes have also been implicated in susceptibility to development or aggressiveness of breast cancer. For example, germline mutations in the ATM gene, localized to chromosome 11q22-23, result in an increased risk of breast cancer among female heterozygote carriers with an estimated relative risk of 3.9 to 6.4; it is unclear, however, if mutations in the ATM gene itself contribute to breast cancer.

Normal allelic variation in a variety of genes, as opposed to frank mutation, may also influence susceptibility to developing breast carcinoma and the propensity for the disease to progress. Such polymorphisms may thus explain why particular women or ethnic groups who do not otherwise bear mutations in genes known to be linked to breast cancer are at greater risk, especially in the context of exposure to environmental agents and other nonhereditary risk factors. Polymorphically expressed genes may code for enzymes that metabolize estrogens or detoxify drugs and environmental carcinogens.

For example, molecular epidemiologic studies of cancer of the breast have examined associations with p450 cytochrome genotypes including CYPlAl, CYP2D6, and CYP17. The CYPlAl gene, located on chromosome 15q, encodes the enzyme aryl hydrocarbon hydroxylase (AHH), present in breast tissue, and which catabolizes polycyclic aromatic hydrocarbons and arylamines. AHH is strongly inducible, i.e., greater enzymatic activity is seen with greater exposure to substrates. AHH catalyzes the monooxygenation of polycyclic aromatic hydrocarbons to phenolic products and epoxides that may be carcinogenic. AHH is also involved in the conversion of estrogen to hydroxylated conjugated estrogens such as 2-hydroxyestradiol.

Three polymorphisms in the CYPlAl gene have been identified: an MspI RFLP of the 3′ end of the gene (MspI); an adenine to guanine mutation in exon 7, causing an isoleucine to valine substitution (Ile-Val); and a polymorphism of the CYPlAl gene identified among Negroids. The frequencies of the MspI and Ile-Val polymorphisms vary considerably by race, being higher among Japanese and Hawaiian populations as compared with Caucasians and Negroids.

The CYP2D6 gene is located on chromosome 22q and encodes the enzyme debrisoquine hydroxylase, which metabolizes a variety of drugs and other xenobiotics. Like other polymorphically expressed p450 enzymes, it may activate procarcinogens or, conversely, detoxify carcinogens. A number of alleles have been characterized at the CYP2D6 locus. The “poor metabolizer” phenotype (CYP2D6 mutant/mutant genotype), which is rare in Asians, occurs in about 5% to 10% of Caucasians and in 2% of Negroids.

As another example, the N-acetyl transferase-1 (NAT1) and N-acetyl transferase-2 (NAT2) genes are located on chromosome 8q. Allelic variation in the NAT genes may contribute to variation in populations as to the susceptibility of individuals to development of breast carcinoma, particularly in the context of exposure to compounds present in tobacco. NAT2 detoxifies or, conversely, activates aromatic amines found in tobacco smoke such as 4-aminobiphenyl. Both phenotypic assays and genotypic assays for NAT2 can be used to classify individuals as rapid or slow acetylators. Genetic variants of the NAT2 gene have been cloned and 6 alleles at this locus have been identified: the Fl allele confers the fast acetylator phenotype. The distribution of NAT1 and NAT2 alleles differs widely between racial and ethnic groups.

As yet another example, the glutathione S-transferase-M1(GSTM1) gene is located on chromosome 1 and the gene for glutathione S-transferase-T1 (GSTT1) is located on chromosome 11q. A glutathione S-transferase-P1 (GSTP1) gene has also been identified. Glutathione S-transferases detoxify a variety of carcinogens and cytotoxic drugs (for example, benzo(a)pyrene, monohalomethanes such as methyl chloride, ethylene oxide, pesticides, and solvents used in industry) by catalyzing the conjugation of a glutathione moiety to the substrate. Allelic variation in the glutathione-S-transferase genes may contribute to variation in populations as to the susceptibility of individuals to development of breast carcinoma, particularly in the context of exposure to environmental toxins. Individuals homozygous for deletions in the GSTM1, GSTT1, or GSTP1 genes may have a higher risk of cancer of the breast and other sites because of their impaired ability to metabolize and eliminate carcinogens.

GSTM1 is polymorphically expressed and 3 alleles at the GSTM1 locus have been identified: GSTM1-0 (homozygous deletion genotype), GSTMla, and GSTMlb. The null allele (GSTM1-0) is present in about 38% to 67% of Caucasians and 22% to 35% of Negroids. GSTM is not expressed in breast tissue at high levels. Two functionally different genotypes at the GSTT1 locus have been described: GSTT1-0 (homozygous deletion genotype) and GSTT1-1 (genotypes with 1 or 2 undeleted alleles). A polymorphism of the GSTP1 gene, A313G (changing codon 105 from Ile to Val), has been identified. The GSTT1-0 allele has been associated with accelerated age of first breast cancer diagnosis as compared with the GSTT1-1 allele.

Many other genes have been suggested to be involved in the development and/or progression of breast cancer, either as a result of gain of function or loss of function mutations, or as a result of normal allelic variation within different populations. A nonexhaustive list of such genes, each followed by the gene's chromosomal location, if known, follows: AMPH 7p14-p13; AMPHL (BIN1 , SH3P9) 2q14; API4 (survivin, SVV) 17q25(?); ARHA (ARH12, RhoA) 3p21.3; ARHC (RhoC) 1p21-p13; ATM (ATA, ATC) 11q22.3; BAG19p12; BARD1 2q34-q35; BCAR1 16q23.1; BCAR2; BCAR3 (NSP2); BCAS1 (NABC1, AIBC1) 20q13.2-q13.3; BRCA1 17q21; BRCA2 13q12.3; CCND1 (D11S287E, Cyclin D, PRAD1) 11q13; CD44 (MDU3, HA, MDU2) 11pter-p13; CD9 (p24, MIC3, BA2) 12p13; CDKN1B (KIP1, P27) 12p13; CDKN2A (P16, INK4A, MTS1) 9p21; COMT 22q11.2; COT (MAP3K8, TPL-2, EST) 10p11.2; CSK (c-src) 15q23-q25; CTSD (CPSD) 11p15.5; CYP17 10q24.3; CYP19 15q21.1; CYP1A1 (CYP1) 15q22-q24; CYP1B1 (GLC3A) 2p22-p21; EFNB2 (EPLG5, LERK5, ephrin-B2) 13q33; EIF3S6 (INT6) 8q22-q23; EIF4E (EIF-4E) 4q21-q25; EMS1 11q13; ERBB2 (HER2, NEU) 17q11.2-q12; ERBB3 (HER3) 12q13; ESR1 (ESRA) 6q25.1; ESR2 (ESRB, ERBeta) 14q; FGF8 (AIGF) 10q24; GSTM1 (GST1, MU) 1p13.3; GSTP1 (FAEES3, GST3, PI) 11q13; GSTT1 22q11.23 ?; HRAS llpl5.5; HSPB1 (HSP27) 7q; HSPCA (HSP90A , HSPC1); HSPCB (HSP90B, HSPC2) 6p12; IGF1 12q22-q24.1; IGF1R (JTK13) 15q25-q26; IGF2 11p15.5; IL6 (IFNB2) 7p21; ING1 13q34; KISS1 (KiSS-1) 1q32; KLK3 (PSA, APS) 19q13; LASP1 (MLN50) 17q11-q21.3; LIBC 6q22; MAP2K4 (MKK4, SEKI, JNKK1) 17p11.2; MKI67 (Ki-67) 10q25-qter; MMP11 (STMY3, STR3) 22q11.2; MMP2 (CLG4A, CLG4, GELA) 16q13; MUC1 (PUM, PEM) 1q21; MYC (CMYC, C-MYC) 8q24.12-q24.13; NOTCH4 (INT3, NOTCH3) 6p21.3; PCNA 20p12; PI5 (maspin ) 18q21.3; PLAU (uPA, URK) 10q24; PSEN2 (D21S21, HPS2, BCEI) 21q22.3; RARB (NR1B2, HAP) 3p24; RB1 (Rb) 13q14.2; S100A4 (MTS1, P9KA, metastasin) 1q21; SLC22A1L (BWSCR1A, ORCTL2, IMPT1) 11p15.5; SNCG (BCSG1) 10q23.2-q23.3; SRD5A2 2p23; STIP1 (HSP70) 11?; STK11 (LKB1, PJS) 19p13.3; TFAP2A (AP2, AP2TF) 6p24; TFAP2B (AP2B) 6p12; TFAP2C 20q13.2; TFF1 (D21S21, BCEI) 21q22.3; TGFBR2 3p22; TIMP2 17q25; TP53 (p53 , P53) 17q13.1; TPD52 (D52) 8q21; TPD52L1 (D53, hD53) 6q22-q23; TSG101 11p15.2-p15.1.

The etiology of non-cancerous disorders of the breast may also involve genetic factors. Such disorders include disorders of development, inflammatory diseases of the breast, fibrocystic changes, proliferative breast disease, and non-carcinoma tumors.

Disorders of development of the breast include supernumerary nipples or breasts; accessory axillary breast tissue; congenital inversion of the nipples; and macromastia.

Inflammatory diseases of the breast include acute mastitis; periductal mastitis, also called recurrent subareolar absecess and sqamous metaplasia of the lactiferous ducts; mammary duct ectasia; fat necrosis; and granulomatous mastitis, including granulomatous lobular mastitis. Systemic granulomatous diseases that can affect the breast include Wegener granulomatosis and sarcoidosis.

Proliferative breast diseases include epithelial hyperplasia; sclerosing adenosis; and small duct papillomas. Non-carcinoma tumors include stomal tumors including fibroadenoma and phyllodes tumor, and sarcomas that include angiosarcoma, rhabdomyosarcoma, liposarcoma, leiomyosarcoma, chondrosarcoma and osteosarcoma. Other breast tumors include epithelial cell tumors including large duct papillomas.

The human genome-derived single exon nucleic acid probes and microarrays of the present invention are useful for predicting, diagnosing, grading, staging, monitoring and prognosing diseases of human breast, particularly those diseases with polygenic etiology. With each of the single exon probes described in this Example shown to be expressed at detectable levels in human breast cancer cells, and with about ⅔ of the probes identifying novel genes, single exon microarrays of the present invention that include such probes provide exceptionally high informational content for such studies.

For example, diagnosis, grading, and/or staging of a disease can be based upon the quantitative relatedness of a patient gene expression profile to one or more reference expression profiles known to be characteristic of a given breast disease, or to specific grades or stages thereof.

In one embodiment, the patient gene expression profile is generated by hybridizing nucleic acids obtained directly or indirectly from transcripts expressed in the patient's breast to the genome-derived single exon microarray of the present invention. Reference profiles are obtained similarly by hybridizing nucleic acids from individuals with known disease. Methods for quantitatively relating gene expression profiles, without regard to the function of the protein encoded by the gene, are disclosed in WO 99/58720, incorporated herein by reference in its entirety.

In another approach, the genome-derived single exon probes and microarrays of the present invention can be used to interrogate genomic DNA, rather than pools of expressed message; this latter approach permits predisposition to and/or prognosis of breast disease to be assessed through the massively parallel determination of altered copy number, deletion, or mutation in the patient's genome of exons known to be expressed in human breast. The algorithms set forth in WO 99/58720 can be applied to such genomic profiles without regard to the function of the protein encoded by the interrogated gene.

Table 12, presented herewith in electronic format and incorporated herein by reference in its entirety, presents expression, homology, and functional information for the genome-derived single exon probes that are expressed significantly in BT474 cells, human epithelial cells isolated from a solid, invasive ductal carcinoma of the breast and available commercially from American Type Culture Collection under catalogue number HTB-20.

### EXAMPLE 14

### Genome-derived Single Exon Probes Useful For Measuring Expression of Genes in HBL 100 Cells

Table 13, presented herewith in electronic format and incorporated herein by reference in its entirety, presents expression, homology, and functional information for the genome-derived single exon probes that are expressed significantly in HBL 100 cells, a hormone-sensitive human breast cancer cell line.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. A nucleic acid probe for measuring human gene expression, comprising:
a nucleotide sequence as set forth in any one of SEQ ID NOs: 16,835-33,299, the complement thereof, or a fragment of said SEQ ID NO: or said complement,
wherein said probe is a single exon probe that hybridizes under high stringency conditions to a nucleic acid molecule expressed in human cells or tissues.

2. A single exon nucleic acid probe according to claim 1, wherein:
said nucleotide sequence is selected from the exon SEQ ID NOs: set forth in Table 4, the complement thereof, or a fragment of said SEQ ID NO: or said complement,
and wherein said probe hybridizes under high stringency conditions to a nucleic acid molecule expressed in human brain.

3. A single exon nucleic acid probe according to claim 1, wherein:
said nucleotide sequence is selected from the exon SEQ ID NOs: set forth in Table 5, the complement thereof, or a fragment of said SEQ ID NO: or said complement,
and wherein said probe hybridizes under high stringency conditions to a nucleic acid molecule expressed in human heart.

4. A single exon nucleic acid probe according to claim 1, wherein:
said nucleotide sequence is selected from the exon SEQ ID NOs: set forth in Table 6, the complement thereof, or a fragment of said SEQ ID NO: or said complement,
and wherein said probe hybridizes under high stringency conditions to a nucleic acid molecule expressed in human liver.

5. A single exon nucleic acid probe according to claim 1, wherein:
said nucleotide sequence is selected from the exon SEQ ID NOs: set forth in Table 7, the complement thereof, or a fragment of said SEQ ID NO: or said complement,
and wherein said probe hybridizes under high stringency conditions to a nucleic acid molecule expressed in human fetal liver.

6. A single exon nucleic acid probe according to claim 1, wherein:
said nucleotide sequence is selected from the exon SEQ ID NOs: set forth in Table 8, the complement thereof, or a fragment of said SEQ ID NO: or said complement,
and wherein said probe hybridizes under high stringency conditions to a nucleic acid molecule expressed in human placenta.

7. A single exon nucleic acid probe according to claim 1, wherein:
said nucleotide sequence is selected from the exon SEQ ID NOs: set forth in Table 9, the complement thereof, or a fragment of said SEQ ID NO: or said complement,
and wherein said probe hybridizes under high stringency conditions to a nucleic acid molecule expressed in human lung.

8. A single exon nucleic acid probe according to claim 1, wherein:
said nucleotide sequence is selected from the exon SEQ ID NOs: set forth in Table 10, the complement thereof, or a fragment of said SEQ ID NO: or said complement,
and wherein said probe hybridizes under high stringency conditions to a nucleic acid molecule expressed in human bone marrow.

9. A single exon nucleic acid probe according to claim 1, wherein:
said nucleotide sequence is selected from the exon SEQ ID NOs: set forth in Table 11, the complement thereof, or a fragment of said SEQ ID NO: or said complement,
and wherein said probe hybridizes under high stringency conditions to a nucleic acid molecule expressed in HeLa cells.

10. A single exon nucleic acid probe according to claim 1, wherein:
said nucleotide sequence is selected from the exon SEQ ID NOs: set forth in Table 12, the complement thereof, or a fragment of said SEQ ID NO: or said complement,
and wherein said probe hybridizes under high stringency conditions to a nucleic acid molecule expressed in BT 474 cells.

11. A single exon nucleic acid probe according to claim 1, wherein:
said nucleotide sequence is selected from the exon SEQ ID NOs: set forth in Table 13, the complement thereof, or a fragment of said SEQ ID NO: or said complement,
and wherein said probe hybridizes under high stringency conditions to a nucleic acid molecule expressed in HBL 100 cells.

12. The single exon nucleic acid probe of claim 1, wherein said fragment includes at least 20 contiguous nucleotides of said SEQ ID NO: or the complement thereof.

13. The single exon nucleic acid probe of claim 1, wherein said fragment includes at least 25 contiguous nucleotides of said SEQ ID NO: or the complement thereof.

14. The single exon nucleic acid probe of claim 1, wherein said fragment includes at least 50 contiguous nucleotides of said SEQ ID NO: or the complement thereof.

15. The single exon nucleic acid probe of claim 1, wherein said probe further comprises, contiguous to a first end of said fragment, a first intronic and/or intergenic sequence that is identically contiguous to said fragment in the human genome.

16. The single exon nucleic acid probe of claim 15, wherein said probe further comprises, contiguous to a second end of said fragment, a second intronic and/or intergenLc sequence that is identically contiguous to said fragment in the human genome.

17. The single exon nucleic acid probe of claim 16, wherein said probe comprises a nucleotide sequence selected from any one of SEQ ID NOs: 1-16,834, or the complement thereof.

18. The single exon nucleic acid probe of claim 17, wherein:
said probe comprises a nucleotide sequence selected from the probe SEQ ID NOs: set forth in Table 4, or the complement thereof,
and wherein said probe hybridizes under high stringency conditions to a nucleic acid molecule expressed in human brain.

19. The single exon nucleic acid probe of claim 17, wherein:
said probe comprises a nucleotide sequence selected from the probe SEQ ID NOs: set forth in Table 5, or the complement thereof,
and wherein said probe hybridizes under high stringency conditions to a nucleic acid molecule expressed in human heart.

20. The single exon nucleic acid probe of claim 17, wherein:
said probe comprises a nucleotide sequence selected from the probe SEQ ID NOs: set forth in Table 6, or the complement thereof,
and wherein said probe hybridizes under high stringency conditions to a nucleic acid molecule expressed in human liver.

21. The single exon nucleic acid probe of claim 17, wherein:
said probe comprises a nucleotide sequence selected from the probe SEQ ID NOs: set forth in Table 7, or the complement thereof,
and wherein said probe hybridizes under high stringency conditions to a nucleic acid molecule expressed in human fetal liver.

22. The single exon nucleic acid probe of claim 17, wherein:
said probe comprises a nucleotide sequence selected from the probe SEQ ID NOs: set forth in Table 8, or the complement thereof,
and wherein said probe hybridizes under high stringency conditions to a nucleic acid molecule expressed in human placenta.

23. The single exon nucleic acid probe of claim 17, wherein said probe comprises a nucleotide sequence selected from the probe SEQ ID NOs: set forth in Table 9, or the complement thereof,
and wherein said probe hybridizes under high stringency conditions to a nucleic acid molecule expressed in human lung.

24. The single exon nucleic acid probe of claim 17, wherein:
said probe comprises a nucleotide sequence selected from the probe SEQ ID NOs: set forth in Table 10, or the complement thereof,
and wherein said probe hybridizes under high stringency conditions to a nucleic acid molecule expressed in human bone marrow.

25. The single exon nucleic acid probe of claim 17, wherein:
said probe comprises a nucleotide sequence selected from the probe SEQ ID NOs: set forth in Table 11, the complement thereof, or a fragment of said SEQ ID NO: or said complement,
and wherein said probe hybridizes under high stringency conditions to a nucleic acid molecule expressed in HeLa cells.

26. The single exon nucleic acid probe of claim 17, wherein:
said probe comprises a nucleotide sequence selected from the probe SEQ ID NOs: set forth in Table 12, the complement thereof, or a fragment of said SEQ ID NO: or said complement,
and wherein said probe hybridizes under high stringency conditions to a nucleic acid molecule expressed in BT 474 cells.

27. The single exon nucleic acid probe of claim 17, wherein:
said probe comprises a nucleotide sequence selected from the probe SEQ ID NOs: set forth in Table 13, the complement thereof, or a fragment of said SEQ ID NO: or said complement,
and wherein said probe hybridizes under high stringency conditions to a nucleic acid molecule expressed in HBL 100 cells.

28. The single exon nucleic acid probe of claim 1, wherein said probe is no more than 25 kb in length.

29. The single exon nucleic acid probe of claim 1, wherein said probe is no more than 5 kb in length.

30. The single exon nucleic acid probe of claim 1, wherein said probe lacks prokaryotic and bacteriophage vector sequence.

31. The single exon nucleic acid probe of claim 1, wherein said probe lacks homopolymeric stretches of A or T.

32. A nucleic acid probe for measuring human gene expression, comprising:
a nucleotide sequence that encodes, or the complement of which encodes, at least 8 amino acids of any one of SEQ ID NOs:33,300-49,117, wherein said probe is a single exon probe that hybridizes under high stringency conditions to a nucleic acid molecule expressed in human cells or tissues.

33. A spatially-addressable set of single exon nucleic acid probes for measuring human gene expression, comprising:
a plurality of single exon nucleic acid probes according to claim 1, wherein each of said plurality of probes is separately and addressably isolatable or amplifiable from said plurality.

34. The spatially-addressable set of single exon nucleic acid probes of claim 33, wherein each of said plurality of probes is amplifiable using at least one common primer.

35. The spatially-addressable set of single exon nucleic acid probes of claim 33, wherein said set comprises between 50-20,000 single exon nucleic acid probes.

36. The spatially-addressable set of single exon nucleic acid probes of claim 33, wherein the average length of the single exon nucleic acid probes is between 50 bp and 750 bp.

37. The spatially-addressable set of single exon nucleic acid probes of claim 33, wherein at least 50% of said single exon nucleic acid probes lack prokaryotic and bacteriophage vector sequence.

38. The spatially-addressable set of single exon nucleic acid probes of claim 33, wherein at least 50% of said single exon nucleic acid probes lack homopolymeric stretches of A or T.

39. The spatially-addressable set of single exon nucleic acid probes of claim 33, wherein said probes are addressably disposed upon a substrate.

40. A single exon microarray for measuring human gene expression, comprising:
a spatially-addressable plurality of single exon nucleic acid probes according claim 1.

41. A single exon microarray for measuring human gene expression, comprising:
a spatially-addressable plurality of single exon nucleic acid probes according to any one of claims 2-32.

42. The single exon microarray of claim 40, wherein said plurality of single exon nucleic acid probes averages at least 50 bp in length.

43. The single exon microarray of claim 40, wherein said plurality of single exon nucleic acid probes averages at least 75 bp in length.

44. The single exon nucleic acid microarray of claim 40, wherein said single exon nucleic acid probes are noncovalently bound to the substrate of said microarray.

45. A method of measuring human gene expression, comprising:
contacting the microarray of claim 40 with a first plurality of detectably labeled nucleic acids, said first plurality of nucleic acids being derived from human gene transcripts; and then
measuring the Label detectably bound to each probe of said microarray, said measures of detected label providing a measure of human gene expression.

46. The method of claim 45, further comprising:
contacting said microarray with a second plurality of detectably Labeled nucleic acids;
measuring the label detectably bound to each probe of said microarray; and
comparing said first and second measurements,
said comparative measures of detected label providing a measure of human gene expression.

47. The method of claim 46, wherein said first and second pluralities are distinguishably labeled and simultaneously contacted to said microarray.

48. A vector, comprising: the single exon probe of claim 1.

49. An ORF-encoded peptide, comprising: at least 8 contiguous amino acids of any one of SEQ ID NOs:33,300-49,117 or at least 8 contiguous amino acids of any one of SEQ ID NOs:33,300-49,117 with conservative amino acid substitutions.

50. The ORF-encoded peptide of claim 49, wherein said peptide comprises at least 15 contiguous amino acids of any one of SEQ ID Nos:33,300-49,117 or at least 15 contiguous amino acids of any one of SEQ ID NOs:33,300-49,117 with conservative amino acid substitutions.

51. An isolated antibody, wherein said antibody binds specifically to a peptide according to claim 49.

52. A method of selling and/or licensing single exon probes to a customer desiring to measure gene expression, comprising:
making available for computerized query a database having a plurality of records, each record corresponding to a single exon probe according to claim 1,
wherein said database responds to a customer query by returning to the customer at least one record, or an identifier of said record, that satisfies the customer query criteria, the probes to which said records correspond being available for sale and/or licensing.

53. A method of selling and/or licensing single-exon microarrays to a customer desiring to measure gene expression, comprising:
making available for computerized query a database having a plurality of records, each record corresponding to a microarray according to claim 40,
wherein said database responds to a customer query by returning to the customer at least one record, or an identifier of said record, that satisfies the customer query criteria, the microarrays to which said records correspond being available for sale and/or licensing.

54. A method of providing human gene expression data by subscription, comprising:
making available for computerized query a database having a plurality of records, each record including data on the expression of a single exon probe according to claim 1,
wherein said database responds to a query by a customer having a subscription, but not to a query by a customer lacking such subscription, by returning at least one record, or identifier of said at least one record, that satisfies the customer query criteria.

55. A computer readable storage medium storing instructions that, when executed by a computer, causes the computer to perform the method of any one of claims 52 to 55.

56. A computer system, comprising a processor programmed to perform the method of any one of claims 52 to 55.

57. A computer readable storage medium containing a database having a plurality of records, each record including data on the expression of a single exon probe according to claim 1.

58. A method of financing a company that makes and sells single exon probes, single exon microarrays, or expression data obtained therefrom, comprising:
making known the availability for distribution, sale, and/or license of single exon probes, single exon microarrays, and/or data therefrom; and then
selling stock in said company.
